SEQUENCE LISTING (272 sequences)

SEQ ID NO: 1 - DNA, 657 bp, synthetic construct
  misc_feature 1..657: note = synthesized
  source 1..657: mol_type = other DNA
gatgttgtgatgacccagactccagcctccgtgtctgaacctgtgagagccacagtcaccatcaagtgccaggccagtcagagcattggtgctgatttagcctggtatcagcagaaaccagggcagcctcccaagctcctgatctacagggcatccactctggcctctggggtcccatcgcggttcagcggcagtggatctgggacagagttcactctcaccatcagcgacctggagtgtgccgatgctgccacttactactgtcaatgtatttattatggtactgatgatgtcatataccatactttcggcggagggaccgaggtggtgttcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 2 - DNA, 336 bp, synthetic construct
  misc_feature 1..336: note = synthesized
  source 1..336: mol_type = other DNA
gatgttgtgatgacccagactccagcctccgtgtctgaacctgtgagagccacagtcaccatcaagtgccaggccagtcagagcattggtgctgatttagcctggtatcagcagaaaccagggcagcctcccaagctcctgatctacagggcatccactctggcctctggggtcccatcgcggttcagcggcagtggatctgggacagagttcactctcaccatcagcgacctggagtgtgccgatgctgccacttactactgtcaatgtatttattatggtactgatgatgtcatataccatactttcggcggagggaccgaggtggtgttcaaa

SEQ ID NO: 3 - protein, 219 aa, synthetic construct
  REGION 1..219: note = synthesized
DVVMTQTPASVSEPVRATVTIKCQASQSIGADLAWYQQKPGQPPKLLIYRASTLASGVPSRFSGSGSGTEFTLTISDLECADAATYYCQCIYYGTDDVIYHTFGGGTEVVFKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 112 aa, synthetic construct
  REGION 1..112: note = synthesized
DVVMTQTPASVSEPVRATVTIKCQASQSIGADLAWYQQKPGQPPKLLIYRASTLASGVPSRFSGSGSGTEFTLTISDLECADAATYYCQCIYYGTDDVIYHTFGGGTEVVFK

SEQ ID NO: 5 - DNA, 1332 bp, synthetic construct
  misc_feature 1..1332: note = synthesized
  source 1..1332: mol_type = other DNA
cagtcggtggaggagtccgggggtcgcctggtcacgcctgggacacccctaacactcacctgcacagtatctggattcaccatcagtagccaccacatgatctgggtccgccaggctccaggagaggggctgcaatacatcggattcattaatgatggtgactatacatactacacgaactgggcaaaaggccgattcaccatctccagaacctcgactacggtggacctgaaaatgaccagtctgacagccgcggacacggccacctatttctgtgccagaggggttgatggtactagttatcctggcttgtggggcccaggcaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 6 - DNA, 345 bp, synthetic construct
  misc_feature 1..345: note = synthesized
  source 1..345: mol_type = other DNA
cagtcggtggaggagtccgggggtcgcctggtcacgcctgggacacccctaacactcacctgcacagtatctggattcaccatcagtagccaccacatgatctgggtccgccaggctccaggagaggggctgcaatacatcggattcattaatgatggtgactatacatactacacgaactgggcaaaaggccgattcaccatctccagaacctcgactacggtggacctgaaaatgaccagtctgacagccgcggacacggccacctatttctgtgccagaggggttgatggtactagttatcctggcttgtggggcccaggcaccctggtcaccgtctcgagc

SEQ ID NO: 7 - protein, 444 aa, synthetic construct
  REGION 1..444: note = synthesized
QSVEESGGRLVTPGTPLTLTCTVSGFTISSHHMIWVRQAPGEGLQYIGFINDGDYTYYTNWAKGRFTISRTSTTVDLKMTSLTAADTATYFCARGVDGTSYPGLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 115 aa, synthetic construct
  REGION 1..115: note = synthesized
QSVEESGGRLVTPGTPLTLTCTVSGFTISSHHMIWVRQAPGEGLQYIGFINDGDYTYYTNWAKGRFTISRTSTTVDLKMTSLTAADTATYFCARGVDGTSYPGLWGPGTLVTVSS

SEQ ID NO: 9 - DNA, 657 bp, synthetic construct
  misc_feature 1..657: note = synthesized
  source 1..657: mol_type = other DNA
gacgtcgtgatgacccagactccagcctccgtggaggcagctgtgggaggcacagtcaccatcaagtgccaggccagtcagagcatttacagcaatttagcctggtatcagcagaaaccagggcagcctcccaaactcctaatctattctacatccactctggcatctggggtcccatcgcggttcagcggcagtggatccgggacagagttcactctcaccatcagcgacctggagtgtgccgatgctgccacttactactgtcaaaactattatggtagtagtactgatagttatgggaatcctttcggcggaggcaccgaggtggtgttcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 10 - DNA, 336 bp, synthetic construct
  misc_feature 1..336: note = synthesized
  source 1..336: mol_type = other DNA
gacgtcgtgatgacccagactccagcctccgtggaggcagctgtgggaggcacagtcaccatcaagtgccaggccagtcagagcatttacagcaatttagcctggtatcagcagaaaccagggcagcctcccaaactcctaatctattctacatccactctggcatctggggtcccatcgcggttcagcggcagtggatccgggacagagttcactctcaccatcagcgacctggagtgtgccgatgctgccacttactactgtcaaaactattatggtagtagtactgatagttatgggaatcctttcggcggaggcaccgaggtggtgttcaaa

SEQ ID NO: 11 - protein, 219 aa, synthetic construct
  REGION 1..219: note = synthesized
DVVMTQTPASVEAAVGGTVTIKCQASQSIYSNLAWYQQKPGQPPKLLIYSTSTLASGVPSRFSGSGSGTEFTLTISDLECADAATYYCQNYYGSSTDSYGNPFGGGTEVVFKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 112 aa, synthetic construct
  REGION 1..112: note = synthesized
DVVMTQTPASVEAAVGGTVTIKCQASQSIYSNLAWYQQKPGQPPKLLIYSTSTLASGVPSRFSGSGSGTEFTLTISDLECADAATYYCQNYYGSSTDSYGNPFGGGTEVVFK

SEQ ID NO: 13 - DNA, 1344 bp, synthetic construct
  misc_feature 1..1344: note = synthesized
  source 1..1344: mol_type = other DNA
cagtcgctggaggagtccgggggagacctggtcaagcctgagggatccctgacactcacctgcacaggctctggaatcgacttcagtagcaactacatgtgctgggtccgccaggctccagggaaggggctggagtggatcgcatgcatttatggtgatagtagtgataatagttactccgcgagctgggcgaaagggcgattcaccatctccaaaacctcgtcgaccacggtgactctgcaaatgaccagtctgacagccgcggacacggccacctatttctgtgcgagatctggttatagctatttaggctactttaacttgtggggcccagggaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 14 - DNA, 357 bp, synthetic construct
  misc_feature 1..357: note = synthesized
  source 1..357: mol_type = other DNA
cagtcgctggaggagtccgggggagacctggtcaagcctgagggatccctgacactcacctgcacaggctctggaatcgacttcagtagcaactacatgtgctgggtccgccaggctccagggaaggggctggagtggatcgcatgcatttatggtgatagtagtgataatagttactccgcgagctgggcgaaagggcgattcaccatctccaaaacctcgtcgaccacggtgactctgcaaatgaccagtctgacagccgcggacacggccacctatttctgtgcgagatctggttatagctatttaggctactttaacttgtggggcccagggaccctggtcaccgtctcgagc

SEQ ID NO: 15 - protein, 448 aa, synthetic construct
  REGION 1..448: note = synthesized
QSLEESGGDLVKPEGSLTLTCTGSGIDFSSNYMCWVRQAPGKGLEWIACIYGDSSDNSYSASWAKGRFTISKTSSTTVTLQMTSLTAADTATYFCARSGYSYLGYFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 119 aa, synthetic construct
  REGION 1..119: note = synthesized
QSLEESGGDLVKPEGSLTLTCTGSGIDFSSNYMCWVRQAPGKGLEWIACIYGDSSDNSYSASWAKGRFTISKTSSTTVTLQMTSLTAADTATYFCARSGYSYLGYFNLWGPGTLVTVSS

SEQ ID NO: 17 - DNA, 651 bp, synthetic construct
  misc_feature 1..651: note = synthesized
  source 1..651: mol_type = other DNA
gcgcaagtgctgacccagactgcatcgtccgtgtctgcacctgtgggcggcacagtcaccatcagttgccagtccagtcagactgtttataataacaacttgttatcctggtatcagcagaaaccagggcagcgtcccaagctcctgatctattatgcatccactctggcatttggggtcccatcgcggttcaaaggcagtggatctgggacagagttcactctcaccatcagcgacctggagtgtgacgatgctgccacttacttctgtcaaggcggttatagtggttggatttatgttttcggcggaggcaccgaggtggaggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 18 - DNA, 330 bp, synthetic construct
  misc_feature 1..330: note = synthesized
  source 1..330: mol_type = other DNA
gcgcaagtgctgacccagactgcatcgtccgtgtctgcacctgtgggcggcacagtcaccatcagttgccagtccagtcagactgtttataataacaacttgttatcctggtatcagcagaaaccagggcagcgtcccaagctcctgatctattatgcatccactctggcatttggggtcccatcgcggttcaaaggcagtggatctgggacagagttcactctcaccatcagcgacctggagtgtgacgatgctgccacttacttctgtcaaggcggttatagtggttggatttatgttttcggcggaggcaccgaggtggaggtcaaa

SEQ ID NO: 19 - protein, 217 aa, synthetic construct
  REGION 1..217: note = synthesized
AQVLTQTASSVSAPVGGTVTISCQSSQTVYNNNLLSWYQQKPGQRPKLLIYYASTLAFGVPSRFKGSGSGTEFTLTISDLECDDAATYFCQGGYSGWIYVFGGGTEVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 110 aa, synthetic construct
  REGION 1..110: note = synthesized
AQVLTQTASSVSAPVGGTVTISCQSSQTVYNNNLLSWYQQKPGQRPKLLIYYASTLAFGVPSRFKGSGSGTEFTLTISDLECDDAATYFCQGGYSGWIYVFGGGTEVEVK

SEQ ID NO: 21 - DNA, 1323 bp, synthetic construct
  misc_feature 1..1323: note = synthesized
  source 1..1323: mol_type = other DNA
cagtcgctggaggagtccgggggtcgcctggtcacgcctgggacacccctgacactcacctgcacagcctctggattctccctcagtaactactacatgaactgggtccgccaggctccagggaaggggctggaatacatcggaatcattagtagtagtggtagcgcatactacgcgagctgggctaaaggccgattcaccatctccagaacctcgaccacggtggacctgagaatcaccagtccgacaaccgaggacacggccacctatttctgtgccagaggggactacggcatggacctctggggccaagggaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 22 - DNA, 336 bp, synthetic construct
  misc_feature 1..336: note = synthesized
  source 1..336: mol_type = other DNA
cagtcgctggaggagtccgggggtcgcctggtcacgcctgggacacccctgacactcacctgcacagcctctggattctccctcagtaactactacatgaactgggtccgccaggctccagggaaggggctggaatacatcggaatcattagtagtagtggtagcgcatactacgcgagctgggctaaaggccgattcaccatctccagaacctcgaccacggtggacctgagaatcaccagtccgacaaccgaggacacggccacctatttctgtgccagaggggactacggcatggacctctggggccaagggaccctggtcaccgtctcgagc

SEQ ID NO: 23 - protein, 441 aa, synthetic construct
  REGION 1..441: note = synthesized
QSLEESGGRLVTPGTPLTLTCTASGFSLSNYYMNWVRQAPGKGLEYIGIISSSGSAYYASWAKGRFTISRTSTTVDLRITSPTTEDTATYFCARGDYGMDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 112 aa, synthetic construct
  REGION 1..112: note = synthesized
QSLEESGGRLVTPGTPLTLTCTASGFSLSNYYMNWVRQAPGKGLEYIGIISSSGSAYYASWAKGRFTISRTSTTVDLRITSPTTEDTATYFCARGDYGMDLWGQGTLVTVSS

SEQ ID NO: 25 - DNA, 657 bp, synthetic construct
  misc_feature 1..657: note = synthesized
  source 1..657: mol_type = other DNA
gacgtcgtgatgacccagactccagcctccgtgtctgaacctgtgggaggcgcagtcaccatcaagtgccaggccagtcagagcattggtagtgatttagcctggtatcagcagaaaccagggcagcctcccaagctcctgatctacagggcatccactctggcatctggggtctcatcgcggttcaaaggcagtggatctgggacagagttcactctcaccatcagcgacctggagtgtgccgatgctgccacttactactgtcaatgtatttattatggtagtgatgatgtcatataccatactttcggcggagggaccgaggtggtggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 26 - DNA, 336 bp, synthetic construct
  misc_feature 1..336: note = synthesized
  source 1..336: mol_type = other DNA
gacgtcgtgatgacccagactccagcctccgtgtctgaacctgtgggaggcgcagtcaccatcaagtgccaggccagtcagagcattggtagtgatttagcctggtatcagcagaaaccagggcagcctcccaagctcctgatctacagggcatccactctggcatctggggtctcatcgcggttcaaaggcagtggatctgggacagagttcactctcaccatcagcgacctggagtgtgccgatgctgccacttactactgtcaatgtatttattatggtagtgatgatgtcatataccatactttcggcggagggaccgaggtggtggtcaaa

SEQ ID NO: 27 - protein, 219 aa, synthetic construct
  REGION 1..219: note = synthesized
DVVMTQTPASVSEPVGGAVTIKCQASQSIGSDLAWYQQKPGQPPKLLIYRASTLASGVSSRFKGSGSGTEFTLTISDLECADAATYYCQCIYYGSDDVIYHTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 112 aa, synthetic construct
  REGION 1..112: note = synthesized
DVVMTQTPASVSEPVGGAVTIKCQASQSIGSDLAWYQQKPGQPPKLLIYRASTLASGVSSRFKGSGSGTEFTLTISDLECADAATYYCQCIYYGSDDVIYHTFGGGTEVVVK

SEQ ID NO: 29 - DNA, 1332 bp, synthetic construct
  misc_feature 1..1332: note = synthesized
  source 1..1332: mol_type = other DNA
cagtcggtggaggagtccgggggtcgcctggtcacgcctgggacacccctgacactcacctgcacagtctctggattcactatcaatagctaccacatgatctgggtccgccaggctccaggggaggggctggaatacatcggattcattaatgatggtggtttcacatactacgcgagctgggcaaaaggccgatttatcatctccagaacctcgaccacggtggatctgaaaatgaccagtctgacagtcgcggacacggccacctatttctgtgccagaggggttgatggtactagttatcctgacttatggggcccgggcaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 30 - DNA, 345 bp, synthetic construct
  misc_feature 1..345: note = synthesized
  source 1..345: mol_type = other DNA
cagtcggtggaggagtccgggggtcgcctggtcacgcctgggacacccctgacactcacctgcacagtctctggattcactatcaatagctaccacatgatctgggtccgccaggctccaggggaggggctggaatacatcggattcattaatgatggtggtttcacatactacgcgagctgggcaaaaggccgatttatcatctccagaacctcgaccacggtggatctgaaaatgaccagtctgacagtcgcggacacggccacctatttctgtgccagaggggttgatggtactagttatcctgacttatggggcccgggcaccctggtcaccgtctcgagc

SEQ ID NO: 31 - protein, 444 aa, synthetic construct
  REGION 1..444: note = synthesized
QSVEESGGRLVTPGTPLTLTCTVSGFTINSYHMIWVRQAPGEGLEYIGFINDGGFTYYASWAKGRFIISRTSTTVDLKMTSLTVADTATYFCARGVDGTSYPDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 115 aa, synthetic construct
  REGION 1..115: note = synthesized
QSVEESGGRLVTPGTPLTLTCTVSGFTINSYHMIWVRQAPGEGLEYIGFINDGGFTYYASWAKGRFIISRTSTTVDLKMTSLTVADTATYFCARGVDGTSYPDLWGPGTLVTVSS

SEQ ID NO: 33 - DNA, 654 bp, synthetic construct
  misc_feature 1..654: note = synthesized
  source 1..654: mol_type = other DNA
gccctggtgatgacccagactccatcccctgtgtctgcggctgtgggaggcacagtcaccatcaactgccaggccagtcagagcattggtagtaacttgaactggtatcagcagaaaccagggcagcctcccaagctcctgatttattatgtatccactctggcatctggggtcccatcgcggttcaaaggcagtggatctgggacagagtacactctcaccatcagcggcgtgcagtgtgacgatgctgccacttactactgcctaggtgtttggaattattggggtgatgacggtactgctttcggcggaggcaccgaggtggaggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 34 - DNA, 333 bp, synthetic construct
  misc_feature 1..333: note = synthesized
  source 1..333: mol_type = other DNA
gccctggtgatgacccagactccatcccctgtgtctgcggctgtgggaggcacagtcaccatcaactgccaggccagtcagagcattggtagtaacttgaactggtatcagcagaaaccagggcagcctcccaagctcctgatttattatgtatccactctggcatctggggtcccatcgcggttcaaaggcagtggatctgggacagagtacactctcaccatcagcggcgtgcagtgtgacgatgctgccacttactactgcctaggtgtttggaattattggggtgatgacggtactgctttcggcggaggcaccgaggtggaggtcaaa

SEQ ID NO: 35 - protein, 218 aa, synthetic construct
  REGION 1..218: note = synthesized
ALVMTQTPSPVSAAVGGTVTINCQASQSIGSNLNWYQQKPGQPPKLLIYYVSTLASGVPSRFKGSGSGTEYTLTISGVQCDDAATYYCLGVWNYWGDDGTAFGGGTEVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 111 aa, synthetic construct
  REGION 1..111: note = synthesized
ALVMTQTPSPVSAAVGGTVTINCQASQSIGSNLNWYQQKPGQPPKLLIYYVSTLASGVPSRFKGSGSGTEYTLTISGVQCDDAATYYCLGVWNYWGDDGTAFGGGTEVEVK

SEQ ID NO: 37 - DNA, 1323 bp, synthetic construct
  misc_feature 1..1323: note = synthesized
  source 1..1323: mol_type = other DNA
cagtcgctggaggagtccgggggtcgcctggtcacgcctgggacacccctgacactcacctgcacagcctctgatttctccctcagtacctattatatgagctgggtccgccaggctccagggaaggggctggagtggatcggaagtatttatgatagtggtgccgcatactacgcgacctgggcgaagggccgattcaccatctccagaacgtcgaccacggtggatctgagaatgaccagtctgacagccgcggacacggccacctatttctgtgccagagatcctattaataatgccatctggggccaagggaccctcgtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 38 - DNA, 336 bp, synthetic construct
  misc_feature 1..336: note = synthesized
  source 1..336: mol_type = other DNA
cagtcgctggaggagtccgggggtcgcctggtcacgcctgggacacccctgacactcacctgcacagcctctgatttctccctcagtacctattatatgagctgggtccgccaggctccagggaaggggctggagtggatcggaagtatttatgatagtggtgccgcatactacgcgacctgggcgaagggccgattcaccatctccagaacgtcgaccacggtggatctgagaatgaccagtctgacagccgcggacacggccacctatttctgtgccagagatcctattaataatgccatctggggccaagggaccctcgtcaccgtctcgagc

SEQ ID NO: 39 - protein, 441 aa, synthetic construct
  REGION 1..441: note = synthesized
QSLEESGGRLVTPGTPLTLTCTASDFSLSTYYMSWVRQAPGKGLEWIGSIYDSGAAYYATWAKGRFTISRTSTTVDLRMTSLTAADTATYFCARDPINNAIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 112 aa, synthetic construct
  REGION 1..112: note = synthesized
QSLEESGGRLVTPGTPLTLTCTASDFSLSTYYMSWVRQAPGKGLEWIGSIYDSGAAYYATWAKGRFTISRTSTTVDLRMTSLTAADTATYFCARDPINNAIWGQGTLVTVSS

SEQ ID NO: 41 - DNA, 654 bp, synthetic construct
  misc_feature 1..654: note = synthesized
  source 1..654: mol_type = other DNA
gccctggtgatgacccagactccatcccctgtgtctgcggctgtgggaggcacagtcaccatcaactgccaggccagtcagagtattgctactaacttgaactggtatcagcagaaaccagggcagcctcccaagctcctgatctattatacatccagtctggcatctggggtcccatcgcggttcagcggcagtggatctggcacagagttcactctcaccatcagcggtgtgcagtgtgacgatgctgccacttactactgcctaggtgtttggaattattggggtgatgatggtactgctttcggcggagggaccgaggtggagttcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 42 - DNA, 333 bp, synthetic construct
  misc_feature 1..333: note = synthesized
  source 1..333: mol_type = other DNA
gccctggtgatgacccagactccatcccctgtgtctgcggctgtgggaggcacagtcaccatcaactgccaggccagtcagagtattgctactaacttgaactggtatcagcagaaaccagggcagcctcccaagctcctgatctattatacatccagtctggcatctggggtcccatcgcggttcagcggcagtggatctggcacagagttcactctcaccatcagcggtgtgcagtgtgacgatgctgccacttactactgcctaggtgtttggaattattggggtgatgatggtactgctttcggcggagggaccgaggtggagttcaaa

SEQ ID NO: 43 - protein, 218 aa, synthetic construct
  REGION 1..218: note = synthesized
ALVMTQTPSPVSAAVGGTVTINCQASQSIATNLNWYQQKPGQPPKLLIYYTSSLASGVPSRFSGSGSGTEFTLTISGVQCDDAATYYCLGVWNYWGDDGTAFGGGTEVEFKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 111 aa, synthetic construct
  REGION 1..111: note = synthesized
ALVMTQTPSPVSAAVGGTVTINCQASQSIATNLNWYQQKPGQPPKLLIYYTSSLASGVPSRFSGSGSGTEFTLTISGVQCDDAATYYCLGVWNYWGDDGTAFGGGTEVEFK

SEQ ID NO: 45 - DNA, 1323 bp, synthetic construct
  misc_feature 1..1323: note = synthesized
  source 1..1323: mol_type = other DNA
cagtcgctggaggagtccgggggtcgcctggtcacgcctgggacacccctgacactcacctgcacagcctctgaattctccctcagtagctactacatgagctgggtccgccaggctccagggaaggggctggaatggatcggaagtatttatgctagtggtagcgcatactacgcgagttgggcgaagggccgattcaccatctccagaacgtcgaccacggtggatctgaaaatgaccagtctgacagccgcggacacggccacctatttctgtgtcagagatcctattaacaatgacatctggggcccaggcaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 46 - DNA, 336 bp, synthetic construct
  misc_feature 1..336: note = synthesized
  source 1..336: mol_type = other DNA
cagtcgctggaggagtccgggggtcgcctggtcacgcctgggacacccctgacactcacctgcacagcctctgaattctccctcagtagctactacatgagctgggtccgccaggctccagggaaggggctggaatggatcggaagtatttatgctagtggtagcgcatactacgcgagttgggcgaagggccgattcaccatctccagaacgtcgaccacggtggatctgaaaatgaccagtctgacagccgcggacacggccacctatttctgtgtcagagatcctattaacaatgacatctggggcccaggcaccctggtcaccgtctcgagc

SEQ ID NO: 47 - protein, 441 aa, synthetic construct
  REGION 1..441: note = synthesized
QSLEESGGRLVTPGTPLTLTCTASEFSLSSYYMSWVRQAPGKGLEWIGSIYASGSAYYASWAKGRFTISRTSTTVDLKMTSLTAADTATYFCVRDPINNDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 112 aa, synthetic construct
  REGION 1..112: note = synthesized
QSLEESGGRLVTPGTPLTLTCTASEFSLSSYYMSWVRQAPGKGLEWIGSIYASGSAYYASWAKGRFTISRTSTTVDLKMTSLTAADTATYFCVRDPINNDIWGPGTLVTVSS

SEQ ID NO: 49 - DNA, 654 bp, synthetic construct
  misc_feature 1..654: note = synthesized
  source 1..654: mol_type = other DNA
gcccaagtgctgacccagactccagcctccgtgtctgcaactgtgggaagcacagtcaccatcagttgccagtccagtccgagtgtttataagaacaaccaattagcctggtatcagcagaaaccagggcagcctcccaaactcctgatctatctggcatctactctggcatctggggtcccatcgcggttcaaaggcagtggatctgggacagagttcactctcaccatcagcgacctggagtgtgccgatgctgccacttactactgtgcaggcggttatagtagtagtagtgatactgctttcggcggagggaccgaggtggaggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 50 - DNA, 333 bp, synthetic construct
  misc_feature 1..333: note = synthesized
  source 1..333: mol_type = other DNA
gcccaagtgctgacccagactccagcctccgtgtctgcaactgtgggaagcacagtcaccatcagttgccagtccagtccgagtgtttataagaacaaccaattagcctggtatcagcagaaaccagggcagcctcccaaactcctgatctatctggcatctactctggcatctggggtcccatcgcggttcaaaggcagtggatctgggacagagttcactctcaccatcagcgacctggagtgtgccgatgctgccacttactactgtgcaggcggttatagtagtagtagtgatactgctttcggcggagggaccgaggtggaggtcaaa

SEQ ID NO: 51 - protein, 218 aa, synthetic construct
  REGION 1..218: note = synthesized
AQVLTQTPASVSATVGSTVTISCQSSPSVYKNNQLAWYQQKPGQPPKLLIYLASTLASGVPSRFKGSGSGTEFTLTISDLECADAATYYCAGGYSSSSDTAFGGGTEVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 111 aa, synthetic construct
  REGION 1..111: note = synthesized
AQVLTQTPASVSATVGSTVTISCQSSPSVYKNNQLAWYQQKPGQPPKLLIYLASTLASGVPSRFKGSGSGTEFTLTISDLECADAATYYCAGGYSSSSDTAFGGGTEVEVK

SEQ ID NO: 53 - DNA, 1335 bp, synthetic construct
  misc_feature 1..1335: note = synthesized
  source 1..1335: mol_type = other DNA
cagtcgctggaggagtccgggggtcgcctggtcacgcctgggacacccctgacactcacctgcacagtctctggaatcgacctcagtgcctaccacatgagctgggtccgccaggctccagggaaggggctggaatacatcggaatgattggtagtagtggtaccatacactacgcgaactgggcgaaaggccgattcaccatctccaaaacctcgaccacggtggatctgaaaatcaccagtccgacaaccgaggacacggccacctatttctgtgccagggacagttataatagtgattatgcctttaacttatggggcccagggaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 54 - DNA, 348 bp, synthetic construct
  misc_feature 1..348: note = synthesized
  source 1..348: mol_type = other DNA
cagtcgctggaggagtccgggggtcgcctggtcacgcctgggacacccctgacactcacctgcacagtctctggaatcgacctcagtgcctaccacatgagctgggtccgccaggctccagggaaggggctggaatacatcggaatgattggtagtagtggtaccatacactacgcgaactgggcgaaaggccgattcaccatctccaaaacctcgaccacggtggatctgaaaatcaccagtccgacaaccgaggacacggccacctatttctgtgccagggacagttataatagtgattatgcctttaacttatggggcccagggaccctggtcaccgtctcgagc

SEQ ID NO: 55 - protein, 445 aa, synthetic construct
  REGION 1..445: note = synthesized
QSLEESGGRLVTPGTPLTLTCTVSGIDLSAYHMSWVRQAPGKGLEYIGMIGSSGTIHYANWAKGRFTISKTSTTVDLKITSPTTEDTATYFCARDSYNSDYAFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 116 aa, synthetic construct
  REGION 1..116: note = synthesized
QSLEESGGRLVTPGTPLTLTCTVSGIDLSAYHMSWVRQAPGKGLEYIGMIGSSGTIHYANWAKGRFTISKTSTTVDLKITSPTTEDTATYFCARDSYNSDYAFNLWGPGTLVTVSS

SEQ ID NO: 57 - DNA, 654 bp, synthetic construct
  misc_feature 1..654: note = synthesized
  source 1..654: mol_type = other DNA
gctcaagtgctgacccagactgcatcgcccgtgtctgccgctgtgggaggcacagtcaccatcaactgccagtccagtcagagtgtttatagtaactggttatcctggtatcagcagaaaccagggcagcctcccaagcgcctgatctattctgcatccactctggcatctggggtcccatcgcggttcaaaggcagtggatctgggacacagttcactctcaccatcagcgacctggagtgtgacgatgctgccacttactactgtgcaggcggttataatactgttactgatacttttgctttcggcagaggcaccgaggtggaggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 58 - DNA, 333 bp, synthetic construct
  misc_feature 1..333: note = synthesized
  source 1..333: mol_type = other DNA
gctcaagtgctgacccagactgcatcgcccgtgtctgccgctgtgggaggcacagtcaccatcaactgccagtccagtcagagtgtttatagtaactggttatcctggtatcagcagaaaccagggcagcctcccaagcgcctgatctattctgcatccactctggcatctggggtcccatcgcggttcaaaggcagtggatctgggacacagttcactctcaccatcagcgacctggagtgtgacgatgctgccacttactactgtgcaggcggttataatactgttactgatacttttgctttcggcagaggcaccgaggtggaggtcaaa

SEQ ID NO: 59 - protein, 218 aa, synthetic construct
  REGION 1..218: note = synthesized
AQVLTQTASPVSAAVGGTVTINCQSSQSVYSNWLSWYQQKPGQPPKRLIYSASTLASGVPSRFKGSGSGTQFTLTISDLECDDAATYYCAGGYNTVTDTFAFGRGTEVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 111 aa, synthetic construct
  REGION 1..111: note = synthesized
AQVLTQTASPVSAAVGGTVTINCQSSQSVYSNWLSWYQQKPGQPPKRLIYSASTLASGVPSRFKGSGSGTQFTLTISDLECDDAATYYCAGGYNTVTDTFAFGRGTEVEVK

SEQ ID NO: 61 - DNA, 1326 bp, synthetic construct
  misc_feature 1..1326: note = synthesized
  source 1..1326: mol_type = other DNA
cagtcgttggaggagtccgggggagacctggtcaagcctggggcatccctgacactcacctgcacagcctctggattctccttcagtagcaggcactacatgtgttgggtccgccaggctccaggggaggggctggagtggatcgcatgcatttatactggtagtagtggtactcctcactacgcgagctgggcgaaaggccgattcaccatctcccaaacctcgtcgaccacggtgactctgcaaatgaccagtctgacagccgcggacacggccacctatttctgttcgagcgaaggtaacctgtggggcccaggcaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 62 - DNA, 339 bp, synthetic construct
  misc_feature 1..339: note = synthesized
  source 1..339: mol_type = other DNA
cagtcgttggaggagtccgggggagacctggtcaagcctggggcatccctgacactcacctgcacagcctctggattctccttcagtagcaggcactacatgtgttgggtccgccaggctccaggggaggggctggagtggatcgcatgcatttatactggtagtagtggtactcctcactacgcgagctgggcgaaaggccgattcaccatctcccaaacctcgtcgaccacggtgactctgcaaatgaccagtctgacagccgcggacacggccacctatttctgttcgagcgaaggtaacctgtggggcccaggcaccctggtcaccgtctcgagc

SEQ ID NO: 63 - protein, 442 aa, synthetic construct
  REGION 1..442: note = synthesized
QSLEESGGDLVKPGASLTLTCTASGFSFSSRHYMCWVRQAPGEGLEWIACIYTGSSGTPHYASWAKGRFTISQTSSTTVTLQMTSLTAADTATYFCSSEGNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 113 aa, synthetic construct
  REGION 1..113: note = synthesized
QSLEESGGDLVKPGASLTLTCTASGFSFSSRHYMCWVRQAPGEGLEWIACIYTGSSGTPHYASWAKGRFTISQTSSTTVTLQMTSLTAADTATYFCSSEGNLWGPGTLVTVSS

SEQ ID NO: 65 - DNA, 657 bp, synthetic construct
  misc_feature 1..657: note = synthesized
  source 1..657: mol_type = other DNA
gcccttgtgatgacccagactccagcctccgtgtctgcagctgtgggaggcacagtcaccatcaattgccaggccagtgaggacattgatacctatttagcctggtatcagcagaaaccagggcagcctcccaagctcctgatcttttatgcatccgatctggcatctggggtcccatcgcggttcaaaggcagtggatctgggacagagttcactctcaccatcagcggtgtgcagtgtgacgatgctgccacttactactgtcaaggcggttactatactagtagtgctgatacgaggggtgctttcggcggaggcaccgaggtggtggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 66 - DNA, 336 bp, synthetic construct
  misc_feature 1..336: note = synthesized
  source 1..336: mol_type = other DNA
gcccttgtgatgacccagactccagcctccgtgtctgcagctgtgggaggcacagtcaccatcaattgccaggccagtgaggacattgatacctatttagcctggtatcagcagaaaccagggcagcctcccaagctcctgatcttttatgcatccgatctggcatctggggtcccatcgcggttcaaaggcagtggatctgggacagagttcactctcaccatcagcggtgtgcagtgtgacgatgctgccacttactactgtcaaggcggttactatactagtagtgctgatacgaggggtgctttcggcggaggcaccgaggtggtggtcaaa

SEQ ID NO: 67 - protein, 219 aa, synthetic construct
  REGION 1..219: note = synthesized
ALVMTQTPASVSAAVGGTVTINCQASEDIDTYLAWYQQKPGQPPKLLIFYASDLASGVPSRFKGSGSGTEFTLTISGVQCDDAATYYCQGGYYTSSADTRGAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 112 aa, synthetic construct
  REGION 1..112: note = synthesized
ALVMTQTPASVSAAVGGTVTINCQASEDIDTYLAWYQQKPGQPPKLLIFYASDLASGVPSRFKGSGSGTEFTLTISGVQCDDAATYYCQGGYYTSSADTRGAFGGGTEVVVK

SEQ ID NO: 69 - DNA, 1344 bp, synthetic construct
  misc_feature 1..1344: note = synthesized
  source 1..1344: mol_type = other DNA
cagtcgttggaggagtccgggggagacctggtcaagcctggggcatccctgacactcacctgcacagcctctggattctccttcagtagcaactactggatatgctgggtccgccaggctccagggaaggggctggagtggatcgcatgcatttatgttggtagtagtggtgacacttactacgcgagctgggcgaaaggccgattcaccatctccaaaacctcgtcgaccacggtgactctgcaaatgaccagtctgacagccgcggacacggccacctatttctgtgcgagagatagtagtagttattatatgtttaacttgtggggccaggggaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 70 - DNA, 357 bp, synthetic construct
  misc_feature 1..357: note = synthesized
  source 1..357: mol_type = other DNA
cagtcgttggaggagtccgggggagacctggtcaagcctggggcatccctgacactcacctgcacagcctctggattctccttcagtagcaactactggatatgctgggtccgccaggctccagggaaggggctggagtggatcgcatgcatttatgttggtagtagtggtgacacttactacgcgagctgggcgaaaggccgattcaccatctccaaaacctcgtcgaccacggtgactctgcaaatgaccagtctgacagccgcggacacggccacctatttctgtgcgagagatagtagtagttattatatgtttaacttgtggggccaggggaccctggtcaccgtctcgagc

SEQ ID NO: 71 - protein, 448 aa, synthetic construct
  REGION 1..448: note = synthesized
QSLEESGGDLVKPGASLTLTCTASGFSFSSNYWICWVRQAPGKGLEWIACIYVGSSGDTYYASWAKGRFTISKTSSTTVTLQMTSLTAADTATYFCARDSSSYYMFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 119 aa, synthetic construct
  REGION 1..119: note = synthesized
QSLEESGGDLVKPGASLTLTCTASGFSFSSNYWICWVRQAPGKGLEWIACIYVGSSGDTYYASWAKGRFTISKTSSTTVTLQMTSLTAADTATYFCARDSSSYYMFNLWGQGTLVTVSS

SEQ ID NO: 73 - DNA, 427 bp, synthetic construct
  misc_feature 1..427: note = synthesized
  source 1..427: mol_type = other DNA
tagcgacctggagtgtgacgatgctgccacttactactgtgcaggcggttacaatactgttattgatacttttgctttcggcggaggcaccgaggtggagttcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 74 - DNA, 333 bp, synthetic construct
  misc_feature 1..333: note = synthesized
  source 1..333: mol_type = other DNA
gctcaagtgctgacccagactgcatcgcccgtgtctgccgctgtgggaggcacagtcaccatcaactgccaggccagtcagagtgtttatagtaactggttatcctggtatcagcagaaaccagggcagcctcccaagcgcctgatctattctgcatccactctggcatctggggtcccatcgcggttcaaaggcagtggatctgggacacagttcactctcaccattagcgacctggagtgtgacgatgctgccacttactactgtgcaggcggttacaatactgttattgatacttttgctttcggcggaggcaccgaggtggagttcaaa

SEQ ID NO: 75 - protein, 218 aa, synthetic construct
  REGION 1..218: note = synthesized
AQVLTQTASPVSAAVGGTVTINCQASQSVYSNWLSWYQQKPGQPPKRLIYSASTLASGVPSRFKGSGSGTQFTLTISDLECDDAATYYCAGGYNTVIDTFAFGGGTEVEFKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 111 aa, synthetic construct
  REGION 1..111: note = synthesized
AQVLTQTASPVSAAVGGTVTINCQASQSVYSNWLSWYQQKPGQPPKRLIYSASTLASGVPSRFKGSGSGTQFTLTISDLECDDAATYYCAGGYNTVIDTFAFGGGTEVEFK

SEQ ID NO: 77 - DNA, 1329 bp, synthetic construct
  misc_feature 1..1329: note = synthesized
  source 1..1329: mol_type = other DNA
cagcagcagctggaggagtccgggggaggcctggtcaagcctggaggaaccctgacactcacctgcaaagcctctggaattgacttcagtagtagttactacatgtgctgggtccgccaggctccaggggaggggctggagtggatcgcatgcatttatactggtggtagtggtactcctcactacgcgagctgggcgaaaggccgattcaccatctcccaaacctcgtcgaccacggtgactctgcaaatgaccagtctgacagccgcggacacggccacctatttctgtgcgagagaaggtagcctgtggggccagggcaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 78 - DNA, 342 bp, synthetic construct
  misc_feature 1..342: note = synthesized
  source 1..342: mol_type = other DNA
cagcagcagctggaggagtccgggggaggcctggtcaagcctggaggaaccctgacactcacctgcaaagcctctggaattgacttcagtagtagttactacatgtgctgggtccgccaggctccaggggaggggctggagtggatcgcatgcatttatactggtggtagtggtactcctcactacgcgagctgggcgaaaggccgattcaccatctcccaaacctcgtcgaccacggtgactctgcaaatgaccagtctgacagccgcggacacggccacctatttctgtgcgagagaaggtagcctgtggggccagggcaccctggtcaccgtctcgagc

SEQ ID NO: 79 - protein, 443 aa, synthetic construct
  REGION 1..443: note = synthesized
QQQLEESGGGLVKPGGTLTLTCKASGIDFSSSYYMCWVRQAPGEGLEWIACIYTGGSGTPHYASWAKGRFTISQTSSTTVTLQMTSLTAADTATYFCAREGSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 114 aa, synthetic construct
  REGION 1..114: note = synthesized
QQQLEESGGGLVKPGGTLTLTCKASGIDFSSSYYMCWVRQAPGEGLEWIACIYTGGSGTPHYASWAKGRFTISQTSSTTVTLQMTSLTAADTATYFCAREGSLWGQGTLVTVSS

SEQ ID NO: 81 - DNA, 654 bp, synthetic construct
  misc_feature 1..654: note = synthesized
  source 1..654: mol_type = other DNA
gctcaagtgctgacccagactgcatcgcccgtgtctgccgctgtgggaggcacagtcaccatcaactgccagtccagtcagagtgtttatagtaactggttctcctggtatcagcagaaaccagggcagcctcccaagctcctgatctattctgcatccactctggcatctggggtcccctcgcggtttaaaggcagtggatctgggacacagttcactctcaccatcagcgacctggagtgtgacgatgctgccacttactactgtgcaggcggttacaatactgtaactgatacttttgctttcggcggaggcaccgaggtggagttcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 82 - DNA, 333 bp, synthetic construct
  misc_feature 1..333: note = synthesized
  source 1..333: mol_type = other DNA
gctcaagtgctgacccagactgcatcgcccgtgtctgccgctgtgggaggcacagtcaccatcaactgccagtccagtcagagtgtttatagtaactggttctcctggtatcagcagaaaccagggcagcctcccaagctcctgatctattctgcatccactctggcatctggggtcccctcgcggtttaaaggcagtggatctgggacacagttcactctcaccatcagcgacctggagtgtgacgatgctgccacttactactgtgcaggcggttacaatactgtaactgatacttttgctttcggcggaggcaccgaggtggagttcaaa

SEQ ID NO: 83 - protein, 218 aa, synthetic construct
  REGION 1..218: note = synthesized
AQVLTQTASPVSAAVGGTVTINCQSSQSVYSNWFSWYQQKPGQPPKLLIYSASTLASGVPSRFKGSGSGTQFTLTISDLECDDAATYYCAGGYNTVTDTFAFGGGTEVEFKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 111 aa, synthetic construct
  REGION 1..111: note = synthesized
AQVLTQTASPVSAAVGGTVTINCQSSQSVYSNWFSWYQQKPGQPPKLLIYSASTLASGVPSRFKGSGSGTQFTLTISDLECDDAATYYCAGGYNTVTDTFAFGGGTEVEFK

SEQ ID NO: 85 - DNA, 1326 bp, synthetic construct
  misc_feature 1..1326: note = synthesized
  source 1..1326: mol_type = other DNA
gagtcgttggaggagtccgggggagacctggtcaagcctggggcatccctgacactcacctgcacagcctctggaatcgacttcagtaggagatactacatgtgctgggtccgccaggctccagggaaggggctggagtggatcgcatgcatgtatactggtagccgcgatactcctcactacgcgagctgggcgaaaggccggttcaccatctcccaaacctcgtcgaccacggtgactctgcaaatgaccagtctgacagccgcggacacggccacctatttctgtgcgagagaaggtaacctgtggggcccggggaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 86 - DNA, 339 bp, synthetic construct
  misc_feature 1..339: note = synthesized
  source 1..339: mol_type = other DNA
gagtcgttggaggagtccgggggagacctggtcaagcctggggcatccctgacactcacctgcacagcctctggaatcgacttcagtaggagatactacatgtgctgggtccgccaggctccagggaaggggctggagtggatcgcatgcatgtatactggtagccgcgatactcctcactacgcgagctgggcgaaaggccggttcaccatctcccaaacctcgtcgaccacggtgactctgcaaatgaccagtctgacagccgcggacacggccacctatttctgtgcgagagaaggtaacctgtggggcccggggaccctggtcaccgtctcgagc

SEQ ID NO: 87 - protein, 442 aa, synthetic construct
  REGION 1..442: note = synthesized
ESLEESGGDLVKPGASLTLTCTASGIDFSRRYYMCWVRQAPGKGLEWIACMYTGSRDTPHYASWAKGRFTISQTSSTTVTLQMTSLTAADTATYFCAREGNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 113 aa, synthetic construct
  REGION 1..113: note = synthesized
ESLEESGGDLVKPGASLTLTCTASGIDFSRRYYMCWVRQAPGKGLEWIACMYTGSRDTPHYASWAKGRFTISQTSSTTVTLQMTSLTAADTATYFCAREGNLWGPGTLVTVSS

SEQ ID NO: 89 - DNA, 654 bp, synthetic construct
  misc_feature 1..654: note = synthesized
  source 1..654: mol_type = other DNA
gcccaagtgctgacccagactccatcgtccgtgtctgcagctgtgggaggcacagtcagcatcagttgccagtccagtcagagtgtttatagtaactggttctcctggtatcagcagaaaccagggcagcctcccaagctcctgatctattctgcatccactctggcatctggggtcccctcgcggtttaaaggcagtggatctgggacacagttcactctcaccatcagcgacctggagtgtgacgatgctgccacttactactgtgcaggcggttacaatactgttattgatacttttgctttcggcggaggcaccgaggtggagttcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 90 - DNA, 333 bp, synthetic construct
  misc_feature 1..333: note = synthesized
  source 1..333: mol_type = other DNA
gcccaagtgctgacccagactccatcgtccgtgtctgcagctgtgggaggcacagtcagcatcagttgccagtccagtcagagtgtttatagtaactggttctcctggtatcagcagaaaccagggcagcctcccaagctcctgatctattctgcatccactctggcatctggggtcccctcgcggtttaaaggcagtggatctgggacacagttcactctcaccatcagcgacctggagtgtgacgatgctgccacttactactgtgcaggcggttacaatactgttattgatacttttgctttcggcggaggcaccgaggtggagttcaaa

SEQ ID NO: 91 - protein, 218 aa, synthetic construct
  REGION 1..218: note = synthesized
AQVLTQTPSSVSAAVGGTVSISCQSSQSVYSNWFSWYQQKPGQPPKLLIYSASTLASGVPSRFKGSGSGTQFTLTISDLECDDAATYYCAGGYNTVIDTFAFGGGTEVEFKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 111 aa, synthetic construct
  REGION 1..111: note = synthesized
AQVLTQTPSSVSAAVGGTVSISCQSSQSVYSNWFSWYQQKPGQPPKLLIYSASTLASGVPSRFKGSGSGTQFTLTISDLECDDAATYYCAGGYNTVIDTFAFGGGTEVEFK

SEQ ID NO: 93 - DNA, 1326 bp, synthetic construct
  misc_feature 1..1326: note = synthesized
  source 1..1326: mol_type = other DNA
cagtcgttggaggagtccgggggagacctggtcaagcctggggcatccctgacactcacctgcacagcctctggaatcgacttcagtaggagatactacatgtgctgggtccgccaggctccagggaaggggctggagtggatcgcatgcatatatactggtagccgcgatactcctcactacgcgagctgggcgaaaggccgattcaccatctcccaaacctcgtcgaccacggtgactctgcaaatgaccagtctgacagccgcggacacggccacctatttctgtgcgagagaaggtagcctgtggggccagggcaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 94 - DNA, 339 bp, synthetic construct
  misc_feature 1..339: note = synthesized
  source 1..339: mol_type = other DNA
cagtcgttggaggagtccgggggagacctggtcaagcctggggcatccctgacactcacctgcacagcctctggaatcgacttcagtaggagatactacatgtgctgggtccgccaggctccagggaaggggctggagtggatcgcatgcatatatactggtagccgcgatactcctcactacgcgagctgggcgaaaggccgattcaccatctcccaaacctcgtcgaccacggtgactctgcaaatgaccagtctgacagccgcggacacggccacctatttctgtgcgagagaaggtagcctgtggggccagggcaccctggtcaccgtctcgagc

SEQ ID NO: 95 - protein, 442 aa, synthetic construct
  REGION 1..442: note = synthesized
QSLEESGGDLVKPGASLTLTCTASGIDFSRRYYMCWVRQAPGKGLEWIACIYTGSRDTPHYASWAKGRFTISQTSSTTVTLQMTSLTAADTATYFCAREGSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 113 aa, synthetic construct
  REGION 1..113: note = synthesized
QSLEESGGDLVKPGASLTLTCTASGIDFSRRYYMCWVRQAPGKGLEWIACIYTGSRDTPHYASWAKGRFTISQTSSTTVTLQMTSLTAADTATYFCAREGSLWGQGTLVTVSS

SEQ ID NO: 97 - DNA, 651 bp, synthetic construct
  misc_feature 1..651: note = synthesized
  source 1..651: mol_type = other DNA
gcgcaagtgctgacccagactccatcgcctgtgtctgcagctgtgggaggcacagtcaccatcaattgccagtccagtcagagtgtttatagtaacagcttcttatcctggtatcagcagaaaccagggcagcctcccaagcgcctgatctattctgcatccgatctggcatctggggtcccatcgcggttcaaaggcagtggatctgggacacagttcactctcaccatcagcgacctggagtgtgacgatgctgccacttactactgtgcaggcgtttatagtgataatacttatgttttcggcggaggcaccgaggtggagttcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 98 - DNA, 330 bp, synthetic construct
  misc_feature 1..330: note = synthesized
  source 1..330: mol_type = other DNA
gcgcaagtgctgacccagactccatcgcctgtgtctgcagctgtgggaggcacagtcaccatcaattgccagtccagtcagagtgtttatagtaacagcttcttatcctggtatcagcagaaaccagggcagcctcccaagcgcctgatctattctgcatccgatctggcatctggggtcccatcgcggttcaaaggcagtggatctgggacacagttcactctcaccatcagcgacctggagtgtgacgatgctgccacttactactgtgcaggcgtttatagtgataatacttatgttttcggcggaggcaccgaggtggagttcaaa

SEQ ID NO: 99 - protein, 217 aa, synthetic construct
  REGION 1..217: note = synthesized
AQVLTQTPSPVSAAVGGTVTINCQSSQSVYSNSFLSWYQQKPGQPPKRLIYSASDLASGVPSRFKGSGSGTQFTLTISDLECDDAATYYCAGVYSDNTYVFGGGTEVEFKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 110 aa, synthetic construct
  REGION 1..110: note = synthesized
AQVLTQTPSPVSAAVGGTVTINCQSSQSVYSNSFLSWYQQKPGQPPKRLIYSASDLASGVPSRFKGSGSGTQFTLTISDLECDDAATYYCAGVYSDNTYVFGGGTEVEFK

SEQ ID NO: 101 - DNA, 1329 bp, synthetic construct
  misc_feature 1..1329: note = synthesized
  source 1..1329: mol_type = other DNA
cagtcggtggaggagtccggcggtcgcctggtaaagcctgacgaatccctgacactcacctgcacagcctctggattctccctcagtcactactggatgacttgggtccgacaggctccagggaagggactggaatacatcggattcattaatgttggtggtgacacatcttacgcgagctggtcgaaaggccgattcaccatctccaaggcctcgaccacggtggatctgaagatcagtagtctgacaaccgaggacacggccacctatttctgtggcagaggtggtctgacttttggttgggacttgtggggcccagggaccctcgtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 102 - DNA, 342 bp, synthetic construct
  misc_feature 1..342: note = synthesized
  source 1..342: mol_type = other DNA
cagtcggtggaggagtccggcggtcgcctggtaaagcctgacgaatccctgacactcacctgcacagcctctggattctccctcagtcactactggatgacttgggtccgacaggctccagggaagggactggaatacatcggattcattaatgttggtggtgacacatcttacgcgagctggtcgaaaggccgattcaccatctccaaggcctcgaccacggtggatctgaagatcagtagtctgacaaccgaggacacggccacctatttctgtggcagaggtggtctgacttttggttgggacttgtggggcccagggaccctcgtcaccgtctcgagc

SEQ ID NO: 103 - protein, 444 aa, synthetic construct
  REGION 1..444: note = synthesized
QSVEESGGRLVKPDESLTLTCTASGFSLSHYWMTWVRQAPGKGLEYIGFINVGGDTSYASWSKGRFTISKASTTVDLKISSLTTEDTATYFCGRGGLTFGWDLWGPGTLVTVSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 114 aa, synthetic construct
  REGION 1..114: note = synthesized
QSVEESGGRLVKPDESLTLTCTASGFSLSHYWMTWVRQAPGKGLEYIGFINVGGDTSYASWSKGRFTISKASTTVDLKISSLTTEDTATYFCGRGGLTFGWDLWGPGTLVTVSS

SEQ ID NO: 105 - DNA, 654 bp, synthetic construct
  misc_feature 1..654: note = synthesized
  source 1..654: mol_type = other DNA
gatgttgtgatgacccagactccagcctccgtgtctgaacctgtgggaggcacagtcaccatcaagtgccaggccagtcagaacattaggacttacttatcctggtatcagcagaaaccagggcagcgtcccaagctcctgatctatgctgcagccaatctggcatctggggtcccatcgcggttcagtggcagtagatctgggacagagttcactctcaccatcagcgacctggagtgtgccgatgctgccacttactattgtcagtgtacctatcttggtactgattatgttggcggtgctttcggcggaggcaccgaggtggagttcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 106 - DNA, 333 bp, synthetic construct
  misc_feature 1..333: note = synthesized
  source 1..333: mol_type = other DNA
gatgttgtgatgacccagactccagcctccgtgtctgaacctgtgggaggcacagtcaccatcaagtgccaggccagtcagaacattaggacttacttatcctggtatcagcagaaaccagggcagcgtcccaagctcctgatctatgctgcagccaatctggcatctggggtcccatcgcggttcagtggcagtagatctgggacagagttcactctcaccatcagcgacctggagtgtgccgatgctgccacttactattgtcagtgtacctatcttggtactgattatgttggcggtgctttcggcggaggcaccgaggtggagttcaaa

SEQ ID NO: 107 - protein, 218 aa, synthetic construct
  REGION 1..218: note = synthesized
DVVMTQTPASVSEPVGGTVTIKCQASQNIRTYLSWYQQKPGQRPKLLIYAAANLASGVPSRFSGSRSGTEFTLTISDLECADAATYYCQCTYLGTDYVGGAFGGGTEVEFKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 111 aa, synthetic construct
  REGION 1..111: note = synthesized
DVVMTQTPASVSEPVGGTVTIKCQASQNIRTYLSWYQQKPGQRPKLLIYAAANLASGVPSRFSGSRSGTEFTLTISDLECADAATYYCQCTYLGTDYVGGAFGGGTEVEFK

SEQ ID NO: 109 - DNA, 1329 bp, synthetic construct
  misc_feature 1..1329: note = synthesized
  source 1..1329: mol_type = other DNA
cggtcgctggaggagtccgggggtcgcctggtcacgcctgggacacccctgacactcacctgcacagcctctggattcaccatcagtagctaccacatgcagtgggtccgccaggctccagggaaggggctggaatacatcggaaccattagtagtggtggtaatgtatactacgcgagctgggcgagaggccgattcaccatctccagaccctcgtcgaccacggtggatctgaagatgaccagtctgacaaccgaggacacggccacctatttctgtgccagagactctggttatagtgatcctatgtggggcccgggcaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 110 - DNA, 342 bp, synthetic construct
  misc_feature 1..342: note = synthesized
  source 1..342: mol_type = other DNA
cggtcgctggaggagtccgggggtcgcctggtcacgcctgggacacccctgacactcacctgcacagcctctggattcaccatcagtagctaccacatgcagtgggtccgccaggctccagggaaggggctggaatacatcggaaccattagtagtggtggtaatgtatactacgcgagctgggcgagaggccgattcaccatctccagaccctcgtcgaccacggtggatctgaagatgaccagtctgacaaccgaggacacggccacctatttctgtgccagagactctggttatagtgatcctatgtggggcccgggcaccctggtcaccgtctcgagc

SEQ ID NO: 111 - protein, 443 aa, synthetic construct
  REGION 1..443: note = synthesized
RSLEESGGRLVTPGTPLTLTCTASGFTISSYHMQWVRQAPGKGLEYIGTISSGGNVYYASWARGRFTISRPSSTTVDLKMTSLTTEDTATYFCARDSGYSDPMWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 112 - protein, 114 aa, synthetic construct
  REGION 1..114: note = synthesized
RSLEESGGRLVTPGTPLTLTCTASGFTISSYHMQWVRQAPGKGLEYIGTISSGGNVYYASWARGRFTISRPSSTTVDLKMTSLTTEDTATYFCARDSGYSDPMWGPGTLVTVSS

SEQ ID NO: 113 - DNA, 654 bp, synthetic construct
  misc_feature 1..654: note = synthesized
  source 1..654: mol_type = other DNA
gcagccgtgctgacccagacaccatcgcccgtgtctgcagctgtgggaggcacagtcaccatcagttgccagtccagtcagagtgtttatagtaacaaccaattatcctggtttcagcagaaatcagggcagcctcccaagctcctgatctatgatgcatccaatctggcatctggggtcccatcacggttcagcggcagtggatctgggacacagttcactctcaccatcagcagcgtgcagtgtgacgatgctgccacttaccactgtctaggcggtagtgatgatgatggtgatattgctttcggcggaggcaccgaggtggtgttcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 114 - DNA, 333 bp, synthetic construct
  misc_feature 1..333: note = synthesized
  source 1..333: mol_type = other DNA
gcagccgtgctgacccagacaccatcgcccgtgtctgcagctgtgggaggcacagtcaccatcagttgccagtccagtcagagtgtttatagtaacaaccaattatcctggtttcagcagaaatcagggcagcctcccaagctcctgatctatgatgcatccaatctggcatctggggtcccatcacggttcagcggcagtggatctgggacacagttcactctcaccatcagcagcgtgcagtgtgacgatgctgccacttaccactgtctaggcggtagtgatgatgatggtgatattgctttcggcggaggcaccgaggtggtgttcaaa

SEQ ID NO: 115 - protein, 218 aa, synthetic construct
  REGION 1..218: note = synthesized
AAVLTQTPSPVSAAVGGTVTISCQSSQSVYSNNQLSWFQQKSGQPPKLLIYDASNLASGVPSRFSGSGSGTQFTLTISSVQCDDAATYHCLGGSDDDGDIAFGGGTEVVFKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 111 aa, synthetic construct
  REGION 1..111: note = synthesized
AAVLTQTPSPVSAAVGGTVTISCQSSQSVYSNNQLSWFQQKSGQPPKLLIYDASNLASGVPSRFSGSGSGTQFTLTISSVQCDDAATYHCLGGSDDDGDIAFGGGTEVVFK

SEQ ID NO: 117 - DNA, 1323 bp, synthetic construct
  misc_feature 1..1323: note = synthesized
  source 1..1323: mol_type = other DNA
cagtcactggaggagtccgggggtcgcctggtcacgcctgggacacccctgacactcacctgcacagcctctggattctccctcagtagctactacatgagctgggtccgccaggctccagggaagggactggaatggatcggaagtatttatggtagtggtgccgcatactacgcgagctgggcgaaaggccgattcaccatctccagaacgtcgaccacggtggatctgaaaatgaccagtctgacagccgcggacacggccacctatttctgtgccagagatcctattaacaatgccatgtggggcccaggcaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 118 - DNA, 336 bp, synthetic construct
  misc_feature 1..336: note = synthesized
  source 1..336: mol_type = other DNA
cagtcactggaggagtccgggggtcgcctggtcacgcctgggacacccctgacactcacctgcacagcctctggattctccctcagtagctactacatgagctgggtccgccaggctccagggaagggactggaatggatcggaagtatttatggtagtggtgccgcatactacgcgagctgggcgaaaggccgattcaccatctccagaacgtcgaccacggtggatctgaaaatgaccagtctgacagccgcggacacggccacctatttctgtgccagagatcctattaacaatgccatgtggggcccaggcaccctggtcaccgtctcgagc

SEQ ID NO: 119 - protein, 441 aa, synthetic construct
  REGION 1..441: note = synthesized
QSLEESGGRLVTPGTPLTLTCTASGFSLSSYYMSWVRQAPGKGLEWIGSIYGSGAAYYASWAKGRFTISRTSTTVDLKMTSLTAADTATYFCARDPINNAMWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - protein, 112 aa, synthetic construct
  REGION 1..112: note = synthesized
QSLEESGGRLVTPGTPLTLTCTASGFSLSSYYMSWVRQAPGKGLEWIGSIYGSGAAYYASWAKGRFTISRTSTTVDLKMTSLTAADTATYFCARDPINNAMWGPGTLVTVSS

SEQ ID NO: 121 - DNA, 657 bp, synthetic construct
  misc_feature 1..657: note = synthesized
  source 1..657: mol_type = other DNA
gcccttgtgatgacccagactccagcctccgtgtctgcagctgtgggaggcacagtcaccatcaattgccaggccagtgaggacattgataactatttagcctggtatcagcagaaaccagggcagcctcccaagctcctgatcttttatgcatccgatctggcatctggggtcccatcgcggttctcaggcagtggatctgggacacagttcactctcaccatcagcggtgtgcagtgtgacgatgctgccacttactactgtcaaggcggttactatactagtagtactgatacgaggggtgctttcggcggaggcaccgaggtggtggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 122 - DNA, 336 bp, synthetic construct
  misc_feature 1..336: note = synthesized
  source 1..336: mol_type = other DNA
gcccttgtgatgacccagactccagcctccgtgtctgcagctgtgggaggcacagtcaccatcaattgccaggccagtgaggacattgataactatttagcctggtatcagcagaaaccagggcagcctcccaagctcctgatcttttatgcatccgatctggcatctggggtcccatcgcggttctcaggcagtggatctgggacacagttcactctcaccatcagcggtgtgcagtgtgacgatgctgccacttactactgtcaaggcggttactatactagtagtactgatacgaggggtgctttcggcggaggcaccgaggtggtggtcaaa

SEQ ID NO: 123 - protein, 219 aa, synthetic construct
  REGION 1..219: note = synthesized
ALVMTQTPASVSAAVGGTVTINCQASEDIDNYLAWYQQKPGQPPKLLIFYASDLASGVPSRFSGSGSGTQFTLTISGVQCDDAATYYCQGGYYTSSTDTRGAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 112 aa, synthetic construct
  REGION 1..112: note = synthesized
ALVMTQTPASVSAAVGGTVTINCQASEDIDNYLAWYQQKPGQPPKLLIFYASDLASGVPSRFSGSGSGTQFTLTISGVQCDDAATYYCQGGYYTSSTDTRGAFGGGTEVVVK

SEQ ID NO: 125 - DNA, 1344 bp, synthetic construct
  misc_feature 1..1344: note = synthesized
  source 1..1344: mol_type = other DNA
cagtcattggaggaggccgggggagacctggtcaagcctggggcatccctgacactcacctgcacagcctctggcttctccttcactagcagctactacatgtgctgggtccgccaggctccagggaaggggctggagtggatcgcatgcatttatactggtagtagtggtagcacttactacgcgaactgggcgaaaggccgattcaccatctccaaaacctcgtcgaccacggtgactctgcaaatgaccagtctgacagccgcggacacggccacctatttctgtgcgagagatagtagtagttattatatgtttaacttgtggggccagggcaccctcgtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 126 - DNA, 357 bp, synthetic construct
  misc_feature 1..357: note = synthesized
  source 1..357: mol_type = other DNA
cagtcattggaggaggccgggggagacctggtcaagcctggggcatccctgacactcacctgcacagcctctggcttctccttcactagcagctactacatgtgctgggtccgccaggctccagggaaggggctggagtggatcgcatgcatttatactggtagtagtggtagcacttactacgcgaactgggcgaaaggccgattcaccatctccaaaacctcgtcgaccacggtgactctgcaaatgaccagtctgacagccgcggacacggccacctatttctgtgcgagagatagtagtagttattatatgtttaacttgtggggccagggcaccctcgtcaccgtctcgagc

SEQ ID NO: 127 - protein, 448 aa, synthetic construct
  REGION 1..448: note = synthesized
QSLEEAGGDLVKPGASLTLTCTASGFSFTSSYYMCWVRQAPGKGLEWIACIYTGSSGSTYYANWAKGRFTISKTSSTTVTLQMTSLTAADTATYFCARDSSSYYMFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - protein, 119 aa, synthetic construct
  REGION 1..119: note = synthesized
QSLEEAGGDLVKPGASLTLTCTASGFSFTSSYYMCWVRQAPGKGLEWIACIYTGSSGSTYYANWAKGRFTISKTSSTTVTLQMTSLTAADTATYFCARDSSSYYMFNLWGQGTLVTVSS

SEQ ID NO: 129 - DNA, 654 bp, synthetic construct
  misc_feature 1..654: note = synthesized
  source 1..654: mol_type = other DNA
gcagccgtgctgacccagactccatcttccacgtctgcagctgtgggaggcacagtcaccatcagttgccagtccagtcagagtgtttataataacaatgctttagcctggtatcagaaaaaaccaggacagcctcccaagctcctgatctatttggcttccactctggcatctggggtcccatcgcggttcagcggcagtggatctgggacacagttcactctcaccatcagcgacctggagtgtgacgatgctgccacttattattgtctaggtgtttataatgatgatgttgataatggtttcggcggaggcaccgaggtggtgttcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 130 - DNA, 333 bp, synthetic construct
  misc_feature 1..333: note = synthesized
  source 1..333: mol_type = other DNA
gcagccgtgctgacccagactccatcttccacgtctgcagctgtgggaggcacagtcaccatcagttgccagtccagtcagagtgtttataataacaatgctttagcctggtatcagaaaaaaccaggacagcctcccaagctcctgatctatttggcttccactctggcatctggggtcccatcgcggttcagcggcagtggatctgggacacagttcactctcaccatcagcgacctggagtgtgacgatgctgccacttattattgtctaggtgtttataatgatgatgttgataatggtttcggcggaggcaccgaggtggtgttcaaa

SEQ ID NO: 131 - protein, 218 aa, synthetic construct
  REGION 1..218: note = synthesized
AAVLTQTPSSTSAAVGGTVTISCQSSQSVYNNNALAWYQKKPGQPPKLLIYLASTLASGVPSRFSGSGSGTQFTLTISDLECDDAATYYCLGVYNDDVDNGFGGGTEVVFKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 111 aa, synthetic construct
  REGION 1..111: note = synthesized
AAVLTQTPSSTSAAVGGTVTISCQSSQSVYNNNALAWYQKKPGQPPKLLIYLASTLASGVPSRFSGSGSGTQFTLTISDLECDDAATYYCLGVYNDDVDNGFGGGTEVVFK

SEQ ID NO: 133 - DNA, 1332 bp, synthetic construct
  misc_feature 1..1332: note = synthesized
  source 1..1332: mol_type = other DNA
cagtcgctggaggagtccgggggtcgcctggtcacgcctggaggatccctgacactcacctgcaccgtctctggattctccctcaataacaatgcaataagctgggtccgccaggctccagggaaggggctggaatgggttgcaatcatacaaaatactggtaccacagactacgcgaggtgggcgaaaggccgattcaccatctccaaaacctcgaccacggtggatttgaaaatcaacagtccgacaaccgaggacacggccacctatttctgtggcagagggtatggttttgagtcggagcttgtcatctggggcccgggcaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 134 - DNA, 345 bp, synthetic construct
  misc_feature 1..345: note = synthesized
  source 1..345: mol_type = other DNA
cagtcgctggaggagtccgggggtcgcctggtcacgcctggaggatccctgacactcacctgcaccgtctctggattctccctcaataacaatgcaataagctgggtccgccaggctccagggaaggggctggaatgggttgcaatcatacaaaatactggtaccacagactacgcgaggtgggcgaaaggccgattcaccatctccaaaacctcgaccacggtggatttgaaaatcaacagtccgacaaccgaggacacggccacctatttctgtggcagagggtatggttttgagtcggagcttgtcatctggggcccgggcaccctggtcaccgtctcgagc

SEQ ID NO: 135 - protein, 444 aa, synthetic construct
  REGION 1..444: note = synthesized
QSLEESGGRLVTPGGSLTLTCTVSGFSLNNNAISWVRQAPGKGLEWVAIIQNTGTTDYARWAKGRFTISKTSTTVDLKINSPTTEDTATYFCGRGYGFESELVIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 136 - protein, 115 aa, synthetic construct
  REGION 1..115: note = synthesized
QSLEESGGRLVTPGGSLTLTCTVSGFSLNNNAISWVRQAPGKGLEWVAIIQNTGTTDYARWAKGRFTISKTSTTVDLKINSPTTEDTATYFCGRGYGFESELVIWGPGTLVTVSS

SEQ ID NO: 137 - DNA, 657 bp, synthetic construct
  misc_feature 1..657: note = synthesized
  source 1..657: mol_type = other DNA
gacgttgtgatgacccagtctccatcttccgtgtctgcatctgtaggagacagagtcaccatcacctgtcaggccagtcagagcattggtgctgatttagcctggtatcagcagaaaccagggaaagcccctaagctcctgatctatagggcatccactctggcctctggggtcccatcaaggttcagcggcagtggatctgggacagatttcactctcaccatcagcagcctgcagcctgaagattttgcaacttactattgtcaagctatttattatggtactgatgatgtcatataccatactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 138 - DNA, 336 bp, synthetic construct
  misc_feature 1..336: note = synthesized
  source 1..336: mol_type = other DNA
gacgttgtgatgacccagtctccatcttccgtgtctgcatctgtaggagacagagtcaccatcacctgtcaggccagtcagagcattggtgctgatttagcctggtatcagcagaaaccagggaaagcccctaagctcctgatctatagggcatccactctggcctctggggtcccatcaaggttcagcggcagtggatctgggacagatttcactctcaccatcagcagcctgcagcctgaagattttgcaacttactattgtcaagctatttattatggtactgatgatgtcatataccatactttcggcggagggaccaaggtggagatcaaa

SEQ ID NO: 139 - protein, 219 aa, synthetic construct
  REGION 1..219: note = synthesized
DVVMTQSPSSVSASVGDRVTITCQASQSIGADLAWYQQKPGKAPKLLIYRASTLASGVPSRFSGSGSGTDFTLTISSLQPEDFATYYCQAIYYGTDDVIYHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 112 aa, synthetic construct
  REGION 1..112: note = synthesized
DVVMTQSPSSVSASVGDRVTITCQASQSIGADLAWYQQKPGKAPKLLIYRASTLASGVPSRFSGSGSGTDFTLTISSLQPEDFATYYCQAIYYGTDDVIYHTFGGGTKVEIK

SEQ ID NO: 141 - DNA, 1341 bp, synthetic construct
  misc_feature 1..1341: note = synthesized
  source 1..1341: mol_type = other DNA
gaggtgcagctggtggagtctgggggaggcttggtccagcctggggggtccctgagactctcctgtgcagcctctggattcaccatcagtagccaccacatgatctgggtccgccaggctccagggaaggggctgcaatacatcggattcattaatgatggtgactatacatactacacgaactccgcaaaaggcagattcaccatctccagagacaattccaagaacacgctgtatcttcaaatgaacagcctgagagccgaggacacggctgtgtattactgtgcgagaggggttgatggtactagttatcctggcttgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 142 - DNA, 354 bp, synthetic construct
  misc_feature 1..354: note = synthesized
  source 1..354: mol_type = other DNA
gaggtgcagctggtggagtctgggggaggcttggtccagcctggggggtccctgagactctcctgtgcagcctctggattcaccatcagtagccaccacatgatctgggtccgccaggctccagggaaggggctgcaatacatcggattcattaatgatggtgactatacatactacacgaactccgcaaaaggcagattcaccatctccagagacaattccaagaacacgctgtatcttcaaatgaacagcctgagagccgaggacacggctgtgtattactgtgcgagaggggttgatggtactagttatcctggcttgtggggccagggaaccctggtcaccgtctcgagc

SEQ ID NO: 143 - protein, 447 aa, synthetic construct
  REGION 1..447: note = synthesized
EVQLVESGGGLVQPGGSLRLSCAASGFTISSHHMIWVRQAPGKGLQYIGFINDGDYTYYTNSAKGRFTISRDNSKNTLYLQMNSLRAEDTAVYYCARGVDGTSYPGLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 144 - protein, 118 aa, synthetic construct
  REGION 1..118: note = synthesized
EVQLVESGGGLVQPGGSLRLSCAASGFTISSHHMIWVRQAPGKGLQYIGFINDGDYTYYTNSAKGRFTISRDNSKNTLYLQMNSLRAEDTAVYYCARGVDGTSYPGLWGQGTLVTVSS

SEQ ID NO: 145 - DNA, 657 bp, synthetic construct
  misc_feature 1..657: note = synthesized
  source 1..657: mol_type = other DNA
gacatccagatgacccagtctccatcctccctgtctgcatctgtaggagacagagtcaccatcacttgccaggccagtcagagcatttacagcaatttagcctggtatcagcagaaaccagggaaagttcctaagctcctgatctattctacatccactctggcatctggggtcccatctcggttcagtggcagtggatctgggacagatttcactctcaccatcagcagcctgcagcctgaagatgttgcaacttattactgtcaaaactattatggtagtagtactgatagttatgggaatcctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 146 - DNA, 336 bp, synthetic construct
  misc_feature 1..336: note = synthesized
  source 1..336: mol_type = other DNA
gacatccagatgacccagtctccatcctccctgtctgcatctgtaggagacagagtcaccatcacttgccaggccagtcagagcatttacagcaatttagcctggtatcagcagaaaccagggaaagttcctaagctcctgatctattctacatccactctggcatctggggtcccatctcggttcagtggcagtggatctgggacagatttcactctcaccatcagcagcctgcagcctgaagatgttgcaacttattactgtcaaaactattatggtagtagtactgatagttatgggaatcctttcggcggagggaccaaggtggagatcaaa

SEQ ID NO: 147 - protein, 219 aa, synthetic construct
  REGION 1..219: note = synthesized
DIQMTQSPSSLSASVGDRVTITCQASQSIYSNLAWYQQKPGKVPKLLIYSTSTLASGVPSRFSGSGSGTDFTLTISSLQPEDVATYYCQNYYGSSTDSYGNPFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 112 aa, synthetic construct
  REGION 1..112: note = synthesized
DIQMTQSPSSLSASVGDRVTITCQASQSIYSNLAWYQQKPGKVPKLLIYSTSTLASGVPSRFSGSGSGTDFTLTISSLQPEDVATYYCQNYYGSSTDSYGNPFGGGTKVEIK

SEQ ID NO: 149 - DNA, 1350 bp, synthetic construct
  misc_feature 1..1350: note = synthesized
  source 1..1350: mol_type = other DNA
gaggtgcagctggtggagtctgggggaggcttggtccagcctggggggtccctgagactctcctgtgcagcctctggaatcgacttcagtagcaactacatgtgctgggtccgccaggctccagggaaggggctggagtggatcgcatgcatttatggtgatagtagtgataatagttactccgcgagctccgctaaaggcagattcaccatctccagagacaattccaagaacacgctgtatcttcaaatgaacagcctgagagccgaggacacggctgtgtattactgtgcgagatctggttatagctatttaggctactttaacttgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 150 - DNA, 363 bp, synthetic construct
  misc_feature 1..363: note = synthesized
  source 1..363: mol_type = other DNA
gaggtgcagctggtggagtctgggggaggcttggtccagcctggggggtccctgagactctcctgtgcagcctctggaatcgacttcagtagcaactacatgtgctgggtccgccaggctccagggaaggggctggagtggatcgcatgcatttatggtgatagtagtgataatagttactccgcgagctccgctaaaggcagattcaccatctccagagacaattccaagaacacgctgtatcttcaaatgaacagcctgagagccgaggacacggctgtgtattactgtgcgagatctggttatagctatttaggctactttaacttgtggggccagggaaccctggtcaccgtctcgagc

SEQ ID NO: 151 - protein, 450 aa, synthetic construct
  REGION 1..450: note = synthesized
EVQLVESGGGLVQPGGSLRLSCAASGIDFSSNYMCWVRQAPGKGLEWIACIYGDSSDNSYSASSAKGRFTISRDNSKNTLYLQMNSLRAEDTAVYYCARSGYSYLGYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 152 - protein, 121 aa, synthetic construct
  REGION 1..121: note = synthesized
EVQLVESGGGLVQPGGSLRLSCAASGIDFSSNYMCWVRQAPGKGLEWIACIYGDSSDNSYSASSAKGRFTISRDNSKNTLYLQMNSLRAEDTAVYYCARSGYSYLGYFNLWGQGTLVTVSS

SEQ ID NO: 153 - DNA, 651 bp, synthetic construct
  misc_feature 1..651: note = synthesized
  source 1..651: mol_type = other DNA
gacatccagatgacccagtctccatcctccctgtctgcatctgtaggagacagagtcaccatcacttgccagtccagtcagactgtttataataacaacttgttatcctggtatcagcagaaaccagggaaagttcctaagctcctgatctattatgcatccactctggcatttggggtcccatctcggttcagtggcagtggatctgggacagatttcactctcaccatcagcagcctgcagcctgaagatgttgcaacttattactgtcaaggcggttatagtggttggatttatgttttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 154 - DNA, 330 bp, synthetic construct
  misc_feature 1..330: note = synthesized
  source 1..330: mol_type = other DNA
gacatccagatgacccagtctccatcctccctgtctgcatctgtaggagacagagtcaccatcacttgccagtccagtcagactgtttataataacaacttgttatcctggtatcagcagaaaccagggaaagttcctaagctcctgatctattatgcatccactctggcatttggggtcccatctcggttcagtggcagtggatctgggacagatttcactctcaccatcagcagcctgcagcctgaagatgttgcaacttattactgtcaaggcggttatagtggttggatttatgttttcggcggagggaccaaggtggagatcaaa

SEQ ID NO: 155 - protein, 217 aa, synthetic construct
  REGION 1..217: note = synthesized
DIQMTQSPSSLSASVGDRVTITCQSSQTVYNNNLLSWYQQKPGKVPKLLIYYASTLAFGVPSRFSGSGSGTDFTLTISSLQPEDVATYYCQGGYSGWIYVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 110 aa, synthetic construct
  REGION 1..110: note = synthesized
DIQMTQSPSSLSASVGDRVTITCQSSQTVYNNNLLSWYQQKPGKVPKLLIYYASTLAFGVPSRFSGSGSGTDFTLTISSLQPEDVATYYCQGGYSGWIYVFGGGTKVEIK

SEQ ID NO: 157 - DNA, 1329 bp, synthetic construct
  misc_feature 1..1329: note = synthesized
  source 1..1329: mol_type = other DNA
cagtcgctggtggagtctgggggaggcttggtccagcctggggggtccctgagactctcctgtgcagcctctggattctccctcagtaactactacatgaactgggtccgccaggctccagggaaggggctggagtacatcggaatcattagtagtagtggtagcgcatactacgcgagctccgctaaaggcagattcaccatctccagagacaattccaagaacacgctgtatcttcaaatgaacagcctgagagccgaggacacggctgtgtattactgtgcgagaggggactacggcatggacctctggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 158 - DNA, 342 bp, synthetic construct
  misc_feature 1..342: note = synthesized
  source 1..342: mol_type = other DNA
cagtcgctggtggagtctgggggaggcttggtccagcctggggggtccctgagactctcctgtgcagcctctggattctccctcagtaactactacatgaactgggtccgccaggctccagggaaggggctggagtacatcggaatcattagtagtagtggtagcgcatactacgcgagctccgctaaaggcagattcaccatctccagagacaattccaagaacacgctgtatcttcaaatgaacagcctgagagccgaggacacggctgtgtattactgtgcgagaggggactacggcatggacctctggggccagggaaccctggtcaccgtctcgagc

SEQ ID NO: 159 - protein, 443 aa, synthetic construct
  REGION 1..443: note = synthesized
QSLVESGGGLVQPGGSLRLSCAASGFSLSNYYMNWVRQAPGKGLEYIGIISSSGSAYYASSAKGRFTISRDNSKNTLYLQMNSLRAEDTAVYYCARGDYGMDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 160 - protein, 114 aa, synthetic construct
  REGION 1..114: note = synthesized
QSLVESGGGLVQPGGSLRLSCAASGFSLSNYYMNWVRQAPGKGLEYIGIISSSGSAYYASSAKGRFTISRDNSKNTLYLQMNSLRAEDTAVYYCARGDYGMDLWGQGTLVTVSS

SEQ ID NO: 161 - DNA, 657 bp, synthetic construct
  misc_feature 1..657: note = synthesized
  source 1..657: mol_type = other DNA
gacgttgtgatgacccagtctccatcttccgtgtctgcatctgtaggagacagagtcaccatcacctgtcaggccagtcagagcattggtagtgatttagcctggtatcagcagaaaccagggaaagcccctaagctcctgatctatagggcatccactctggcatctggggtcccatcaaggttcagcggcagtggatctgggacagatttcactctcaccatcagcagcctgcagcctgaagattttgcaacttactattgtcaatccatttattatggtagtgatgatgtcatataccatactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 162 - DNA, 336 bp, synthetic construct
  misc_feature 1..336: note = synthesized
  source 1..336: mol_type = other DNA
gacgttgtgatgacccagtctccatcttccgtgtctgcatctgtaggagacagagtcaccatcacctgtcaggccagtcagagcattggtagtgatttagcctggtatcagcagaaaccagggaaagcccctaagctcctgatctatagggcatccactctggcatctggggtcccatcaaggttcagcggcagtggatctgggacagatttcactctcaccatcagcagcctgcagcctgaagattttgcaacttactattgtcaatccatttattatggtagtgatgatgtcatataccatactttcggcggagggaccaaggtggagatcaaa

SEQ ID NO: 163 - protein, 219 aa, synthetic construct
  REGION 1..219: note = synthesized
DVVMTQSPSSVSASVGDRVTITCQASQSIGSDLAWYQQKPGKAPKLLIYRASTLASGVPSRFSGSGSGTDFTLTISSLQPEDFATYYCQSIYYGSDDVIYHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 112 aa, synthetic construct
  REGION 1..112: note = synthesized
DVVMTQSPSSVSASVGDRVTITCQASQSIGSDLAWYQQKPGKAPKLLIYRASTLASGVPSRFSGSGSGTDFTLTISSLQPEDFATYYCQSIYYGSDDVIYHTFGGGTKVEIK

SEQ ID NO: 165 - DNA, 1341 bp, synthetic construct
  misc_feature 1..1341: note = synthesized
  source 1..1341: mol_type = other DNA
gaggtgcagctggtggagtctgggggaggcttggtccagcctggggggtccctgagactctcctgtgcagcctctggattcactatcaatagctaccacatgatctgggtccgccaggctccagggaaggggctggagtacatcggattcattaatgatggtggtttcacatactacgcgagctccgctaaaggcagattcaccatctccagagacaattccaagaacacgctgtatcttcaaatgaacagcctgagagccgaggacacggctgtgtattactgtgcgagaggggttgatggtactagttatcctgacttatggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 166 - DNA, 354 bp, synthetic construct
  misc_feature 1..354: note = synthesized
  source 1..354: mol_type = other DNA
gaggtgcagctggtggagtctgggggaggcttggtccagcctggggggtccctgagactctcctgtgcagcctctggattcactatcaatagctaccacatgatctgggtccgccaggctccagggaaggggctggagtacatcggattcattaatgatggtggtttcacatactacgcgagctccgctaaaggcagattcaccatctccagagacaattccaagaacacgctgtatcttcaaatgaacagcctgagagccgaggacacggctgtgtattactgtgcgagaggggttgatggtactagttatcctgacttatggggccagggaaccctggtcaccgtctcgagc

SEQ ID NO: 167 - protein, 447 aa, synthetic construct
  REGION 1..447: note = synthesized
EVQLVESGGGLVQPGGSLRLSCAASGFTINSYHMIWVRQAPGKGLEYIGFINDGGFTYYASSAKGRFTISRDNSKNTLYLQMNSLRAEDTAVYYCARGVDGTSYPDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 168 - protein, 118 aa, synthetic construct
  REGION 1..118: note = synthesized
EVQLVESGGGLVQPGGSLRLSCAASGFTINSYHMIWVRQAPGKGLEYIGFINDGGFTYYASSAKGRFTISRDNSKNTLYLQMNSLRAEDTAVYYCARGVDGTSYPDLWGQGTLVTVSS

SEQ ID NO: 169 - DNA, 654 bp, synthetic construct
  misc_feature 1..654: note = synthesized
  source 1..654: mol_type = other DNA
gacatccagatgacccagtctccttccaccctgtctgcatctgtaggagacagagtcaccatcacttgccaggccagtcagagcattggtagtaacttgaactggtatcagcagaaaccagggaaagcccctaagctcctgatctattatgtatccactctggcatctggggtcccatcaaggttcagcggcagtggatctgggacagaattcactctcaccatcagcagcctgcagcctgatgattttgcaacttattactgcctaggtgtttggaattattggggtgatgacggtactgctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 170 - DNA, 333 bp, synthetic construct
  misc_feature 1..333: note = synthesized
  source 1..333: mol_type = other DNA
gacatccagatgacccagtctccttccaccctgtctgcatctgtaggagacagagtcaccatcacttgccaggccagtcagagcattggtagtaacttgaactggtatcagcagaaaccagggaaagcccctaagctcctgatctattatgtatccactctggcatctggggtcccatcaaggttcagcggcagtggatctgggacagaattcactctcaccatcagcagcctgcagcctgatgattttgcaacttattactgcctaggtgtttggaattattggggtgatgacggtactgctttcggcggagggaccaaggtggagatcaaa

SEQ ID NO: 171 - protein, 218 aa, synthetic construct
  REGION 1..218: note = synthesized
DIQMTQSPSTLSASVGDRVTITCQASQSIGSNLNWYQQKPGKAPKLLIYYVSTLASGVPSRFSGSGSGTEFTLTISSLQPDDFATYYCLGVWNYWGDDGTAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 111 aa, synthetic construct
  REGION 1..111: note = synthesized
DIQMTQSPSTLSASVGDRVTITCQASQSIGSNLNWYQQKPGKAPKLLIYYVSTLASGVPSRFSGSGSGTEFTLTISSLQPDDFATYYCLGVWNYWGDDGTAFGGGTKVEIK

SEQ ID NO: 173 - DNA, 1332 bp, synthetic construct
  misc_feature 1..1332: note = synthesized
  source 1..1332: mol_type = other DNA
gaggtgcagctggtggagtctgggggaggcttggtccagcctggggggtccctgagactctcctgtgcagcctctggattctccctcagtacctattatatgagctgggtccgccaggctccagggaaggggctggagtggatcggaagtatttatgatagtggtgccgcatactacgcgacctccgcgaagggcagattcaccatctccagagacaattccaagaacacgctgtatcttcaaatgaacagcctgagagccgaggacacggctgtgtattactgtgcgagagatcctattaataatgccatctggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 174 - DNA, 345 bp, synthetic construct
  misc_feature 1..345: note = synthesized
  source 1..345: mol_type = other DNA
gaggtgcagctggtggagtctgggggaggcttggtccagcctggggggtccctgagactctcctgtgcagcctctggattctccctcagtacctattatatgagctgggtccgccaggctccagggaaggggctggagtggatcggaagtatttatgatagtggtgccgcatactacgcgacctccgcgaagggcagattcaccatctccagagacaattccaagaacacgctgtatcttcaaatgaacagcctgagagccgaggacacggctgtgtattactgtgcgagagatcctattaataatgccatctggggccagggaaccctggtcaccgtctcgagc

SEQ ID NO: 175 - protein, 444 aa, synthetic construct
  REGION 1..444: note = synthesized
EVQLVESGGGLVQPGGSLRLSCAASGFSLSTYYMSWVRQAPGKGLEWIGSIYDSGAAYYATSAKGRFTISRDNSKNTLYLQMNSLRAEDTAVYYCARDPINNAIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 176 - protein, 115 aa, synthetic construct
  REGION 1..115: note = synthesized
EVQLVESGGGLVQPGGSLRLSCAASGFSLSTYYMSWVRQAPGKGLEWIGSIYDSGAAYYATSAKGRFTISRDNSKNTLYLQMNSLRAEDTAVYYCARDPINNAIWGQGTLVTVSS

SEQ ID NO: 177 - DNA, 654 bp, synthetic construct
  misc_feature 1..654: note = synthesized
  source 1..654: mol_type = other DNA
gacatccagatgacccagtctccttccaccctgtctgcatctgtaggagacagagtcaccatcacttgccaggccagtcagagtattgctactaacttgaactggtatcagcagaaaccagggaaagcccctaagctcctgatctattatacatccagtctggcatctggggtcccatcaaggttcagcggcagtggatctgggacagaattcactctcaccatcagcagcctgcagcctgatgattttgcaacttattactgcctaggtgtttggaattattggggtgatgatggtactgctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 178 - DNA, 333 bp, synthetic construct
  misc_feature 1..333: note = synthesized
  source 1..333: mol_type = other DNA
gacatccagatgacccagtctccttccaccctgtctgcatctgtaggagacagagtcaccatcacttgccaggccagtcagagtattgctactaacttgaactggtatcagcagaaaccagggaaagcccctaagctcctgatctattatacatccagtctggcatctggggtcccatcaaggttcagcggcagtggatctgggacagaattcactctcaccatcagcagcctgcagcctgatgattttgcaacttattactgcctaggtgtttggaattattggggtgatgatggtactgctttcggcggagggaccaaggtggagatcaaa

SEQ ID NO: 179 - protein, 218 aa, synthetic construct
  REGION 1..218: note = synthesized
DIQMTQSPSTLSASVGDRVTITCQASQSIATNLNWYQQKPGKAPKLLIYYTSSLASGVPSRFSGSGSGTEFTLTISSLQPDDFATYYCLGVWNYWGDDGTAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - protein, 111 aa, synthetic construct
  REGION 1..111: note = synthesized
DIQMTQSPSTLSASVGDRVTITCQASQSIATNLNWYQQKPGKAPKLLIYYTSSLASGVPSRFSGSGSGTEFTLTISSLQPDDFATYYCLGVWNYWGDDGTAFGGGTKVEIK

SEQ ID NO: 181 - DNA, 1332 bp, synthetic construct
  misc_feature 1..1332: note = synthesized
  source 1..1332: mol_type = other DNA
gaggtgcagctggtggagtctgggggaggcttggtccagcctggggggtccctgagactctcctgtgcagcctctggattctccctcagtagctactacatgagctgggtccgccaggctccagggaaggggctggagtggatcggaagtatttatgctagtggtagcgcatactacgcgagttccgcgaagggcagattcaccatctccagagacaattccaagaacacgctgtatcttcaaatgaacagcctgagagccgaggacacggctgtgtattactgtgcgagagatcctattaacaatgacatctggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 182 - DNA, 345 bp, synthetic construct
  misc_feature 1..345: note = synthesized
  source 1..345: mol_type = other DNA
gaggtgcagctggtggagtctgggggaggcttggtccagcctggggggtccctgagactctcctgtgcagcctctggattctccctcagtagctactacatgagctgggtccgccaggctccagggaaggggctggagtggatcggaagtatttatgctagtggtagcgcatactacgcgagttccgcgaagggcagattcaccatctccagagacaattccaagaacacgctgtatcttcaaatgaacagcctgagagccgaggacacggctgtgtattactgtgcgagagatcctattaacaatgacatctggggccagggaaccctggtcaccgtctcgagc

SEQ ID NO: 183 - protein, 444 aa, synthetic construct
  REGION 1..444: note = synthesized
EVQLVESGGGLVQPGGSLRLSCAASGFSLSSYYMSWVRQAPGKGLEWIGSIYASGSAYYASSAKGRFTISRDNSKNTLYLQMNSLRAEDTAVYYCARDPINNDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 184 - protein, 115 aa, synthetic construct
  REGION 1..115: note = synthesized
EVQLVESGGGLVQPGGSLRLSCAASGFSLSSYYMSWVRQAPGKGLEWIGSIYASGSAYYASSAKGRFTISRDNSKNTLYLQMNSLRAEDTAVYYCARDPINNDIWGQGTLVTVSS

SEQ ID NO: 185 - DNA, 654 bp, synthetic construct
  misc_feature 1..654: note = synthesized
  source 1..654: mol_type = other DNA
gacatccagatgacccagtctccatcttccgtgtctgcatctgtaggagacagagtcaccatcacttgtcagtccagtccgagtgtttataagaacaaccaattagcctggtatcagcagaaaccagggaaagcccctaagctcctgatctatctggcatctactctggcatctggggtcccatcaaggttcagcggcagtggatctgggacagatttcactctcaccatcagcagcctgcagcctgaagattttgcaacttactattgtgcaggcggttatagtagtagtagtgatactgctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 186 - DNA, 333 bp, synthetic construct
  misc_feature 1..333: note = synthesized
  source 1..333: mol_type = other DNA
gacatccagatgacccagtctccatcttccgtgtctgcatctgtaggagacagagtcaccatcacttgtcagtccagtccgagtgtttataagaacaaccaattagcctggtatcagcagaaaccagggaaagcccctaagctcctgatctatctggcatctactctggcatctggggtcccatcaaggttcagcggcagtggatctgggacagatttcactctcaccatcagcagcctgcagcctgaagattttgcaacttactattgtgcaggcggttatagtagtagtagtgatactgctttcggcggagggaccaaggtggagatcaaa

SEQ ID NO: 187 - protein, 218 aa, synthetic construct
  REGION 1..218: note = synthesized
DIQMTQSPSSVSASVGDRVTITCQSSPSVYKNNQLAWYQQKPGKAPKLLIYLASTLASGVPSRFSGSGSGTDFTLTISSLQPEDFATYYCAGGYSSSSDTAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 188 - protein, 111 aa, synthetic construct
  REGION 1..111: note = synthesized
DIQMTQSPSSVSASVGDRVTITCQSSPSVYKNNQLAWYQQKPGKAPKLLIYLASTLASGVPSRFSGSGSGTDFTLTISSLQPEDFATYYCAGGYSSSSDTAFGGGTKVEIK

SEQ ID NO: 189 - DNA, 1270 bp, synthetic construct
  misc_feature 1..1270: note = synthesized
  source 1..1270: mol_type = other DNA
tggaatcgacctcagtgcctaccacatgagctgggtccgccaggctccagggaaggggctggagtacatcgggatgattggtagtagtggtaccatacactacgcgaactccgcgaaaggcagattcaccatctccaaagacaataccaagaacacggtggatcttcaaatgaacagcctgagagccgaggacacggctgtgtattactgtgcgagagacagttataatagtgattatgcctttaacttatggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 190 - DNA, 357 bp, synthetic construct
  misc_feature 1..357: note = synthesized
  source 1..357: mol_type = other DNA
gaggtgcagctggtggagtctgggggaggcttggtccagcctggggggtccctgagactctcctgtacagcctctggaatcgacctcagtgcctaccacatgagctgggtccgccaggctccagggaaggggctggagtacatcgggatgattggtagtagtggtaccatacactacgcgaactccgcgaaaggcagattcaccatctccaaagacaataccaagaacacggtggatcttcaaatgaacagcctgagagccgaggacacggctgtgtattactgtgcgagagacagttataatagtgattatgcctttaacttatggggccagggaaccctggtcaccgtctcgagc

SEQ ID NO: 191 - protein, 448 aa, synthetic construct
  REGION 1..448: note = synthesized
EVQLVESGGGLVQPGGSLRLSCTASGIDLSAYHMSWVRQAPGKGLEYIGMIGSSGTIHYANSAKGRFTISKDNTKNTVDLQMNSLRAEDTAVYYCARDSYNSDYAFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 192 - protein, 119 aa, synthetic construct
  REGION 1..119: note = synthesized
EVQLVESGGGLVQPGGSLRLSCTASGIDLSAYHMSWVRQAPGKGLEYIGMIGSSGTIHYANSAKGRFTISKDNTKNTVDLQMNSLRAEDTAVYYCARDSYNSDYAFNLWGQGTLVTVSS

SEQ ID NO: 193 - DNA, 653 bp, synthetic construct
  misc_feature 1..653: note = synthesized
  source 1..653: mol_type = other DNA
gacatccagatgacccagtctccttccaccctgtctgcatctgtaggagacagagtcaccatcacttgccagtccagtcagagtgtttatagtaactggttatcctggtatcagcagaaaccagggaaagcccctaagagactgatctattctgcatccactctggcatctggggtcccatcaaggttcagcggcagtggatctgggacagaattcactctcaccatcagcagcctgcagcctgatgattttgcaacttattactgcgcaggcggttataatactgttactgatacttttgctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtg

SEQ ID NO: 194 - DNA, 333 bp, synthetic construct
  misc_feature 1..333: note = synthesized
  source 1..333: mol_type = other DNA
gacatccagatgacccagtctccttccaccctgtctgcatctgtaggagacagagtcaccatcacttgccagtccagtcagagtgtttatagtaactggttatcctggtatcagcagaaaccagggaaagcccctaagagactgatctattctgcatccactctggcatctggggtcccatcaaggttcagcggcagtggatctgggacagaattcactctcaccatcagcagcctgcagcctgatgattttgcaacttattactgcgcaggcggttataatactgttactgatacttttgctttcggcggagggaccaaggtggagatcaaa

SEQ ID NO: 195 - protein, 218 aa, synthetic construct
  REGION 1..218: note = synthesized
DIQMTQSPSTLSASVGDRVTITCQSSQSVYSNWLSWYQQKPGKAPKRLIYSASTLASGVPSRFSGSGSGTEFTLTISSLQPDDFATYYCAGGYNTVTDTFAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 196 - protein, 111 aa, synthetic construct
  REGION 1..111: note = synthesized
DIQMTQSPSTLSASVGDRVTITCQSSQSVYSNWLSWYQQKPGKAPKRLIYSASTLASGVPSRFSGSGSGTEFTLTISSLQPDDFATYYCAGGYNTVTDTFAFGGGTKVEIK

SEQ ID NO: 197 - DNA, 1332 bp, synthetic construct
  misc_feature 1..1332: note = synthesized
  source 1..1332: mol_type = other DNA
gaggtgcagctgttggagtctgggggaggcttggtacagcctggggggtccctgagactctcctgtacagcctctggattctccttcagtagcaggcactacatgtgttgggtccgccaggctccagggaaggggctggagtggatcgcatgcatttatactggtagtagtggtactcctcactacgcgagctccgcgaaaggccggttcaccatctcccaagacaattccaagaacacggtgactctgcaaatgaacagcctgagagccgaggacacggccgtatattactgtgcgagagaaggtaacctgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 198 - DNA, 345 bp, synthetic construct
  misc_feature 1..345: note = synthesized
  source 1..345: mol_type = other DNA
gaggtgcagctgttggagtctgggggaggcttggtacagcctggggggtccctgagactctcctgtacagcctctggattctccttcagtagcaggcactacatgtgttgggtccgccaggctccagggaaggggctggagtggatcgcatgcatttatactggtagtagtggtactcctcactacgcgagctccgcgaaaggccggttcaccatctcccaagacaattccaagaacacggtgactctgcaaatgaacagcctgagagccgaggacacggccgtatattactgtgcgagagaaggtaacctgtggggccagggaaccctggtcaccgtctcgagc

SEQ ID NO: 199 - protein, 444 aa, synthetic construct
  REGION 1..444: note = synthesized
EVQLLESGGGLVQPGGSLRLSCTASGFSFSSRHYMCWVRQAPGKGLEWIACIYTGSSGTPHYASSAKGRFTISQDNSKNTVTLQMNSLRAEDTAVYYCAREGNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 200 - protein, 115 aa, synthetic construct
  REGION 1..115: note = synthesized
EVQLLESGGGLVQPGGSLRLSCTASGFSFSSRHYMCWVRQAPGKGLEWIACIYTGSSGTPHYASSAKGRFTISQDNSKNTVTLQMNSLRAEDTAVYYCAREGNLWGQGTLVTVSS

SEQ ID NO: 201 - DNA, 657 bp, synthetic construct
  misc_feature 1..657: note = synthesized
  source 1..657: mol_type = other DNA
gcccttgtgatgacccagtctccttccaccctgtctgcatctgtaggagacagagtcaccatcaattgccaggccagtgaggacattgatacctatttagcctggtatcagcagaaaccagggaaagcccctaagctcctgatcttttatgcatccgatctggcatctggggtcccatcaaggttcagcggcagtggatctgggacagaattcactctcaccatcagcagcctgcagcctgatgattttgcaacttattactgccaaggcggttactatactagtagtgctgatacgaggggtgctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 202 - DNA, 336 bp, synthetic construct
  misc_feature 1..336: note = synthesized
  source 1..336: mol_type = other DNA
gcccttgtgatgacccagtctccttccaccctgtctgcatctgtaggagacagagtcaccatcaattgccaggccagtgaggacattgatacctatttagcctggtatcagcagaaaccagggaaagcccctaagctcctgatcttttatgcatccgatctggcatctggggtcccatcaaggttcagcggcagtggatctgggacagaattcactctcaccatcagcagcctgcagcctgatgattttgcaacttattactgccaaggcggttactatactagtagtgctgatacgaggggtgctttcggcggagggaccaaggtggagatcaaa

SEQ ID NO: 203 - protein, 219 aa, synthetic construct
  REGION 1..219: note = synthesized
ALVMTQSPSTLSASVGDRVTINCQASEDIDTYLAWYQQKPGKAPKLLIFYASDLASGVPSRFSGSGSGTEFTLTISSLQPDDFATYYCQGGYYTSSADTRGAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 204 - protein, 112 aa, synthetic construct
  REGION 1..112: note = synthesized
ALVMTQSPSTLSASVGDRVTINCQASEDIDTYLAWYQQKPGKAPKLLIFYASDLASGVPSRFSGSGSGTEFTLTISSLQPDDFATYYCQGGYYTSSADTRGAFGGGTKVEIK

SEQ ID NO: 205 - DNA, 1347 bp, synthetic construct
  misc_feature 1..1347: note = synthesized
  source 1..1347: mol_type = other DNA
cagtcgctggtggagtctgggggaggcttggtacagcctggggggtccctgagactctcctgtgcagcctctggattctccttcagtagcaactactggatatgctgggtccgccaggctccagggaaggggctggagtggatcgcatgcatttatgttggtagtagtggtgacacttactacgcgagctccgcgaaaggccggttcaccatctccagagacaattccaagaacacgctgtatctgcaaatgaacagcctgagagccgaggacacggccgtatattactgtgcgagagatagtagtagttattatatgtttaacttgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 206 - DNA, 360 bp, synthetic construct
  misc_feature 1..360: note = synthesized
  source 1..360: mol_type = other DNA
cagtcgctggtggagtctgggggaggcttggtacagcctggggggtccctgagactctcctgtgcagcctctggattctccttcagtagcaactactggatatgctgggtccgccaggctccagggaaggggctggagtggatcgcatgcatttatgttggtagtagtggtgacacttactacgcgagctccgcgaaaggccggttcaccatctccagagacaattccaagaacacgctgtatctgcaaatgaacagcctgagagccgaggacacggccgtatattactgtgcgagagatagtagtagttattatatgtttaacttgtggggccagggaaccctggtcaccgtctcgagc

SEQ ID NO: 207 - protein, 449 aa, synthetic construct
  REGION 1..449: note = synthesized
QSLVESGGGLVQPGGSLRLSCAASGFSFSSNYWICWVRQAPGKGLEWIACIYVGSSGDTYYASSAKGRFTISRDNSKNTLYLQMNSLRAEDTAVYYCARDSSSYYMFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 208 - protein, 120 aa, synthetic construct
  REGION 1..120: note = synthesized
QSLVESGGGLVQPGGSLRLSCAASGFSFSSNYWICWVRQAPGKGLEWIACIYVGSSGDTYYASSAKGRFTISRDNSKNTLYLQMNSLRAEDTAVYYCARDSSSYYMFNLWGQGTLVTVSS

SEQ ID NO: 209 - DNA, 654 bp, synthetic construct
  misc_feature 1..654: note = synthesized
  source 1..654: mol_type = other DNA
gacatccagatgacccagtctccttccaccctgtctgcatctgtaggagacagagtcaccatcacttgccaggccagtcagagtgtttatagtaactggttatcctggtatcagcagaaaccagggaaagcccctaagctcctgatctattctgcatccactctggcatctggggtcccatcaaggttcagcggcagtggatctgggacagaattcactctcaccatcagcagcctgcagcctgatgattttgcaacttattactgcgcaggcggttacaatactgtaattgatacttttgctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 210 - DNA, 333 bp, synthetic construct
  misc_feature 1..333: note = synthesized
  source 1..333: mol_type = other DNA
gacatccagatgacccagtctccttccaccctgtctgcatctgtaggagacagagtcaccatcacttgccaggccagtcagagtgtttatagtaactggttatcctggtatcagcagaaaccagggaaagcccctaagctcctgatctattctgcatccactctggcatctggggtcccatcaaggttcagcggcagtggatctgggacagaattcactctcaccatcagcagcctgcagcctgatgattttgcaacttattactgcgcaggcggttacaatactgtaattgatacttttgctttcggcggagggaccaaggtggagatcaaa

SEQ ID NO: 211 - protein, 218 aa, synthetic construct
  REGION 1..218: note = synthesized
DIQMTQSPSTLSASVGDRVTITCQASQSVYSNWLSWYQQKPGKAPKLLIYSASTLASGVPSRFSGSGSGTEFTLTISSLQPDDFATYYCAGGYNTVIDTFAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 212 - protein, 111 aa, synthetic construct
  REGION 1..111: note = synthesized
DIQMTQSPSTLSASVGDRVTITCQASQSVYSNWLSWYQQKPGKAPKLLIYSASTLASGVPSRFSGSGSGTEFTLTISSLQPDDFATYYCAGGYNTVIDTFAFGGGTKVEIK

SEQ ID NO: 213 - DNA, 1332 bp, synthetic construct
  misc_feature 1..1332: note = synthesized
  source 1..1332: mol_type = other DNA
gaggtgcagctgttggagtctgggggaggcttggtacagcctggggggtccctgagactctcctgtgcagcctctggaatcgacttcagtagtagttactacatgtgctgggtccgccaggctccagggaaggggctggagtggatcgcatgcatttatactggtggtagtggtactcctcactacgcgagctccgcgaaaggccggttcaccatctccagagacaattccaagaacacgctgtatctgcaaatgaacagcctgagagccgaggacacggccgtatattactgtgcgagagaaggtagcctgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 214 - DNA, 345 bp, synthetic construct
  misc_feature 1..345: note = synthesized
  source 1..345: mol_type = other DNA
gaggtgcagctgttggagtctgggggaggcttggtacagcctggggggtccctgagactctcctgtgcagcctctggaatcgacttcagtagtagttactacatgtgctgggtccgccaggctccagggaaggggctggagtggatcgcatgcatttatactggtggtagtggtactcctcactacgcgagctccgcgaaaggccggttcaccatctccagagacaattccaagaacacgctgtatctgcaaatgaacagcctgagagccgaggacacggccgtatattactgtgcgagagaaggtagcctgtggggccagggaaccctggtcaccgtctcgagc

SEQ ID NO: 215 - protein, 444 aa, synthetic construct
  REGION 1..444: note = synthesized
EVQLLESGGGLVQPGGSLRLSCAASGIDFSSSYYMCWVRQAPGKGLEWIACIYTGGSGTPHYASSAKGRFTISRDNSKNTLYLQMNSLRAEDTAVYYCAREGSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 216 - protein, 115 aa, synthetic construct
  REGION 1..115: note = synthesized
EVQLLESGGGLVQPGGSLRLSCAASGIDFSSSYYMCWVRQAPGKGLEWIACIYTGGSGTPHYASSAKGRFTISRDNSKNTLYLQMNSLRAEDTAVYYCAREGSLWGQGTLVTVSS

SEQ ID NO: 217 - DNA, 654 bp, synthetic construct
  misc_feature 1..654: note = synthesized
  source 1..654: mol_type = other DNA
gacatccagatgacccagtctccttccaccctgtctgcatctgtaggagacagagtcaccatcacttgccagtccagtcagagtgtttatagtaactggttctcctggtatcagcagaaaccagggaaagcccctaagctcctgatctattctgcatccactctggcatctggggtcccatcaaggttcagcggcagtggatctgggacagaattcactctcaccatcagcagcctgcagcctgatgattttgcaacttattactgcgcaggcggttacaatactgtaactgatacttttgctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 218 - DNA, 333 bp, synthetic construct
  misc_feature 1..333: note = synthesized
  source 1..333: mol_type = other DNA
gacatccagatgacccagtctccttccaccctgtctgcatctgtaggagacagagtcaccatcacttgccagtccagtcagagtgtttatagtaactggttctcctggtatcagcagaaaccagggaaagcccctaagctcctgatctattctgcatccactctggcatctggggtcccatcaaggttcagcggcagtggatctgggacagaattcactctcaccatcagcagcctgcagcctgatgattttgcaacttattactgcgcaggcggttacaatactgtaactgatacttttgctttcggcggagggaccaaggtggagatcaaa

SEQ ID NO: 219 - protein, 218 aa, synthetic construct
  REGION 1..218: note = synthesized
DIQMTQSPSTLSASVGDRVTITCQSSQSVYSNWFSWYQQKPGKAPKLLIYSASTLASGVPSRFSGSGSGTEFTLTISSLQPDDFATYYCAGGYNTVTDTFAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - protein, 111 aa, synthetic construct
  REGION 1..111: note = synthesized
DIQMTQSPSTLSASVGDRVTITCQSSQSVYSNWFSWYQQKPGKAPKLLIYSASTLASGVPSRFSGSGSGTEFTLTISSLQPDDFATYYCAGGYNTVTDTFAFGGGTKVEIK

SEQ ID NO: 221 - DNA, 1332 bp, synthetic construct
  misc_feature 1..1332: note = synthesized
  source 1..1332: mol_type = other DNA
gaggtgcagctgttggagtctgggggaggcttggtacagcctggggggtccctgagactctcctgtgcagcctctggaatcgacttcagtaggagatactacatgtgctgggtccgccaggctccagggaaggggctggagtggatcgcatgcatgtatactggtagccgcgatactcctcactacgcgagctccgcgaaaggccggttcaccatctccagagacaattccaagaacacgctgtatctgcaaatgaacagcctgagagccgaggacacggccgtatattactgtgcgagagaaggtaacctgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 222 - DNA, 345 bp, synthetic construct
  misc_feature 1..345: note = synthesized
  source 1..345: mol_type = other DNA
gaggtgcagctgttggagtctgggggaggcttggtacagcctggggggtccctgagactctcctgtgcagcctctggaatcgacttcagtaggagatactacatgtgctgggtccgccaggctccagggaaggggctggagtggatcgcatgcatgtatactggtagccgcgatactcctcactacgcgagctccgcgaaaggccggttcaccatctccagagacaattccaagaacacgctgtatctgcaaatgaacagcctgagagccgaggacacggccgtatattactgtgcgagagaaggtaacctgtggggccagggaaccctggtcaccgtctcgagc

SEQ ID NO: 223 - protein, 444 aa, synthetic construct
  REGION 1..444: note = synthesized
EVQLLESGGGLVQPGGSLRLSCAASGIDFSRRYYMCWVRQAPGKGLEWIACMYTGSRDTPHYASSAKGRFTISRDNSKNTLYLQMNSLRAEDTAVYYCAREGNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 224 - protein, 115 aa, synthetic construct
  REGION 1..115: note = synthesized
EVQLLESGGGLVQPGGSLRLSCAASGIDFSRRYYMCWVRQAPGKGLEWIACMYTGSRDTPHYASSAKGRFTISRDNSKNTLYLQMNSLRAEDTAVYYCAREGNLWGQGTLVTVSS

SEQ ID NO: 225 - DNA, 654 bp, synthetic construct
  misc_feature 1..654: note = synthesized
  source 1..654: mol_type = other DNA
gacatccagatgacccagtctccttccaccctgtctgcatctgtaggagacagagtcaccatcacttgccagtccagtcagagtgtttatagtaactggttctcctggtatcagcagaaaccagggaaagcccctaagctcctgatctattctgcatccactctggcatctggggtcccatcaaggttcagcggcagtggatctgggacagaattcactctcaccatcagcagcctgcagcctgatgattttgcaacttattactgcgcaggcggttacaatactgttattgatacttttgctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 226 - DNA, 333 bp, synthetic construct
  misc_feature 1..333: note = synthesized
  source 1..333: mol_type = other DNA
gacatccagatgacccagtctccttccaccctgtctgcatctgtaggagacagagtcaccatcacttgccagtccagtcagagtgtttatagtaactggttctcctggtatcagcagaaaccagggaaagcccctaagctcctgatctattctgcatccactctggcatctggggtcccatcaaggttcagcggcagtggatctgggacagaattcactctcaccatcagcagcctgcagcctgatgattttgcaacttattactgcgcaggcggttacaatactgttattgatacttttgctttcggcggagggaccaaggtggagatcaaa

SEQ ID NO: 227 - protein, 218 aa, synthetic construct
  REGION 1..218: note = synthesized
DIQMTQSPSTLSASVGDRVTITCQSSQSVYSNWFSWYQQKPGKAPKLLIYSASTLASGVPSRFSGSGSGTEFTLTISSLQPDDFATYYCAGGYNTVIDTFAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 111 aa, synthetic construct
  REGION 1..111: note = synthesized
DIQMTQSPSTLSASVGDRVTITCQSSQSVYSNWFSWYQQKPGKAPKLLIYSASTLASGVPSRFSGSGSGTEFTLTISSLQPDDFATYYCAGGYNTVIDTFAFGGGTKVEIK

SEQ ID NO: 229 - DNA, 1332 bp, synthetic construct
  misc_feature 1..1332: note = synthesized
  source 1..1332: mol_type = other DNA
gaggtgcagctgttggagtctgggggaggcttggtacagcctggggggtccctgagactctcctgtgcagcctctggaatcgacttcagtaggagatactacatgtgctgggtccgccaggctccagggaaggggctggagtggatcgcatgcatatatactggtagccgcgatactcctcactacgcgagctccgcgaaaggccggttcaccatctccagagacaattccaagaacacgctgtatctgcaaatgaacagcctgagagccgaggacacggccgtatattactgtgcgagagaaggtagcctgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 230 - DNA, 345 bp, synthetic construct
  misc_feature 1..345: note = synthesized
  source 1..345: mol_type = other DNA
gaggtgcagctgttggagtctgggggaggcttggtacagcctggggggtccctgagactctcctgtgcagcctctggaatcgacttcagtaggagatactacatgtgctgggtccgccaggctccagggaaggggctggagtggatcgcatgcatatatactggtagccgcgatactcctcactacgcgagctccgcgaaaggccggttcaccatctccagagacaattccaagaacacgctgtatctgcaaatgaacagcctgagagccgaggacacggccgtatattactgtgcgagagaaggtagcctgtggggccagggaaccctggtcaccgtctcgagc

SEQ ID NO: 231 - protein, 444 aa, synthetic construct
  REGION 1..444: note = synthesized
EVQLLESGGGLVQPGGSLRLSCAASGIDFSRRYYMCWVRQAPGKGLEWIACIYTGSRDTPHYASSAKGRFTISRDNSKNTLYLQMNSLRAEDTAVYYCAREGSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 232 - protein, 115 aa, synthetic construct
  REGION 1..115: note = synthesized
EVQLLESGGGLVQPGGSLRLSCAASGIDFSRRYYMCWVRQAPGKGLEWIACIYTGSRDTPHYASSAKGRFTISRDNSKNTLYLQMNSLRAEDTAVYYCAREGSLWGQGTLVTVSS

SEQ ID NO: 233 - DNA, 651 bp, synthetic construct
  misc_feature 1..651: note = synthesized
  source 1..651: mol_type = other DNA
gacatccagatgacccagtctccttccaccctgtctgcatctgtaggagacagagtcaccatcacttgccagtccagtcagagtgtttatagtaacagcttcttatcctggtatcagcagaaaccagggaaagcccctaagctcctgatctattctgcatccgatctggcatctggggtcccatcaaggttcagcggcagtggatctgggacagaattcactctcaccatcagcagcctgcagcctgatgattttgcaacttattactgcgcaggcgtttatagtgataatacttatgttttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 234 - DNA, 330 bp, synthetic construct
  misc_feature 1..330: note = synthesized
  source 1..330: mol_type = other DNA
gacatccagatgacccagtctccttccaccctgtctgcatctgtaggagacagagtcaccatcacttgccagtccagtcagagtgtttatagtaacagcttcttatcctggtatcagcagaaaccagggaaagcccctaagctcctgatctattctgcatccgatctggcatctggggtcccatcaaggttcagcggcagtggatctgggacagaattcactctcaccatcagcagcctgcagcctgatgattttgcaacttattactgcgcaggcgtttatagtgataatacttatgttttcggcggagggaccaaggtggagatcaaa

SEQ ID NO: 235 - protein, 217 aa, synthetic construct
  REGION 1..217: note = synthesized
DIQMTQSPSTLSASVGDRVTITCQSSQSVYSNSFLSWYQQKPGKAPKLLIYSASDLASGVPSRFSGSGSGTEFTLTISSLQPDDFATYYCAGVYSDNTYVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 110 aa, synthetic construct
  REGION 1..110: note = synthesized
DIQMTQSPSTLSASVGDRVTITCQSSQSVYSNSFLSWYQQKPGKAPKLLIYSASDLASGVPSRFSGSGSGTEFTLTISSLQPDDFATYYCAGVYSDNTYVFGGGTKVEIK

SEQ ID NO: 237 - DNA, 1338 bp, synthetic construct
  misc_feature 1..1338: note = synthesized
  source 1..1338: mol_type = other DNA
gaggtgcagctggtggagtctgggggaggcttggtacagccagggccgtccctgagactctcctgtacagcttctggattctccctcagtcactactggatgacttgggtccgccaggctccagggaaggggctggagtacatcggattcattaatgttggtggtgacacatcttacgcgagctctgtgaaaggcagattcaccatctcaagagatgattccaaaagcatcgcctatctgcaaatgaacagcctgaaaaccgaggacacagccgtgtattactgtggcagaggtggtctgacttttggttgggacttgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 238 - DNA, 351 bp, synthetic construct
  misc_feature 1..351: note = synthesized
  source 1..351: mol_type = other DNA
gaggtgcagctggtggagtctgggggaggcttggtacagccagggccgtccctgagactctcctgtacagcttctggattctccctcagtcactactggatgacttgggtccgccaggctccagggaaggggctggagtacatcggattcattaatgttggtggtgacacatcttacgcgagctctgtgaaaggcagattcaccatctcaagagatgattccaaaagcatcgcctatctgcaaatgaacagcctgaaaaccgaggacacagccgtgtattactgtggcagaggtggtctgacttttggttgggacttgtggggccagggaaccctggtcaccgtctcgagc

SEQ ID NO: 239 - protein, 446 aa, synthetic construct
  REGION 1..446: note = synthesized
EVQLVESGGGLVQPGPSLRLSCTASGFSLSHYWMTWVRQAPGKGLEYIGFINVGGDTSYASSVKGRFTISRDDSKSIAYLQMNSLKTEDTAVYYCGRGGLTFGWDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 240 - protein, 117 aa, synthetic construct
  REGION 1..117: note = synthesized
EVQLVESGGGLVQPGPSLRLSCTASGFSLSHYWMTWVRQAPGKGLEYIGFINVGGDTSYASSVKGRFTISRDDSKSIAYLQMNSLKTEDTAVYYCGRGGLTFGWDLWGQGTLVTVSS

SEQ ID NO: 241 - DNA, 654 bp, synthetic construct
  misc_feature 1..654: note = synthesized
  source 1..654: mol_type = other DNA
gacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 242 - DNA, 333 bp, synthetic construct
  misc_feature 1..333: note = synthesized
  source 1..333: mol_type = other DNA
gacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaa

SEQ ID NO: 243 - protein, 218 aa, synthetic construct
  REGION 1..218: note = synthesized
DVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 111 aa, synthetic construct
  REGION 1..111: note = synthesized
DVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 245 - DNA, 1332 bp, synthetic construct
  misc_feature 1..1332: note = synthesized
  source 1..1332: mol_type = other DNA
cggtcgctggtggagtctgggggaggcttggtccagcctggggggtccctgagactctcctgtacagcctctggattcaccatcagtagctaccacatgcagtgggtccgccaggctccagggaaggggctggagtacatcggaaccattagtagtggtggtaatgtatactacgcgagctccgcgagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 246 - DNA, 345 bp, synthetic construct
  misc_feature 1..345: note = synthesized
  source 1..345: mol_type = other DNA
cggtcgctggtggagtctgggggaggcttggtccagcctggggggtccctgagactctcctgtacagcctctggattcaccatcagtagctaccacatgcagtgggtccgccaggctccagggaaggggctggagtacatcggaaccattagtagtggtggtaatgtatactacgcgagctccgcgagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcgagc

SEQ ID NO: 247 - protein, 444 aa, synthetic construct
  REGION 1..444: note = synthesized
RSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 248 - protein, 115 aa, synthetic construct
  REGION 1..115: note = synthesized
RSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSS

SEQ ID NO: 249 - DNA, 654 bp, synthetic construct
  misc_feature 1..654: note = synthesized
  source 1..654: mol_type = other DNA
gacatccagatgacccagtctccttccaccctgtctgcatctgtaggagacagagtcaccatcacttgccaggccagtcagagcattagtactaacttgaactggtatcagcagaaaccagggaaagcccctaagctcctgatctattatgcatccagtctggcatctggggtcccatcaaggttcagcggcagtggatctgggacagaattcactctcaccatcagcagcctgcagcctgatgattttgcaacttattactgcctaggtgtttggaattattggggtgatgatggtactgatttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 250 - DNA, 333 bp, synthetic construct
  misc_feature 1..333: note = synthesized
  source 1..333: mol_type = other DNA
gacatccagatgacccagtctccttccaccctgtctgcatctgtaggagacagagtcaccatcacttgccaggccagtcagagcattagtactaacttgaactggtatcagcagaaaccagggaaagcccctaagctcctgatctattatgcatccagtctggcatctggggtcccatcaaggttcagcggcagtggatctgggacagaattcactctcaccatcagcagcctgcagcctgatgattttgcaacttattactgcctaggtgtttggaattattggggtgatgatggtactgatttcggcggagggaccaaggtggagatcaaa

SEQ ID NO: 251 - protein, 218 aa, synthetic construct
  REGION 1..218: note = synthesized
DIQMTQSPSTLSASVGDRVTITCQASQSISTNLNWYQQKPGKAPKLLIYYASSLASGVPSRFSGSGSGTEFTLTISSLQPDDFATYYCLGVWNYWGDDGTDFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 252 - protein, 111 aa, synthetic construct
  REGION 1..111: note = synthesized
DIQMTQSPSTLSASVGDRVTITCQASQSISTNLNWYQQKPGKAPKLLIYYASSLASGVPSRFSGSGSGTEFTLTISSLQPDDFATYYCLGVWNYWGDDGTDFGGGTKVEIK

SEQ ID NO: 253 - DNA, 1332 bp, synthetic construct
  misc_feature 1..1332: note = synthesized
  source 1..1332: mol_type = other DNA
gaggtgcagctggtggagtctgggggaggcttggtccagcctggggggtccctgagactctcctgtgcagcctctggattctccctcagtagctactacatgagctgggtccgccaggctccagggaaggggctggagtggatcggaagtatttatggtagtggtgccgcatactacgcgagctccgctaaaggcagattcaccatctccagagacaattccaagaacacgctgtatcttcaaatgaacagcctgagagccgaggacacggctgtgtattactgtgcgagagatcctattaacaatgccatgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 254 - DNA, 345 bp, synthetic construct
  misc_feature 1..345: note = synthesized
  source 1..345: mol_type = other DNA
gaggtgcagctggtggagtctgggggaggcttggtccagcctggggggtccctgagactctcctgtgcagcctctggattctccctcagtagctactacatgagctgggtccgccaggctccagggaaggggctggagtggatcggaagtatttatggtagtggtgccgcatactacgcgagctccgctaaaggcagattcaccatctccagagacaattccaagaacacgctgtatcttcaaatgaacagcctgagagccgaggacacggctgtgtattactgtgcgagagatcctattaacaatgccatgtggggccagggaaccctggtcaccgtctcgagc

SEQ ID NO: 255 - protein, 444 aa, synthetic construct
  REGION 1..444: note = synthesized
EVQLVESGGGLVQPGGSLRLSCAASGFSLSSYYMSWVRQAPGKGLEWIGSIYGSGAAYYASSAKGRFTISRDNSKNTLYLQMNSLRAEDTAVYYCARDPINNAMWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 256 - protein, 115 aa, synthetic construct
  REGION 1..115: note = synthesized
EVQLVESGGGLVQPGGSLRLSCAASGFSLSSYYMSWVRQAPGKGLEWIGSIYGSGAAYYASSAKGRFTISRDNSKNTLYLQMNSLRAEDTAVYYCARDPINNAMWGQGTLVTVSS

SEQ ID NO: 257 - DNA, 657 bp, synthetic construct
  misc_feature 1..657: note = synthesized
  source 1..657: mol_type = other DNA
gcccttgtgatgacccagtctccttccaccctgtctgcatctgtaggagacagagtcaccatcaattgccaggccagtgaggacattgataactatttagcctggtatcagcagaaaccagggaaagcccctaagctcctgatcttttatgcatccgatctggcatctggggtcccatcaaggttcagcggcagtggatctgggacagaattcactctcaccatcagcagcctgcagcctgatgattttgcaacttattactgccaaggcggttactatactagtagtactgatacgaggggtgctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 258 - DNA, 336 bp, synthetic construct
  misc_feature 1..336: note = synthesized
  source 1..336: mol_type = other DNA
gcccttgtgatgacccagtctccttccaccctgtctgcatctgtaggagacagagtcaccatcaattgccaggccagtgaggacattgataactatttagcctggtatcagcagaaaccagggaaagcccctaagctcctgatcttttatgcatccgatctggcatctggggtcccatcaaggttcagcggcagtggatctgggacagaattcactctcaccatcagcagcctgcagcctgatgattttgcaacttattactgccaaggcggttactatactagtagtactgatacgaggggtgctttcggcggagggaccaaggtggagatcaaa

SEQ ID NO: 259 - protein, 219 aa, synthetic construct
  REGION 1..219: note = synthesized
ALVMTQSPSTLSASVGDRVTINCQASEDIDNYLAWYQQKPGKAPKLLIFYASDLASGVPSRFSGSGSGTEFTLTISSLQPDDFATYYCQGGYYTSSTDTRGAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 260 - protein, 112 aa, synthetic construct
  REGION 1..112: note = synthesized
ALVMTQSPSTLSASVGDRVTINCQASEDIDNYLAWYQQKPGKAPKLLIFYASDLASGVPSRFSGSGSGTEFTLTISSLQPDDFATYYCQGGYYTSSTDTRGAFGGGTKVEIK

SEQ ID NO: 261 - DNA, 1347 bp, synthetic construct
  misc_feature 1..1347: note = synthesized
  source 1..1347: mol_type = other DNA
cagtcgctggtggagtctgggggaggcttggtacagcctggggggtccctgagactctcctgtgcagcctctggattctccttcactagcagctactacatgtgctgggtccgccaggctccagggaaggggctggagtggatcgcatgcatttatactggtagtagtggtagcacttactacgcgaactccgcgaaaggccggttcaccatctccagagacaattccaagaacacgctgtatctgcaaatgaacagcctgagagccgaggacacggccgtatattactgtgcgagagatagtagtagttattatatgtttaacttgtggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 262 - DNA, 360 bp, synthetic construct
  misc_feature 1..360: note = synthesized
  source 1..360: mol_type = other DNA
cagtcgctggtggagtctgggggaggcttggtacagcctggggggtccctgagactctcctgtgcagcctctggattctccttcactagcagctactacatgtgctgggtccgccaggctccagggaaggggctggagtggatcgcatgcatttatactggtagtagtggtagcacttactacgcgaactccgcgaaaggccggttcaccatctccagagacaattccaagaacacgctgtatctgcaaatgaacagcctgagagccgaggacacggccgtatattactgtgcgagagatagtagtagttattatatgtttaacttgtggggccagggaaccctggtcaccgtctcgagc

SEQ ID NO: 263 - protein, 449 aa, synthetic construct
  REGION 1..449: note = synthesized
QSLVESGGGLVQPGGSLRLSCAASGFSFTSSYYMCWVRQAPGKGLEWIACIYTGSSGSTYYANSAKGRFTISRDNSKNTLYLQMNSLRAEDTAVYYCARDSSSYYMFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 264 - protein, 120 aa, synthetic construct
  REGION 1..120: note = synthesized
QSLVESGGGLVQPGGSLRLSCAASGFSFTSSYYMCWVRQAPGKGLEWIACIYTGSSGSTYYANSAKGRFTISRDNSKNTLYLQMNSLRAEDTAVYYCARDSSSYYMFNLWGQGTLVTVSS

SEQ ID NO: 265 - DNA, 654 bp, synthetic construct
  misc_feature 1..654: note = synthesized
  source 1..654: mol_type = other DNA
gacatccagatgacccagtctccatcctccctgtctgcatctgtaggagacagagtcaccatcacttgccagtccagtcagagtgtttataataacaatgctttagcctggtatcagcagaaaccagggaaagttcctaagctcctgatctatttggcttccactctggcatctggggtcccatctcggttcagtggcagtggatctgggacagatttcactctcaccatcagcagcctgcagcctgaagatgttgcaacttattactgtctaggtgtttataatgatgatgttgataatggtttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 266 - DNA, 333 bp, synthetic construct
  misc_feature 1..333: note = synthesized
  source 1..333: mol_type = other DNA
gacatccagatgacccagtctccatcctccctgtctgcatctgtaggagacagagtcaccatcacttgccagtccagtcagagtgtttataataacaatgctttagcctggtatcagcagaaaccagggaaagttcctaagctcctgatctatttggcttccactctggcatctggggtcccatctcggttcagtggcagtggatctgggacagatttcactctcaccatcagcagcctgcagcctgaagatgttgcaacttattactgtctaggtgtttataatgatgatgttgataatggtttcggcggagggaccaaggtggagatcaaa

SEQ ID NO: 267 - protein, 218 aa, synthetic construct
  REGION 1..218: note = synthesized
DIQMTQSPSSLSASVGDRVTITCQSSQSVYNNNALAWYQQKPGKVPKLLIYLASTLASGVPSRFSGSGSGTDFTLTISSLQPEDVATYYCLGVYNDDVDNGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 268 - protein, 111 aa, synthetic construct
  REGION 1..111: note = synthesized
DIQMTQSPSSLSASVGDRVTITCQSSQSVYNNNALAWYQQKPGKVPKLLIYLASTLASGVPSRFSGSGSGTDFTLTISSLQPEDVATYYCLGVYNDDVDNGFGGGTKVEIK

SEQ ID NO: 269 - DNA, 1341 bp, synthetic construct
  misc_feature 1..1341: note = synthesized
  source 1..1341: mol_type = other DNA
gaggtgcagctggtggagtctgggggaggcttggtccagcctggggggtccctgagactctcctgtgcagcctctggattctccctcaataacaatgcaataagctgggtccgccaggctccagggaaggggctggagtgggttgcaatcatacaaaatactggtaccacagactacgcgaggtccgctaaaggcagattcaccatctccagagacaattccaagaacacgctgtatcttcaaatgaacagcctgagagccgaggacacggctgtgtattactgtgcgagagggtatggttttgagtcggagcttgtcatctggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 270 - DNA, 354 bp, synthetic construct
  misc_feature 1..354: note = synthesized
  source 1..354: mol_type = other DNA
gaggtgcagctggtggagtctgggggaggcttggtccagcctggggggtccctgagactctcctgtgcagcctctggattctccctcaataacaatgcaataagctgggtccgccaggctccagggaaggggctggagtgggttgcaatcatacaaaatactggtaccacagactacgcgaggtccgctaaaggcagattcaccatctccagagacaattccaagaacacgctgtatcttcaaatgaacagcctgagagccgaggacacggctgtgtattactgtgcgagagggtatggttttgagtcggagcttgtcatctggggccagggaaccctggtcaccgtctcgagc

SEQ ID NO: 271 - protein, 447 aa, synthetic construct
  REGION 1..447: note = synthesized
EVQLVESGGGLVQPGGSLRLSCAASGFSLNNNAISWVRQAPGKGLEWVAIIQNTGTTDYARSAKGRFTISRDNSKNTLYLQMNSLRAEDTAVYYCARGYGFESELVIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 272 - protein, 118 aa, synthetic construct
  REGION 1..118: note = synthesized
EVQLVESGGGLVQPGGSLRLSCAASGFSLNNNAISWVRQAPGKGLEWVAIIQNTGTTDYARSAKGRFTISRDNSKNTLYLQMNSLRAEDTAVYYCARGYGFESELVIWGQGTLVTVSS

(19) United States
(12) Patent Application Publication
(10) Pub. No.: US 2024/0067741 A1
(43) Pub. Date: Feb. 29, 2024

(54) ANTI-4-1BB ANTIBODIES AND METHODS OF MAKING AND USING THEREOF

(71) Applicants: SYSTIMMUNE, INC., Redmond, WA (US); BAILI-BIO (CHENGDU) PHARMACEUTICAL CO., LTD., Chengdu (CN)

(72) Inventors: Ole OLSEN, Everett, WA (US); Dong XIA, Redmond, WA (US); David JELLYMAN, Duvall, WA (US); Brian KOVACEVICH, Snohomish, WA (US); Bill BRADY, Bothell, MA (US); Blair RENSHAW, Renton, WA (US); Zeren GAO, Redmond, WA (US); Yi ZHU, Chengdu (CN)

(21) Appl. No.: 18/358,834

(22) Filed: Jul. 25, 2023

Related U.S. Application Data
(62) Division of application No. 16/615,119, filed on Nov. 19, 2019.

(51) Int. Cl. C07K 16/28; A61K 39/395; A61K 45/06

(52) U.S. Cl. CPC C07K 16/2878; A61K 39/3955; A61K 45/06; C07K 16/2803; A61K 2039/505

(57) ABSTRACT

The application provides anti-4-1BB monoclonal antibodies, antigen binding portions thereof, therapeutic compositions thereof and/or nucleic acid encoding the same, and their use to upregulate the function of T-cells to enhance cell-mediated immune responses in the treatment of cancer and other T-cell dysfunctional disorders.

### CROSS REFERENCE TO RELATED APPLICATIONS

This application is a divisional application of U.S. application Ser. No. 16/615,119, filed Nov. 19, 2019, which claims the benefit of U.S. Provisional Patent Application No. 62/551,065, filed Aug. 28, 2017, U.S. Provisional Patent Application No. 62/551,032 filed Aug. 28, 2017, U.S. Provisional Patent Application No. 62/524,554 filed Jun. 25, 2017, U.S. Provisional Patent Application No. 62/524,557 filed Jun. 25, 2017, U.S. Provisional Patent Application No. 62/524,558 filed Jun. 25, 2017, U.S. Provisional Patent Application No. 62/545,603 filed Aug. 15, 2017, U.S. Provisional Patent Application No. 62/551,032 filed Aug. 28, 2017, and U.S. Provisional Patent Application No. 62/551,035 filed Aug. 28, 2017, the entire disclosures of which are expressly incorporated by reference herein.

### SEQUENCE LISTING

The content of the ASCII text file of the sequence listing named “028_ST25”, which is 400 kb in size was created on Aug. 16, 2018 and electronically submitted via EFS-Web on Nov. 19, 2019, is incorporated herein by reference in its entirety.

### TECHNICAL FIELD

The present disclosure generally relates to the technical field of antibodies, and more particularly relates to making and using anti-4-1BB antibodies, and applications of the antibodies in cancer treatment and therapy.

### BACKGROUND

Cancer is a major health problem across the world. In the United States alone it is estimated that in 2016 there were 1,685,210 new cases of cancer diagnosed and 595,690 deaths from the disease (http://www.cancer.gov). As such, any pharmaceutical agent that can reduce the severity or mortality rate from cancer is desirable.

In the immune system, resting T-cells can be activated to respond to antigen through a primary signal delivered through the T-cell receptor (TCR) by foreign antigen peptides presented by antigen-presenting cells (APCs). In addition to this primary signal, there are secondary positive and negative co-stimulatory signals that further influence the response of the T-cells. A secondary positive signal is required for full T-cell activation ((Lafferty et al., Ausl. J. Exp. Biol. Med. Sci. 53: 27-42 (1975)). Negative secondary signals can result in T-cell suppression and tolerance.

4-1BB is a co-stimulatory immune checkpoint molecule with the ability to active T cells. 4-1BB, also named as CD137, tumour necrosis factor receptor superfamily member 9 (TNFRSF9), and induced by lymphocyte activation (ILA), is a member of the tumour necrosis factor (TNF) receptor family. It has been reported that Crosslinking of CD137 enhances T cell proliferation, IL-2 secretion, survival and cytolytic activity. Further, it can enhance immune activity to eliminate tumours in mice.

Pfizer's Utomilumab (PF-05082566) targets 4-1BB, when in combination with Merck's Keytruda, stimulates a more intense immune system attack on cancers in a small clinical trial (https://www.reuters.com/article/us-health-cancer-pfizer-immunotherapy-idUSKCNOY92W2). And seven clinical trials of PF-05082566 are ongoing. (www.clinicaltrials.gov).

### SUMMARY

The present disclosure provides, among others, anti-4-1BB monoclonal antibodies, antigen binding portions thereof, therapeutic compositions thereof and/or nucleic acid encoding the same, and their use to upregulate the function of T-cells to enhance cell-mediated immune responses in the treatment of cancer and other T-cell dysfunctional disorders.

In one embodiment, an isolated monoclonal antibody (mAb) or antigen-binding fragment that binds specifically to human or cynomolgus 4-1BB is provided. In one embodiment, the isolated mAb or antigen-binding fragment include an amino acid sequence having a percentage homology with SEQ ID NO:4, SEQ ID NO:8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:68, SEQ ID NO:72, SEQ ID NO:76, SEQ ID NO:80, SEQ ID NO:84, SEQ ID NO:88, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:128, SEQ ID NO:132, SEQ ID NO:136, SEQ ID NO:140, SEQ ID NO:144, SEQ ID NO:148, SEQ ID NO:152, SEQ ID NO:156, SEQ ID NO:160, SEQ ID NO:164, SEQ ID NO:168, SEQ ID NO:172, SEQ ID NO:176, SEQ ID NO:180, SEQ ID NO:184, SEQ ID NO:188, SEQ ID NO:192, SEQ ID NO:196, SEQ ID NO:200, SEQ ID NO:204, SEQ ID NO:208, SEQ ID NO:212, SEQ ID NO:216, SEQ ID NO:220, SEQ ID NO:224, SEQ ID NO:228, SEQ ID NO:232, SEQ ID NO:236, SEQ ID NO:240, SEQ ID NO:244, SEQ ID NO:248, SEQ ID NO:252, SEQ ID NO:256, SEQ ID NO:260, SEQ ID NO:264, SEQ ID NO:268, or SEQ ID NO:272. The percentage homology is not less than 70%, 80%, 90%, 95%, 98%, or 99%.

In one embodiment, the isolated mAb or antigen-binding fragment, has a binding affinity to human or cynomolgus 4-1BB with a Kd not greater than 30 nM, 40 nM, 50 nM, 60 nM, 70 nM, 80 nM, 90 nM, or 100 nM.

In one embodiment, the isolated mAb or antigen-binding fragment exhibits one or more functional properties such as, without limitation, high affinity binding to human or cynomolgus 4-1BB, inhibiting human or cynomolgus 4-1BB activity, induction of apoptosis, regulation of EGFR signalling pathway, upregulation of EMT genes, enhancing T cell activation, stimulating antibody response, reversing the suppressive function of an immunosuppressive cell, or a combination thereof. In one embodiment, the immunosuppressive cell comprises a regulatory cell. In one embodiment, the isolated mAb or antigen-binding fragment enhances T-cell activation via mechanisms or pathways including T-cell proliferation, IFN-γ and/or IL-2 secretion, or a combination thereof.

In one embodiment, the isolated mAb or antigen-binding fragment comprises a human framework region. In one embodiment, the isolated mAb or antigen-binding fragment is a humanized antibody, a chimeric antibody, or a recombinant antibody.

In one embodiment, the isolated mAb or antigen-binding fragment is an IgG. In one embodiment, the antigen-binding fragment is a Fv, a Fab, a F(ab′)2, a scFV or a scFV2 fragment. In one embodiment, the isolated mAb is a bispecific antibody, tri-specific antibody, or multi-specific antibody.

In one embodiment, the isolated mAb or antigen-binding fragment may include an IgG1 heavy chain. In one embodiment, the IgG1 heavy chain comprises an amino acid sequence having a percentage homology with SEQ ID NO:7, SEQ ID NO:15, SEQ ID NO:23, SEQ ID NO:31, SEQ ID NO:39, SEQ ID NO:47, SEQ ID NO:55, SEQ ID NO:63, SEQ ID NO:71, SEQ ID NO:79, SEQ ID NO:87, SEQ ID NO:95, SEQ ID NO:103, SEQ ID NO:111, SEQ ID NO:119, SEQ ID NO:127, SEQ ID NO:135, SEQ ID NO:143, SEQ ID NO:151, SEQ ID NO:159, SEQ ID NO:167, SEQ ID NO:175, SEQ ID NO:183, SEQ ID NO:191, SEQ ID NO:199, SEQ ID NO:207, SEQ ID NO:215, SEQ ID NO:223, SEQ ID NO:231, SEQ ID NO:239, SEQ ID NO:247, SEQ ID NO:255, SEQ ID NO:263, or SEQ ID NO:271. The percentage homology is not less than 70%, 80%, 90%, 95%, 98%, or 99%.

In one embodiment, the isolated mAb or antigen-binding fragment may include a kappa light chain. In one embodiment, the kappa light chain comprises an amino acid sequence having a percentage homology with SEQ ID NO:3, SEQ ID NO:11, SEQ ID NO:19, SEQ ID NO:27, SEQ ID NO:35, SEQ ID NO:43, SEQ ID NO:51, SEQ ID NO:59, SEQ ID NO:67, SEQ ID NO:75, SEQ ID NO:83, SEQ ID NO:91, SEQ ID NO:99, SEQ ID NO:107, SEQ ID NO:115, SEQ ID NO:123, SEQ ID NO:131, SEQ ID NO:139, SEQ ID NO:147, SEQ ID NO:155, SEQ ID NO:163, SEQ ID NO:171, SEQ ID NO:179, SEQ ID NO:187, SEQ ID NO:195, SEQ ID NO:203, SEQ ID NO:211, SEQ ID NO:219, SEQ ID NO:1 SEQ ID NO:227, SEQ ID NO:235, SEQ ID NO:243, SEQ ID NO:257, or SEQ ID NO:263. The percentage homology is not less than 70%, 80%, 90%, 95%, 98%, or 99%.

In one embodiment, the isolated mAb or antigen-binding fragment may include a variable light chain. In one embodiment, the variable light chain comprises an amino acid sequence having a percentage homology with SEQ ID NO:4, SEQ ID NO:12, SEQ ID NO:20, SEQ ID NO:28, SEQ ID NO:36, SEQ ID NO:44, SEQ ID NO:52, SEQ ID NO:60, SEQ ID NO: 68, SEQ ID NO:76, SEQ ID NO:84, SEQ ID NO:92, SEQ ID NO:100, SEQ ID NO:108, SEQ ID NO:116, SEQ ID NO:124, SEQ ID NO:132, SEQ ID NO:140, SEQ ID NO:148, SEQ ID NO:156, SEQ ID NO:164, SEQ ID NO:172, SEQ ID NO:180, SEQ ID NO:188, SEQ ID NO:196, SEQ ID NO:204, SEQ ID NO:212, SEQ ID NO:220, SEQ ID NO:228, SEQ ID NO:236, SEQ ID NO:244, SEQ ID NO:252, SEQ ID NO:260, or SEQ ID NO:268. The percentage homology is not less than 70%, 80%, 90%, 95%, 98%, or 99%.

In one embodiment, the isolated mAb or antigen-binding fragment may include a variable heavy chain. In one embodiment, the variable heavy chain comprises an amino acid sequence having a percentage homology with SEQ ID NO:8, SEQ ID NO:16, SEQ ID NO:24, SEQ ID NO:32, SEQ ID NO:40, SEQ ID NO:48, SEQ ID NO:56, SEQ ID NO:64, SEQ ID NO:72, SEQ ID NO:80, SEQ ID NO:88, SEQ ID NO:96, SEQ ID NO:104, SEQ ID NO:112, SEQ ID NO:120, SEQ ID NO:128, SEQ ID NO:136, SEQ ID NO:144, SEQ ID NO:152, SEQ ID NO:160, SEQ ID NO:168, SEQ ID NO:176, SEQ ID NO:184, SEQ ID NO:192, SEQ ID NO:200, SEQ ID NO:208, SEQ ID NO:216, SEQ ID NO:224, SEQ ID NO:232, SEQ ID NO:240, SEQ ID NO:248, SEQ ID NO:256, SEQ ID NO:264, or SEQ ID NO:272. The percentage homology is not less than 70%, 80%, 90%, 95%, 98%, or 99%.

The application further provides isolated nucleic acids encoding at least a portion of the isolated mAb or antigen-binding fragment disclosed herein. In one embodiments, the nucleic acid encodes an IgG1 heavy chain that includes an amino acid sequence having a percentage homology with SEQ ID NO:7, SEQ ID NO:15, SEQ ID NO:23, SEQ ID NO:31, SEQ ID NO:39, SEQ ID NO:47, SEQ ID NO:55, SEQ ID NO:63, SEQ ID NO:71, SEQ ID NO:79, SEQ ID NO:87, SEQ ID NO:95, SEQ ID NO:103, SEQ ID NO:111, SEQ ID NO:119, SEQ ID NO:127, SEQ ID NO:135, SEQ ID NO:143, SEQ ID NO:151, SEQ ID NO:159, SEQ ID NO:167, SEQ ID NO:175, SEQ ID NO:183, SEQ ID NO:191, SEQ ID NO:199, SEQ ID NO:207, SEQ ID NO:215, SEQ ID NO:223, SEQ ID NO:231, SEQ ID NO:239, SEQ ID NO:247, SEQ ID NO:255, SEQ ID NO:263, or SEQ ID NO:271. In one embodiment, the nucleic acid encodes a kappa light chain that comprises an amino acid sequence having a percentage homology with SEQ ID NO:3, SEQ ID NO:11, SEQ ID NO:19, SEQ ID NO:27, SEQ ID NO:35, SEQ ID NO:43, SEQ ID NO:51, SEQ ID NO:59, SEQ ID NO:67, SEQ ID NO:75, SEQ ID NO:83, SEQ ID NO:91, SEQ ID NO:99, SEQ ID NO:107, SEQ ID NO:115, SEQ ID NO:123, SEQ ID NO:131, SEQ ID NO:139, SEQ ID NO:147, SEQ ID NO:155, SEQ ID NO:163, SEQ ID NO:171, SEQ ID NO:179, SEQ ID NO:187, SEQ ID NO:195, SEQ ID NO:203, SEQ ID NO:211, SEQ ID NO:219, SEQ ID NO:1 SEQ ID NO:227, SEQ ID NO:235, SEQ ID NO:243, SEQ ID NO:257, or SEQ ID NO:263. In one embodiment, the nucleic acid encodes a variable light chain that comprises an amino acid sequence having a percentage homology with SEQ ID NO:4, SEQ ID NO:12, SEQ ID NO:20, SEQ ID NO:28, SEQ ID NO:36, SEQ ID NO:44, SEQ ID NO:52, SEQ ID NO:60, SEQ ID NO: 68, SEQ ID NO:76, SEQ ID NO:84, SEQ ID NO:92, SEQ ID NO:100, SEQ ID NO:108, SEQ ID NO:116, SEQ ID NO:124, SEQ ID NO:132, SEQ ID NO:140, SEQ ID NO:148, SEQ ID NO:156, SEQ ID NO:164, SEQ ID NO:172, SEQ ID NO:180, SEQ ID NO:188, SEQ ID NO:196, SEQ ID NO:204, SEQ ID NO:212, SEQ ID NO:220, SEQ ID NO:228, SEQ ID NO:236, SEQ ID NO:244, SEQ ID NO:252, SEQ ID NO:260, or SEQ ID NO:268. In one embodiment, the nucleic acid encodes a variable heavy chain that comprises an amino acid sequence having a percentage homology with SEQ ID NO:8, SEQ ID NO:16, SEQ ID NO:24, SEQ ID NO:32, SEQ ID NO:40, SEQ ID NO:48, SEQ ID NO:56, SEQ ID NO:64, SEQ ID NO:72, SEQ ID NO:80, SEQ ID NO:88, SEQ ID NO:96, SEQ ID NO:104, SEQ ID NO:112, SEQ ID NO:120, SEQ ID NO:128, SEQ ID NO:136, SEQ ID NO:144, SEQ ID NO:152, SEQ ID NO:160, SEQ ID NO:168, SEQ ID NO:176, SEQ ID NO:184, SEQ ID NO:192, SEQ ID NO:200, SEQ ID NO:208, SEQ ID NO:216, SEQ ID NO:224, SEQ ID NO:232, SEQ ID NO:240, SEQ ID NO:248, SEQ ID NO:256, SEQ ID NO:264, or SEQ ID NO:272. The percentage homology is not less than 70%, 80%, 90%, 95%, 98%, or 99%.

In one embodiment, an expression vector is provided comprising at least one of the isolated nucleic acids described infra. In one embodiment, the expression vector is expressible in a cell.

In one embodiment, a host cell is provided, comprising at least one of the nucleic acids that is described infra. In one embodiment, a host cell is provided, comprising the expression vector described infra. In one embodiment, the host cell can be a prokaryotic cell or a eukaryotic cell.

The application further provides methods for producing the isolated mAb or antigen-binding fragment disclosed herein. In one embodiment, the method uses the host cell described above. In one embodiment, the method includes the steps of providing a host cell that contains an expression vector expressible in the host cell, wherein the expression vector comprises at least one of the nucleic acids that is described infra, and culturing the host cells to produce an antibody by the expression of the nucleic acids.

The application further provides an immuno-conjugate that comprises a drug unit or an imaging agent linked to an isolated mAb or antigen-binding fragment with the sequences disclosed herein through a linker. The linker may be cleavable or noncleavable. In one embodiment, the linker is a chemical linker. In one embodiment, the linker comprises a covalent bond such as an ester bond, an ether bond, an amine bond, an amide bond, a disulphide bond, an imide bond, a sulfone bond, a phosphate bond, a phosphorus ester bond, a peptide bond, a hydrazone bond or a combination thereof. In one embodiment, the linker comprises a hydrophobic poly(ethylene glycol) linker. In one embodiment, the linker comprises a peptide bond.

In one embodiment, the drug unit in the immuno-conjugate comprises a chemotherapeutic agent, a growth inhibitory agent, a cytotoxic agent from class of calicheamicin, an antimitotic agent, a toxin, a radioactive isotope, a therapeutic agent, or a combination thereof. In one embodiment, the therapeutic agent comprises an antibody, an enzyme, or a combination thereof. In one embodiment, the drug unit comprises a calicheamicin, ozogamicin, monomethyl auristatin E, emtansine, a derivative or a combination thereof.

In one embodiment, the drug unit is selected from a cytotoxic agent, an immune regulatory reagent, an imaging agent or a combination thereof. In one embodiment, the cytotoxic agent is selected from a growth inhibitory agent or a chemotherapeutic agent from a class of tubulin binders, DNA intercalators, DNA alkylators, enzyme inhibitors, immune modulators, antimetabolite agents, radioactive isotopes, or a combination thereof. In one embodiment, the cytotoxic agent is selected from a calicheamicin, ozogamicin, monomethyl auristatin E, emtansine, a derivative or a combination thereof. In one embodiment, the immune regulatory reagents activate or suppress immune cells, T cell, NK cell, B cell, macrophage, or dendritic cell.

In one embodiment, the imaging agent may be a radionuclide, a florescent agent, a quantum dots, or a combination thereof.

The application further provides pharmaceutical compositions. In one embodiment, a pharmaceutical composition comprises the isolated mAb or antigen-binding disclosed herein and a pharmaceutically acceptable carrier. In one embodiment, a pharmaceutical composition comprises the immuno-conjugate and pharmaceutically acceptable carrier.

In a further aspect, the application provides methods for treating cancer. In one embodiment, the method comprising administering to the subject an effective amount of the isolated mAb or antigen-binding fragment with the sequences disclosed herein. In one embodiment, the method includes directly injecting into the tumour site an effective amount of the monoclonal antibodies, the antigen-binding fragment thereof, and the immuno-conjugates and disclosed herein.

In some embodiments of the disclosure, the cancer has cells that express 4-1BB. Example cancer can be treated using the disclosed mAbs or their antigen-binding fragments include without limitation breast cancer, colorectal cancer, pancreatic cancer, head and neck cancer, melanoma, ovarian cancer, prostate cancer, non-small lung cell cancer, glioma, esophageal cancer, nasopharyngeal cancer, anal cancer, rectal cancer, gastric cancer, bladder cancer, cervical cancer, or brain cancer.

In one embodiment, the method further includes co-administering an effective amount of a therapeutic agent. In one embodiment, the therapeutic agent can include an antibody, a chemotherapy agent, an enzyme, or a combination thereof. In one embodiment, the therapeutic agent may be capecitabine, cisplatin, trastuzumab, fulvestrant, tamoxifen, letrozole, exemestane, anastrozole, aminoglutethimide, testolactone, vorozole, formestane, fadrozole, letrozole, erlotinib, lafatinib, dasatinib, gefitinib, imatinib, pazopinib, lapatinib, sunitinib, nilotinib, sorafenib, nab-palitaxel, calicheamicin, antimitotic agent, monomethyl auristatin E, emtansine, ozogamicin, or a derivative or a combination thereof.

The subject receiving the treatment may be a human. In one embodiment, a solution is provided that comprises an effective concentration of the isolated mAb or an antigen-binding disclosed herein, wherein the solution is blood plasma in a subject.

Still other embodiments will become readily apparent to those skilled in the art from the following detailed description, wherein are described embodiments by way of illustrating the best mode contemplated. As will be realized, other and different embodiments are possible and the embodiments' several details are capable of modifications in various obvious respects, all without departing from their spirit and the scope. Accordingly, the drawings and detailed description are to be regarded as illustrative in nature and not as restrictive.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 Immunization schedule for rabbit cohorts no. 1 and 2 with human 41BB or cynomolgus 41BB transiently transfected HEK293 cells.

FIG. 2 41BB-specific rabbit IgG antibodies in B cell culture supernatant enhance activation of anti-CD3 activated human T cells and corresponding binding of chimeric rabbit/human IgG 41BB-specific antibodies rescued from the parental B cell culture wells to recombinant human and cynomolgus 41BB protein.

FIG. 3 Chimeric rabbit/human IgG antibodies specific for 41BB enhance T cell activation, proliferation, and gamma interferon secretion.

FIG. 4. Octet analysis of dissociation rate for humanized 41BB-specific IgG antibodies.

FIG. 5. Humanized 41BB-specific IgG antibodies enhance T cell activation, proliferation, and gamma interferon secretion.

FIG. 6. Serum from rabbits immunized with human or cynomolgus 41BB expressing HEK293's analyzed for 41BB-specific IgG antibody binding to parental CHO or human or cynomolgus 41BB-transfected CHO cells.[

FIG. 7. Correlation of 41BB-specific IgG antibody in B cell culture supernatant enhancement of Gamma interferon secretion versus proliferation of human T cells.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The disclosure provides, among others, isolated antibodies, methods of making such antibodies, bispecific or multi-specific molecules, antibody-drug conjugates and/or immuno-conjugates composed from such antibodies or antigen binding fragments and pharmaceutical compositions containing the antibodies, bispecific or multi-specific molecules, antibody-drug conjugates and/or immuno-conjugates.

In one aspect, the disclosure provides isolated monoclonal antibodies that bind to human or cynomolgus 4-1BB. The antibodies may exhibit one or more desirable functional properties, such as high affinity binding to 4-1BB, the ability to enhance T cell activation including proliferation, IFN-γ and/or CD3+ T cell proliferation, IL-2 secretion, survival and cytolytic activity, the ability to stimulate antibody responses and/or the ability to reverse the suppressive function of immunosuppressive cells, such as T regulatory cells. In addition, the antibodies of the disclosure are derived from specific heavy and light chain amino acid sequences and/or structural features such as complementarity determining regions (CDRs) composed of specific amino acid sequences.

The term “antibody” is used in the broadest sense and specifically covers single monoclonal antibodies (including agonist and antagonist antibodies), antibody compositions with polyepitopic specificity, as well as antibody fragments (e.g., Fab, F(ab′)₂, and Fv), so long as they exhibit the desired biological activity. In some embodiments, the antibody may be monoclonal, polyclonal, chimeric, single chain, bispecific or bi-effective, simianized, human and humanized antibodies as well as active fragments thereof. Examples of active fragments of molecules that bind to known antigens include Fab, F(ab′)₂, scFv and Fv fragments, including the products of an Fab immunoglobulin expression library and epitope-binding fragments of any of the antibodies and fragments mentioned above. In some embodiments, antibody may include immunoglobulin molecules and immunologically active portions of immunoglobulin molecules, i.e. molecules that contain a binding site that immunospecifically bind an antigen. The immunoglobulin can be of any type (IgG, IgM, IgD, IgE, IgA and IgY) or class (IgG1, IgG2, IgG3, IgG4, IgA1 and IgA2) or subclasses of immunoglobulin molecule. In one embodiment, the antibody may be whole antibodies and any antigen-binding fragment derived from the whole antibodies. A typical antibody refers to heterotetrameric protein comprising typically of two heavy (H) chains and two light (L) chains. Each heavy chain is comprised of a heavy chain variable domain (abbreviated as VH) and a heavy chain constant domain. Each light chain is comprised of a light chain variable domain (abbreviated as VL) and a light chain constant domain. The VH and VL regions can be further subdivided into domains of hypervariable complementarity determining regions (CDR), and more conserved regions called framework regions (FR). Each variable domain (either VH or VL) is typically composed of three CDRs and four FRs, arranged in the following order: FR1, CDR1, FR2, CDR2, FR3, CDR3, FR4 from amino-terminus to carboxy-terminus. Within the variable regions of the light and heavy chains there are binding regions that interacts with the antigen.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler & Milstein, Nature, 256:495 (1975), or may be made by recombinant DNA methods (see, e.g., U.S. Pat. No. 4,816,567).

The monoclonal antibodies may include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 [1984]).

Monoclonal antibodies can be produced using various methods including mouse hybridoma or phage display (see Siegel. Transfus. Clin. Biol. 9:15-22 (2002) for a review) or from molecular cloning of antibodies directly from primary B cells (see Tiller. New Biotechnol. 28:453-7 (2011)). In the present disclosure antibodies were created by the immunization of rabbits with cells transiently expressing human or cynomolgus 4-1BB on the cell surface. Rabbits are known to create antibodies of high affinity, diversity and specificity (Weber et al. Exp. Mol. Med. 49:e305). B cells from immunized animals were cultured in vitro and screened for the production of anti-4-1BB antibodies. The antibody variable genes were isolated using recombinant DNA techniques and the resulting antibodies were expressed recombinantly and further screened for desired features such as the ability to enhance human T-cell activation. This general method of antibody discovery is similar to that described in Seeber et al. PLOS One. 9:e86184 (2014).

The term “antigen- or epitope-binding portion or fragment” refers to fragments of an antibody that are capable of binding to an antigen (4-1BB in this case). These fragments may be capable of the antigen-binding function and additional functions of the intact antibody. Examples of binding fragments include, but are not limited to, a single-chain Fv fragment (scFv) consisting of the VL and VH domains of a single arm of an antibody connected in a single polypeptide chain by a synthetic linker or a Fab fragment which is a monovalent fragment consisting of the VL, constant light (CL), VH and constant heavy 1 (CH1) domains. Antibody fragments can be even smaller subfragments and can consist of domains as small as a single CDR domain, in particular the CDR3 regions from either the VL and/or VH domains (for example see Beiboer et al., J. Mol. Biol. 296:833-49 (2000)). Antibody fragments are produced using conventional methods known to those skilled in the art. The antibody fragments are can be screened for utility using the same techniques employed with intact antibodies.

The “antigen- or epitope-binding fragments” can be derived from an antibody of the present disclosure by a number of art-known techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. For further description of general techniques for the isolation of active fragments of antibodies, see for example, Khaw, B. A. et al. J. Nucl. Med. 23:1011-1019 (1982); Rousseaux et al. Methods Enzymology, 121:663-69, Academic Press, 1986.

Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab′) 2 fragment that has two antigen combining sites and is still capable of cross-linking antigen.

The Fab fragment may contain the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′) 2 antibody fragments originally were produced as pairs of Fab′ fragments which have hinge cysteines between them. Other, chemical couplings of antibody fragments are also known.

“Fv” is the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The “light chains” of antibodies (immunoglobulins) from any vertebrate species can be assigned to one of two clearly distinct types, called kappa and lambda (A), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called a, delta, epsilon, γ, and μ, respectively. The subunit structures and three-dimensional configurations of different classes of immunoglobulins are well known.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity. Methods to obtain “humanized antibodies” are well known to those skilled in the art. (see, e.g., Queen et al., Proc. Natl Acad Sci USA, 86:10029-10032 (1989), Hodgson et al., Bio/Technology, 9:421 (1991)).

The terms “polypeptide”, “peptide”, and “protein”, as used herein, are interchangeable and are defined to mean a biomolecule composed of amino acids linked by a peptide bond.

The terms “a”, “an” and “the” as used herein are defined to mean “one or more” and include the plural unless the context is inappropriate.

By “isolated” is meant a biological molecule free from at least some of the components with which it naturally occurs. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities.

“Recombinant” means the antibodies are generated using recombinant nucleic acid techniques in exogeneous host cells.

The term “antigen” refers to an entity or fragment thereof which can induce an immune response in an organism, particularly an animal, more particularly a mammal including a human. The term includes immunogens and regions thereof responsible for antigenicity or antigenic determinants.

Also as used herein, the term “immunogenic” refers to substances which elicit or enhance the production of antibodies, T-cells or other reactive immune cells directed against an immunogenic agent and contribute to an immune response in humans or animals. An immune response occurs when an individual produces sufficient antibodies, T-cells and other reactive immune cells against administered immunogenic compositions of the present disclosure to moderate or alleviate the disorder to be treated.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

“Homology” between two sequences is determined by sequence identity. If two sequences which are to be compared with each other differ in length, sequence identity preferably relates to the percentage of the nucleotide residues of the shorter sequence which are identical with the nucleotide residues of the longer sequence. Sequence identity can be determined conventionally with the use of computer programs. The deviations appearing in the comparison between a given sequence and the above-described sequences of the disclosure may be caused for instance by addition, deletion, substitution, insertion or recombination.

In another aspect, the application provides pharmaceutical composition including the mAbs and their antigen-binding fragments disclosed herein. In one embodiment, the pharmaceutical composition includes the mAbs or their antigen-binding fragments and a pharmaceutically acceptable carrier. In one embodiment, the pharmaceutical composition includes the immuno-conjugate and a pharmaceutically acceptable carrier. Formulation of the pharmaceutical composition according to the disclosure can be accomplished according to standard methodology know to those of ordinary skill in the art.

The antibodies or immuno-conjugates according to the disclosure can be prepared in a physiologically acceptable formulation and may comprise a pharmaceutically acceptable carrier, diluent and/or excipient using known techniques. For example, the antibody according to the disclosure may include any functionally equivalent antibody or functional parts thereof, in particular, the monoclonal antibody including any functionally equivalent antibody or functional parts thereof is combined with a pharmaceutically acceptable carrier, diluent and/or excipient to form a therapeutic composition. Suitable pharmaceutical carriers, diluents and/or excipients are well known in the art and include, for example, phosphate buffered saline solutions, water, emulsions such as oil/water emulsions.

“Pharmaceutically acceptable” refers to those compounds, materials, compositions, and dosage forms which are, within the scope of sound medical judgment, suitable for use contact with the tissues of human beings or animals without excessive toxicity, irritation, or other problem or complication, commensurate with a reasonable benefit/risk ratio.

In one embodiment, the pharmaceutical composition may comprise proteinaceous carriers such as, for example, serum albumin or immunoglobulin, particularly of human origin. Further biologically active agents may be present in the pharmaceutical composition of the disclosure dependent on the intended use. Proteinaceous pharmaceutically active matter may be present in amounts between 1 ng and 10 mg per dose. Generally, the regime of administration should be in the range of between 0.1 μg and 10 mg of the antibody according to the disclosure, particularly in a range 1.0 μg to 1.0 mg, and more particularly in a range of between 1.0 μg and 100 μg, with all individual numbers falling within these ranges also being part of the disclosure. If the administration occurs through continuous infusion a more proper dosage may be in the range of between 0.01 μg and 10 mg units per kilogram of body weight per hour with all individual numbers falling within these ranges also being part of the disclosure.

In a further aspect, the disclosure provide method for treating cancer using anti-4-1BB antibodies or other molecules containing the antigen-binding portion of an anti-4-1BB antibody. In one embodiment, the method is used to inhibit growth of tumour cells. In one embodiment, the method is used to stimulate a protective autoimmune response, to modify an immune response or to stimulate antigen-specific immune responses.

In one embodiment, the method for treating cancer includes administering to a subject in need of such treatment an effective amount of the mAbs, their antigen-binding fragments as disclosed herein. In one embodiment, the composition may be administered in combination with other compositions comprising a biologically active substance or compound. In one embodiment, the biologically active substance may include capecitabine, cisplatin, trastuzumab, fulvestrant, tamoxifen, letrozole, exemestane, anastrozole, aminoglutethimide, testolactone, vorozole, formestane, fadrozole, letrozole, erlotinib, lafatinib, dasatinib, gefitinib, imatinib, pazopinib, lapatinib, sunitinib, nilotinib, sorafenib, nab-palitaxel, a derivative or a combination thereof.

The compositions of the present disclosure may be administered to a subject in the form of a solid, liquid or aerosol at a suitable, pharmaceutically effective dose. Examples of solid compositions include pills, creams, and implantable dosage units. Pills may be administered orally. Therapeutic creams may be administered topically. Implantable dosage units may be administered locally, for example, at a tumour site, or may be implanted for systematic release of the therapeutic composition, for example, subcutaneously. Examples of liquid compositions include formulations adapted for injection intramuscularly, subcutaneously, intravenously, intra-arterially, and formulations for topical and intraocular administration. Examples of aerosol formulations include inhaler formulations for administration to the lungs.

The compositions may be administered by standard routes of administration. For example, the composition may be administered by topical, oral, rectal, nasal, interdermal, intraperitoneal, or parenteral (for example, intravenous, subcutaneous, or intramuscular) routes. In one embodiment, administration may be parenterally, e.g. intravenously. Preparations for parenteral administration include sterile aqueous or non-aqueous solutions, suspensions and emulsions. Non-aqueous solvents include without being limited to it, propylene glycol, polyethylene glycol, vegetable oil such as olive oil, and injectable organic esters such as ethyl oleate. Aqueous solvents may be chosen from the group consisting of water, alcohol/aqueous solutions, emulsions or suspensions including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose) and others. Preservatives may also be present such as, for example, antimicrobials, anti-oxidants, chelating agents, inert gases, etc.

In one embodiment, the composition may be incorporated into sustained release matrices such as biodegradable polymers, the polymers being implanted in the vicinity of where delivery is desired, for example, at the site of a tumour. The method includes administration of a single dose, administration of repeated doses at predetermined time intervals, and sustained administration for a predetermined period of time.

It is known to those of ordinary skill in the art that the dosage of the composition will depend on various factors such as, for example, the condition of being treated, the particular composition used, and other clinical factors such as weight, size, sex and general health condition of the patient, body surface area, the particular compound or composition to be administered, other drugs being administered concurrently, and the route of administration.

In one embodiment, the term “therapeutically effective amount” refers to the amount of antibody which, when administered to a human or animal, elicits a response which is sufficient to result in a therapeutic effect in said human or animal. The effective amount is readily determined by one of ordinary skill in the art following routine procedures.

Varieties of cancer may be treated using the mAbs, their antigen-binding fragments, and compositions disclosed herein. Example cancer including without limitation breast cancer, colorectal cancer, pancreatic cancer, head and neck cancer, melanoma, ovarian cancer, prostate cancer, non-small lung cell cancer, glioma, esophageal cancer, nasopharyngeal cancer, anal cancer, rectal cancer, gastric cancer, bladder cancer, cervical cancer, or brain cancer. In one embodiment, the cancer may express 4-1BB genes. Inhibition of 4-1BB activity with anti-4-1BB monoclonal antibodies or antigen-binding fragment provides therapeutic effect. In one embodiment, administering a therapeutically effective amount of composition comprising anti-4-1BB monoclonal antibodies or antigen-binding fragment may cure, prevent, ameliorate, and delay the development or metastasis of cancers, through the effect of the drug unit.

The present disclosure may be understood more readily by reference to the following detailed description of specific embodiments included herein. Although the present disclosure has been described with reference to specific details of certain embodiments thereof, it is not intended that such details should be regarded as limitations upon the scope of the disclosure.

### EXAMPLES

### Example 1: Generation of Anti-4-1BB Antibodies

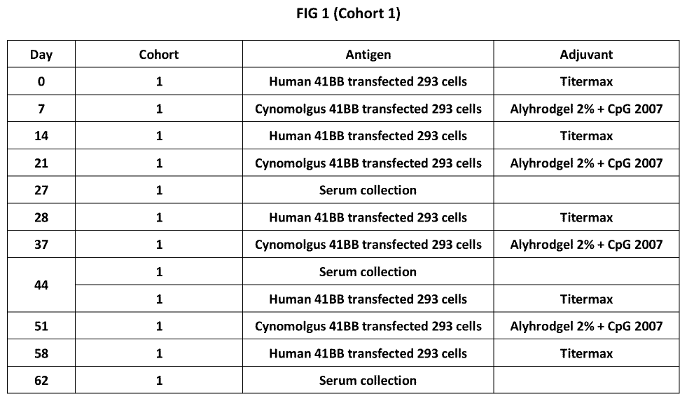

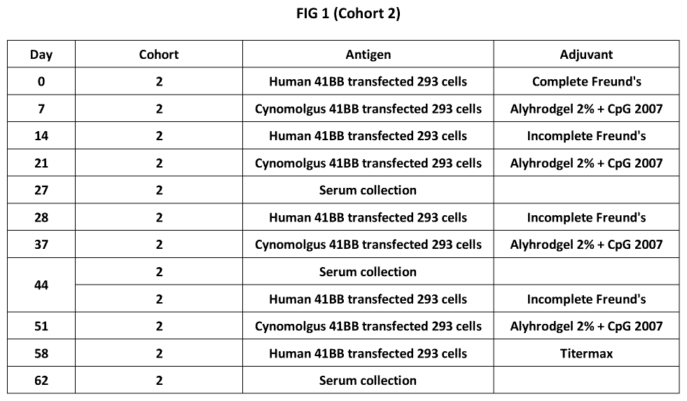

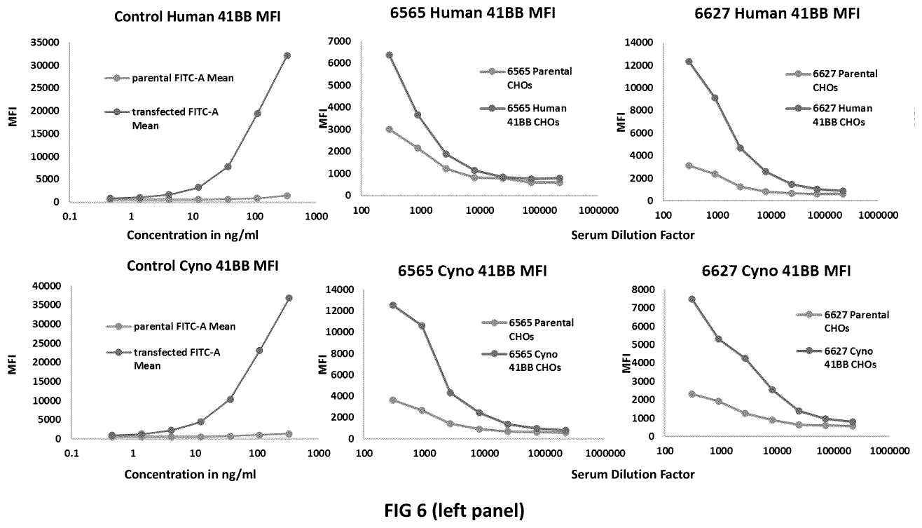

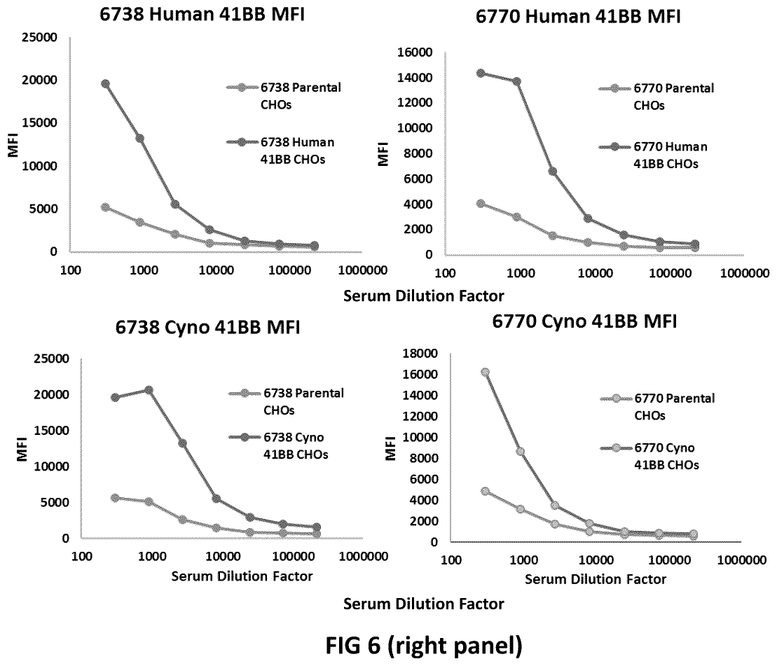

Monoclonal antibodies against human or cynomolgus 4-1BB were developed by immunizing New Zealand white rabbits. As shown in FIG. 1, animals were immunized with recombinant human embryonic kidney 293 (HEK) cells transiently transfected with human or cynomolgus 4-1BB mixed 1:1 v/v with Titermax Gold (Cohort 1) or Complete or incomplete Freund's adjuvant (Cohort 2) alternating with Alhydrogel 2% (Alum) plus CpG 2007 and were performed by subcutaneous injection. Blood samples were drawn at different timepoints during the immunization schedule as shown in FIG. 1. Serum which was drawn from all 4 animals on day 62 was analyzed for human or cynomolgus 4-1BB-specific IgG by FACS analysis as shown in FIG. 6. All 4 animals developed human or cynomolgus-specific IgG after immunization.

Antigen-specific B cells were labeled with biotinylated human and cynomolgus 4-1BB recombinant protein followed by streptavidin-alexafluor647 and then sorted into multiple 96 well tissue culture plates at 1 antigen-specific IgG+ B cell per well and cultured for 9 days to allow their differentiation into plasma cells and for secretion of antibodies. The supernatants from these B cell culture (BCC) plates were screened by ELISA and functional assay for the presence of 4-1BB-specific antibodies in the assays as listed below:

Binding to Recombinant Human and Cynomolgus 4-1BB—IgG ELISA

Enhancement of CD3-Induced T Cell Activation of Purified Human CD3+ T Cells—Detection of Gamma Interferon Secretion by ELISA and Quantitation of CD3+ T Cell Proliferation by Alamar Blue Cell Viability Assay

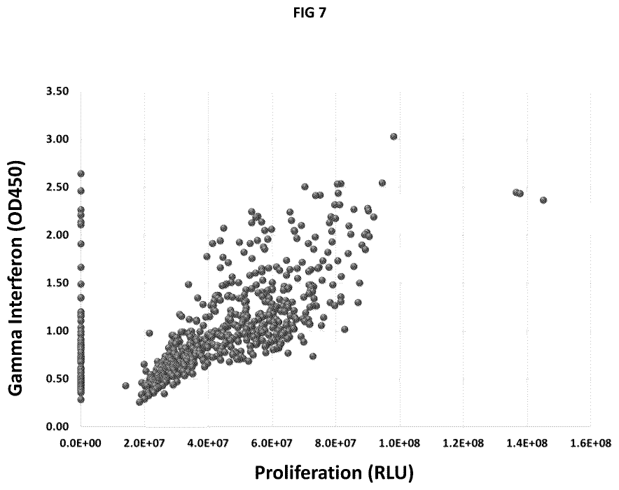

A total of 693 wells from the BCC plates had human and cynomolgus 4-1BB-specific IgG. The 4-1BB-specific antibody from these 693 BCC wells were then analyzed for agonist activity to enhance anti-CD3-induced activation of CD3+ human T cells. A fixed concentration of a control anti-human CD3-specific antibody was combined with a fixed concentration of 4-1BB-specific antibody from the BCC supernatants and then captured on and ELISA plate that was previously coated with a goat anti-rabbit IgG Fc polyclonal antibody. Purified human CD3+ T cells were added at 100,000 per each assay well and the plate was incubated for 5 days. On day 5 of the assay the contents of each assay well were collected and analyzed for the amount of gamma interferon secreted into the culture supernatant and for the number of CD3+ T cells in each assay well which is shown in FIG. 7. From these data the best 92 BCC wells were advanced to molecular rescue of the rabbit antibody heavy and light chain variable regions by RT-PCR.

On day 9 of B cell culture the supernatants were separated from the B cells and stored in a separate plate for later analysis. RNAlater tissue storage reagent was added to each well in the B cell culture plate to preserve the RNA in the B cells for RT-PCR amplification of antibody variable regions. The set of 92 BCC wells were advanced to molecular “rescue” of the antibody variable regions. The light and heavy chain variable sequences were amplified by multiplex RT-PCR using degenerate primers designed to anneal to leader sequences and the constant regions of rabbit IgG and rabbit kappa sequences. Secondary PCR was performed separately for the light and heavy chains using nested primers containing restriction sites. Amplicons from the variable heavy chain PCR were cloned into an expression vector containing human IgG1. Light chain amplicons were cloned into an expression vector containing human IgK. Resulting clones were sequenced and analyzed.

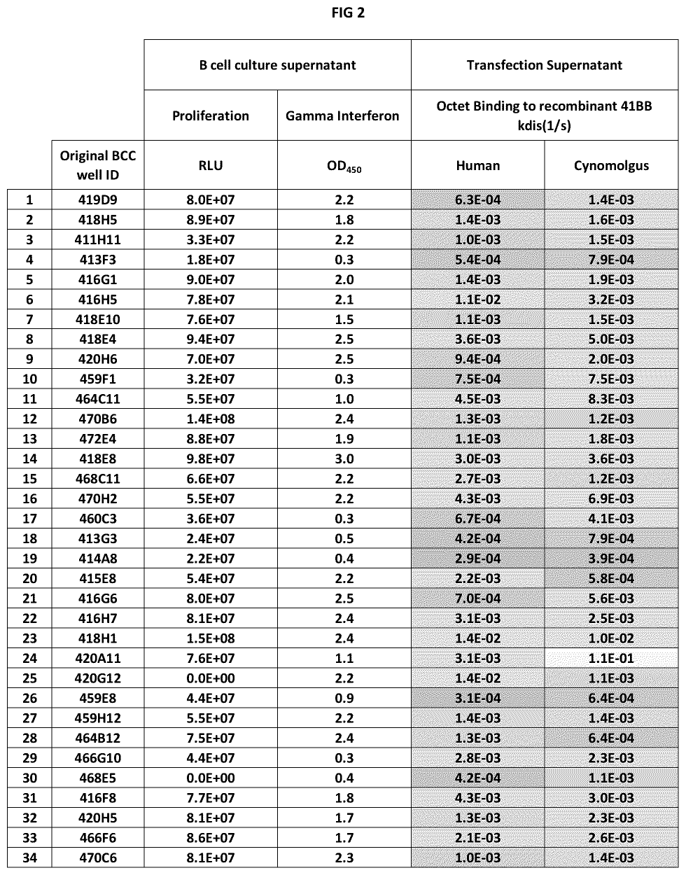

The heavy and light chain expression plasm ids generated from each well were transiently co-transfected to produce rabbit/human chimeric antibodies. Recombinant antibody supernatants were confirmed to contain antibodies specific for human and cynomolgus 4-1BB using bio-layer interferometry analysis on a ForteBio Octet Red 96 instrument. Anti-human Fc biosensors (Pall ForteBio) were used to capture antibodies in the supernatants. Association to human or cynomolgus 4-1BB was observed by real-time interferometry by placing the biosensors in wells containing recombinant human or cynomolgus 4-1BB extracellular domain protein. Dissociation was measured after transfer of the biosensors into wells containing 10× kinetics buffer (Pall ForteBio). The software provided by the manufacturer was used to analyze the interferometry data. A summary of the primary BCC screening data and the corresponding screening data for 34 recombinant chimeric rabbit/human IgG antibodies is shown in FIG. 2. From the original 92 BCC wells 34 chimeric antibodies were rescued and were shown to bind human and cynomolgus recombinant 4-1BB protein (FIG. 2)

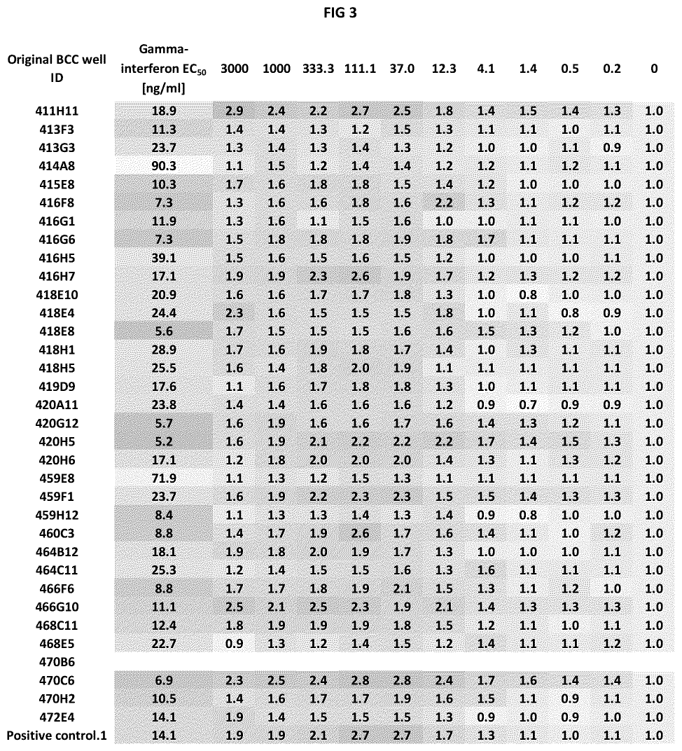

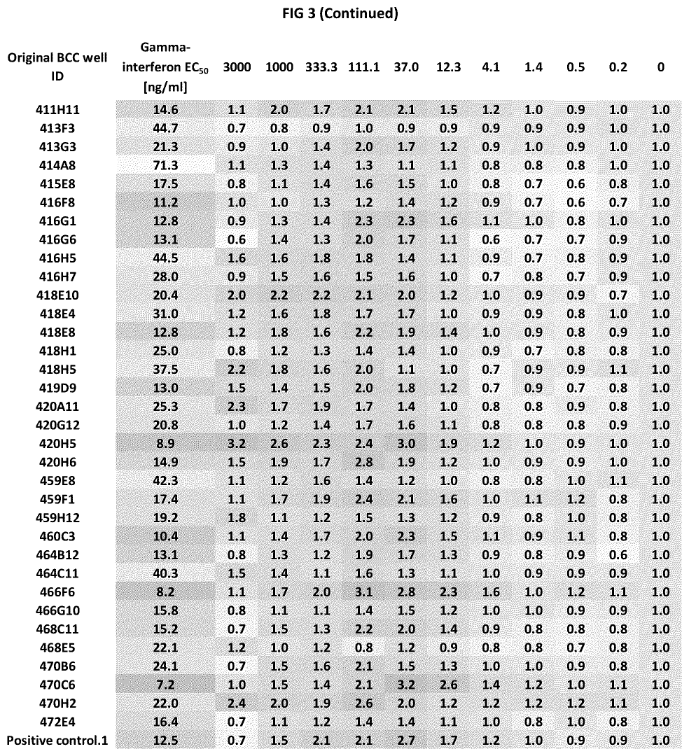

The panel of 34 chimeric rabbit/human IgG antibodies specific for human and cynomolgus 4-1BB were then assayed for agonist activity which enhanced the anti-CD3-induced activation of human CD3+ T cells. As shown in FIG. 3, EC₅₀ for secretion of gamma interferon were calculated for each chimeric antibody as well as the fold-over-background of the enhancement of proliferation of human CD3+ T cells. Proliferation of human CD3+ T cells with anti-CD3 antibody alone, without additional 4-1BB antibody, is set to the background value of 1.0 and then the fold-over-background is calculated at different concentrations of the chimeric 4-1BB antibodies are tested. From these data 18 chimeric 4-1BB-specific antibodies were advanced to humanization.

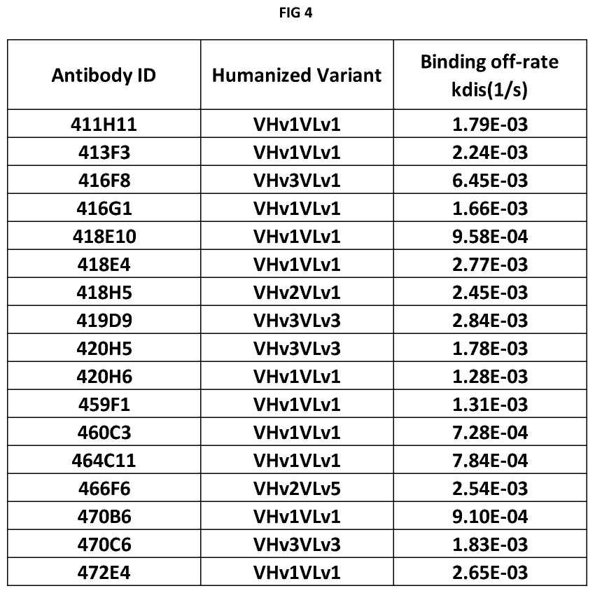

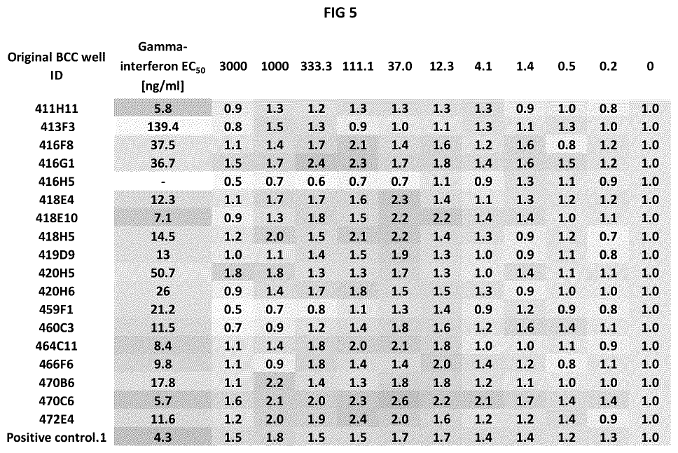

From the panel of 34 chimeric rabbit/human IgG antibodies specific for human and cynomolgus 4-1BB that were advanced to humanization 18 were successfully humanized as shown in FIG. 4 where binding to human and recombinant 4-1BB by octet assay was performed. These 18 humanized antibodies specific for human or cynomolgus 4-1BB were then assayed for agonist activity which enhanced the anti-CD3-induced activation of human CD3+ T cells. As shown in FIG. 5, EC₅₀ for secretion of gamma interferon were calculated for each humanized antibody as well as the fold-over-background of the enhancement of proliferation of human CD3+ T cells. Proliferation of human CD3+ T cells with anti-CD3 antibody alone, without additional 4-1BB antibody, is set to the background value of 1.0 and then the fold-over-background is calculated at different concentrations of the humanized 4-1BB antibodies are tested. From these data 17 of the 18 humanized 4-1BB-specific antibodies showed enhancement of anti-CD3-induced activation of human CD3+ T cells.

While the disclosure has been particularly shown and described as referenced to the embodiments thereof, those skilled in the art will understand that the foregoing and other changes in form and detail may be made therein without departing from the spirit and scope. All references cited or referred to in this disclosure are hereby incorporated by reference in their entireties.

SEQUENCE LISTING
411H11CHIMERICLIGHTCHAINFULL-LENGTHNUCLEOTIDESEQUENCE
SEQ ID NO: 1
GATGTTGTGATGACCCAGACTCCAGCCTCCGTGTCTGAACCTGTGAGAGCCACAGTCACCATCAAGTGCCAGGCCA
GTCAGAGCATTGGTGCTGATTTAGCCTGGTATCAGCAGAAACCAGGGCAGCCTCCCAAGCTCCTGATCTACAGGG
CATCCACTCTGGCCTCTGGGGTCCCATCGCGGTTCAGCGGCAGTGGATCTGGGACAGAGTTCACTCTCACCATCAG
CGACCTGGAGTGTGCCGATGCTGCCACTTACTACTGTCAATGTATTTATTATGGTACTGATGATGTCATATACCATA
CTTTCGGCGGAGGGACCGAGGTGGTGTTCAAACGTACGGTGGCTGCACCATCTGTCTTCATCTTCCCGCCATCTGA
TGAGCAGTTGAAATCTGGAACTGCCTCTGTTGTGTGCCTGCTGAATAACTTCTATCCCAGAGAGGCCAAAGTACAG
TGGAAGGTGGATAACGCCCTCCAATCGGGTAACTCCCAGGAGAGTGTCACAGAGCAGGACAGCAAGGACAGCAC
CTACAGCCTCAGCAGCACCCTGACGCTGAGCAAAGCAGACTACGAGAAACACAAAGTCTACGCCTGCGAAGTCAC
CCATCAGGGCCTGAGCTCGCCCGTCACAAAGAGCTTCAACAGGGGAGAGTGT
411H11CHIMERICLIGHTCHAINVARIABLELIGHTCHAINNUCLEOTIDESEQUENCE
SEQ ID NO: 2
GATGTTGTGATGACCCAGACTCCAGCCTCCGTGTCTGAACCTGTGAGAGCCACAGTCACCATCAAGTGCCAGGCCA
GTCAGAGCATTGGTGCTGATTTAGCCTGGTATCAGCAGAAACCAGGGCAGCCTCCCAAGCTCCTGATCTACAGGG
CATCCACTCTGGCCTCTGGGGTCCCATCGCGGTTCAGCGGCAGTGGATCTGGGACAGAGTTCACTCTCACCATCAG
CGACCTGGAGTGTGCCGATGCTGCCACTTACTACTGTCAATGTATTTATTATGGTACTGATGATGTCATATACCATA
CTTTCGGCGGAGGGACCGAGGTGGTGTTCAAA
411H11CHIMERICLIGHTCHAINFULL-LENGTHAMINOACIDSEQUENCE.HUMANKAPPACONSTANT
DOMAINISUNDERLINED
SEQ ID NO: 3
DVVMTQTPASVSEPVRATVTIKCQASQSIGADLAWYQQKPGQPPKLLIYRASTLASGVPSRFSGSGSGTEFTLTISDLEC
ADAATYYCQCIYYGTDDVIYHTFGGGTEVVFKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
411H11CHIMERICLIGHTCHAINVARIABLELIGHTCHAINAMINOACIDSEQUENCE.COMPIMENTARITY
DETERMININGREGIONSAREUNDERLINED
SEQ ID NO: 4
DVVMTQTPASVSEPVRATVTIKCQASQSIGADLAWYQQKPGQPPKLLIYRASTLASGVPSRFSGSGSGTEFTLTISDLEC
ADAATYYCQCIYYGTDDVIYHTFGGGTEVVFK
411H11CHIMERICHEAVYCHAINFULL-LENGTHNUCLEOTIDESEQUENCE
SEQ ID NO: 5
CAGTCGGTGGAGGAGTCCGGGGGTCGCCTGGTCACGCCTGGGACACCCCTAACACTCACCTGCACAGTATCTGGA
TTCACCATCAGTAGCCACCACATGATCTGGGTCCGCCAGGCTCCAGGAGAGGGGCTGCAATACATCGGATTCATTA
ATGATGGTGACTATACATACTACACGAACTGGGCAAAAGGCCGATTCACCATCTCCAGAACCTCGACTACGGTGG
ACCTGAAAATGACCAGTCTGACAGCCGCGGACACGGCCACCTATTTCTGTGCCAGAGGGGTTGATGGTACTAGTT
ATCCTGGCTTGTGGGGCCCAGGCACCCTGGTCACCGTCTCGAGCGCTAGCACCAAGGGCCCATCGGTCTTCCCCCT
GGCACCCTCCTCCAAGAGCACCTCTGGGGGCACAGCGGCCCTGGGCTGCCTGGTCAAGGACTACTTCCCCGAACC
GGTGACGGTGTCGTGGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACCCAGACCTACATCTGCAACGTGAAT
CACAAGCCCAGCAACACCAAGGTGGACAAGAGAGTTGAGCCCAAATCTTGTGACAAAACTCACACATGCCCACCG
TGCCCAGCACCTGAAGCCGCGGGGGCACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTGTGG
TCAGCGTCCTCACCGTCCTGCACCAGGACTGGCTGAATGGCAAGGAGTACAAGTGCGCGGTCTCCAACAAAGCCC
TCCCAGCCCCCATCGAGAAAACCATCTCCAAAGCCAAAGGGCAGCCCCGAGAACCACAGGTGTACACCCTGCCCC
CATCCCGGGATGAGCTGACCAAGAACCAGGTCAGCCTGACCTGCCTGGTCAAAGGCTTCTATCCCAGCGACATCG
CCGTGGAGTGGGAGAGCAATGGGCAGCCGGAGAACAACTACAAGACCACGCCTCCCGTGCTGGACTCCGACGGC
TCCTTCTTCCTCTATAGCAAGCTCACCGTGGACAAGAGCAGGTGGCAGCAGGGGAACGTCTTCTCATGCTCCGTGA
TGCATGAGGCTCTGCACAACCACTACACGCAGAAGAGCCTCTCCCTGTCTCCGGGT
411H11CHIMERICHEAVYCHAINVARIABLEHEAVYCHAINNUCLEOTIDESEQUENCE
SEQ ID NO: 6
CAGTCGGTGGAGGAGTCCGGGGGTCGCCTGGTCACGCCTGGGACACCCCTAACACTCACCTGCACAGTATCTGGA
TTCACCATCAGTAGCCACCACATGATCTGGGTCCGCCAGGCTCCAGGAGAGGGGCTGCAATACATCGGATTCATTA
ATGATGGTGACTATACATACTACACGAACTGGGCAAAAGGCCGATTCACCATCTCCAGAACCTCGACTACGGTGG
ACCTGAAAATGACCAGTCTGACAGCCGCGGACACGGCCACCTATTTCTGTGCCAGAGGGGTTGATGGTACTAGTT
ATCCTGGCTTGTGGGGCCCAGGCACCCTGGTCACCGTCTCGAGC
411H11CHIMERICHEAVYCHAINFULL-LENGTHAMINOACIDSEQUENCE.HUMANGAMMA-1
CONSTANTDOMAINISUNDERLINED
SEQ ID NO: 7
QSVEESGGRLVTPGTPLTLTCTVSGFTISSHHMIWVRQAPGEGLQYIGFINDGDYTYYTNWAKGRFTISRTSTTVDLKM
TSLTAADTATYFCARGVDGTSYPGLWGPGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKRVEPKSCDKTHTCPPCPAPEAAGAPSV
FLFPPKPKDTLMISRTPEVTCVVVDVSHEDPEVKFNWYVDGVEVHNAKTKPREEQYNSTYRVVSVLTVLHQDWLNGKE
YKCAVSNKALPAPIEKTISKAKGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG
411H11CHIMERICHEAVYCHAINVARIABLEHEAVYCHAINAMINOACIDSEQUENCE.
COMPLIMENTARITYDETERMININGREGIONSAREUNDERLINED
SEQ ID NO: 8
QSVEESGGRLVTPGTPLTLTCTVSGFTISSHHMIWVRQAPGEGLQYIGFINDGDYTYYTNWAKGRFTISRTSTTVDLKM
TSLTAADTATYFCARGVDGTSYPGLWGPGTLVTVSS
413F3CHIMERICLIGHTCHAINFULL-LENGTHNUCLEOTIDESEQUENCE
SEQ ID NO: 9
GACGTCGTGATGACCCAGACTCCAGCCTCCGTGGAGGCAGCTGTGGGAGGCACAGTCACCATCAAGTGCCAGGC
CAGTCAGAGCATTTACAGCAATTTAGCCTGGTATCAGCAGAAACCAGGGCAGCCTCCCAAACTCCTAATCTATTCT
ACATCCACTCTGGCATCTGGGGTCCCATCGCGGTTCAGCGGCAGTGGATCCGGGACAGAGTTCACTCTCACCATCA
GCGACCTGGAGTGTGCCGATGCTGCCACTTACTACTGTCAAAACTATTATGGTAGTAGTACTGATAGTTATGGGAA
TCCTTTCGGCGGAGGCACCGAGGTGGTGTTCAAACGTACGGTGGCTGCACCATCTGTCTTCATCTTCCCGCCATCT
GATGAGCAGTTGAAATCTGGAACTGCCTCTGTTGTGTGCCTGCTGAATAACTTCTATCCCAGAGAGGCCAAAGTAC
AGTGGAAGGTGGATAACGCCCTCCAATCGGGTAACTCCCAGGAGAGTGTCACAGAGCAGGACAGCAAGGACAGC
ACCTACAGCCTCAGCAGCACCCTGACGCTGAGCAAAGCAGACTACGAGAAACACAAAGTCTACGCCTGCGAAGTC
ACCCATCAGGGCCTGAGCTCGCCCGTCACAAAGAGCTTCAACAGGGGAGAGTGT
413F3CHIMERICLIGHTCHAINVARIABLELIGHTCHAINNUCLEOTIDESEQUENCE
SEQ ID NO: 10
GACGTCGTGATGACCCAGACTCCAGCCTCCGTGGAGGCAGCTGTGGGAGGCACAGTCACCATCAAGTGCCAGGC
CAGTCAGAGCATTTACAGCAATTTAGCCTGGTATCAGCAGAAACCAGGGCAGCCTCCCAAACTCCTAATCTATTCT
ACATCCACTCTGGCATCTGGGGTCCCATCGCGGTTCAGCGGCAGTGGATCCGGGACAGAGTTCACTCTCACCATCA
GCGACCTGGAGTGTGCCGATGCTGCCACTTACTACTGTCAAAACTATTATGGTAGTAGTACTGATAGTTATGGGAA
TCCTTTCGGCGGAGGCACCGAGGTGGTGTTCAAA
413F3CHIMERICLIGHTCHAINFULL-LENGTHAMINOACIDSEQUENCE.HUMANKAPPACONSTANT
DOMAINISUNDERLINED
SEQ ID NO: 11
DVVMTQTPASVEAAVGGTVTIKCQASQSIYSNLAWYQQKPGQPPKLLIYSTSTLASGVPSRFSGSGSGTEFTLTISDLEC
ADAATYYCQNYYGSSTDSYGNPFGGGTEVVFKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
413F3CHIMERICLIGHTCHAINVARIABLELIGHTCHAINAMINOACIDSEQUENCE.COMPIMENTARITY
DETERMININGREGIONSAREUNDERLINED
SEQ ID NO: 12
DVVMTQTPASVEAAVGGTVTIKCQASQSIYSNLAWYQQKPGQPPKLLIYSTSTLASGVPSRFSGSGSGTEFTLTISDLEC
ADAATYYCQNYYGSSTDSYGNPFGGGTEVVFK
413F3CHIMERICHEAVYCHAINFULL-LENGTHNUCLEOTIDESEQUENCE
SEQ ID NO: 13
CAGTCGCTGGAGGAGTCCGGGGGAGACCTGGTCAAGCCTGAGGGATCCCTGACACTCACCTGCACAGGCTCTGG
AATCGACTTCAGTAGCAACTACATGTGCTGGGTCCGCCAGGCTCCAGGGAAGGGGCTGGAGTGGATCGCATGCAT
TTATGGTGATAGTAGTGATAATAGTTACTCCGCGAGCTGGGCGAAAGGGCGATTCACCATCTCCAAAACCTCGTCG
ACCACGGTGACTCTGCAAATGACCAGTCTGACAGCCGCGGACACGGCCACCTATTTCTGTGCGAGATCTGGTTATA
GCTATTTAGGCTACTTTAACTTGTGGGGCCCAGGGACCCTGGTCACCGTCTCGAGCGCTAGCACCAAGGGCCCATC
GGTCTTCCCCCTGGCACCCTCCTCCAAGAGCACCTCTGGGGGCACAGCGGCCCTGGGCTGCCTGGTCAAGGACTA
CTTCCCCGAACCGGTGACGGTGTCGTGGAACTCAGGCGCCCTGACCAGCGGCGTGCACACCTTCCCGGCTGTCCTA
CAGTCCTCAGGACTCTACTCCCTCAGCAGCGTGGTGACCGTGCCCTCCAGCAGCTTGGGCACCCAGACCTACATCT
GCAACGTGAATCACAAGCCCAGCAACACCAAGGTGGACAAGAGAGTTGAGCCCAAATCTTGTGACAAAACTCACA
CATGCCCACCGTGCCCAGCACCTGAAGCCGCGGGGGCACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTGAGGTCAAGTT
CAACTGGTACGTGGACGGCGTGGAGGTGCATAATGCCAAGACAAAGCCGCGGGAGGAGCAGTACAACAGCACG
TACCGTGTGGTCAGCGTCCTCACCGTCCTGCACCAGGACTGGCTGAATGGCAAGGAGTACAAGTGCGCGGTCTCC
AACAAAGCCCTCCCAGCCCCCATCGAGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGACCTGCCTGGTCAAAGGCTTCTATCCCA
GCGACATCGCCGTGGAGTGGGAGAGCAATGGGCAGCCGGAGAACAACTACAAGACCACGCCTCCCGTGCTGGAC
TCCGACGGCTCCTTCTTCCTCTATAGCAAGCTCACCGTGGACAAGAGCAGGTGGCAGCAGGGGAACGTCTTCTCAT
GCTCCGTGATGCATGAGGCTCTGCACAACCACTACACGCAGAAGAGCCTCTCCCTGTCTCCGGGT
413F3CHIMERICHEAVYCHAINVARIABLEHEAVYCHAINNUCLEOTIDESEQUENCE
SEQ ID NO: 14
CAGTCGCTGGAGGAGTCCGGGGGAGACCTGGTCAAGCCTGAGGGATCCCTGACACTCACCTGCACAGGCTCTGG
AATCGACTTCAGTAGCAACTACATGTGCTGGGTCCGCCAGGCTCCAGGGAAGGGGCTGGAGTGGATCGCATGCAT
TTATGGTGATAGTAGTGATAATAGTTACTCCGCGAGCTGGGCGAAAGGGCGATTCACCATCTCCAAAACCTCGTCG
ACCACGGTGACTCTGCAAATGACCAGTCTGACAGCCGCGGACACGGCCACCTATTTCTGTGCGAGATCTGGTTATA
GCTATTTAGGCTACTTTAACTTGTGGGGCCCAGGGACCCTGGTCACCGTCTCGAGC
413F3CHIMERICHEAVYCHAINFULL-LENGTHAMINOACIDSEQUENCE.HUMANGAMMA-1CONSTANT
DOMAINISUNDERLINED
SEQ ID NO: 15
QSLEESGGDLVKPEGSLTLTCTGSGIDFSSNYMCWVRQAPGKGLEWIACIYGDSSDNSYSASWAKGRFTISKTSSTTVTL
QMTSLTAADTATYFCARSGYSYLGYFNLWGPGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVDKRVEPKSCDKTHTCPPCPAPEAAG
APSVFLFPPKPKDTLMISRTPEVTCVVVDVSHEDPEVKFNWYVDGVEVHNAKTKPREEQYNSTYRVVSVLTVLHQDWL
NGKEYKCAVSNKALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPG
413F3CHIMERICHEAVYCHAINVARIABLEHEAVYCHAINAMINOACIDSEQUENCE.COMPLIMENTARITY
DETERMININGREGIONSAREUNDERLINED
SEQ ID NO: 16
QSLEESGGDLVKPEGSLTLTCTGSGIDFSSNYMCWVRQAPGKGLEWIACIYGDSSDNSYSASWAKGRFTISKTSSTTV
TLQMTSLTAADTATYFCARSGYSYLGYFNLWGPGTLVTVSS
416F8CHIMERICLIGHTCHAINFULL-LENGTHNUCLEOTIDESEQUENCE
SEQ ID NO: 17
GCGCAAGTGCTGACCCAGACTGCATCGTCCGTGTCTGCACCTGTGGGCGGCACAGTCACCATCAGTTGCCAGTCCA
GTCAGACTGTTTATAATAACAACTTGTTATCCTGGTATCAGCAGAAACCAGGGCAGCGTCCCAAGCTCCTGATCTA
TTATGCATCCACTCTGGCATTTGGGGTCCCATCGCGGTTCAAAGGCAGTGGATCTGGGACAGAGTTCACTCTCACC
ATCAGCGACCTGGAGTGTGACGATGCTGCCACTTACTTCTGTCAAGGCGGTTATAGTGGTTGGATTTATGTTTTCG
GCGGAGGCACCGAGGTGGAGGTCAAACGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGGAA
GGTGGATAACGCCCTCCAATCGGGTAACTCCCAGGAGAGTGTCACAGAGCAGGACAGCAAGGACAGCACCTACA
GCCTCAGCAGCACCCTGACGCTGAGCAAAGCAGACTACGAGAAACACAAAGTCTACGCCTGCGAAGTCACCCATC
AGGGCCTGAGCTCGCCCGTCACAAAGAGCTTCAACAGGGGAGAGTGT
416F8CHIMERICLIGHTCHAINVARIABLELIGHTCHAINNUCLEOTIDESEQUENCE
SEQ ID NO: 18
GCGCAAGTGCTGACCCAGACTGCATCGTCCGTGTCTGCACCTGTGGGCGGCACAGTCACCATCAGTTGCCAGTCCA
GTCAGACTGTTTATAATAACAACTTGTTATCCTGGTATCAGCAGAAACCAGGGCAGCGTCCCAAGCTCCTGATCTA
TTATGCATCCACTCTGGCATTTGGGGTCCCATCGCGGTTCAAAGGCAGTGGATCTGGGACAGAGTTCACTCTCACC
ATCAGCGACCTGGAGTGTGACGATGCTGCCACTTACTTCTGTCAAGGCGGTTATAGTGGTTGGATTTATGTTTTCG
GCGGAGGCACCGAGGTGGAGGTCAAA
416F8CHIMERICLIGHTCHAINFULL-LENGTHAMINOACIDSEQUENCE.HUMANKAPPACONSTANT
DOMAINISUNDERLINED
SEQ ID NO: 19
AQVLTQTASSVSAPVGGTVTISCQSSQTVYNNNLLSWYQQKPGQRPKLLIYYASTLAFGVPSRFKGSGSGTEFTLTISDLE
CDDAATYFCQGGYSGWIYVFGGGTEVEVKRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC
416F8CHIMERICLIGHTCHAINVARIABLELIGHTCHAINAMINOACIDSEQUENCE.COMPIMENTARITY
DETERMININGREGIONSAREUNDERLINED
SEQ ID NO: 20
AQVLTQTASSVSAPVGGTVTISCQSSQTVYNNNLLSWYQQKPGQRPKLLIYYASTLAFGVPSRFKGSGSGTEFTLTI
SDLECDDAATYFCQGGYSGWIYVFGGGTEVEVK
416F8CHIMERICHEAVYCHAINFULL-LENGTHNUCLEOTIDESEQUENCE
SEQ ID NO: 21
CAGTCGCTGGAGGAGTCCGGGGGTCGCCTGGTCACGCCTGGGACACCCCTGACACTCACCTGCACAGCCTCTGGA
TTCTCCCTCAGTAACTACTACATGAACTGGGTCCGCCAGGCTCCAGGGAAGGGGCTGGAATACATCGGAATCATTA
GTAGTAGTGGTAGCGCATACTACGCGAGCTGGGCTAAAGGCCGATTCACCATCTCCAGAACCTCGACCACGGTGG
ACCTGAGAATCACCAGTCCGACAACCGAGGACACGGCCACCTATTTCTGTGCCAGAGGGGACTACGGCATGGACC
TCTGGGGCCAAGGGACCCTGGTCACCGTCTCGAGCGCTAGCACCAAGGGCCCATCGGTCTTCCCCCTGGCACCCTC
CTCCAAGAGCACCTCTGGGGGCACAGCGGCCCTGGGCTGCCTGGTCAAGGACTACTTCCCCGAACCGGTGACGGT
GTCGTGGAACTCAGGCGCCCTGACCAGCGGCGTGCACACCTTCCCGGCTGTCCTACAGTCCTCAGGACTCTACTCC
CTCAGCAGCGTGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACCGTGCCCAGCA
CCTGAAGCCGCGGGGGCACCGTCAGTCTTCCTCTTCCCCCCAAAACCCAAGGACACCCTCATGATCTCCCGGACCC
CTGAGGTCACATGCGTGGTGGTGGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGCACGTACCGTGTGGTCAGCGTCCT
CACCGTCCTGCACCAGGACTGGCTGAATGGCAAGGAGTACAAGTGCGCGGTCTCCAACAAAGCCCTCCCAGCCCC
CATCGAGAAAACCATCTCCAAAGCCAAAGGGCAGCCCCGAGAACCACAGGTGTACACCCTGCCCCCATCCCGGGA
TGAGCTGACCAAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGCTCCTTCTTCCT
CTATAGCAAGCTCACCGTGGACAAGAGCAGGTGGCAGCAGGGGAACGTCTTCTCATGCTCCGTGATGCATGAGGC
TCTGCACAACCACTACACGCAGAAGAGCCTCTCCCTGTCTCCGGGT
416F8CHIMERICHEAVYCHAINVARIABLEHEAVYCHAINNUCLEOTIDESEQUENCE
SEQ ID NO: 22
CAGTCGCTGGAGGAGTCCGGGGGTCGCCTGGTCACGCCTGGGACACCCCTGACACTCACCTGCACAGCCTCTGGA
TTCTCCCTCAGTAACTACTACATGAACTGGGTCCGCCAGGCTCCAGGGAAGGGGCTGGAATACATCGGAATCATTA
GTAGTAGTGGTAGCGCATACTACGCGAGCTGGGCTAAAGGCCGATTCACCATCTCCAGAACCTCGACCACGGTGG
ACCTGAGAATCACCAGTCCGACAACCGAGGACACGGCCACCTATTTCTGTGCCAGAGGGGACTACGGCATGGACC
TCTGGGGCCAAGGGACCCTGGTCACCGTCTCGAGC
416F8 CHIMERIC HEAVY CHAIN FULL-LENGTH AMINO ACID SEQUENCE. HUMAN GAMMA-1 CONSTANT
DOMAINISUNDERLINED
SEQ ID NO: 23
QSLEESGGRLVTPGTPLTLTCTASGFSLSNYYMNWVRQAPGKGLEYIGIISSSGSAYYASWAKGRFTISRTSTTVDLRITSP
TTEDTATYFCARGDYGMDLWGQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKRVEPKSCDKTHTCPPCPAPEAAGAPSVFLFPPK
PKDTLMISRTPEVTCVVVDVSHEDPEVKFNWYVDGVEVHNAKTKPREEQYNSTYRVVSVLTVLHQDWLNGKEYKCAV
SNKALPAPIEKTISKAKGQPREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPG
416F8CHIMERICHEAVYCHAINVARIABLEHEAVYCHAINAMINOACIDSEQUENCE.COMPLIMENTARITY
DETERMININGREGIONSAREUNDERLINED
SEQ ID NO: 24
QSLEESGGRLVTPGTPLTLTCTASGFSLSNYYMNWVRQAPGKGLEYIGIISSSGSAYYASWAKGRFTISRTSTTVDLRITSP
TTEDTATYFCARGDYGMDLWGQGTLVTVSS
416G1CHIMERICLIGHTCHAINFULL-LENGTHNUCLEOTIDESEQUENCE
SEQ ID NO: 25
GACGTCGTGATGACCCAGACTCCAGCCTCCGTGTCTGAACCTGTGGGAGGCGCAGTCACCATCAAGTGCCAGGCC
AGTCAGAGCATTGGTAGTGATTTAGCCTGGTATCAGCAGAAACCAGGGCAGCCTCCCAAGCTCCTGATCTACAGG
GCATCCACTCTGGCATCTGGGGTCTCATCGCGGTTCAAAGGCAGTGGATCTGGGACAGAGTTCACTCTCACCATCA
GCGACCTGGAGTGTGCCGATGCTGCCACTTACTACTGTCAATGTATTTATTATGGTAGTGATGATGTCATATACCAT
ACTTTCGGCGGAGGGACCGAGGTGGTGGTCAAACGTACGGTGGCTGCACCATCTGTCTTCATCTTCCCGCCATCTG
ATGAGCAGTTGAAATCTGGAACTGCCTCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCACAGAGCAGGACAGCAAGGACAGCA
CCTACAGCCTCAGCAGCACCCTGACGCTGAGCAAAGCAGACTACGAGAAACACAAAGTCTACGCCTGCGAAGTCA
CCCATCAGGGCCTGAGCTCGCCCGTCACAAAGAGCTTCAACAGGGGAGAGTGT
416G1CHIMERICLIGHTCHAINVARIABLELIGHTCHAINNUCLEOTIDESEQUENCE
SEQ ID NO: 26
GACGTCGTGATGACCCAGACTCCAGCCTCCGTGTCTGAACCTGTGGGAGGCGCAGTCACCATCAAGTGCCAGGCC
AGTCAGAGCATTGGTAGTGATTTAGCCTGGTATCAGCAGAAACCAGGGCAGCCTCCCAAGCTCCTGATCTACAGG
GCATCCACTCTGGCATCTGGGGTCTCATCGCGGTTCAAAGGCAGTGGATCTGGGACAGAGTTCACTCTCACCATCA
GCGACCTGGAGTGTGCCGATGCTGCCACTTACTACTGTCAATGTATTTATTATGGTAGTGATGATGTCATATACCAT
ACTTTCGGCGGAGGGACCGAGGTGGTGGTCAAA
416G1CHIMERICLIGHTCHAINFULL-LENGTHAMINOACIDSEQUENCE.HUMANKAPPACONSTANT
DOMAINISUNDERLINED
SEQ ID NO: 27
DVVMTQTPASVSEPVGGAVTIKCQASQSIGSDLAWYQQKPGQPPKLLIYRASTLASGVSSRFKGSGSGTEFTLTISDLEC
ADAATYYCQCIYYGSDDVIYHTFGGGTEVVVKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
416G1CHIMERICLIGHTCHAINVARIABLELIGHTCHAINAMINOACIDSEQUENCE.COMPIMENTARITY
DETERMININGREGIONSAREUNDERLINED
SEQ ID NO: 28
DVVMTQTPASVSEPVGGAVTIKCQASQSIGSDLAWYQQKPGQPPKLLIYRASTLASGVSSRFKGSGSGTEFTLTISDLEC
ADAATYYCQCIYYGSDDVIYHTFGGGTEVVVK
416G1CHIMERICHEAVYCHAINFULL-LENGTHNUCLEOTIDESEQUENCE
SEQ ID NO: 29
CAGTCGGTGGAGGAGTCCGGGGGTCGCCTGGTCACGCCTGGGACACCCCTGACACTCACCTGCACAGTCTCTGGA
TTCACTATCAATAGCTACCACATGATCTGGGTCCGCCAGGCTCCAGGGGAGGGGCTGGAATACATCGGATTCATTA
ATGATGGTGGTTTCACATACTACGCGAGCTGGGCAAAAGGCCGATTTATCATCTCCAGAACCTCGACCACGGTGG
ATCTGAAAATGACCAGTCTGACAGTCGCGGACACGGCCACCTATTTCTGTGCCAGAGGGGTTGATGGTACTAGTT
ATCCTGACTTATGGGGCCCGGGCACCCTGGTCACCGTCTCGAGCGCTAGCACCAAGGGCCCATCGGTCTTCCCCCT
GGCACCCTCCTCCAAGAGCACCTCTGGGGGCACAGCGGCCCTGGGCTGCCTGGTCAAGGACTACTTCCCCGAACC
GGTGACGGTGTCGTGGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACCCAGACCTACATCTGCAACGTGAAT
CACAAGCCCAGCAACACCAAGGTGGACAAGAGAGTTGAGCCCAAATCTTGTGACAAAACTCACACATGCCCACCG
TGCCCAGCACCTGAAGCCGCGGGGGCACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTGTGG
TCAGCGTCCTCACCGTCCTGCACCAGGACTGGCTGAATGGCAAGGAGTACAAGTGCGCGGTCTCCAACAAAGCCC
TCCCAGCCCCCATCGAGAAAACCATCTCCAAAGCCAAAGGGCAGCCCCGAGAACCACAGGTGTACACCCTGCCCC
CATCCCGGGATGAGCTGACCAAGAACCAGGTCAGCCTGACCTGCCTGGTCAAAGGCTTCTATCCCAGCGACATCG
CCGTGGAGTGGGAGAGCAATGGGCAGCCGGAGAACAACTACAAGACCACGCCTCCCGTGCTGGACTCCGACGGC
TCCTTCTTCCTCTATAGCAAGCTCACCGTGGACAAGAGCAGGTGGCAGCAGGGGAACGTCTTCTCATGCTCCGTGA
TGCATGAGGCTCTGCACAACCACTACACGCAGAAGAGCCTCTCCCTGTCTCCGGGT
416G1CHIMERICHEAVYCHAINVARIABLEHEAVYCHAINNUCLEOTIDESEQUENCE
SEQ ID NO: 30
CAGTCGGTGGAGGAGTCCGGGGGTCGCCTGGTCACGCCTGGGACACCCCTGACACTCACCTGCACAGTCTCTGGA
TTCACTATCAATAGCTACCACATGATCTGGGTCCGCCAGGCTCCAGGGGAGGGGCTGGAATACATCGGATTCATTA
ATGATGGTGGTTTCACATACTACGCGAGCTGGGCAAAAGGCCGATTTATCATCTCCAGAACCTCGACCACGGTGG
ATCTGAAAATGACCAGTCTGACAGTCGCGGACACGGCCACCTATTTCTGTGCCAGAGGGGTTGATGGTACTAGTT
ATCCTGACTTATGGGGCCCGGGCACCCTGGTCACCGTCTCGAGC
416G1CHIMERICHEAVYCHAINFULL-LENGTHAMINOACIDSEQUENCE.HUMANGAMMA-1CONSTANT
DOMAINISUNDERLINED
SEQ ID NO: 31
QSVEESGGRLVTPGTPLTLTCTVSGFTINSYHMIWVRQAPGEGLEYIGFINDGGFTYYASWAKGRFIISRTSTTVDLKMTS
LTVADTATYFCARGVDGTSYPDLWGPGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKRVEPKSCDKTHTCPPCPAPEAAGAPSVFLF
PPKPKDTLMISRTPEVTCVVVDVSHEDPEVKFNWYVDGVEVHNAKTKPREEQYNSTYRVVSVLTVLHQDWLNGKEYK
CAVSNKALPAPIEKTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPG
416G1CHIMERICHEAVYCHAINVARIABLEHEAVYCHAINAMINOACIDSEQUENCE.COMPLIMENTARITY
DETERMININGREGIONSAREUNDERLINED
SEQ ID NO: 32
QSVEESGGRLVTPGTPLTLTCTVSGFTINSYHMIWVRQAPGEGLEYIGFINDGGFTYYASWAKGRFIISRTSTTVDLKMTS
LTVADTATYFCARGVDGTSYPDLWGPGTLVTVSS
418E10CHIMERICLIGHTCHAINFULL-LENGTHNUCLEOTIDESEQUENCE
SEQ ID NO: 33
GCCCTGGTGATGACCCAGACTCCATCCCCTGTGTCTGCGGCTGTGGGAGGCACAGTCACCATCAACTGCCAGGCC
AGTCAGAGCATTGGTAGTAACTTGAACTGGTATCAGCAGAAACCAGGGCAGCCTCCCAAGCTCCTGATTTATTATG
TATCCACTCTGGCATCTGGGGTCCCATCGCGGTTCAAAGGCAGTGGATCTGGGACAGAGTACACTCTCACCATCAG
CGGCGTGCAGTGTGACGATGCTGCCACTTACTACTGCCTAGGTGTTTGGAATTATTGGGGTGATGACGGTACTGCT
TTCGGCGGAGGCACCGAGGTGGAGGTCAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAGAGCAGGACAGCAAGGACAGCACCT
ACAGCCTCAGCAGCACCCTGACGCTGAGCAAAGCAGACTACGAGAAACACAAAGTCTACGCCTGCGAAGTCACCC
ATCAGGGCCTGAGCTCGCCCGTCACAAAGAGCTTCAACAGGGGAGAGTGT
418E10CHIMERICLIGHTCHAINVARIABLELIGHTCHAINNUCLEOTIDESEQUENCE
SEQ ID NO: 34
GCCCTGGTGATGACCCAGACTCCATCCCCTGTGTCTGCGGCTGTGGGAGGCACAGTCACCATCAACTGCCAGGCC
AGTCAGAGCATTGGTAGTAACTTGAACTGGTATCAGCAGAAACCAGGGCAGCCTCCCAAGCTCCTGATTTATTATG
TATCCACTCTGGCATCTGGGGTCCCATCGCGGTTCAAAGGCAGTGGATCTGGGACAGAGTACACTCTCACCATCAG
CGGCGTGCAGTGTGACGATGCTGCCACTTACTACTGCCTAGGTGTTTGGAATTATTGGGGTGATGACGGTACTGCT
TTCGGCGGAGGCACCGAGGTGGAGGTCAAA
418E10CHIMERICLIGHTCHAINFULL-LENGTHAMINOACIDSEQUENCE.HUMANKAPPACONSTANT
DOMAINISUNDERLINED
SEQ ID NO: 35
ALVMTQTPSPVSAAVGGTVTINCQASQSIGSNLNWYQQKPGQPPKLLIYYVSTLASGVPSRFKGSGSGTEYTLTISGVQ
CDDAATYYCLGVWNYWGDDGTAFGGGTEVEVKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNRGEC
418E10CHIMERICLIGHTCHAINVARIABLELIGHTCHAINAMINOACIDSEQUENCE.COMPIMENTARITY
DETERMININGREGIONSAREUNDERLINED
SEQ ID NO: 36
ALVMTQTPSPVSAAVGGTVTINCQASQSIGSNLNWYQQKPGQPPKLLIYYVSTLASGVPSRFKGSGSGTEYTLTISGVQ
CDDAATYYCLGVWNYWGDDGTAFGGGTEVEVK
418E10CHIMERICHEAVYCHAINFULL-LENGTHNUCLEOTIDESEQUENCE
SEQ ID NO: 37
CAGTCGCTGGAGGAGTCCGGGGGTCGCCTGGTCACGCCTGGGACACCCCTGACACTCACCTGCACAGCCTCTGAT
TTCTCCCTCAGTACCTATTATATGAGCTGGGTCCGCCAGGCTCCAGGGAAGGGGCTGGAGTGGATCGGAAGTATT
TATGATAGTGGTGCCGCATACTACGCGACCTGGGCGAAGGGCCGATTCACCATCTCCAGAACGTCGACCACGGTG
GATCTGAGAATGACCAGTCTGACAGCCGCGGACACGGCCACCTATTTCTGTGCCAGAGATCCTATTAATAATGCCA
TCTGGGGCCAAGGGACCCTCGTCACCGTCTCGAGCGCTAGCACCAAGGGCCCATCGGTCTTCCCCCTGGCACCCTC
CTCCAAGAGCACCTCTGGGGGCACAGCGGCCCTGGGCTGCCTGGTCAAGGACTACTTCCCCGAACCGGTGACGGT
GTCGTGGAACTCAGGCGCCCTGACCAGCGGCGTGCACACCTTCCCGGCTGTCCTACAGTCCTCAGGACTCTACTCC
CTCAGCAGCGTGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACCGTGCCCAGCA
CCTGAAGCCGCGGGGGCACCGTCAGTCTTCCTCTTCCCCCCAAAACCCAAGGACACCCTCATGATCTCCCGGACCC
CTGAGGTCACATGCGTGGTGGTGGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGCACGTACCGTGTGGTCAGCGTCCT
CACCGTCCTGCACCAGGACTGGCTGAATGGCAAGGAGTACAAGTGCGCGGTCTCCAACAAAGCCCTCCCAGCCCC
CATCGAGAAAACCATCTCCAAAGCCAAAGGGCAGCCCCGAGAACCACAGGTGTACACCCTGCCCCCATCCCGGGA
TGAGCTGACCAAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGCTCCTTCTTCCT
CTATAGCAAGCTCACCGTGGACAAGAGCAGGTGGCAGCAGGGGAACGTCTTCTCATGCTCCGTGATGCATGAGGC
TCTGCACAACCACTACACGCAGAAGAGCCTCTCCCTGTCTCCGGGT
418E10CHIMERICHEAVYCHAINVARIABLEHEAVYCHAINNUCLEOTIDESEQUENCE
SEQ ID NO: 38
CAGTCGCTGGAGGAGTCCGGGGGTCGCCTGGTCACGCCTGGGACACCCCTGACACTCACCTGCACAGCCTCTGAT
TTCTCCCTCAGTACCTATTATATGAGCTGGGTCCGCCAGGCTCCAGGGAAGGGGCTGGAGTGGATCGGAAGTATT
TATGATAGTGGTGCCGCATACTACGCGACCTGGGCGAAGGGCCGATTCACCATCTCCAGAACGTCGACCACGGTG
GATCTGAGAATGACCAGTCTGACAGCCGCGGACACGGCCACCTATTTCTGTGCCAGAGATCCTATTAATAATGCCA
TCTGGGGCCAAGGGACCCTCGTCACCGTCTCGAGC
418E10CHIMERICHEAVYCHAINFULL-LENGTHAMINOACIDSEQUENCE.HUMANGAMMA-1
CONSTANTDOMAINISUNDERLINED
SEQ ID NO: 39
QSLEESGGRLVTPGTPLTLTCTASDFSLSTYYMSWVRQAPGKGLEWIGSIYDSGAAYYATWAKGRFTISRTSTTVDLRM
TSLTAADTATYFCARDPINNAIWGQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKRVEPKSCDKTHTCPPCPAPEAAGAPSVFLFPP
KPKDTLMISRTPEVTCVVVDVSHEDPEVKFNWYVDGVEVHNAKTKPREEQYNSTYRVVSVLTVLHQDWLNGKEYKCA
VSNKALPAPIEKTISKAKGQPREPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPG
418E10CHIMERICHEAVYCHAINVARIABLEHEAVYCHAINAMINOACIDSEQUENCE.
COMPLIMENTARITYDETERMININGREGIONSAREUNDERLINED
SEQ ID NO: 40
QSLEESGGRLVTPGTPLTLTCTASDFSLSTYYMSWVRQAPGKGLEWIGSIYDSGAAYYATWAKGRFTISRTSTTVDLRM
TSLTAADTATYFCARDPINNAIWGQGTLVTVSS
418E4CHIMERICLIGHTCHAINFULL-LENGTHNUCLEOTIDESEQUENCE
SEQ ID NO: 41
GCCCTGGTGATGACCCAGACTCCATCCCCTGTGTCTGCGGCTGTGGGAGGCACAGTCACCATCAACTGCCAGGCC
AGTCAGAGTATTGCTACTAACTTGAACTGGTATCAGCAGAAACCAGGGCAGCCTCCCAAGCTCCTGATCTATTATA
CATCCAGTCTGGCATCTGGGGTCCCATCGCGGTTCAGCGGCAGTGGATCTGGCACAGAGTTCACTCTCACCATCAG
CGGTGTGCAGTGTGACGATGCTGCCACTTACTACTGCCTAGGTGTTTGGAATTATTGGGGTGATGATGGTACTGCT
TTCGGCGGAGGGACCGAGGTGGAGTTCAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAGAGCAGGACAGCAAGGACAGCACCT
ACAGCCTCAGCAGCACCCTGACGCTGAGCAAAGCAGACTACGAGAAACACAAAGTCTACGCCTGCGAAGTCACCC
ATCAGGGCCTGAGCTCGCCCGTCACAAAGAGCTTCAACAGGGGAGAGTGT
418E4CHIMERICLIGHTCHAINVARIABLELIGHTCHAINNUCLEOTIDESEQUENCE
SEQ ID NO: 42
GCCCTGGTGATGACCCAGACTCCATCCCCTGTGTCTGCGGCTGTGGGAGGCACAGTCACCATCAACTGCCAGGCC
AGTCAGAGTATTGCTACTAACTTGAACTGGTATCAGCAGAAACCAGGGCAGCCTCCCAAGCTCCTGATCTATTATA
CATCCAGTCTGGCATCTGGGGTCCCATCGCGGTTCAGCGGCAGTGGATCTGGCACAGAGTTCACTCTCACCATCAG
CGGTGTGCAGTGTGACGATGCTGCCACTTACTACTGCCTAGGTGTTTGGAATTATTGGGGTGATGATGGTACTGCT
TTCGGCGGAGGGACCGAGGTGGAGTTCAAA
418E4CHIMERICLIGHTCHAINFULL-LENGTHAMINOACIDSEQUENCE.HUMANKAPPACONSTANT
DOMAINISUNDERLINED
SEQ ID NO: 43
ALVMTQTPSPVSAAVGGTVTINCQASQSIATNLNWYQQKPGQPPKLLIYYTSSLASGVPSRFSGSGSGTEFTLTISGVQC
DDAATYYCLGVWNYWGDDGTAFGGGTEVEFKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSSPVTKSFNRGEC
418E4CHIMERICLIGHTCHAINVARIABLELIGHTCHAINAMINOACIDSEQUENCE.COMPIMENTARITY
DETERMININGREGIONSAREUNDERLINED
SEQ ID NO: 44
ALVMTQTPSPVSAAVGGTVTINCQASQSIATNLNWYQQKPGQPPKLLIYYTSSLASGVPSRFSGSGSGTEFTLTISGVQC
DDAATYYCLGVWNYWGDDGTAFGGGTEVEFK
418E4CHIMERICHEAVYCHAINFULL-LENGTHNUCLEOTIDESEQUENCE
SEQ ID NO: 45
CAGTCGCTGGAGGAGTCCGGGGGTCGCCTGGTCACGCCTGGGACACCCCTGACACTCACCTGCACAGCCTCTGAA
TTCTCCCTCAGTAGCTACTACATGAGCTGGGTCCGCCAGGCTCCAGGGAAGGGGCTGGAATGGATCGGAAGTATT
TATGCTAGTGGTAGCGCATACTACGCGAGTTGGGCGAAGGGCCGATTCACCATCTCCAGAACGTCGACCACGGTG
GATCTGAAAATGACCAGTCTGACAGCCGCGGACACGGCCACCTATTTCTGTGTCAGAGATCCTATTAACAATGACA
TCTGGGGCCCAGGCACCCTGGTCACCGTCTCGAGCGCTAGCACCAAGGGCCCATCGGTCTTCCCCCTGGCACCCTC
CTCCAAGAGCACCTCTGGGGGCACAGCGGCCCTGGGCTGCCTGGTCAAGGACTACTTCCCCGAACCGGTGACGGT
GTCGTGGAACTCAGGCGCCCTGACCAGCGGCGTGCACACCTTCCCGGCTGTCCTACAGTCCTCAGGACTCTACTCC
CTCAGCAGCGTGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACCGTGCCCAGCA
CCTGAAGCCGCGGGGGCACCGTCAGTCTTCCTCTTCCCCCCAAAACCCAAGGACACCCTCATGATCTCCCGGACCC
CTGAGGTCACATGCGTGGTGGTGGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGCACGTACCGTGTGGTCAGCGTCCT
CACCGTCCTGCACCAGGACTGGCTGAATGGCAAGGAGTACAAGTGCGCGGTCTCCAACAAAGCCCTCCCAGCCCC
CATCGAGAAAACCATCTCCAAAGCCAAAGGGCAGCCCCGAGAACCACAGGTGTACACCCTGCCCCCATCCCGGGA
TGAGCTGACCAAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGCTCCTTCTTCCT
CTATAGCAAGCTCACCGTGGACAAGAGCAGGTGGCAGCAGGGGAACGTCTTCTCATGCTCCGTGATGCATGAGGC
TCTGCACAACCACTACACGCAGAAGAGCCTCTCCCTGTCTCCGGGT
418E4CHIMERICHEAVYCHAINVARIABLEHEAVYCHAINNUCLEOTIDESEQUENCE
SEQ ID NO: 46
CAGTCGCTGGAGGAGTCCGGGGGTCGCCTGGTCACGCCTGGGACACCCCTGACACTCACCTGCACAGCCTCTGAA
TTCTCCCTCAGTAGCTACTACATGAGCTGGGTCCGCCAGGCTCCAGGGAAGGGGCTGGAATGGATCGGAAGTATT
TATGCTAGTGGTAGCGCATACTACGCGAGTTGGGCGAAGGGCCGATTCACCATCTCCAGAACGTCGACCACGGTG
GATCTGAAAATGACCAGTCTGACAGCCGCGGACACGGCCACCTATTTCTGTGTCAGAGATCCTATTAACAATGACA
TCTGGGGCCCAGGCACCCTGGTCACCGTCTCGAGC
418E4 CHIMERIC HEAVY CHAIN FULL-LENGTH AMINO ACID SEQUENCE. HUMAN GAMMA-1 CONSTANT
DOMAINISUNDERLINED
SEQ ID NO: 47
QSLEESGGRLVTPGTPLTLTCTASEFSLSSYYMSWVRQAPGKGLEWIGSIYASGSAYYASWAKGRFTISRTSTTVDLKMT
SLTAADTATYFCVRDPINNDIWGPGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKRVEPKSCDKTHTCPPCPAPEAAGAPSVFLFPPK
PKDTLMISRTPEVTCVVVDVSHEDPEVKFNWYVDGVEVHNAKTKPREEQYNSTYRVVSVLTVLHQDWLNGKEYKCAV
SNKALPAPIEKTISKAKGQPREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPG
418E4CHIMERICHEAVYCHAINVARIABLEHEAVYCHAINAMINOACIDSEQUENCE.COMPLIMENTARITY
DETERMININGREGIONSAREUNDERLINED
SEQ ID NO: 48
QSLEESGGRLVTPGTPLTLTCTASEFSLSSYYMSWVRQAPGKGLEWIGSIYASGSAYYASWAKGRFTISRTSTTVDLKMT
SLTAADTATYFCVRDPINNDIWGPGTLVTVSS
418H5CHIMERICLIGHTCHAINFULL-LENGTHNUCLEOTIDESEQUENCE
SEQ ID NO: 49
GCCCAAGTGCTGACCCAGACTCCAGCCTCCGTGTCTGCAACTGTGGGAAGCACAGTCACCATCAGTTGCCAGTCCA
GTCCGAGTGTTTATAAGAACAACCAATTAGCCTGGTATCAGCAGAAACCAGGGCAGCCTCCCAAACTCCTGATCTA
TCTGGCATCTACTCTGGCATCTGGGGTCCCATCGCGGTTCAAAGGCAGTGGATCTGGGACAGAGTTCACTCTCACC
ATCAGCGACCTGGAGTGTGCCGATGCTGCCACTTACTACTGTGCAGGCGGTTATAGTAGTAGTAGTGATACTGCTT
TCGGCGGAGGGACCGAGGTGGAGGTCAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAGAGCAGGACAGCAAGGACAGCACCT
ACAGCCTCAGCAGCACCCTGACGCTGAGCAAAGCAGACTACGAGAAACACAAAGTCTACGCCTGCGAAGTCACCC
ATCAGGGCCTGAGCTCGCCCGTCACAAAGAGCTTCAACAGGGGAGAGTGT
418H5CHIMERICLIGHTCHAINVARIABLELIGHTCHAINNUCLEOTIDESEQUENCE
SEQ ID NO: 50
GCCCAAGTGCTGACCCAGACTCCAGCCTCCGTGTCTGCAACTGTGGGAAGCACAGTCACCATCAGTTGCCAGTCCA
GTCCGAGTGTTTATAAGAACAACCAATTAGCCTGGTATCAGCAGAAACCAGGGCAGCCTCCCAAACTCCTGATCTA
TCTGGCATCTACTCTGGCATCTGGGGTCCCATCGCGGTTCAAAGGCAGTGGATCTGGGACAGAGTTCACTCTCACC
ATCAGCGACCTGGAGTGTGCCGATGCTGCCACTTACTACTGTGCAGGCGGTTATAGTAGTAGTAGTGATACTGCTT
TCGGCGGAGGGACCGAGGTGGAGGTCAAA
418H5CHIMERICLIGHTCHAINFULL-LENGTHAMINOACIDSEQUENCE.HUMANKAPPACONSTANT
DOMAINISUNDERLINED
SEQ ID NO: 51
AQVLTQTPASVSATVGSTVTISCQSSPSVYKNNQLAWYQQKPGQPPKLLIYLASTLASGVPSRFKGSGSGTEFTLTISDLE
CADAATYYCAGGYSSSSDTAFGGGTEVEVKRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC
418H5CHIMERICLIGHTCHAINVARIABLELIGHTCHAINAMINOACIDSEQUENCE.COMPIMENTARITY
DETERMININGREGIONSAREUNDERLINED
SEQ ID NO: 52
AQVLTQTPASVSATVGSTVTISCQSSPSVYKNNQLAWYQQKPGQPPKLLIYLASTLASGVPSRFKGSGSGTEFTLTISDLE
CADAATYYCAGGYSSSSDTAFGGGTEVEVK
418H5CHIMERICHEAVYCHAINFULL-LENGTHNUCLEOTIDESEQUENCE
SEQ ID NO: 53
CAGTCGCTGGAGGAGTCCGGGGGTCGCCTGGTCACGCCTGGGACACCCCTGACACTCACCTGCACAGTCTCTGGA
ATCGACCTCAGTGCCTACCACATGAGCTGGGTCCGCCAGGCTCCAGGGAAGGGGCTGGAATACATCGGAATGATT
GGTAGTAGTGGTACCATACACTACGCGAACTGGGCGAAAGGCCGATTCACCATCTCCAAAACCTCGACCACGGTG
GATCTGAAAATCACCAGTCCGACAACCGAGGACACGGCCACCTATTTCTGTGCCAGGGACAGTTATAATAGTGATT
ATGCCTTTAACTTATGGGGCCCAGGGACCCTGGTCACCGTCTCGAGCGCTAGCACCAAGGGCCCATCGGTCTTCCC
CCTGGCACCCTCCTCCAAGAGCACCTCTGGGGGCACAGCGGCCCTGGGCTGCCTGGTCAAGGACTACTTCCCCGA
ACCGGTGACGGTGTCGTGGAACTCAGGCGCCCTGACCAGCGGCGTGCACACCTTCCCGGCTGTCCTACAGTCCTC
AGGACTCTACTCCCTCAGCAGCGTGGTGACCGTGCCCTCCAGCAGCTTGGGCACCCAGACCTACATCTGCAACGTG
AATCACAAGCCCAGCAACACCAAGGTGGACAAGAGAGTTGAGCCCAAATCTTGTGACAAAACTCACACATGCCCA
CCGTGCCCAGCACCTGAAGCCGCGGGGGCACCGTCAGTCTTCCTCTTCCCCCCAAAACCCAAGGACACCCTCATGA
TCTCCCGGACCCCTGAGGTCACATGCGTGGTGGTGGACGTGAGCCACGAAGACCCTGAGGTCAAGTTCAACTGGT
ACGTGGACGGCGTGGAGGTGCATAATGCCAAGACAAAGCCGCGGGAGGAGCAGTACAACAGCACGTACCGTGT
GGTCAGCGTCCTCACCGTCCTGCACCAGGACTGGCTGAATGGCAAGGAGTACAAGTGCGCGGTCTCCAACAAAGC
CCTCCCAGCCCCCATCGAGAAAACCATCTCCAAAGCCAAAGGGCAGCCCCGAGAACCACAGGTGTACACCCTGCC
CCCATCCCGGGATGAGCTGACCAAGAACCAGGTCAGCCTGACCTGCCTGGTCAAAGGCTTCTATCCCAGCGACATC
GCCGTGGAGTGGGAGAGCAATGGGCAGCCGGAGAACAACTACAAGACCACGCCTCCCGTGCTGGACTCCGACGG
CTCCTTCTTCCTCTATAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGT
418H5CHIMERICHEAVYCHAINVARIABLEHEAVYCHAINNUCLEOTIDESEQUENCE
SEQ ID NO: 54
CAGTCGCTGGAGGAGTCCGGGGGTCGCCTGGTCACGCCTGGGACACCCCTGACACTCACCTGCACAGTCTCTGGA
ATCGACCTCAGTGCCTACCACATGAGCTGGGTCCGCCAGGCTCCAGGGAAGGGGCTGGAATACATCGGAATGATT
GGTAGTAGTGGTACCATACACTACGCGAACTGGGCGAAAGGCCGATTCACCATCTCCAAAACCTCGACCACGGTG
GATCTGAAAATCACCAGTCCGACAACCGAGGACACGGCCACCTATTTCTGTGCCAGGGACAGTTATAATAGTGATT
ATGCCTTTAACTTATGGGGCCCAGGGACCCTGGTCACCGTCTCGAGC
418H5 CHIMERIC HEAVY CHAIN FULL-LENGTH AMINO ACID SEQUENCE. HUMAN GAMMA-1 CONSTANT
DOMAINISUNDERLINED
SEQ ID NO: 55
QSLEESGGRLVTPGTPLTLTCTVSGIDLSAYHMSWVRQAPGKGLEYIGMIGSSGTIHYANWAKGRFTISKTSTTVDLKITS
PTTEDTATYFCARDSYNSDYAFNLWGPGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKRVEPKSCDKTHTCPPCPAPEAAGAPSVFL
FPPKPKDTLMISRTPEVTCVVVDVSHEDPEVKFNWYVDGVEVHNAKTKPREEQYNSTYRVVSVLTVLHQDWLNGKEY
KCAVSNKALPAPIEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPG
418H5CHIMERICHEAVYCHAINVARIABLEHEAVYCHAINAMINOACIDSEQUENCE.COMPLIMENTARITY
DETERMININGREGIONSAREUNDERLINED
SEQ ID NO: 56
QSLEESGGRLVTPGTPLTLTCTVSGIDLSAYHMSWVRQAPGKGLEYIGMIGSSGTIHYANWAKGRFTISKTSTTVDLKITS
PTTEDTATYFCARDSYNSDYAFNLWGPGTLVTVSS
419D9CHIMERICLIGHTCHAINFULL-LENGTHNUCLEOTIDESEQUENCE
SEQ ID NO: 57
GCTCAAGTGCTGACCCAGACTGCATCGCCCGTGTCTGCCGCTGTGGGAGGCACAGTCACCATCAACTGCCAGTCCA
GTCAGAGTGTTTATAGTAACTGGTTATCCTGGTATCAGCAGAAACCAGGGCAGCCTCCCAAGCGCCTGATCTATTC
TGCATCCACTCTGGCATCTGGGGTCCCATCGCGGTTCAAAGGCAGTGGATCTGGGACACAGTTCACTCTCACCATC
AGCGACCTGGAGTGTGACGATGCTGCCACTTACTACTGTGCAGGCGGTTATAATACTGTTACTGATACTTTTGCTTT
CGGCAGAGGCACCGAGGTGGAGGTCAAACGTACGGTGGCTGCACCATCTGTCTTCATCTTCCCGCCATCTGATGA
GCAGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGT
419D9CHIMERICLIGHTCHAINVARIABLELIGHTCHAINNUCLEOTIDESEQUENCE
SEQ ID NO: 58
GCTCAAGTGCTGACCCAGACTGCATCGCCCGTGTCTGCCGCTGTGGGAGGCACAGTCACCATCAACTGCCAGTCCA
GTCAGAGTGTTTATAGTAACTGGTTATCCTGGTATCAGCAGAAACCAGGGCAGCCTCCCAAGCGCCTGATCTATTC
TGCATCCACTCTGGCATCTGGGGTCCCATCGCGGTTCAAAGGCAGTGGATCTGGGACACAGTTCACTCTCACCATC
AGCGACCTGGAGTGTGACGATGCTGCCACTTACTACTGTGCAGGCGGTTATAATACTGTTACTGATACTTTTGCTTT
CGGCAGAGGCACCGAGGTGGAGGTCAAA
419D9CHIMERICLIGHTCHAINFULL-LENGTHAMINOACIDSEQUENCE.HUMANKAPPACONSTANT
DOMAINISUNDERLINED
SEQ ID NO: 59
AQVLTQTASPVSAAVGGTVTINCQSSQSVYSNWLSWYQQKPGQPPKRLIYSASTLASGVPSRFKGSGSGTQFTLTISDL
ECDDAATYYCAGGYNTVTDTFAFGRGTEVEVKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
419D9CHIMERICLIGHTCHAINVARIABLELIGHTCHAINAMINOACIDSEQUENCE.COMPIMENTARITY
DETERMININGREGIONSAREUNDERLINED
SEQ ID NO: 60
AQVLTQTASPVSAAVGGTVTINCQSSQSVYSNWLSWYQQKPGQPPKRLIYSASTLASGVPSRFKGSGSGTQFTLTISDL
ECDDAATYYCAGGYNTVTDTFAFGRGTEVEVK
419D9CHIMERICHEAVYCHAINFULL-LENGTHNUCLEOTIDESEQUENCE
SEQ ID NO: 61
CAGTCGTTGGAGGAGTCCGGGGGAGACCTGGTCAAGCCTGGGGCATCCCTGACACTCACCTGCACAGCCTCTGGA
TTCTCCTTCAGTAGCAGGCACTACATGTGTTGGGTCCGCCAGGCTCCAGGGGAGGGGCTGGAGTGGATCGCATGC
ATTTATACTGGTAGTAGTGGTACTCCTCACTACGCGAGCTGGGCGAAAGGCCGATTCACCATCTCCCAAACCTCGT
CGACCACGGTGACTCTGCAAATGACCAGTCTGACAGCCGCGGACACGGCCACCTATTTCTGTTCGAGCGAAGGTA
ACCTGTGGGGCCCAGGCACCCTGGTCACCGTCTCGAGCGCTAGCACCAAGGGCCCATCGGTCTTCCCCCTGGCACC
CTCCTCCAAGAGCACCTCTGGGGGCACAGCGGCCCTGGGCTGCCTGGTCAAGGACTACTTCCCCGAACCGGTGAC
GGTGTCGTGGAACTCAGGCGCCCTGACCAGCGGCGTGCACACCTTCCCGGCTGTCCTACAGTCCTCAGGACTCTAC
TCCCTCAGCAGCGTGGTGACCGTGCCCTCCAGCAGCTTGGGCACCCAGACCTACATCTGCAACGTGAATCACAAGC
CCAGCAACACCAAGGTGGACAAGAGAGTTGAGCCCAAATCTTGTGACAAAACTCACACATGCCCACCGTGCCCAG
CACCTGAAGCCGCGGGGGCACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGTGTGGTCAGCGTC
CTCACCGTCCTGCACCAGGACTGGCTGAATGGCAAGGAGTACAAGTGCGCGGTCTCCAACAAAGCCCTCCCAGCC
CCCATCGAGAAAACCATCTCCAAAGCCAAAGGGCAGCCCCGAGAACCACAGGTGTACACCCTGCCCCCATCCCGG
GATGAGCTGACCAAGAACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCCGACGGCTCCTTCTTC
CTCTATAGCAAGCTCACCGTGGACAAGAGCAGGTGGCAGCAGGGGAACGTCTTCTCATGCTCCGTGATGCATGAG
GCTCTGCACAACCACTACACGCAGAAGAGCCTCTCCCTGTCTCCGGGT
419D9CHIMERICHEAVYCHAINVARIABLEHEAVYCHAINNUCLEOTIDESEQUENCE
SEQ ID NO: 62
CAGTCGTTGGAGGAGTCCGGGGGAGACCTGGTCAAGCCTGGGGCATCCCTGACACTCACCTGCACAGCCTCTGGA
TTCTCCTTCAGTAGCAGGCACTACATGTGTTGGGTCCGCCAGGCTCCAGGGGAGGGGCTGGAGTGGATCGCATGC
ATTTATACTGGTAGTAGTGGTACTCCTCACTACGCGAGCTGGGCGAAAGGCCGATTCACCATCTCCCAAACCTCGT
CGACCACGGTGACTCTGCAAATGACCAGTCTGACAGCCGCGGACACGGCCACCTATTTCTGTTCGAGCGAAGGTA
ACCTGTGGGGCCCAGGCACCCTGGTCACCGTCTCGAGC
419D9CHIMERICHEAVYCHAINFULL-LENGTHAMINOACIDSEQUENCE.HUMANGAMMA-1CONSTANT
DOMAINISUNDERLINED
SEQ ID NO: 63
QSLEESGGDLVKPGASLTLTCTASGFSFSSRHYMCWVRQAPGEGLEWIACIYTGSSGTPHYASWAKGRFTISQTSSTTV
TLQMTSLTAADTATYFCSSEGNLWGPGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKRVEPKSCDKTHTCPPCPAPEAAGAPSVFLE
PPKPKDTLMISRTPEVTCVVVDVSHEDPEVKFNWYVDGVEVHNAKTKPREEQYNSTYRVVSVLTVLHQDWLNGKEYK
CAVSNKALPAPIEKTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPG
419D9CHIMERICHEAVYCHAINVARIABLEHEAVYCHAINAMINOACIDSEQUENCE.COMPLIMENTARITY
DETERMININGREGIONSAREUNDERLINED
SEQ ID NO: 64
QSLEESGGDLVKPGASLTLTCTASGFSFSSRHYMCWVRQAPGEGLEWIACIYTGSSGTPHYASWAKGRFTISQTSSTTV
TLQMTSLTAADTATYFCSSEGNLWGPGTLVTVSS
420H5CHIMERICLIGHTCHAINFULL-LENGTHNUCLEOTIDESEQUENCE
SEQ ID NO: 65
GCCCTTGTGATGACCCAGACTCCAGCCTCCGTGTCTGCAGCTGTGGGAGGCACAGTCACCATCAATTGCCAGGCCA
GTGAGGACATTGATACCTATTTAGCCTGGTATCAGCAGAAACCAGGGCAGCCTCCCAAGCTCCTGATCTTTTATGC
ATCCGATCTGGCATCTGGGGTCCCATCGCGGTTCAAAGGCAGTGGATCTGGGACAGAGTTCACTCTCACCATCAGC
GGTGTGCAGTGTGACGATGCTGCCACTTACTACTGTCAAGGCGGTTACTATACTAGTAGTGCTGATACGAGGGGT
GCTTTCGGCGGAGGCACCGAGGTGGTGGTCAAACGTACGGTGGCTGCACCATCTGTCTTCATCTTCCCGCCATCTG
ATGAGCAGTTGAAATCTGGAACTGCCTCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCACAGAGCAGGACAGCAAGGACAGCA
CCTACAGCCTCAGCAGCACCCTGACGCTGAGCAAAGCAGACTACGAGAAACACAAAGTCTACGCCTGCGAAGTCA
CCCATCAGGGCCTGAGCTCGCCCGTCACAAAGAGCTTCAACAGGGGAGAGTGT
420H5CHIMERICLIGHTCHAINVARIABLELIGHTCHAINNUCLEOTIDESEQUENCE
SEQ ID NO: 66
GCCCTTGTGATGACCCAGACTCCAGCCTCCGTGTCTGCAGCTGTGGGAGGCACAGTCACCATCAATTGCCAGGCCA
GTGAGGACATTGATACCTATTTAGCCTGGTATCAGCAGAAACCAGGGCAGCCTCCCAAGCTCCTGATCTTTTATGC
ATCCGATCTGGCATCTGGGGTCCCATCGCGGTTCAAAGGCAGTGGATCTGGGACAGAGTTCACTCTCACCATCAGC
GGTGTGCAGTGTGACGATGCTGCCACTTACTACTGTCAAGGCGGTTACTATACTAGTAGTGCTGATACGAGGGGT
GCTTTCGGCGGAGGCACCGAGGTGGTGGTCAAA
420H5CHIMERICLIGHTCHAINFULL-LENGTHAMINOACIDSEQUENCE.HUMANKAPPACONSTANT
DOMAINISUNDERLINED
SEQ ID NO: 67
ALVMTQTPASVSAAVGGTVTINCQASEDIDTYLAWYQQKPGQPPKLLIFYASDLASGVPSRFKGSGSGTEFTLTISGVQC
DDAATYYCQGGYYTSSADTRGAFGGGTEVVVKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
420H5CHIMERICLIGHTCHAINVARIABLELIGHTCHAINAMINOACIDSEQUENCE.COMPIMENTARITY
DETERMININGREGIONSAREUNDERLINED
SEQ ID NO: 68
ALVMTQTPASVSAAVGGTVTINCQASEDIDTYLAWYQQKPGQPPKLLIFYASDLASGVPSRFKGSGSGTEFTLTISGVQC
DDAATYYCQGGYYTSSADTRGAFGGGTEVVVK
420H5CHIMERICHEAVYCHAINFULL-LENGTHNUCLEOTIDESEQUENCE
SEQ ID NO: 69
CAGTCGTTGGAGGAGTCCGGGGGAGACCTGGTCAAGCCTGGGGCATCCCTGACACTCACCTGCACAGCCTCTGGA
TTCTCCTTCAGTAGCAACTACTGGATATGCTGGGTCCGCCAGGCTCCAGGGAAGGGGCTGGAGTGGATCGCATGC
ATTTATGTTGGTAGTAGTGGTGACACTTACTACGCGAGCTGGGCGAAAGGCCGATTCACCATCTCCAAAACCTCGT
CGACCACGGTGACTCTGCAAATGACCAGTCTGACAGCCGCGGACACGGCCACCTATTTCTGTGCGAGAGATAGTA
GTAGTTATTATATGTTTAACTTGTGGGGCCAGGGGACCCTGGTCACCGTCTCGAGCGCTAGCACCAAGGGCCCATC
GGTCTTCCCCCTGGCACCCTCCTCCAAGAGCACCTCTGGGGGCACAGCGGCCCTGGGCTGCCTGGTCAAGGACTA
CTTCCCCGAACCGGTGACGGTGTCGTGGAACTCAGGCGCCCTGACCAGCGGCGTGCACACCTTCCCGGCTGTCCTA
CAGTCCTCAGGACTCTACTCCCTCAGCAGCGTGGTGACCGTGCCCTCCAGCAGCTTGGGCACCCAGACCTACATCT
GCAACGTGAATCACAAGCCCAGCAACACCAAGGTGGACAAGAGAGTTGAGCCCAAATCTTGTGACAAAACTCACA
CATGCCCACCGTGCCCAGCACCTGAAGCCGCGGGGGCACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTGAGGTCAAGTT
CAACTGGTACGTGGACGGCGTGGAGGTGCATAATGCCAAGACAAAGCCGCGGGAGGAGCAGTACAACAGCACG
TACCGTGTGGTCAGCGTCCTCACCGTCCTGCACCAGGACTGGCTGAATGGCAAGGAGTACAAGTGCGCGGTCTCC
AACAAAGCCCTCCCAGCCCCCATCGAGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGACCTGCCTGGTCAAAGGCTTCTATCCCA
GCGACATCGCCGTGGAGTGGGAGAGCAATGGGCAGCCGGAGAACAACTACAAGACCACGCCTCCCGTGCTGGAC
TCCGACGGCTCCTTCTTCCTCTATAGCAAGCTCACCGTGGACAAGAGCAGGTGGCAGCAGGGGAACGTCTTCTCAT
GCTCCGTGATGCATGAGGCTCTGCACAACCACTACACGCAGAAGAGCCTCTCCCTGTCTCCGGGT
420H5CHIMERICHEAVYCHAINVARIABLEHEAVYCHAINNUCLEOTIDESEQUENCE
SEQ ID NO: 70
CAGTCGTTGGAGGAGTCCGGGGGAGACCTGGTCAAGCCTGGGGCATCCCTGACACTCACCTGCACAGCCTCTGGA
TTCTCCTTCAGTAGCAACTACTGGATATGCTGGGTCCGCCAGGCTCCAGGGAAGGGGCTGGAGTGGATCGCATGC
ATTTATGTTGGTAGTAGTGGTGACACTTACTACGCGAGCTGGGCGAAAGGCCGATTCACCATCTCCAAAACCTCGT
CGACCACGGTGACTCTGCAAATGACCAGTCTGACAGCCGCGGACACGGCCACCTATTTCTGTGCGAGAGATAGTA
GTAGTTATTATATGTTTAACTTGTGGGGCCAGGGGACCCTGGTCACCGTCTCGAGC
420H5CHIMERICHEAVYCHAINFULL-LENGTHAMINOACIDSEQUENCE.HUMANGAMMA-1CONSTANT
DOMAINISUNDERLINED
SEQ ID NO: 71
QSLEESGGDLVKPGASLTLTCTASGFSFSSNYWICWVRQAPGKGLEWIACIYVGSSGDTYYASWAKGRFTISKTSSTTVT
LQMTSLTAADTATYFCARDSSSYYMFNLWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVDKRVEPKSCDKTHTCPPCPAPEAAG
APSVFLFPPKPKDTLMISRTPEVTCVVVDVSHEDPEVKFNWYVDGVEVHNAKTKPREEQYNSTYRVVSVLTVLHQDWL
NGKEYKCAVSNKALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPG
420H5CHIMERICHEAVYCHAINVARIABLEHEAVYCHAINAMINOACIDSEQUENCE.COMPLIMENTARITY
DETERMININGREGIONSAREUNDERLINED
SEQ ID NO: 72
QSLEESGGDLVKPGASLTLTCTASGFSFSSNYWICWVRQAPGKGLEWIACIYVGSSGDTYYASWAKGRFTISKTSSTTVT
LQMTSLTAADTATYFCARDSSSYYMFNLWGQGTLVTVSS
420H6CHIMERICLIGHTCHAINFULL-LENGTHNUCLEOTIDESEQUENCE
SEQ ID NO: 73
GCTCAAGTGCTGACCCAGACTGCATCGCCCGTGTCTGCCGCTGTGGGAGGCACAGTCACCATCAACTGCCAGGCC
AGTCAGAGTGTTTATAGTAACTGGTTATCCTGGTATCAGCAGAAACCAGGGCAGCCTCCCAAGCGCCTGATCTATT
CTGCATCCACTCTGGCATCTGGGGTCCCATCGCGGTTCAAAGGCAGTGGATCTGGGACACAGTTCACTCTCACCAT
TAGCGACCTGGAGTGTGACGATGCTGCCACTTACTACTGTGCAGGCGGTTACAATACTGTTATTGATACTTTTGCTT
TCGGCGGAGGCACCGAGGTGGAGTTCAAACGTACGGTGGCTGCACCATCTGTCTTCATCTTCCCGCCATCTGATGA
GCAGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGT
420H6CHIMERICLIGHTCHAINVARIABLELIGHTCHAINNUCLEOTIDESEQUENCE
SEQ ID NO: 74
GCTCAAGTGCTGACCCAGACTGCATCGCCCGTGTCTGCCGCTGTGGGAGGCACAGTCACCATCAACTGCCAGGCC
AGTCAGAGTGTTTATAGTAACTGGTTATCCTGGTATCAGCAGAAACCAGGGCAGCCTCCCAAGCGCCTGATCTATT
CTGCATCCACTCTGGCATCTGGGGTCCCATCGCGGTTCAAAGGCAGTGGATCTGGGACACAGTTCACTCTCACCAT
TAGCGACCTGGAGTGTGACGATGCTGCCACTTACTACTGTGCAGGCGGTTACAATACTGTTATTGATACTTTTGCTT
TCGGCGGAGGCACCGAGGTGGAGTTCAAA
420H6CHIMERICLIGHTCHAINFULL-LENGTHAMINOACIDSEQUENCE.HUMANKAPPACONSTANT
DOMAINISUNDERLINED
SEQ ID NO: 75
AQVLTQTASPVSAAVGGTVTINCQASQSVYSNWLSWYQQKPGQPPKRLIYSASTLASGVPSRFKGSGSGTQFTLTISDL
ECDDAATYYCAGGYNTVIDTFAFGGGTEVEFKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
420H6CHIMERICLIGHTCHAINVARIABLELIGHTCHAINAMINOACIDSEQUENCE.COMPIMENTARITY
DETERMININGREGIONSAREUNDERLINED
SEQ ID NO: 76
AQVLTQTASPVSAAVGGTVTINCQASQSVYSNWLSWYQQKPGQPPKRLIYSASTLASGVPSRFKGSGSGTQFTLTISDL
ECDDAATYYCAGGYNTVIDTFAFGGGTEVEFK
420H6CHIMERICHEAVYCHAINFULL-LENGTHNUCLEOTIDESEQUENCE
SEQ ID NO: 77
CAGCAGCAGCTGGAGGAGTCCGGGGGAGGCCTGGTCAAGCCTGGAGGAACCCTGACACTCACCTGCAAAGCCTC
TGGAATTGACTTCAGTAGTAGTTACTACATGTGCTGGGTCCGCCAGGCTCCAGGGGAGGGGCTGGAGTGGATCGC
ATGCATTTATACTGGTGGTAGTGGTACTCCTCACTACGCGAGCTGGGCGAAAGGCCGATTCACCATCTCCCAAACC
TCGTCGACCACGGTGACTCTGCAAATGACCAGTCTGACAGCCGCGGACACGGCCACCTATTTCTGTGCGAGAGAA
GGTAGCCTGTGGGGCCAGGGCACCCTGGTCACCGTCTCGAGCGCTAGCACCAAGGGCCCATCGGTCTTCCCCCTG
GCACCCTCCTCCAAGAGCACCTCTGGGGGCACAGCGGCCCTGGGCTGCCTGGTCAAGGACTACTTCCCCGAACCG
GTGACGGTGTCGTGGAACTCAGGCGCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACCCAGACCTACATCTGCAACGTGAATC
ACAAGCCCAGCAACACCAAGGTGGACAAGAGAGTTGAGCCCAAATCTTGTGACAAAACTCACACATGCCCACCGT
GCCCAGCACCTGAAGCCGCGGGGGCACCGTCAGTCTTCCTCTTCCCCCCAAAACCCAAGGACACCCTCATGATCTC
CCGGACCCCTGAGGTCACATGCGTGGTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGTACAACAGCACGTACCGTGTGGTC
AGCGTCCTCACCGTCCTGCACCAGGACTGGCTGAATGGCAAGGAGTACAAGTGCGCGGTCTCCAACAAAGCCCTC
CCAGCCCCCATCGAGAAAACCATCTCCAAAGCCAAAGGGCAGCCCCGAGAACCACAGGTGTACACCCTGCCCCCA
TCCCGGGATGAGCTGACCAAGAACCAGGTCAGCCTGACCTGCCTGGTCAAAGGCTTCTATCCCAGCGACATCGCC
GTGGAGTGGGAGAGCAATGGGCAGCCGGAGAACAACTACAAGACCACGCCTCCCGTGCTGGACTCCGACGGCTC
CTTCTTCCTCTATAGCAAGCTCACCGTGGACAAGAGCAGGTGGCAGCAGGGGAACGTCTTCTCATGCTCCGTGATG
CATGAGGCTCTGCACAACCACTACACGCAGAAGAGCCTCTCCCTGTCTCCGGGT
420H6CHIMERICHEAVYCHAINVARIABLEHEAVYCHAINNUCLEOTIDESEQUENCE
SEQ ID NO: 78
CAGCAGCAGCTGGAGGAGTCCGGGGGAGGCCTGGTCAAGCCTGGAGGAACCCTGACACTCACCTGCAAAGCCTC
TGGAATTGACTTCAGTAGTAGTTACTACATGTGCTGGGTCCGCCAGGCTCCAGGGGAGGGGCTGGAGTGGATCGC
ATGCATTTATACTGGTGGTAGTGGTACTCCTCACTACGCGAGCTGGGCGAAAGGCCGATTCACCATCTCCCAAACC
TCGTCGACCACGGTGACTCTGCAAATGACCAGTCTGACAGCCGCGGACACGGCCACCTATTTCTGTGCGAGAGAA
GGTAGCCTGTGGGGCCAGGGCACCCTGGTCACCGTCTCGAGC
420H6CHIMERICHEAVYCHAINFULL-LENGTHAMINOACIDSEQUENCE.HUMANGAMMA-1CONSTANT
DOMAINISUNDERLINED
SEQ ID NO: 79
QQQLEESGGGLVKPGGTLTLTCKASGIDFSSSYYMCWVRQAPGEGLEWIACIYTGGSGTPHYASWAKGRFTISQTSSTT
VTLQMTSLTAADTATYFCAREGSLWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKRVEPKSCDKTHTCPPCPAPEAAGAPSVFL
FPPKPKDTLMISRTPEVTCVVVDVSHEDPEVKFNWYVDGVEVHNAKTKPREEQYNSTYRVVSVLTVLHQDWLNGKEY
KCAVSNKALPAPIEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPG
420H6CHIMERICHEAVYCHAINVARIABLEHEAVYCHAINAMINOACIDSEQUENCE.COMPLIMENTARITY
DETERMININGREGIONSAREUNDERLINED
SEQ ID NO: 80
QQQLEESGGGLVKPGGTLTLTCKASGIDFSSSYYMCWVRQAPGEGLEWIACIYTGGSGTPHYASWAKGRFTISQTSSTT
VTLQMTSLTAADTATYFCAREGSLWGQGTLVTVSS
459F1CHIMERICLIGHTCHAINFULL-LENGTHNUCLEOTIDESEQUENCE
SEQ ID NO: 81
GCTCAAGTGCTGACCCAGACTGCATCGCCCGTGTCTGCCGCTGTGGGAGGCACAGTCACCATCAACTGCCAGTCCA
GTCAGAGTGTTTATAGTAACTGGTTCTCCTGGTATCAGCAGAAACCAGGGCAGCCTCCCAAGCTCCTGATCTATTCT
GCATCCACTCTGGCATCTGGGGTCCCCTCGCGGTTTAAAGGCAGTGGATCTGGGACACAGTTCACTCTCACCATCA
GCGACCTGGAGTGTGACGATGCTGCCACTTACTACTGTGCAGGCGGTTACAATACTGTAACTGATACTTTTGCTTTC
GGCGGAGGCACCGAGGTGGAGTTCAAACGTACGGTGGCTGCACCATCTGTCTTCATCTTCCCGCCATCTGATGAG
CAGTTGAAATCTGGAACTGCCTCTGTTGTGTGCCTGCTGAATAACTTCTATCCCAGAGAGGCCAAAGTACAGTGGA
AGGTGGATAACGCCCTCCAATCGGGTAACTCCCAGGAGAGTGTCACAGAGCAGGACAGCAAGGACAGCACCTAC
AGCCTCAGCAGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAGAGTGT
459F1CHIMERICLIGHTCHAINVARIABLELIGHTCHAINNUCLEOTIDESEQUENCE
SEQ ID NO: 82
GCTCAAGTGCTGACCCAGACTGCATCGCCCGTGTCTGCCGCTGTGGGAGGCACAGTCACCATCAACTGCCAGTCCA
GTCAGAGTGTTTATAGTAACTGGTTCTCCTGGTATCAGCAGAAACCAGGGCAGCCTCCCAAGCTCCTGATCTATTCT
GCATCCACTCTGGCATCTGGGGTCCCCTCGCGGTTTAAAGGCAGTGGATCTGGGACACAGTTCACTCTCACCATCA
GCGACCTGGAGTGTGACGATGCTGCCACTTACTACTGTGCAGGCGGTTACAATACTGTAACTGATACTTTTGCTTTC
GGCGGAGGCACCGAGGTGGAGTTCAAA
459F1CHIMERICLIGHTCHAINFULL-LENGTHAMINOACIDSEQUENCE.HUMANKAPPACONSTANT
DOMAINISUNDERLINED
SEQ ID NO: 83
AQVLTQTASPVSAAVGGTVTINCQSSQSVYSNWFSWYQQKPGQPPKLLIYSASTLASGVPSRFKGSGSGTQFTLTISDLE
CDDAATYYCAGGYNTVTDTFAFGGGTEVEFKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
459F1CHIMERICLIGHTCHAINVARIABLELIGHTCHAINAMINOACIDSEQUENCE.COMPIMENTARITY
DETERMININGREGIONSAREUNDERLINED
SEQ ID NO: 84
AQVLTQTASPVSAAVGGTVTINCQSSQSVYSNWFSWYQQKPGQPPKLLIYSASTLASGVPSRFKGSGSGTQFTLTISDLE
CDDAATYYCAGGYNTVTDTFAFGGGTEVEFK
459F1CHIMERICHEAVYCHAINFULL-LENGTHNUCLEOTIDESEQUENCE
SEQ ID NO: 85
GAGTCGTTGGAGGAGTCCGGGGGAGACCTGGTCAAGCCTGGGGCATCCCTGACACTCACCTGCACAGCCTCTGG
AATCGACTTCAGTAGGAGATACTACATGTGCTGGGTCCGCCAGGCTCCAGGGAAGGGGCTGGAGTGGATCGCAT
GCATGTATACTGGTAGCCGCGATACTCCTCACTACGCGAGCTGGGCGAAAGGCCGGTTCACCATCTCCCAAACCTC
GTCGACCACGGTGACTCTGCAAATGACCAGTCTGACAGCCGCGGACACGGCCACCTATTTCTGTGCGAGAGAAGG
TAACCTGTGGGGCCCGGGGACCCTGGTCACCGTCTCGAGCGCTAGCACCAAGGGCCCATCGGTCTTCCCCCTGGC
ACCCTCCTCCAAGAGCACCTCTGGGGGCACAGCGGCCCTGGGCTGCCTGGTCAAGGACTACTTCCCCGAACCGGT
GACGGTGTCGTGGAACTCAGGCGCCCTGACCAGCGGCGTGCACACCTTCCCGGCTGTCCTACAGTCCTCAGGACT
CTACTCCCTCAGCAGCGTGGTGACCGTGCCCTCCAGCAGCTTGGGCACCCAGACCTACATCTGCAACGTGAATCAC
AAGCCCAGCAACACCAAGGTGGACAAGAGAGTTGAGCCCAAATCTTGTGACAAAACTCACACATGCCCACCGTGC
CCAGCACCTGAAGCCGCGGGGGCACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGGTCA
GCGTCCTCACCGTCCTGCACCAGGACTGGCTGAATGGCAAGGAGTACAAGTGCGCGGTCTCCAACAAAGCCCTCC
CAGCCCCCATCGAGAAAACCATCTCCAAAGCCAAAGGGCAGCCCCGAGAACCACAGGTGTACACCCTGCCCCCAT
CCCGGGATGAGCTGACCAAGAACCAGGTCAGCCTGACCTGCCTGGTCAAAGGCTTCTATCCCAGCGACATCGCCG
TGGAGTGGGAGAGCAATGGGCAGCCGGAGAACAACTACAAGACCACGCCTCCCGTGCTGGACTCCGACGGCTCC
TTCTTCCTCTATAGCAAGCTCACCGTGGACAAGAGCAGGTGGCAGCAGGGGAACGTCTTCTCATGCTCCGTGATGC
ATGAGGCTCTGCACAACCACTACACGCAGAAGAGCCTCTCCCTGTCTCCGGGT
459F1CHIMERICHEAVYCHAINVARIABLEHEAVYCHAINNUCLEOTIDESEQUENCE
SEQ ID NO: 86
GAGTCGTTGGAGGAGTCCGGGGGAGACCTGGTCAAGCCTGGGGCATCCCTGACACTCACCTGCACAGCCTCTGG
AATCGACTTCAGTAGGAGATACTACATGTGCTGGGTCCGCCAGGCTCCAGGGAAGGGGCTGGAGTGGATCGCAT
GCATGTATACTGGTAGCCGCGATACTCCTCACTACGCGAGCTGGGCGAAAGGCCGGTTCACCATCTCCCAAACCTC
GTCGACCACGGTGACTCTGCAAATGACCAGTCTGACAGCCGCGGACACGGCCACCTATTTCTGTGCGAGAGAAGG
TAACCTGTGGGGCCCGGGGACCCTGGTCACCGTCTCGAGC
459F1 CHIMERIC HEAVY CHAIN FULL-LENGTH AMINO ACID SEQUENCE. HUMAN GAMMA-1 CONSTANT
DOMAINISUNDERLINED
SEQ ID NO: 87
ESLEESGGDLVKPGASLTLTCTASGIDFSRRYYMCWVRQAPGKGLEWIACMYTGSRDTPHYASWAKGRFTISQTSSTTV
TLQMTSLTAADTATYFCAREGNLWGPGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKRVEPKSCDKTHTCPPCPAPEAAGAPSVFLE
PPKPKDTLMISRTPEVTCVVVDVSHEDPEVKFNWYVDGVEVHNAKTKPREEQYNSTYRVVSVLTVLHQDWLNGKEYK
CAVSNKALPAPIEKTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPG
459F1CHIMERICHEAVYCHAINVARIABLEHEAVYCHAINAMINOACIDSEQUENCE.COMPLIMENTARITY
DETERMININGREGIONSAREUNDERLINED
SEQ ID NO: 88
ESLEESGGDLVKPGASLTLTCTASGIDFSRRYYMCWVRQAPGKGLEWIACMYTGSRDTPHYASWAKGRFTISQTSSTTV
TLQMTSLTAADTATYFCAREGNLWGPGTLVTVSS
460C3CHIMERICLIGHTCHAINFULL-LENGTHNUCLEOTIDESEQUENCE
SEQ ID NO: 89
GCCCAAGTGCTGACCCAGACTCCATCGTCCGTGTCTGCAGCTGTGGGAGGCACAGTCAGCATCAGTTGCCAGTCC
AGTCAGAGTGTTTATAGTAACTGGTTCTCCTGGTATCAGCAGAAACCAGGGCAGCCTCCCAAGCTCCTGATCTATT
CTGCATCCACTCTGGCATCTGGGGTCCCCTCGCGGTTTAAAGGCAGTGGATCTGGGACACAGTTCACTCTCACCAT
CAGCGACCTGGAGTGTGACGATGCTGCCACTTACTACTGTGCAGGCGGTTACAATACTGTTATTGATACTTTTGCTT
TCGGCGGAGGCACCGAGGTGGAGTTCAAACGTACGGTGGCTGCACCATCTGTCTTCATCTTCCCGCCATCTGATGA
GCAGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGT
460C3CHIMERICLIGHTCHAINVARIABLELIGHTCHAINNUCLEOTIDESEQUENCE
SEQ ID NO: 90
GCCCAAGTGCTGACCCAGACTCCATCGTCCGTGTCTGCAGCTGTGGGAGGCACAGTCAGCATCAGTTGCCAGTCC
AGTCAGAGTGTTTATAGTAACTGGTTCTCCTGGTATCAGCAGAAACCAGGGCAGCCTCCCAAGCTCCTGATCTATT
CTGCATCCACTCTGGCATCTGGGGTCCCCTCGCGGTTTAAAGGCAGTGGATCTGGGACACAGTTCACTCTCACCAT
CAGCGACCTGGAGTGTGACGATGCTGCCACTTACTACTGTGCAGGCGGTTACAATACTGTTATTGATACTTTTGCTT
TCGGCGGAGGCACCGAGGTGGAGTTCAAA
460C3CHIMERICLIGHTCHAINFULL-LENGTHAMINOACIDSEQUENCE.HUMANKAPPACONSTANT
DOMAINISUNDERLINED
SEQ ID NO: 91
AQVLTQTPSSVSAAVGGTVSISCQSSQSVYSNWFSWYQQKPGQPPKLLIYSASTLASGVPSRFKGSGSGTQFTLTISDLE
CDDAATYYCAGGYNTVIDTFAFGGGTEVEFKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC
460C3CHIMERICLIGHTCHAINVARIABLELIGHTCHAINAMINOACIDSEQUENCE.COMPIMENTARITY
DETERMININGREGIONSAREUNDERLINED
SEQ ID NO: 92
AQVLTQTPSSVSAAVGGTVSISCQSSQSVYSNWFSWYQQKPGQPPKLLIYSASTLASGVPSRFKGSGSGTQFTLTISDLE
CDDAATYYCAGGYNTVIDTFAFGGGTEVEFK
460C3CHIMERICHEAVYCHAINFULL-LENGTHNUCLEOTIDESEQUENCE
SEQ ID NO: 93
CAGTCGTTGGAGGAGTCCGGGGGAGACCTGGTCAAGCCTGGGGCATCCCTGACACTCACCTGCACAGCCTCTGGA
ATCGACTTCAGTAGGAGATACTACATGTGCTGGGTCCGCCAGGCTCCAGGGAAGGGGCTGGAGTGGATCGCATG
CATATATACTGGTAGCCGCGATACTCCTCACTACGCGAGCTGGGCGAAAGGCCGATTCACCATCTCCCAAACCTCG
TCGACCACGGTGACTCTGCAAATGACCAGTCTGACAGCCGCGGACACGGCCACCTATTTCTGTGCGAGAGAAGGT
AGCCTGTGGGGCCAGGGCACCCTGGTCACCGTCTCGAGCGCTAGCACCAAGGGCCCATCGGTCTTCCCCCTGGCA
CCCTCCTCCAAGAGCACCTCTGGGGGCACAGCGGCCCTGGGCTGCCTGGTCAAGGACTACTTCCCCGAACCGGTG
ACGGTGTCGTGGAACTCAGGCGCCCTGACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAGACCTACATCTGCAACGTGAATCACAA
GCCCAGCAACACCAAGGTGGACAAGAGAGTTGAGCCCAAATCTTGTGACAAAACTCACACATGCCCACCGTGCCC
AGCACCTGAAGCCGCGGGGGCACCGTCAGTCTTCCTCTTCCCCCCAAAACCCAAGGACACCCTCATGATCTCCCGG
ACCCCTGAGGTCACATGCGTGGTGGTGGACGTGAGCCACGAAGACCCTGAGGTCAAGTTCAACTGGTACGTGGA
CGGCGTGGAGGTGCATAATGCCAAGACAAAGCCGCGGGAGGAGCAGTACAACAGCACGTACCGTGTGGTCAGC
GTCCTCACCGTCCTGCACCAGGACTGGCTGAATGGCAAGGAGTACAAGTGCGCGGTCTCCAACAAAGCCCTCCCA
GCCCCCATCGAGAAAACCATCTCCAAAGCCAAAGGGCAGCCCCGAGAACCACAGGTGTACACCCTGCCCCCATCC
CGGGATGAGCTGACCAAGAACCAGGTCAGCCTGACCTGCCTGGTCAAAGGCTTCTATCCCAGCGACATCGCCGTG
GAGTGGGAGAGCAATGGGCAGCCGGAGAACAACTACAAGACCACGCCTCCCGTGCTGGACTCCGACGGCTCCTT
CTTCCTCTATAGCAAGCTCACCGTGGACAAGAGCAGGTGGCAGCAGGGGAACGTCTTCTCATGCTCCGTGATGCA
TGAGGCTCTGCACAACCACTACACGCAGAAGAGCCTCTCCCTGTCTCCGGGT
460C3CHIMERICHEAVYCHAINVARIABLEHEAVYCHAINNUCLEOTIDESEQUENCE
SEQ ID NO: 94
CAGTCGTTGGAGGAGTCCGGGGGAGACCTGGTCAAGCCTGGGGCATCCCTGACACTCACCTGCACAGCCTCTGGA
ATCGACTTCAGTAGGAGATACTACATGTGCTGGGTCCGCCAGGCTCCAGGGAAGGGGCTGGAGTGGATCGCATG
CATATATACTGGTAGCCGCGATACTCCTCACTACGCGAGCTGGGCGAAAGGCCGATTCACCATCTCCCAAACCTCG
TCGACCACGGTGACTCTGCAAATGACCAGTCTGACAGCCGCGGACACGGCCACCTATTTCTGTGCGAGAGAAGGT
AGCCTGTGGGGCCAGGGCACCCTGGTCACCGTCTCGAGC
460C3CHIMERICHEAVYCHAINFULL-LENGTHAMINOACIDSEQUENCE.HUMANGAMMA-1CONSTANT
DOMAINISUNDERLINED
SEQ ID NO: 95
QSLEESGGDLVKPGASLTLTCTASGIDFSRRYYMCWVRQAPGKGLEWIACIYTGSRDTPHYASWAKGRFTISQTSSTTV
TLQMTSLTAADTATYFCAREGSLWGQGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKRVEPKSCDKTHTCPPCPAPEAAGAPSVFLF
PPKPKDTLMISRTPEVTCVVVDVSHEDPEVKFNWYVDGVEVHNAKTKPREEQYNSTYRVVSVLTVLHQDWLNGKEYK
CAVSNKALPAPIEKTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPG
460C3CHIMERICHEAVYCHAINVARIABLEHEAVYCHAINAMINOACIDSEQUENCE.COMPLIMENTARITY
DETERMININGREGIONSAREUNDERLINED
SEQ ID NO: 96
QSLEESGGDLVKPGASLTLTCTASGIDFSRRYYMCWVRQAPGKGLEWIACIYTGSRDTPHYASWAKGRFTISQTSSTTV
TLQMTSLTAADTATYFCAREGSLWGQGTLVTVSS
464D11CHIMERICLIGHTCHAINFULL-LENGTHNUCLEOTIDESEQUENCE
SEQ ID NO: 97
GCGCAAGTGCTGACCCAGACTCCATCGCCTGTGTCTGCAGCTGTGGGAGGCACAGTCACCATCAATTGCCAGTCCA
GTCAGAGTGTTTATAGTAACAGCTTCTTATCCTGGTATCAGCAGAAACCAGGGCAGCCTCCCAAGCGCCTGATCTA
TTCTGCATCCGATCTGGCATCTGGGGTCCCATCGCGGTTCAAAGGCAGTGGATCTGGGACACAGTTCACTCTCACC
ATCAGCGACCTGGAGTGTGACGATGCTGCCACTTACTACTGTGCAGGCGTTTATAGTGATAATACTTATGTTTTCG
GCGGAGGCACCGAGGTGGAGTTCAAACGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGGAA
GGTGGATAACGCCCTCCAATCGGGTAACTCCCAGGAGAGTGTCACAGAGCAGGACAGCAAGGACAGCACCTACA
GCCTCAGCAGCACCCTGACGCTGAGCAAAGCAGACTACGAGAAACACAAAGTCTACGCCTGCGAAGTCACCCATC
AGGGCCTGAGCTCGCCCGTCACAAAGAGCTTCAACAGGGGAGAGTGT
464D11CHIMERICLIGHTCHAINVARIABLELIGHTCHAINNUCLEOTIDESEQUENCE
SEQ ID NO: 98
GCGCAAGTGCTGACCCAGACTCCATCGCCTGTGTCTGCAGCTGTGGGAGGCACAGTCACCATCAATTGCCAGTCCA
GTCAGAGTGTTTATAGTAACAGCTTCTTATCCTGGTATCAGCAGAAACCAGGGCAGCCTCCCAAGCGCCTGATCTA
TTCTGCATCCGATCTGGCATCTGGGGTCCCATCGCGGTTCAAAGGCAGTGGATCTGGGACACAGTTCACTCTCACC
ATCAGCGACCTGGAGTGTGACGATGCTGCCACTTACTACTGTGCAGGCGTTTATAGTGATAATACTTATGTTTTCG
GCGGAGGCACCGAGGTGGAGTTCAAA
464D11CHIMERICLIGHTCHAINFULL-LENGTHAMINOACIDSEQUENCE.HUMANKAPPACONSTANT
DOMAINISUNDERLINED
SEQ ID NO: 99
AQVLTQTPSPVSAAVGGTVTINCQSSQSVYSNSFLSWYQQKPGQPPKRLIYSASDLASGVPSRFKGSGSGTQFTLTISDL
ECDDAATYYCAGVYSDNTYVFGGGTEVEFKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC
464D11CHIMERICLIGHTCHAINVARIABLELIGHTCHAINAMINOACIDSEQUENCE.COMPIMENTARITY
DETERMININGREGIONSAREUNDERLINED
SEQ ID NO: 100
AQVLTQTPSPVSAAVGGTVTINCQSSQSVYSNSFLSWYQQKPGQPPKRLIYSASDLASGVPSRFKGSGSGTQFTLTISDL
ECDDAATYYCAGVYSDNTYVFGGGTEVEFK
464D11CHIMERICHEAVYCHAINFULL-LENGTHNUCLEOTIDESEQUENCE
SEQ ID NO: 101
CAGTCGGTGGAGGAGTCCGGCGGTCGCCTGGTAAAGCCTGACGAATCCCTGACACTCACCTGCACAGCCTCTGGA
TTCTCCCTCAGTCACTACTGGATGACTTGGGTCCGACAGGCTCCAGGGAAGGGACTGGAATACATCGGATTCATTA
ATGTTGGTGGTGACACATCTTACGCGAGCTGGTCGAAAGGCCGATTCACCATCTCCAAGGCCTCGACCACGGTGG
ATCTGAAGATCAGTAGTCTGACAACCGAGGACACGGCCACCTATTTCTGTGGCAGAGGTGGTCTGACTTTTGGTTG
GGACTTGTGGGGCCCAGGGACCCTCGTCACCGTCTCGAGCGCTAGCACCAAGGGCCCATCGGTCTTCCCCCTGGC
ACCCTCCTCCAAGAGCACCTCTGGGGGCACAGCGGCCCTGGGCTGCCTGGTCAAGGACTACTTCCCCGAACCGGT
GACGGTGTCGTGGAACTCAGGCGCCCTGACCAGCGGCGTGCACACCTTCCCGGCTGTCCTACAGTCCTCAGGACT
CTACTCCCTCAGCAGCGTGGTGACCGTGCCCTCCAGCAGCTTGGGCACCCAGACCTACATCTGCAACGTGAATCAC
AAGCCCAGCAACACCAAGGTGGACAAGAGAGTTGAGCCCAAATCTTGTGACAAAACTCACACATGCCCACCGTGC
CCAGCACCTGAAGCCGCGGGGGCACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGGTCA
GCGTCCTCACCGTCCTGCACCAGGACTGGCTGAATGGCAAGGAGTACAAGTGCGCGGTCTCCAACAAAGCCCTCC
CAGCCCCCATCGAGAAAACCATCTCCAAAGCCAAAGGGCAGCCCCGAGAACCACAGGTGTACACCCTGCCCCCAT
CCCGGGATGAGCTGACCAAGAACCAGGTCAGCCTGACCTGCCTGGTCAAAGGCTTCTATCCCAGCGACATCGCCG
TGGAGTGGGAGAGCAATGGGCAGCCGGAGAACAACTACAAGACCACGCCTCCCGTGCTGGACTCCGACGGCTCC
TTCTTCCTCTATAGCAAGCTCACCGTGGACAAGAGCAGGTGGCAGCAGGGGAACGTCTTCTCATGCTCCGTGATGC
ATGAGGCTCTGCACAACCACTACACGCAGAAGAGCCTCTCCCTGTCTCCGGGT
464D11CHIMERICHEAVYCHAINVARIABLEHEAVYCHAINNUCLEOTIDESEQUENCE
SEQ ID NO: 102
CAGTCGGTGGAGGAGTCCGGCGGTCGCCTGGTAAAGCCTGACGAATCCCTGACACTCACCTGCACAGCCTCTGGA
TTCTCCCTCAGTCACTACTGGATGACTTGGGTCCGACAGGCTCCAGGGAAGGGACTGGAATACATCGGATTCATTA
ATGTTGGTGGTGACACATCTTACGCGAGCTGGTCGAAAGGCCGATTCACCATCTCCAAGGCCTCGACCACGGTGG
ATCTGAAGATCAGTAGTCTGACAACCGAGGACACGGCCACCTATTTCTGTGGCAGAGGTGGTCTGACTTTTGGTTG
GGACTTGTGGGGCCCAGGGACCCTCGTCACCGTCTCGAGC
464D11CHIMERICHEAVYCHAINFULL-LENGTHAMINOACIDSEQUENCE.HUMANGAMMA-1
CONSTANTDOMAINISUNDERLINED
SEQ ID NO: 103
QSVEESGGRLVKPDESLTLTCTASGFSLSHYWMTWVRQAPGKGLEYIGFINVGGDTSYASWSKGRFTISKASTTVDLKIS
SLTTEDTATYFCGRGGLTFGWDLWGPGTLVTVSSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKRVEPKSCDKTHTCPPCPAPEAAGAPSVFL
FPPKPKDTLMISRTPEVTCVVVDVSHEDPEVKFNWYVDGVEVHNAKTKPREEQYNSTYRVVSVLTVLHQDWLNGKEY
KCAVSNKALPAPIEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPG
464D11CHIMERICHEAVYCHAINVARIABLEHEAVYCHAINAMINOACIDSEQUENCE.
COMPLIMENTARITYDETERMININGREGIONSAREUNDERLINED
SEQ ID NO: 104
QSVEESGGRLVKPDESLTLTCTASGFSLSHYWMTWVRQAPGKGLEYIGFINVGGDTSYASWSKGRFTISKASTTVDLKIS
SLTTEDTATYFCGRGGLTFGWDLWGPGTLVTVSS
466F6CHIMERICLIGHTCHAINFULL-LENGTHNUCLEOTIDESEQUENCE
SEQ ID NO: 105
GATGTTGTGATGACCCAGACTCCAGCCTCCGTGTCTGAACCTGTGGGAGGCACAGTCACCATCAAGTGCCAGGCC
AGTCAGAACATTAGGACTTACTTATCCTGGTATCAGCAGAAACCAGGGCAGCGTCCCAAGCTCCTGATCTATGCTG
CAGCCAATCTGGCATCTGGGGTCCCATCGCGGTTCAGTGGCAGTAGATCTGGGACAGAGTTCACTCTCACCATCAG
CGACCTGGAGTGTGCCGATGCTGCCACTTACTATTGTCAGTGTACCTATCTTGGTACTGATTATGTTGGCGGTGCTT
TCGGCGGAGGCACCGAGGTGGAGTTCAAACGTACGGTGGCTGCACCATCTGTCTTCATCTTCCCGCCATCTGATGA
GCAGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGT
466F6CHIMERICLIGHTCHAINVARIABLELIGHTCHAINNUCLEOTIDESEQUENCE
SEQ ID NO: 106
GATGTTGTGATGACCCAGACTCCAGCCTCCGTGTCTGAACCTGTGGGAGGCACAGTCACCATCAAGTGCCAGGCC
AGTCAGAACATTAGGACTTACTTATCCTGGTATCAGCAGAAACCAGGGCAGCGTCCCAAGCTCCTGATCTATGCTG
CAGCCAATCTGGCATCTGGGGTCCCATCGCGGTTCAGTGGCAGTAGATCTGGGACAGAGTTCACTCTCACCATCAG
CGACCTGGAGTGTGCCGATGCTGCCACTTACTATTGTCAGTGTACCTATCTTGGTACTGATTATGTTGGCGGTGCTT
TCGGCGGAGGCACCGAGGTGGAGTTCAAA
466F6CHIMERICLIGHTCHAINFULL-LENGTHAMINOACIDSEQUENCE.HUMANKAPPACONSTANT
DOMAINISUNDERLINED
SEQ ID NO: 107
DVVMTQTPASVSEPVGGTVTIKCQASQNIRTYLSWYQQKPGQRPKLLIYAAANLASGVPSRFSGSRSGTEFTLTISDLEC
ADAATYYCQCTYLGTDYVGGAFGGGTEVEFKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
466F6CHIMERICLIGHTCHAINVARIABLELIGHTCHAINAMINOACIDSEQUENCE.COMPIMENTARITY
DETERMININGREGIONSAREUNDERLINED
SEQ ID NO: 108
DVVMTQTPASVSEPVGGTVTIKCQASQNIRTYLSWYQQKPGQRPKLLIYAAANLASGVPSRFSGSRSGTEFTLTISDLEC
ADAATYYCQCTYLGTDYVGGAFGGGTEVEFK
466F6CHIMERICHEAVYCHAINFULL-LENGTHNUCLEOTIDESEQUENCE
SEQ ID NO: 109
CGGTCGCTGGAGGAGTCCGGGGGTCGCCTGGTCACGCCTGGGACACCCCTGACACTCACCTGCACAGCCTCTGGA
TTCACCATCAGTAGCTACCACATGCAGTGGGTCCGCCAGGCTCCAGGGAAGGGGCTGGAATACATCGGAACCATT
AGTAGTGGTGGTAATGTATACTACGCGAGCTGGGCGAGAGGCCGATTCACCATCTCCAGACCCTCGTCGACCACG
GTGGATCTGAAGATGACCAGTCTGACAACCGAGGACACGGCCACCTATTTCTGTGCCAGAGACTCTGGTTATAGT
GATCCTATGTGGGGCCCGGGCACCCTGGTCACCGTCTCGAGCGCTAGCACCAAGGGCCCATCGGTCTTCCCCCTG
GCACCCTCCTCCAAGAGCACCTCTGGGGGCACAGCGGCCCTGGGCTGCCTGGTCAAGGACTACTTCCCCGAACCG
GTGACGGTGTCGTGGAACTCAGGCGCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACCCAGACCTACATCTGCAACGTGAATC
ACAAGCCCAGCAACACCAAGGTGGACAAGAGAGTTGAGCCCAAATCTTGTGACAAAACTCACACATGCCCACCGT
GCCCAGCACCTGAAGCCGCGGGGGCACCGTCAGTCTTCCTCTTCCCCCCAAAACCCAAGGACACCCTCATGATCTC
CCGGACCCCTGAGGTCACATGCGTGGTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGTACAACAGCACGTACCGTGTGGTC
AGCGTCCTCACCGTCCTGCACCAGGACTGGCTGAATGGCAAGGAGTACAAGTGCGCGGTCTCCAACAAAGCCCTC
CCAGCCCCCATCGAGAAAACCATCTCCAAAGCCAAAGGGCAGCCCCGAGAACCACAGGTGTACACCCTGCCCCCA
TCCCGGGATGAGCTGACCAAGAACCAGGTCAGCCTGACCTGCCTGGTCAAAGGCTTCTATCCCAGCGACATCGCC
GTGGAGTGGGAGAGCAATGGGCAGCCGGAGAACAACTACAAGACCACGCCTCCCGTGCTGGACTCCGACGGCTC
CTTCTTCCTCTATAGCAAGCTCACCGTGGACAAGAGCAGGTGGCAGCAGGGGAACGTCTTCTCATGCTCCGTGATG
CATGAGGCTCTGCACAACCACTACACGCAGAAGAGCCTCTCCCTGTCTCCGGGT
466F6CHIMERICHEAVYCHAINVARIABLEHEAVYCHAINNUCLEOTIDESEQUENCE
SEQ ID NO: 110
CGGTCGCTGGAGGAGTCCGGGGGTCGCCTGGTCACGCCTGGGACACCCCTGACACTCACCTGCACAGCCTCTGGA
TTCACCATCAGTAGCTACCACATGCAGTGGGTCCGCCAGGCTCCAGGGAAGGGGCTGGAATACATCGGAACCATT
AGTAGTGGTGGTAATGTATACTACGCGAGCTGGGCGAGAGGCCGATTCACCATCTCCAGACCCTCGTCGACCACG
GTGGATCTGAAGATGACCAGTCTGACAACCGAGGACACGGCCACCTATTTCTGTGCCAGAGACTCTGGTTATAGT
GATCCTATGTGGGGCCCGGGCACCCTGGTCACCGTCTCGAGC
466F6CHIMERICHEAVYCHAINFULL-LENGTHAMINOACIDSEQUENCE.HUMANGAMMA-1CONSTANT
DOMAINISUNDERLINED
SEQ ID NO: 111
RSLEESGGRLVTPGTPLTLTCTASGFTISSYHMQWVRQAPGKGLEYIGTISSGGNVYYASWARGRFTISRPSSTTVDLKM
TSLTTEDTATYFCARDSGYSDPMWGPGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKRVEPKSCDKTHTCPPCPAPEAAGAPSVFLF
PPKPKDTLMISRTPEVTCVVVDVSHEDPEVKFNWYVDGVEVHNAKTKPREEQYNSTYRVVSVLTVLHQDWLNGKEYK
CAVSNKALPAPIEKTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPG
466F6CHIMERICHEAVYCHAINVARIABLEHEAVYCHAINAMINOACIDSEQUENCE.COMPLIMENTARITY
DETERMININGREGIONSAREUNDERLINED
SEQ ID NO: 112
RSLEESGGRLVTPGTPLTLTCTASGFTISSYHMQWVRQAPGKGLEYIGTISSGGNVYYASWARGRFTISRPSSTTVDLKM
TSLTTEDTATYFCARDSGYSDPMWGPGTLVTVSS
470B6CHIMERICLIGHTCHAINFULL-LENGTHNUCLEOTIDESEQUENCE
SEQ ID NO: 113
GCAGCCGTGCTGACCCAGACACCATCGCCCGTGTCTGCAGCTGTGGGAGGCACAGTCACCATCAGTTGCCAGTCC
AGTCAGAGTGTTTATAGTAACAACCAATTATCCTGGTTTCAGCAGAAATCAGGGCAGCCTCCCAAGCTCCTGATCT
ATGATGCATCCAATCTGGCATCTGGGGTCCCATCACGGTTCAGCGGCAGTGGATCTGGGACACAGTTCACTCTCAC
CATCAGCAGCGTGCAGTGTGACGATGCTGCCACTTACCACTGTCTAGGCGGTAGTGATGATGATGGTGATATTGCT
TTCGGCGGAGGCACCGAGGTGGTGTTCAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAGAGCAGGACAGCAAGGACAGCACCT
ACAGCCTCAGCAGCACCCTGACGCTGAGCAAAGCAGACTACGAGAAACACAAAGTCTACGCCTGCGAAGTCACCC
ATCAGGGCCTGAGCTCGCCCGTCACAAAGAGCTTCAACAGGGGAGAGTGT
470B6CHIMERICLIGHTCHAINVARIABLELIGHTCHAINNUCLEOTIDESEQUENCE
SEQ ID NO: 114
GCAGCCGTGCTGACCCAGACACCATCGCCCGTGTCTGCAGCTGTGGGAGGCACAGTCACCATCAGTTGCCAGTCC
AGTCAGAGTGTTTATAGTAACAACCAATTATCCTGGTTTCAGCAGAAATCAGGGCAGCCTCCCAAGCTCCTGATCT
ATGATGCATCCAATCTGGCATCTGGGGTCCCATCACGGTTCAGCGGCAGTGGATCTGGGACACAGTTCACTCTCAC
CATCAGCAGCGTGCAGTGTGACGATGCTGCCACTTACCACTGTCTAGGCGGTAGTGATGATGATGGTGATATTGCT
TTCGGCGGAGGCACCGAGGTGGTGTTCAAA
470B6CHIMERICLIGHTCHAINFULL-LENGTHAMINOACIDSEQUENCE.HUMANKAPPACONSTANT
DOMAINISUNDERLINED
SEQ ID NO: 115
AAVLTQTPSPVSAAVGGTVTISCQSSQSVYSNNQLSWFQQKSGQPPKLLIYDASNLASGVPSRFSGSGSGTQFTLTISSV
QCDDAATYHCLGGSDDDGDIAFGGGTEVVFKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
470B6CHIMERICLIGHTCHAINVARIABLELIGHTCHAINAMINOACIDSEQUENCE.COMPIMENTARITY
DETERMININGREGIONSAREUNDERLINED
SEQ ID NO: 116
AAVLTQTPSPVSAAVGGTVTISCQSSQSVYSNNQLSWFQQKSGQPPKLLIYDASNLASGVPSRFSGSGSGTQFTLTISSV
QCDDAATYHCLGGSDDDGDIAFGGGTEVVFK
470B6CHIMERICHEAVYCHAINFULL-LENGTHNUCLEOTIDESEQUENCE
SEQ ID NO: 117
CAGTCACTGGAGGAGTCCGGGGGTCGCCTGGTCACGCCTGGGACACCCCTGACACTCACCTGCACAGCCTCTGGA
TTCTCCCTCAGTAGCTACTACATGAGCTGGGTCCGCCAGGCTCCAGGGAAGGGACTGGAATGGATCGGAAGTATT
TATGGTAGTGGTGCCGCATACTACGCGAGCTGGGCGAAAGGCCGATTCACCATCTCCAGAACGTCGACCACGGTG
GATCTGAAAATGACCAGTCTGACAGCCGCGGACACGGCCACCTATTTCTGTGCCAGAGATCCTATTAACAATGCCA
TGTGGGGCCCAGGCACCCTGGTCACCGTCTCGAGCGCTAGCACCAAGGGCCCATCGGTCTTCCCCCTGGCACCCTC
CTCCAAGAGCACCTCTGGGGGCACAGCGGCCCTGGGCTGCCTGGTCAAGGACTACTTCCCCGAACCGGTGACGGT
GTCGTGGAACTCAGGCGCCCTGACCAGCGGCGTGCACACCTTCCCGGCTGTCCTACAGTCCTCAGGACTCTACTCC
CTCAGCAGCGTGGTGACCGTGCCCTCCAGCAGCTTGGGCACCCAGACCTACATCTGCAACGTGAATCACAAGCCC
AGCAACACCAAGGTGGACAAGAGAGTTGAGCCCAAATCTTGTGACAAAACTCACACATGCCCACCGTGCCCAGCA
CCTGAAGCCGCGGGGGCACCGTCAGTCTTCCTCTTCCCCCCAAAACCCAAGGACACCCTCATGATCTCCCGGACCC
CTGAGGTCACATGCGTGGTGGTGGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGCACGTACCGTGTGGTCAGCGTCCT
CACCGTCCTGCACCAGGACTGGCTGAATGGCAAGGAGTACAAGTGCGCGGTCTCCAACAAAGCCCTCCCAGCCCC
CATCGAGAAAACCATCTCCAAAGCCAAAGGGCAGCCCCGAGAACCACAGGTGTACACCCTGCCCCCATCCCGGGA
TGAGCTGACCAAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGCTCCTTCTTCCT
CTATAGCAAGCTCACCGTGGACAAGAGCAGGTGGCAGCAGGGGAACGTCTTCTCATGCTCCGTGATGCATGAGGC
TCTGCACAACCACTACACGCAGAAGAGCCTCTCCCTGTCTCCGGGT
470B6CHIMERICHEAVYCHAINVARIABLEHEAVYCHAINNUCLEOTIDESEQUENCE
SEQ ID NO: 118
CAGTCACTGGAGGAGTCCGGGGGTCGCCTGGTCACGCCTGGGACACCCCTGACACTCACCTGCACAGCCTCTGGA
TTCTCCCTCAGTAGCTACTACATGAGCTGGGTCCGCCAGGCTCCAGGGAAGGGACTGGAATGGATCGGAAGTATT
TATGGTAGTGGTGCCGCATACTACGCGAGCTGGGCGAAAGGCCGATTCACCATCTCCAGAACGTCGACCACGGTG
GATCTGAAAATGACCAGTCTGACAGCCGCGGACACGGCCACCTATTTCTGTGCCAGAGATCCTATTAACAATGCCA
TGTGGGGCCCAGGCACCCTGGTCACCGTCTCGAGC
470B6 CHIMERIC HEAVY CHAIN FULL-LENGTH AMINO ACID SEQUENCE. HUMAN GAMMA-1 CONSTANT
DOMAINISUNDERLINED
SEQ ID NO: 119
QSLEESGGRLVTPGTPLTLTCTASGFSLSSYYMSWVRQAPGKGLEWIGSIYGSGAAYYASWAKGRFTISRTSTTVDLKMT
SLTAADTATYFCARDPINNAMWGPGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKRVEPKSCDKTHTCPPCPAPEAAGAPSVFLFPP
KPKDTLMISRTPEVTCVVVDVSHEDPEVKFNWYVDGVEVHNAKTKPREEQYNSTYRVVSVLTVLHQDWLNGKEYKCA
VSNKALPAPIEKTISKAKGQPREPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPG
470B6CHIMERICHEAVYCHAINVARIABLEHEAVYCHAINAMINOACIDSEQUENCE.COMPLIMENTARITY
DETERMININGREGIONSAREUNDERLINED
SEQ ID NO: 120
QSLEESGGRLVTPGTPLTLTCTASGFSLSSYYMSWVRQAPGKGLEWIGSIYGSGAAYYASWAKGRFTISRTSTTVDLKMT
SLTAADTATYFCARDPINNAMWGPGTLVTVSS
470C6CHIMERICLIGHTCHAINFULL-LENGTHNUCLEOTIDESEQUENCE
SEQ ID NO: 121
GCCCTTGTGATGACCCAGACTCCAGCCTCCGTGTCTGCAGCTGTGGGAGGCACAGTCACCATCAATTGCCAGGCCA
GTGAGGACATTGATAACTATTTAGCCTGGTATCAGCAGAAACCAGGGCAGCCTCCCAAGCTCCTGATCTITTATGC
ATCCGATCTGGCATCTGGGGTCCCATCGCGGTTCTCAGGCAGTGGATCTGGGACACAGTTCACTCTCACCATCAGC
GGTGTGCAGTGTGACGATGCTGCCACTTACTACTGTCAAGGCGGTTACTATACTAGTAGTACTGATACGAGGGGT
GCTTTCGGCGGAGGCACCGAGGTGGTGGTCAAACGTACGGTGGCTGCACCATCTGTCTTCATCTTCCCGCCATCTG
ATGAGCAGTTGAAATCTGGAACTGCCTCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCACAGAGCAGGACAGCAAGGACAGCA
CCTACAGCCTCAGCAGCACCCTGACGCTGAGCAAAGCAGACTACGAGAAACACAAAGTCTACGCCTGCGAAGTCA
CCCATCAGGGCCTGAGCTCGCCCGTCACAAAGAGCTTCAACAGGGGAGAGTGT
470C6CHIMERICLIGHTCHAINVARIABLELIGHTCHAINNUCLEOTIDESEQUENCE
SEQ ID NO: 122
GCCCTTGTGATGACCCAGACTCCAGCCTCCGTGTCTGCAGCTGTGGGAGGCACAGTCACCATCAATTGCCAGGCCA
GTGAGGACATTGATAACTATTTAGCCTGGTATCAGCAGAAACCAGGGCAGCCTCCCAAGCTCCTGATCTITTATGC
ATCCGATCTGGCATCTGGGGTCCCATCGCGGTTCTCAGGCAGTGGATCTGGGACACAGTTCACTCTCACCATCAGC
GGTGTGCAGTGTGACGATGCTGCCACTTACTACTGTCAAGGCGGTTACTATACTAGTAGTACTGATACGAGGGGT
GCTTTCGGCGGAGGCACCGAGGTGGTGGTCAAA
470C6CHIMERICLIGHTCHAINFULL-LENGTHAMINOACIDSEQUENCE.HUMANKAPPACONSTANT
DOMAINISUNDERLINED
SEQ ID NO: 123
ALVMTQTPASVSAAVGGTVTINCQASEDIDNYLAWYQQKPGQPPKLLIFYASDLASGVPSRFSGSGSGTQFTLTISGVQ
CDDAATYYCQGGYYTSSTDTRGAFGGGTEVVVKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSSPVTKSFNRGEC
470C6CHIMERICLIGHTCHAINVARIABLELIGHTCHAINAMINOACIDSEQUENCE.COMPIMENTARITY
DETERMININGREGIONSAREUNDERLINED
SEQ ID NO: 124
ALVMTQTPASVSAAVGGTVTINCQASEDIDNYLAWYQQKPGQPPKLLIFYASDLASGVPSRFSGSGSGTQFTLTISGVQ
CDDAATYYCQGGYYTSSTDTRGAFGGGTEVVVK
470C6CHIMERICHEAVYCHAINFULL-LENGTHNUCLEOTIDESEQUENCE
SEQ ID NO: 125
CAGTCATTGGAGGAGGCCGGGGGAGACCTGGTCAAGCCTGGGGCATCCCTGACACTCACCTGCACAGCCTCTGGC
TTCTCCTTCACTAGCAGCTACTACATGTGCTGGGTCCGCCAGGCTCCAGGGAAGGGGCTGGAGTGGATCGCATGC
ATTTATACTGGTAGTAGTGGTAGCACTTACTACGCGAACTGGGCGAAAGGCCGATTCACCATCTCCAAAACCTCGT
CGACCACGGTGACTCTGCAAATGACCAGTCTGACAGCCGCGGACACGGCCACCTATTTCTGTGCGAGAGATAGTA
GTAGTTATTATATGTTTAACTTGTGGGGCCAGGGCACCCTCGTCACCGTCTCGAGCGCTAGCACCAAGGGCCCATC
GGTCTTCCCCCTGGCACCCTCCTCCAAGAGCACCTCTGGGGGCACAGCGGCCCTGGGCTGCCTGGTCAAGGACTA
CTTCCCCGAACCGGTGACGGTGTCGTGGAACTCAGGCGCCCTGACCAGCGGCGTGCACACCTTCCCGGCTGTCCTA
CAGTCCTCAGGACTCTACTCCCTCAGCAGCGTGGTGACCGTGCCCTCCAGCAGCTTGGGCACCCAGACCTACATCT
GCAACGTGAATCACAAGCCCAGCAACACCAAGGTGGACAAGAGAGTTGAGCCCAAATCTTGTGACAAAACTCACA
CATGCCCACCGTGCCCAGCACCTGAAGCCGCGGGGGCACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTGAGGTCAAGTT
CAACTGGTACGTGGACGGCGTGGAGGTGCATAATGCCAAGACAAAGCCGCGGGAGGAGCAGTACAACAGCACG
TACCGTGTGGTCAGCGTCCTCACCGTCCTGCACCAGGACTGGCTGAATGGCAAGGAGTACAAGTGCGCGGTCTCC
AACAAAGCCCTCCCAGCCCCCATCGAGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGACCTGCCTGGTCAAAGGCTTCTATCCCA
GCGACATCGCCGTGGAGTGGGAGAGCAATGGGCAGCCGGAGAACAACTACAAGACCACGCCTCCCGTGCTGGAC
TCCGACGGCTCCTTCTTCCTCTATAGCAAGCTCACCGTGGACAAGAGCAGGTGGCAGCAGGGGAACGTCTTCTCAT
GCTCCGTGATGCATGAGGCTCTGCACAACCACTACACGCAGAAGAGCCTCTCCCTGTCTCCGGGT
470C6CHIMERICHEAVYCHAINVARIABLEHEAVYCHAINNUCLEOTIDESEQUENCE
SEQ ID NO: 126
CAGTCATTGGAGGAGGCCGGGGGAGACCTGGTCAAGCCTGGGGCATCCCTGACACTCACCTGCACAGCCTCTGGC
TTCTCCTTCACTAGCAGCTACTACATGTGCTGGGTCCGCCAGGCTCCAGGGAAGGGGCTGGAGTGGATCGCATGC
ATTTATACTGGTAGTAGTGGTAGCACTTACTACGCGAACTGGGCGAAAGGCCGATTCACCATCTCCAAAACCTCGT
CGACCACGGTGACTCTGCAAATGACCAGTCTGACAGCCGCGGACACGGCCACCTATTTCTGTGCGAGAGATAGTA
GTAGTTATTATATGTTTAACTTGTGGGGCCAGGGCACCCTCGTCACCGTCTCGAGC
470C6CHIMERICHEAVYCHAINFULL-LENGTHAMINOACIDSEQUENCE.HUMANGAMMA-1CONSTANT
DOMAINISUNDERLINED
SEQ ID NO: 127
QSLEEAGGDLVKPGASLTLTCTASGFSFTSSYYMCWVRQAPGKGLEWIACIYTGSSGSTYYANWAKGRFTISKTSSTTVT
LQMTSLTAADTATYFCARDSSSYYMFNLWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVDKRVEPKSCDKTHTCPPCPAPEAAG
APSVFLEPPKPKDTLMISRTPEVTCVVVDVSHEDPEVKFNWYVDGVEVHNAKTKPREEQYNSTYRVVSVLTVLHQDWL
NGKEYKCAVSNKALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPG
470C6CHIMERICHEAVYCHAINVARIABLEHEAVYCHAINAMINOACIDSEQUENCE.COMPLIMENTARITY
DETERMININGREGIONSAREUNDERLINED
SEQ ID NO: 128
QSLEEAGGDLVKPGASLTLTCTASGFSFTSSYYMCWVRQAPGKGLEWIACIYTGSSGSTYYANWAKGRFTISKTSSTTVT
LQMTSLTAADTATYFCARDSSSYYMFNLWGQGTLVTVSS
472E4CHIMERICLIGHTCHAINFULL-LENGTHNUCLEOTIDESEQUENCE
SEQ ID NO: 129
GCAGCCGTGCTGACCCAGACTCCATCTTCCACGTCTGCAGCTGTGGGAGGCACAGTCACCATCAGTTGCCAGTCCA
GTCAGAGTGTTTATAATAACAATGCTTTAGCCTGGTATCAGAAAAAACCAGGACAGCCTCCCAAGCTCCTGATCTA
TTTGGCTTCCACTCTGGCATCTGGGGTCCCATCGCGGTTCAGCGGCAGTGGATCTGGGACACAGTTCACTCTCACC
ATCAGCGACCTGGAGTGTGACGATGCTGCCACTTATTATTGTCTAGGTGTTTATAATGATGATGTTGATAATGGTTT
CGGCGGAGGCACCGAGGTGGTGTTCAAACGTACGGTGGCTGCACCATCTGTCTTCATCTTCCCGCCATCTGATGAG
CAGTTGAAATCTGGAACTGCCTCTGTTGTGTGCCTGCTGAATAACTTCTATCCCAGAGAGGCCAAAGTACAGTGGA
AGGTGGATAACGCCCTCCAATCGGGTAACTCCCAGGAGAGTGTCACAGAGCAGGACAGCAAGGACAGCACCTAC
AGCCTCAGCAGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAGAGTGT
472E4CHIMERICLIGHTCHAINVARIABLELIGHTCHAINNUCLEOTIDESEQUENCE
SEQ ID NO: 130
GCAGCCGTGCTGACCCAGACTCCATCTTCCACGTCTGCAGCTGTGGGAGGCACAGTCACCATCAGTTGCCAGTCCA
GTCAGAGTGTTTATAATAACAATGCTTTAGCCTGGTATCAGAAAAAACCAGGACAGCCTCCCAAGCTCCTGATCTA
TTTGGCTTCCACTCTGGCATCTGGGGTCCCATCGCGGTTCAGCGGCAGTGGATCTGGGACACAGTTCACTCTCACC
ATCAGCGACCTGGAGTGTGACGATGCTGCCACTTATTATTGTCTAGGTGTTTATAATGATGATGTTGATAATGGTTT
CGGCGGAGGCACCGAGGTGGTGTTCAAA
472E4CHIMERICLIGHTCHAINFULL-LENGTHAMINOACIDSEQUENCE.HUMANKAPPACONSTANT
DOMAINISUNDERLINED
SEQ ID NO: 131
AAVLTQTPSSTSAAVGGTVTISCQSSQSVYNNNALAWYQKKPGQPPKLLIYLASTLASGVPSRFSGSGSGTQFTLTISDLE
CDDAATYYCLGVYNDDVDNGFGGGTEVVFKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
472E4CHIMERICLIGHTCHAINVARIABLELIGHTCHAINAMINOACIDSEQUENCE.COMPIMENTARITY
DETERMININGREGIONSAREUNDERLINED
SEQ ID NO: 132
AAVLTQTPSSTSAAVGGTVTISCQSSQSVYNNNALAWYQKKPGQPPKLLIYLASTLASGVPSRFSGSGSGTQFTLTISDLE
CDDAATYYCLGVYNDDVDNGFGGGTEVVFK
472E4CHIMERICHEAVYCHAINFULL-LENGTHNUCLEOTIDESEQUENCE
SEQ ID NO: 133
CAGTCGCTGGAGGAGTCCGGGGGTCGCCTGGTCACGCCTGGAGGATCCCTGACACTCACCTGCACCGTCTCTGGA
TTCTCCCTCAATAACAATGCAATAAGCTGGGTCCGCCAGGCTCCAGGGAAGGGGCTGGAATGGGTTGCAATCATA
CAAAATACTGGTACCACAGACTACGCGAGGTGGGCGAAAGGCCGATTCACCATCTCCAAAACCTCGACCACGGTG
GATTTGAAAATCAACAGTCCGACAACCGAGGACACGGCCACCTATTTCTGTGGCAGAGGGTATGGTTTTGAGTCG
GAGCTTGTCATCTGGGGCCCGGGCACCCTGGTCACCGTCTCGAGCGCTAGCACCAAGGGCCCATCGGTCTTCCCCC
TGGCACCCTCCTCCAAGAGCACCTCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACCCAGACCTACATCTGCAACGTGAA
TCACAAGCCCAGCAACACCAAGGTGGACAAGAGAGTTGAGCCCAAATCTTGTGACAAAACTCACACATGCCCACC
GTGCCCAGCACCTGAAGCCGCGGGGGCACCGTCAGTCTTCCTCTTCCCCCCAAAACCCAAGGACACCCTCATGATC
TCCCGGACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTGTGG
TCAGCGTCCTCACCGTCCTGCACCAGGACTGGCTGAATGGCAAGGAGTACAAGTGCGCGGTCTCCAACAAAGCCC
TCCCAGCCCCCATCGAGAAAACCATCTCCAAAGCCAAAGGGCAGCCCCGAGAACCACAGGTGTACACCCTGCCCC
CATCCCGGGATGAGCTGACCAAGAACCAGGTCAGCCTGACCTGCCTGGTCAAAGGCTTCTATCCCAGCGACATCG
CCGTGGAGTGGGAGAGCAATGGGCAGCCGGAGAACAACTACAAGACCACGCCTCCCGTGCTGGACTCCGACGGC
TCCTTCTTCCTCTATAGCAAGCTCACCGTGGACAAGAGCAGGTGGCAGCAGGGGAACGTCTTCTCATGCTCCGTGA
TGCATGAGGCTCTGCACAACCACTACACGCAGAAGAGCCTCTCCCTGTCTCCGGGT
472E4CHIMERICHEAVYCHAINVARIABLEHEAVYCHAINNUCLEOTIDESEQUENCE
SEQ ID NO: 134
CAGTCGCTGGAGGAGTCCGGGGGTCGCCTGGTCACGCCTGGAGGATCCCTGACACTCACCTGCACCGTCTCTGGA
TTCTCCCTCAATAACAATGCAATAAGCTGGGTCCGCCAGGCTCCAGGGAAGGGGCTGGAATGGGTTGCAATCATA
CAAAATACTGGTACCACAGACTACGCGAGGTGGGCGAAAGGCCGATTCACCATCTCCAAAACCTCGACCACGGTG
GATTTGAAAATCAACAGTCCGACAACCGAGGACACGGCCACCTATTTCTGTGGCAGAGGGTATGGTTTTGAGTCG
GAGCTTGTCATCTGGGGCCCGGGCACCCTGGTCACCGTCTCGAGC
472E4 CHIMERIC HEAVY CHAIN FULL-LENGTH AMINO ACID SEQUENCE. HUMAN GAMMA-1 CONSTANT
DOMAINISUNDERLINED
SEQ ID NO: 135
QSLEESGGRLVTPGGSLTLTCTVSGFSLNNNAISWVRQAPGKGLEWVAIIQNTGTTDYARWAKGRFTISKTSTTVDLKIN
SPTTEDTATYFCGRGYGFESELVIWGPGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKRVEPKSCDKTHTCPPCPAPEAAGAPSVFLF
PPKPKDTLMISRTPEVTCVVVDVSHEDPEVKFNWYVDGVEVHNAKTKPREEQYNSTYRVVSVLTVLHQDWLNGKEYK
CAVSNKALPAPIEKTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPG
472E4CHIMERICHEAVYCHAINVARIABLEHEAVYCHAINAMINOACIDSEQUENCE.COMPLIMENTARITY
DETERMININGREGIONSAREUNDERLINED
SEQ ID NO: 136
QSLEESGGRLVTPGGSLTLTCTVSGFSLNNNAISWVRQAPGKGLEWVAIIQNTGTTDYARWAKGRFTISKTSTTVDLKIN
SPTTEDTATYFCGRGYGFESELVIWGPGTLVTVSS
411H11HUMANIZEDLIGHTCHAINFULL-LENGTHNUCLEOTIDESEQUENCE
SEQ ID NO: 137
GACGTTGTGATGACCCAGTCTCCATCTTCCGTGTCTGCATCTGTAGGAGACAGAGTCACCATCACCTGTCAGGCCA
GTCAGAGCATTGGTGCTGATTTAGCCTGGTATCAGCAGAAACCAGGGAAAGCCCCTAAGCTCCTGATCTATAGGG
CATCCACTCTGGCCTCTGGGGTCCCATCAAGGTTCAGCGGCAGTGGATCTGGGACAGATTTCACTCTCACCATCAG
CAGCCTGCAGCCTGAAGATTTTGCAACTTACTATTGTCAAGCTATTTATTATGGTACTGATGATGTCATATACCATA
CTTTCGGCGGAGGGACCAAGGTGGAGATCAAACGTACGGTGGCTGCACCATCTGTCTTCATCTTCCCGCCATCTGA
TGAGCAGTTGAAATCTGGAACTGCCTCTGTTGTGTGCCTGCTGAATAACTTCTATCCCAGAGAGGCCAAAGTACAG
TGGAAGGTGGATAACGCCCTCCAATCGGGTAACTCCCAGGAGAGTGTCACAGAGCAGGACAGCAAGGACAGCAC
CTACAGCCTCAGCAGCACCCTGACGCTGAGCAAAGCAGACTACGAGAAACACAAAGTCTACGCCTGCGAAGTCAC
CCATCAGGGCCTGAGCTCGCCCGTCACAAAGAGCTTCAACAGGGGAGAGTGT
411H11HUMANIZEDLIGHTCHAINVARIABLELIGHTCHAINNUCLEOTIDESEQUENCE
SEQ ID NO: 138
GACGTTGTGATGACCCAGTCTCCATCTTCCGTGTCTGCATCTGTAGGAGACAGAGTCACCATCACCTGTCAGGCCA
GTCAGAGCATTGGTGCTGATTTAGCCTGGTATCAGCAGAAACCAGGGAAAGCCCCTAAGCTCCTGATCTATAGGG
CATCCACTCTGGCCTCTGGGGTCCCATCAAGGTTCAGCGGCAGTGGATCTGGGACAGATTTCACTCTCACCATCAG
CAGCCTGCAGCCTGAAGATTTTGCAACTTACTATTGTCAAGCTATTTATTATGGTACTGATGATGTCATATACCATA
CTTTCGGCGGAGGGACCAAGGTGGAGATCAAA
411H11 HUMANIZED LIGHT CHAIN FULL-LENGTH AMINO ACID SEQUENCE. HUMAN KAPPA CONSTANT
DOMAINISUNDERLINED
SEQ ID NO: 139
DVVMTQSPSSVSASVGDRVTITCQASQSIGADLAWYQQKPGKAPKLLIYRASTLASGVPSRFSGSGSGTDFTLTISSLQP
EDFATYYCQAIYYGTDDVIYHTFGGGTKVEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC
411H11HUMANIZEDLIGHTCHAINVARIABLELIGHTCHAINAMINOACIDSEQUENCE.
COMPIMENTARITYDETERMININGREGIONSAREUNDERLINED
SEQ ID NO: 140
DVVMTQSPSSVSASVGDRVTITCQASQSIGADLAWYQQKPGKAPKLLIYRASTLASGVPSRFSGSGSGTDFTLTISSLQP
EDFATYYCQAIYYGTDDVIYHTFGGGTKVEIK
411H11HUMANIZEDHEAVYCHAINFULL-LENGTHNUCLEOTIDESEQUENCE
SEQ ID NO: 141
GAGGTGCAGCTGGTGGAGTCTGGGGGAGGCTTGGTCCAGCCTGGGGGGTCCCTGAGACTCTCCTGTGCAGCCTC
TGGATTCACCATCAGTAGCCACCACATGATCTGGGTCCGCCAGGCTCCAGGGAAGGGGCTGCAATACATCGGATT
CATTAATGATGGTGACTATACATACTACACGAACTCCGCAAAAGGCAGATTCACCATCTCCAGAGACAATTCCAAG
AACACGCTGTATCTTCAAATGAACAGCCTGAGAGCCGAGGACACGGCTGTGTATTACTGTGCGAGAGGGGTTGAT
GGTACTAGTTATCCTGGCTTGTGGGGCCAGGGAACCCTGGTCACCGTCTCGAGCGCTAGCACCAAGGGCCCATCG
GTCTTCCCCCTGGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGCAGCTTGGGCACCCAGACCTACATCTG
CAACGTGAATCACAAGCCCAGCAACACCAAGGTGGACAAGAGAGTTGAGCCCAAATCTTGTGACAAAACTCACAC
ATGCCCACCGTGCCCAGCACCTGAAGCCGCGGGGGCACCGTCAGTCTTCCTCTTCCCCCCAAAACCCAAGGACACC
CTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGT
ACCGTGTGGTCAGCGTCCTCACCGTCCTGCACCAGGACTGGCTGAATGGCAAGGAGTACAAGTGCGCGGTCTCCA
ACAAAGCCCTCCCAGCCCCCATCGAGAAAACCATCTCCAAAGCCAAAGGGCAGCCCCGAGAACCACAGGTGTACA
CCCTGCCCCCATCCCGGGATGAGCTGACCAAGAACCAGGTCAGCCTGACCTGCCTGGTCAAAGGCTTCTATCCCAG
CGACATCGCCGTGGAGTGGGAGAGCAATGGGCAGCCGGAGAACAACTACAAGACCACGCCTCCCGTGCTGGACT
CCGACGGCTCCTTCTTCCTCTATAGCAAGCTCACCGTGGACAAGAGCAGGTGGCAGCAGGGGAACGTCTTCTCAT
GCTCCGTGATGCATGAGGCTCTGCACAACCACTACACGCAGAAGAGCCTCTCCCTGTCTCCGGGT
411H11HUMANIZEDHEAVYCHAINVARIABLEHEAVYCHAINNUCLEOTIDESEQUENCE
SEQ ID NO: 142
GAGGTGCAGCTGGTGGAGTCTGGGGGAGGCTTGGTCCAGCCTGGGGGGTCCCTGAGACTCTCCTGTGCAGCCTC
TGGATTCACCATCAGTAGCCACCACATGATCTGGGTCCGCCAGGCTCCAGGGAAGGGGCTGCAATACATCGGATT
CATTAATGATGGTGACTATACATACTACACGAACTCCGCAAAAGGCAGATTCACCATCTCCAGAGACAATTCCAAG
AACACGCTGTATCTTCAAATGAACAGCCTGAGAGCCGAGGACACGGCTGTGTATTACTGTGCGAGAGGGGTTGAT
GGTACTAGTTATCCTGGCTTGTGGGGCCAGGGAACCCTGGTCACCGTCTCGAGC
411H11HUMANIZEDHEAVYCHAINFULL-LENGTHAMINOACIDSEQUENCE.HUMANGAMMA-1
CONSTANTDOMAINISUNDERLINED
SEQ ID NO: 143
EVQLVESGGGLVQPGGSLRLSCAASGFTISSHHMIWVRQAPGKGLQYIGFINDGDYTYYTNSAKGRFTISRDNSKNTLYL
QMNSLRAEDTAVYYCARGVDGTSYPGLWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVDKRVEPKSCDKTHTCPPCPAPEAAG
APSVFLEPPKPKDTLMISRTPEVTCVVVDVSHEDPEVKFNWYVDGVEVHNAKTKPREEQYNSTYRVVSVLTVLHQDWL
NGKEYKCAVSNKALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPG
411H11HUMANIZEDHEAVYCHAINVARIABLEHEAVYCHAINAMINOACIDSEQUENCE.
COMPLIMENTARITYDETERMININGREGIONSAREUNDERLINED
SEQ ID NO: 144
EVQLVESGGGLVQPGGSLRLSCAASGFTISSHHMIWVRQAPGKGLQYIGFINDGDYTYYTNSAKGRFTISRDNSKNTLYL
QMNSLRAEDTAVYYCARGVDGTSYPGLWGQGTLVTVSS
413F3HUMANIZEDLIGHTCHAINFULL-LENGTHNUCLEOTIDESEQUENCE
SEQ ID NO: 145
GACATCCAGATGACCCAGTCTCCATCCTCCCTGTCTGCATCTGTAGGAGACAGAGTCACCATCACTTGCCAGGCCA
GTCAGAGCATTTACAGCAATTTAGCCTGGTATCAGCAGAAACCAGGGAAAGTTCCTAAGCTCCTGATCTATTCTAC
ATCCACTCTGGCATCTGGGGTCCCATCTCGGTTCAGTGGCAGTGGATCTGGGACAGATTTCACTCTCACCATCAGC
AGCCTGCAGCCTGAAGATGTTGCAACTTATTACTGTCAAAACTATTATGGTAGTAGTACTGATAGTTATGGGAATC
CTTTCGGCGGAGGGACCAAGGTGGAGATCAAACGTACGGTGGCTGCACCATCTGTCTTCATCTTCCCGCCATCTGA
TGAGCAGTTGAAATCTGGAACTGCCTCTGTTGTGTGCCTGCTGAATAACTTCTATCCCAGAGAGGCCAAAGTACAG
TGGAAGGTGGATAACGCCCTCCAATCGGGTAACTCCCAGGAGAGTGTCACAGAGCAGGACAGCAAGGACAGCAC
CTACAGCCTCAGCAGCACCCTGACGCTGAGCAAAGCAGACTACGAGAAACACAAAGTCTACGCCTGCGAAGTCAC
CCATCAGGGCCTGAGCTCGCCCGTCACAAAGAGCTTCAACAGGGGAGAGTGT
413F3HUMANIZEDLIGHTCHAINVARIABLELIGHTCHAINNUCLEOTIDESEQUENCE
SEQ ID NO: 146
GACATCCAGATGACCCAGTCTCCATCCTCCCTGTCTGCATCTGTAGGAGACAGAGTCACCATCACTTGCCAGGCCA
GTCAGAGCATTTACAGCAATTTAGCCTGGTATCAGCAGAAACCAGGGAAAGTTCCTAAGCTCCTGATCTATTCTAC
ATCCACTCTGGCATCTGGGGTCCCATCTCGGTTCAGTGGCAGTGGATCTGGGACAGATTTCACTCTCACCATCAGC
AGCCTGCAGCCTGAAGATGTTGCAACTTATTACTGTCAAAACTATTATGGTAGTAGTACTGATAGTTATGGGAATC
CTTTCGGCGGAGGGACCAAGGTGGAGATCAAA
413F3HUMANIZEDLIGHTCHAINFULL-LENGTHAMINOACIDSEQUENCE.HUMANKAPPACONSTANT
DOMAINISUNDERLINED
SEQ ID NO: 147
DIQMTQSPSSLSASVGDRVTITCQASQSIYSNLAWYQQKPGKVPKLLIYSTSTLASGVPSRFSGSGSGTDFTLTISSLQPED
VATYYCQNYYGSSTDSYGNPFGGGTKVEIKRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC
413F3HUMANIZEDLIGHTCHAINVARIABLELIGHTCHAINAMINOACIDSEQUENCE.COMPIMENTARITY
DETERMININGREGIONSAREUNDERLINED
SEQ ID NO: 148
DIQMTQSPSSLSASVGDRVTITCQASQSIYSNLAWYQQKPGKVPKLLIYSTSTLASGVPSRFSGSGSGTDFTLTISSLQPED
VATYYCQNYYGSSTDSYGNPFGGGTKVEIK
413F3HUMANIZEDHEAVYCHAINFULL-LENGTHNUCLEOTIDESEQUENCE
SEQ ID NO: 149
GAGGTGCAGCTGGTGGAGTCTGGGGGAGGCTTGGTCCAGCCTGGGGGGTCCCTGAGACTCTCCTGTGCAGCCTC
TGGAATCGACTTCAGTAGCAACTACATGTGCTGGGTCCGCCAGGCTCCAGGGAAGGGGCTGGAGTGGATCGCAT
GCATTTATGGTGATAGTAGTGATAATAGTTACTCCGCGAGCTCCGCTAAAGGCAGATTCACCATCTCCAGAGACAA
TTCCAAGAACACGCTGTATCTTCAAATGAACAGCCTGAGAGCCGAGGACACGGCTGTGTATTACTGTGCGAGATCT
GGTTATAGCTATTTAGGCTACTTTAACTTGTGGGGCCAGGGAACCCTGGTCACCGTCTCGAGCGCTAGCACCAAGG
GCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGC
TGTCCTACAGTCCTCAGGACTCTACTCCCTCAGCAGCGTGGTGACCGTGCCCTCCAGCAGCTTGGGCACCCAGACC
TACATCTGCAACGTGAATCACAAGCCCAGCAACACCAAGGTGGACAAGAGAGTTGAGCCCAAATCTTGTGACAAA
ACTCACACATGCCCACCGTGCCCAGCACCTGAAGCCGCGGGGGCACCGTCAGTCTTCCTCTTCCCCCCAAAACCCA
AGGACACCCTCATGATCTCCCGGACCCCTGAGGTCACATGCGTGGTGGTGGACGTGAGCCACGAAGACCCTGAGG
TCAAGTTCAACTGGTACGTGGACGGCGTGGAGGTGCATAATGCCAAGACAAAGCCGCGGGAGGAGCAGTACAAC
AGCACGTACCGTGTGGTCAGCGTCCTCACCGTCCTGCACCAGGACTGGCTGAATGGCAAGGAGTACAAGTGCGCG
GTCTCCAACAAAGCCCTCCCAGCCCCCATCGAGAAAACCATCTCCAAAGCCAAAGGGCAGCCCCGAGAACCACAG
GTGTACACCCTGCCCCCATCCCGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACCACGCCTCCCGTG
CTGGACTCCGACGGCTCCTTCTTCCTCTATAGCAAGCTCACCGTGGACAAGAGCAGGTGGCAGCAGGGGAACGTC
TTCTCATGCTCCGTGATGCATGAGGCTCTGCACAACCACTACACGCAGAAGAGCCTCTCCCTGTCTCCGGGT
413F3HUMANIZEDHEAVYCHAINVARIABLEHEAVYCHAINNUCLEOTIDESEQUENCE
SEQ ID NO: 150
GAGGTGCAGCTGGTGGAGTCTGGGGGAGGCTTGGTCCAGCCTGGGGGGTCCCTGAGACTCTCCTGTGCAGCCTC
TGGAATCGACTTCAGTAGCAACTACATGTGCTGGGTCCGCCAGGCTCCAGGGAAGGGGCTGGAGTGGATCGCAT
GCATTTATGGTGATAGTAGTGATAATAGTTACTCCGCGAGCTCCGCTAAAGGCAGATTCACCATCTCCAGAGACAA
TTCCAAGAACACGCTGTATCTTCAAATGAACAGCCTGAGAGCCGAGGACACGGCTGTGTATTACTGTGCGAGATCT
GGTTATAGCTATTTAGGCTACTTTAACTTGTGGGGCCAGGGAACCCTGGTCACCGTCTCGAGC
413F3HUMANIZEDHEAVYCHAINFULL-LENGTHAMINOACIDSEQUENCE.HUMANGAMMA-1
CONSTANTDOMAINISUNDERLINED
SEQ ID NO: 151
EVQLVESGGGLVQPGGSLRLSCAASGIDFSSNYMCWVRQAPGKGLEWIACIYGDSSDNSYSASSAKGRFTISRDNSKNT
LYLQMNSLRAEDTAVYYCARSGYSYLGYFNLWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKRVEPKSCDKTHTCPPCPAPEAA
GAPSVFLEPPKPKDTLMISRTPEVTCVVVDVSHEDPEVKFNWYVDGVEVHNAKTKPREEQYNSTYRVVSVLTVLHQDW
LNGKEYKCAVSNKALPAPIEKTISKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPG
413F3HUMANIZEDHEAVYCHAINVARIABLEHEAVYCHAINAMINOACIDSEQUENCE.
COMPLIMENTARITYDETERMININGREGIONSAREUNDERLINED
SEQ ID NO: 152
EVQLVESGGGLVQPGGSLRLSCAASGIDFSSNYMCWVRQAPGKGLEWIACIYGDSSDNSYSASSAKGRFTISRDNSKNT
LYLQMNSLRAEDTAVYYCARSGYSYLGYFNLWGQGTLVTVSS
416F8HUMANIZEDLIGHTCHAINFULL-LENGTHNUCLEOTIDESEQUENCE
SEQ ID NO: 153
GACATCCAGATGACCCAGTCTCCATCCTCCCTGTCTGCATCTGTAGGAGACAGAGTCACCATCACTTGCCAGTCCA
GTCAGACTGTTTATAATAACAACTTGTTATCCTGGTATCAGCAGAAACCAGGGAAAGTTCCTAAGCTCCTGATCTAT
TATGCATCCACTCTGGCATTTGGGGTCCCATCTCGGTTCAGTGGCAGTGGATCTGGGACAGATTTCACTCTCACCAT
CAGCAGCCTGCAGCCTGAAGATGTTGCAACTTATTACTGTCAAGGCGGTTATAGTGGTTGGATTTATGTTTTCGGC
GGAGGGACCAAGGTGGAGATCAAACGTACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAGG
TGGATAACGCCCTCCAATCGGGTAACTCCCAGGAGAGTGTCACAGAGCAGGACAGCAAGGACAGCACCTACAGC
CTCAGCAGCACCCTGACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGT
416F8HUMANIZEDLIGHTCHAINVARIABLELIGHTCHAINNUCLEOTIDESEQUENCE
SEQ ID NO: 154
GACATCCAGATGACCCAGTCTCCATCCTCCCTGTCTGCATCTGTAGGAGACAGAGTCACCATCACTTGCCAGTCCA
GTCAGACTGTTTATAATAACAACTTGTTATCCTGGTATCAGCAGAAACCAGGGAAAGTTCCTAAGCTCCTGATCTAT
TATGCATCCACTCTGGCATTTGGGGTCCCATCTCGGTTCAGTGGCAGTGGATCTGGGACAGATTTCACTCTCACCAT
CAGCAGCCTGCAGCCTGAAGATGTTGCAACTTATTACTGTCAAGGCGGTTATAGTGGTTGGATTTATGTTTTCGGC
GGAGGGACCAAGGTGGAGATCAAA
416F8HUMANIZEDLIGHTCHAINFULL-LENGTHAMINOACIDSEQUENCE.HUMANKAPPACONSTANT
DOMAINISUNDERLINED
SEQ ID NO: 155
DIQMTQSPSSLSASVGDRVTITCQSSQTVYNNNLLSWYQQKPGKVPKLLIYYASTLAFGVPSRFSGSGSGTDFTLTISSLQ
PEDVATYYCQGGYSGWIYVFGGGTKVEIKRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC
416F8HUMANIZEDLIGHTCHAINVARIABLELIGHTCHAINAMINOACIDSEQUENCE.COMPIMENTARITY
DETERMININGREGIONSAREUNDERLINED
SEQ ID NO: 156
DIQMTQSPSSLSASVGDRVTITCQSSQTVYNNNLLSWYQQKPGKVPKLLIYYASTLAFGVPSRFSGSGSGTDFTLTISSLQ
PEDVATYYCQGGYSGWIYVFGGGTKVEIK
416F8HUMANIZEDHEAVYCHAINFULL-LENGTHNUCLEOTIDESEQUENCE
SEQ ID NO: 157
CAGTCGCTGGTGGAGTCTGGGGGAGGCTTGGTCCAGCCTGGGGGGTCCCTGAGACTCTCCTGTGCAGCCTCTGGA
TTCTCCCTCAGTAACTACTACATGAACTGGGTCCGCCAGGCTCCAGGGAAGGGGCTGGAGTACATCGGAATCATT
AGTAGTAGTGGTAGCGCATACTACGCGAGCTCCGCTAAAGGCAGATTCACCATCTCCAGAGACAATTCCAAGAAC
ACGCTGTATCTTCAAATGAACAGCCTGAGAGCCGAGGACACGGCTGTGTATTACTGTGCGAGAGGGGACTACGGC
ATGGACCTCTGGGGCCAGGGAACCCTGGTCACCGTCTCGAGCGCTAGCACCAAGGGCCCATCGGTCTTCCCCCTG
GCACCCTCCTCCAAGAGCACCTCTGGGGGCACAGCGGCCCTGGGCTGCCTGGTCAAGGACTACTTCCCCGAACCG
GTGACGGTGTCGTGGAACTCAGGCGCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACCCAGACCTACATCTGCAACGTGAATC
ACAAGCCCAGCAACACCAAGGTGGACAAGAGAGTTGAGCCCAAATCTTGTGACAAAACTCACACATGCCCACCGT
GCCCAGCACCTGAAGCCGCGGGGGCACCGTCAGTCTTCCTCTTCCCCCCAAAACCCAAGGACACCCTCATGATCTC
CCGGACCCCTGAGGTCACATGCGTGGTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGTACAACAGCACGTACCGTGTGGTC
AGCGTCCTCACCGTCCTGCACCAGGACTGGCTGAATGGCAAGGAGTACAAGTGCGCGGTCTCCAACAAAGCCCTC
CCAGCCCCCATCGAGAAAACCATCTCCAAAGCCAAAGGGCAGCCCCGAGAACCACAGGTGTACACCCTGCCCCCA
TCCCGGGATGAGCTGACCAAGAACCAGGTCAGCCTGACCTGCCTGGTCAAAGGCTTCTATCCCAGCGACATCGCC
GTGGAGTGGGAGAGCAATGGGCAGCCGGAGAACAACTACAAGACCACGCCTCCCGTGCTGGACTCCGACGGCTC
CTTCTTCCTCTATAGCAAGCTCACCGTGGACAAGAGCAGGTGGCAGCAGGGGAACGTCTTCTCATGCTCCGTGATG
CATGAGGCTCTGCACAACCACTACACGCAGAAGAGCCTCTCCCTGTCTCCGGGT
416F8HUMANIZEDHEAVYCHAINVARIABLEHEAVYCHAINNUCLEOTIDESEQUENCE
SEQ ID NO: 158
CAGTCGCTGGTGGAGTCTGGGGGAGGCTTGGTCCAGCCTGGGGGGTCCCTGAGACTCTCCTGTGCAGCCTCTGGA
TTCTCCCTCAGTAACTACTACATGAACTGGGTCCGCCAGGCTCCAGGGAAGGGGCTGGAGTACATCGGAATCATT
AGTAGTAGTGGTAGCGCATACTACGCGAGCTCCGCTAAAGGCAGATTCACCATCTCCAGAGACAATTCCAAGAAC
ACGCTGTATCTTCAAATGAACAGCCTGAGAGCCGAGGACACGGCTGTGTATTACTGTGCGAGAGGGGACTACGGC
ATGGACCTCTGGGGCCAGGGAACCCTGGTCACCGTCTCGAGC
416F8HUMANIZEDHEAVYCHAINFULL-LENGTHAMINOACIDSEQUENCE.HUMANGAMMA-1
CONSTANTDOMAINISUNDERLINED
SEQ ID NO: 159
QSLVESGGGLVQPGGSLRLSCAASGFSLSNYYMNWVRQAPGKGLEYIGIISSSGSAYYASSAKGRFTISRDNSKNTLYLQ
MNSLRAEDTAVYYCARGDYGMDLWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKRVEPKSCDKTHTCPPCPAPEAAGAPSV
FLFPPKPKDTLMISRTPEVTCVVVDVSHEDPEVKFNWYVDGVEVHNAKTKPREEQYNSTYRVVSVLTVLHQDWLNGKE
YKCAVSNKALPAPIEKTISKAKGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG
416F8HUMANIZEDHEAVYCHAINVARIABLEHEAVYCHAINAMINOACIDSEQUENCE.
COMPLIMENTARITYDETERMININGREGIONSAREUNDERLINED
SEQ ID NO: 160
QSLVESGGGLVQPGGSLRLSCAASGFSLSNYYMNWVRQAPGKGLEYIGIISSSGSAYYASSAKGRFTISRDNSKNTLYLQ
MNSLRAEDTAVYYCARGDYGMDLWGQGTLVTVSS
416G1HUMANIZEDLIGHTCHAINFULL-LENGTHNUCLEOTIDESEQUENCE
SEQ ID NO: 161
GACGTTGTGATGACCCAGTCTCCATCTTCCGTGTCTGCATCTGTAGGAGACAGAGTCACCATCACCTGTCAGGCCA
GTCAGAGCATTGGTAGTGATTTAGCCTGGTATCAGCAGAAACCAGGGAAAGCCCCTAAGCTCCTGATCTATAGGG
CATCCACTCTGGCATCTGGGGTCCCATCAAGGTTCAGCGGCAGTGGATCTGGGACAGATTTCACTCTCACCATCAG
CAGCCTGCAGCCTGAAGATTTTGCAACTTACTATTGTCAATCCATTTATTATGGTAGTGATGATGTCATATACCATA
CTTTCGGCGGAGGGACCAAGGTGGAGATCAAACGTACGGTGGCTGCACCATCTGTCTTCATCTTCCCGCCATCTGA
TGAGCAGTTGAAATCTGGAACTGCCTCTGTTGTGTGCCTGCTGAATAACTTCTATCCCAGAGAGGCCAAAGTACAG
TGGAAGGTGGATAACGCCCTCCAATCGGGTAACTCCCAGGAGAGTGTCACAGAGCAGGACAGCAAGGACAGCAC
CTACAGCCTCAGCAGCACCCTGACGCTGAGCAAAGCAGACTACGAGAAACACAAAGTCTACGCCTGCGAAGTCAC
CCATCAGGGCCTGAGCTCGCCCGTCACAAAGAGCTTCAACAGGGGAGAGTGT
416G1HUMANIZEDLIGHTCHAINVARIABLELIGHTCHAINNUCLEOTIDESEQUENCE
SEQ ID NO: 162
GACGTTGTGATGACCCAGTCTCCATCTTCCGTGTCTGCATCTGTAGGAGACAGAGTCACCATCACCTGTCAGGCCA
GTCAGAGCATTGGTAGTGATTTAGCCTGGTATCAGCAGAAACCAGGGAAAGCCCCTAAGCTCCTGATCTATAGGG
CATCCACTCTGGCATCTGGGGTCCCATCAAGGTTCAGCGGCAGTGGATCTGGGACAGATTTCACTCTCACCATCAG
CAGCCTGCAGCCTGAAGATTTTGCAACTTACTATTGTCAATCCATTTATTATGGTAGTGATGATGTCATATACCATA
CTTTCGGCGGAGGGACCAAGGTGGAGATCAAA
416G1HUMANIZEDLIGHTCHAINFULL-LENGTHAMINOACIDSEQUENCE.HUMANKAPPACONSTANT
DOMAINISUNDERLINED
SEQ ID NO: 163
DVVMTQSPSSVSASVGDRVTITCQASQSIGSDLAWYQQKPGKAPKLLIYRASTLASGVPSRFSGSGSGTDFTLTISSLQPE
DFATYYCQSIYYGSDDVIYHTFGGGTKVEIKRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC
416G1HUMANIZEDLIGHTCHAINVARIABLELIGHTCHAINAMINOACIDSEQUENCE.COMPIMENTARITY
DETERMININGREGIONSAREUNDERLINED
SEQ ID NO: 164
DVVMTQSPSSVSASVGDRVTITCQASQSIGSDLAWYQQKPGKAPKLLIYRASTLASGVPSRFSGSGSGTDFTLTISSLQPE
DFATYYCQSIYYGSDDVIYHTFGGGTKVEIK
416G1HUMANIZEDHEAVYCHAINFULL-LENGTHNUCLEOTIDESEQUENCE
SEQ ID NO: 165
GAGGTGCAGCTGGTGGAGTCTGGGGGAGGCTTGGTCCAGCCTGGGGGGTCCCTGAGACTCTCCTGTGCAGCCTC
TGGATTCACTATCAATAGCTACCACATGATCTGGGTCCGCCAGGCTCCAGGGAAGGGGCTGGAGTACATCGGATT
CATTAATGATGGTGGTTTCACATACTACGCGAGCTCCGCTAAAGGCAGATTCACCATCTCCAGAGACAATTCCAAG
AACACGCTGTATCTTCAAATGAACAGCCTGAGAGCCGAGGACACGGCTGTGTATTACTGTGCGAGAGGGGTTGAT
GGTACTAGTTATCCTGACTTATGGGGCCAGGGAACCCTGGTCACCGTCTCGAGCGCTAGCACCAAGGGCCCATCG
GTCTTCCCCCTGGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGCAGCTTGGGCACCCAGACCTACATCTG
CAACGTGAATCACAAGCCCAGCAACACCAAGGTGGACAAGAGAGTTGAGCCCAAATCTTGTGACAAAACTCACAC
ATGCCCACCGTGCCCAGCACCTGAAGCCGCGGGGGCACCGTCAGTCTTCCTCTTCCCCCCAAAACCCAAGGACACC
CTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGT
ACCGTGTGGTCAGCGTCCTCACCGTCCTGCACCAGGACTGGCTGAATGGCAAGGAGTACAAGTGCGCGGTCTCCA
ACAAAGCCCTCCCAGCCCCCATCGAGAAAACCATCTCCAAAGCCAAAGGGCAGCCCCGAGAACCACAGGTGTACA
CCCTGCCCCCATCCCGGGATGAGCTGACCAAGAACCAGGTCAGCCTGACCTGCCTGGTCAAAGGCTTCTATCCCAG
CGACATCGCCGTGGAGTGGGAGAGCAATGGGCAGCCGGAGAACAACTACAAGACCACGCCTCCCGTGCTGGACT
CCGACGGCTCCTTCTTCCTCTATAGCAAGCTCACCGTGGACAAGAGCAGGTGGCAGCAGGGGAACGTCTTCTCAT
GCTCCGTGATGCATGAGGCTCTGCACAACCACTACACGCAGAAGAGCCTCTCCCTGTCTCCGGGT
416G1HUMANIZEDHEAVYCHAINVARIABLEHEAVYCHAINNUCLEOTIDESEQUENCE
SEQ ID NO: 166
GAGGTGCAGCTGGTGGAGTCTGGGGGAGGCTTGGTCCAGCCTGGGGGGTCCCTGAGACTCTCCTGTGCAGCCTC
TGGATTCACTATCAATAGCTACCACATGATCTGGGTCCGCCAGGCTCCAGGGAAGGGGCTGGAGTACATCGGATT
CATTAATGATGGTGGTTTCACATACTACGCGAGCTCCGCTAAAGGCAGATTCACCATCTCCAGAGACAATTCCAAG
AACACGCTGTATCTTCAAATGAACAGCCTGAGAGCCGAGGACACGGCTGTGTATTACTGTGCGAGAGGGGTTGAT
GGTACTAGTTATCCTGACTTATGGGGCCAGGGAACCCTGGTCACCGTCTCGAGC
416G1HUMANIZEDHEAVYCHAINFULL-LENGTHAMINOACIDSEQUENCE.HUMANGAMMA-1
CONSTANTDOMAINISUNDERLINED
SEQ ID NO: 167
EVQLVESGGGLVQPGGSLRLSCAASGFTINSYHMIWVRQAPGKGLEYIGFINDGGFTYYASSAKGRFTISRDNSKNTLYL
QMNSLRAEDTAVYYCARGVDGTSYPDLWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVDKRVEPKSCDKTHTCPPCPAPEAAG
APSVFLFPPKPKDTLMISRTPEVTCVVVDVSHEDPEVKFNWYVDGVEVHNAKTKPREEQYNSTYRVVSVLTVLHQDWL
NGKEYKCAVSNKALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPG
416G1HUMANIZEDHEAVYCHAINVARIABLEHEAVYCHAINAMINOACIDSEQUENCE.
COMPLIMENTARITYDETERMININGREGIONSAREUNDERLINED
SEQ ID NO: 168
EVQLVESGGGLVQPGGSLRLSCAASGFTINSYHMIWVRQAPGKGLEYIGFINDGGFTYYASSAKGRFTISRDNSKNTLYL
QMNSLRAEDTAVYYCARGVDGTSYPDLWGQGTLVTVSS
418E10HUMANIZEDLIGHTCHAINFULL-LENGTHNUCLEOTIDESEQUENCE
SEQ ID NO: 169
GACATCCAGATGACCCAGTCTCCTTCCACCCTGTCTGCATCTGTAGGAGACAGAGTCACCATCACTTGCCAGGCCA
GTCAGAGCATTGGTAGTAACTTGAACTGGTATCAGCAGAAACCAGGGAAAGCCCCTAAGCTCCTGATCTATTATGT
ATCCACTCTGGCATCTGGGGTCCCATCAAGGTTCAGCGGCAGTGGATCTGGGACAGAATTCACTCTCACCATCAGC
AGCCTGCAGCCTGATGATTTTGCAACTTATTACTGCCTAGGTGTTTGGAATTATTGGGGTGATGACGGTACTGCTTT
CGGCGGAGGGACCAAGGTGGAGATCAAACGTACGGTGGCTGCACCATCTGTCTTCATCTTCCCGCCATCTGATGA
GCAGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGT
418E10HUMANIZEDLIGHTCHAINVARIABLELIGHTCHAINNUCLEOTIDESEQUENCE
SEQ ID NO: 170
GACATCCAGATGACCCAGTCTCCTTCCACCCTGTCTGCATCTGTAGGAGACAGAGTCACCATCACTTGCCAGGCCA
GTCAGAGCATTGGTAGTAACTTGAACTGGTATCAGCAGAAACCAGGGAAAGCCCCTAAGCTCCTGATCTATTATGT
ATCCACTCTGGCATCTGGGGTCCCATCAAGGTTCAGCGGCAGTGGATCTGGGACAGAATTCACTCTCACCATCAGC
AGCCTGCAGCCTGATGATTTTGCAACTTATTACTGCCTAGGTGTTTGGAATTATTGGGGTGATGACGGTACTGCTTT
CGGCGGAGGGACCAAGGTGGAGATCAAA
418E10HUMANIZEDLIGHTCHAINFULL-LENGTHAMINOACIDSEQUENCE.HUMANKAPPACONSTANT
DOMAINISUNDERLINED
SEQ ID NO: 171
DIQMTQSPSTLSASVGDRVTITCQASQSIGSNLNWYQQKPGKAPKLLIYYVSTLASGVPSRFSGSGSGTEFTLTISSLQPD
DFATYYCLGVWNYWGDDGTAFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
418E10HUMANIZEDLIGHTCHAINVARIABLELIGHTCHAINAMINOACIDSEQUENCE.
COMPIMENTARITYDETERMININGREGIONSAREUNDERLINED
SEQ ID NO: 172
DIQMTQSPSTLSASVGDRVTITCQASQSIGSNLNWYQQKPGKAPKLLIYYVSTLASGVPSRFSGSGSGTEFTLTISSLQPD
DFATYYCLGVWNYWGDDGTAFGGGTKVEIK
418E10HUMANIZEDHEAVYCHAINFULL-LENGTHNUCLEOTIDESEQUENCE
SEQ ID NO: 173
GAGGTGCAGCTGGTGGAGTCTGGGGGAGGCTTGGTCCAGCCTGGGGGGTCCCTGAGACTCTCCTGTGCAGCCTC
TGGATTCTCCCTCAGTACCTATTATATGAGCTGGGTCCGCCAGGCTCCAGGGAAGGGGCTGGAGTGGATCGGAAG
TATTTATGATAGTGGTGCCGCATACTACGCGACCTCCGCGAAGGGCAGATTCACCATCTCCAGAGACAATTCCAAG
AACACGCTGTATCTTCAAATGAACAGCCTGAGAGCCGAGGACACGGCTGTGTATTACTGTGCGAGAGATCCTATT
AATAATGCCATCTGGGGCCAGGGAACCCTGGTCACCGTCTCGAGCGCTAGCACCAAGGGCCCATCGGTCTTCCCC
CTGGCACCCTCCTCCAAGAGCACCTCTGGGGGCACAGCGGCCCTGGGCTGCCTGGTCAAGGACTACTTCCCCGAA
CCGGTGACGGTGTCGTGGAACTCAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCACCCAGACCTACATCTGCAACGTGA
ATCACAAGCCCAGCAACACCAAGGTGGACAAGAGAGTTGAGCCCAAATCTTGTGACAAAACTCACACATGCCCAC
CGTGCCCAGCACCTGAAGCCGCGGGGGCACCGTCAGTCTTCCTCTTCCCCCCAAAACCCAAGGACACCCTCATGAT
CTCCCGGACCCCTGAGGTCACATGCGTGGTGGTGGACGTGAGCCACGAAGACCCTGAGGTCAAGTTCAACTGGTA
CGTGGACGGCGTGGAGGTGCATAATGCCAAGACAAAGCCGCGGGAGGAGCAGTACAACAGCACGTACCGTGTG
GTCAGCGTCCTCACCGTCCTGCACCAGGACTGGCTGAATGGCAAGGAGTACAAGTGCGCGGTCTCCAACAAAGCC
CTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATC
GCCGTGGAGTGGGAGAGCAATGGGCAGCCGGAGAACAACTACAAGACCACGCCTCCCGTGCTGGACTCCGACGG
CTCCTTCTTCCTCTATAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGT
418E10HUMANIZEDHEAVYCHAINVARIABLEHEAVYCHAINNUCLEOTIDESEQUENCE
SEQ ID NO: 174
GAGGTGCAGCTGGTGGAGTCTGGGGGAGGCTTGGTCCAGCCTGGGGGGTCCCTGAGACTCTCCTGTGCAGCCTC
TGGATTCTCCCTCAGTACCTATTATATGAGCTGGGTCCGCCAGGCTCCAGGGAAGGGGCTGGAGTGGATCGGAAG
TATTTATGATAGTGGTGCCGCATACTACGCGACCTCCGCGAAGGGCAGATTCACCATCTCCAGAGACAATTCCAAG
AACACGCTGTATCTTCAAATGAACAGCCTGAGAGCCGAGGACACGGCTGTGTATTACTGTGCGAGAGATCCTATT
AATAATGCCATCTGGGGCCAGGGAACCCTGGTCACCGTCTCGAGC
418E10HUMANIZEDHEAVYCHAINFULL-LENGTHAMINOACIDSEQUENCE.HUMANGAMMA-1
CONSTANTDOMAINISUNDERLINED
SEQ ID NO: 175
EVQLVESGGGLVQPGGSLRLSCAASGFSLSTYYMSWVRQAPGKGLEWIGSIYDSGAAYYATSAKGRFTISRDNSKNTLY
LQMNSLRAEDTAVYYCARDPINNAIWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKRVEPKSCDKTHTCPPCPAPEAAGAPSV
FLFPPKPKDTLMISRTPEVTCVVVDVSHEDPEVKFNWYVDGVEVHNAKTKPREEQYNSTYRVVSVLTVLHQDWLNGKE
YKCAVSNKALPAPIEKTISKAKGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG
418E10HUMANIZEDHEAVYCHAINVARIABLEHEAVYCHAINAMINOACIDSEQUENCE.
COMPLIMENTARITYDETERMININGREGIONSAREUNDERLINED
SEQ ID NO: 176
EVQLVESGGGLVQPGGSLRLSCAASGFSLSTYYMSWVRQAPGKGLEWIGSIYDSGAAYYATSAKGRFTISRDNSKNTLY
LQMNSLRAEDTAVYYCARDPINNAIWGQGTLVTVSS
418E4HUMANIZEDLIGHTCHAINFULL-LENGTHNUCLEOTIDESEQUENCE
SEQ ID NO: 177
GACATCCAGATGACCCAGTCTCCTTCCACCCTGTCTGCATCTGTAGGAGACAGAGTCACCATCACTTGCCAGGCCA
GTCAGAGTATTGCTACTAACTTGAACTGGTATCAGCAGAAACCAGGGAAAGCCCCTAAGCTCCTGATCTATTATAC
ATCCAGTCTGGCATCTGGGGTCCCATCAAGGTTCAGCGGCAGTGGATCTGGGACAGAATTCACTCTCACCATCAGC
AGCCTGCAGCCTGATGATTTTGCAACTTATTACTGCCTAGGTGTTTGGAATTATTGGGGTGATGATGGTACTGCTTT
CGGCGGAGGGACCAAGGTGGAGATCAAACGTACGGTGGCTGCACCATCTGTCTTCATCTTCCCGCCATCTGATGA
GCAGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGT
418E4HUMANIZEDLIGHTCHAINVARIABLELIGHTCHAINNUCLEOTIDESEQUENCE
SEQ ID NO: 178
GACATCCAGATGACCCAGTCTCCTTCCACCCTGTCTGCATCTGTAGGAGACAGAGTCACCATCACTTGCCAGGCCA
GTCAGAGTATTGCTACTAACTTGAACTGGTATCAGCAGAAACCAGGGAAAGCCCCTAAGCTCCTGATCTATTATAC
ATCCAGTCTGGCATCTGGGGTCCCATCAAGGTTCAGCGGCAGTGGATCTGGGACAGAATTCACTCTCACCATCAGC
AGCCTGCAGCCTGATGATTTTGCAACTTATTACTGCCTAGGTGTTTGGAATTATTGGGGTGATGATGGTACTGCTTT
CGGCGGAGGGACCAAGGTGGAGATCAAA
418E4HUMANIZEDLIGHTCHAINFULL-LENGTHAMINOACIDSEQUENCE.HUMANKAPPACONSTANT
DOMAINISUNDERLINED
SEQ ID NO: 179
DIQMTQSPSTLSASVGDRVTITCQASQSIATNLNWYQQKPGKAPKLLIYYTSSLASGVPSRFSGSGSGTEFTLTISSLQPD
DFATYYCLGVWNYWGDDGTAFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
418E4HUMANIZEDLIGHTCHAINVARIABLELIGHTCHAINAMINOACIDSEQUENCE.COMPIMENTARITY
DETERMININGREGIONSAREUNDERLINED
SEQ ID NO: 180
DIQMTQSPSTLSASVGDRVTITCQASQSIATNLNWYQQKPGKAPKLLIYYTSSLASGVPSRFSGSGSGTEFTLTISSLQPD
DFATYYCLGVWNYWGDDGTAFGGGTKVEIK
418E4HUMANIZEDHEAVYCHAINFULL-LENGTHNUCLEOTIDESEQUENCE
SEQ ID NO: 181
GAGGTGCAGCTGGTGGAGTCTGGGGGAGGCTTGGTCCAGCCTGGGGGGTCCCTGAGACTCTCCTGTGCAGCCTC
TGGATTCTCCCTCAGTAGCTACTACATGAGCTGGGTCCGCCAGGCTCCAGGGAAGGGGCTGGAGTGGATCGGAA
GTATTTATGCTAGTGGTAGCGCATACTACGCGAGTTCCGCGAAGGGCAGATTCACCATCTCCAGAGACAATTCCAA
GAACACGCTGTATCTTCAAATGAACAGCCTGAGAGCCGAGGACACGGCTGTGTATTACTGTGCGAGAGATCCTAT
TAACAATGACATCTGGGGCCAGGGAACCCTGGTCACCGTCTCGAGCGCTAGCACCAAGGGCCCATCGGTCTTCCC
CCTGGCACCCTCCTCCAAGAGCACCTCTGGGGGCACAGCGGCCCTGGGCTGCCTGGTCAAGGACTACTTCCCCGA
ACCGGTGACGGTGTCGTGGAACTCAGGCGCCCTGACCAGCGGCGTGCACACCTTCCCGGCTGTCCTACAGTCCTC
AGGACTCTACTCCCTCAGCAGCGTGGTGACCGTGCCCTCCAGCAGCTTGGGCACCCAGACCTACATCTGCAACGTG
AATCACAAGCCCAGCAACACCAAGGTGGACAAGAGAGTTGAGCCCAAATCTTGTGACAAAACTCACACATGCCCA
CCGTGCCCAGCACCTGAAGCCGCGGGGGCACCGTCAGTCTTCCTCTTCCCCCCAAAACCCAAGGACACCCTCATGA
TCTCCCGGACCCCTGAGGTCACATGCGTGGTGGTGGACGTGAGCCACGAAGACCCTGAGGTCAAGTTCAACTGGT
ACGTGGACGGCGTGGAGGTGCATAATGCCAAGACAAAGCCGCGGGAGGAGCAGTACAACAGCACGTACCGTGT
GGTCAGCGTCCTCACCGTCCTGCACCAGGACTGGCTGAATGGCAAGGAGTACAAGTGCGCGGTCTCCAACAAAGC
CCTCCCAGCCCCCATCGAGAAAACCATCTCCAAAGCCAAAGGGCAGCCCCGAGAACCACAGGTGTACACCCTGCC
CCCATCCCGGGATGAGCTGACCAAGAACCAGGTCAGCCTGACCTGCCTGGTCAAAGGCTTCTATCCCAGCGACATC
GCCGTGGAGTGGGAGAGCAATGGGCAGCCGGAGAACAACTACAAGACCACGCCTCCCGTGCTGGACTCCGACGG
CTCCTTCTTCCTCTATAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGT
418E4HUMANIZEDHEAVYCHAINVARIABLEHEAVYCHAINNUCLEOTIDESEQUENCE
SEQ ID NO: 182
GAGGTGCAGCTGGTGGAGTCTGGGGGAGGCTTGGTCCAGCCTGGGGGGTCCCTGAGACTCTCCTGTGCAGCCTC
TGGATTCTCCCTCAGTAGCTACTACATGAGCTGGGTCCGCCAGGCTCCAGGGAAGGGGCTGGAGTGGATCGGAA
GTATTTATGCTAGTGGTAGCGCATACTACGCGAGTTCCGCGAAGGGCAGATTCACCATCTCCAGAGACAATTCCAA
GAACACGCTGTATCTTCAAATGAACAGCCTGAGAGCCGAGGACACGGCTGTGTATTACTGTGCGAGAGATCCTAT
TAACAATGACATCTGGGGCCAGGGAACCCTGGTCACCGTCTCGAGC
418E4HUMANIZEDHEAVYCHAINFULL-LENGTHAMINOACIDSEQUENCE.HUMANGAMMA-1
CONSTANTDOMAINISUNDERLINED
SEQ ID NO: 183
EVQLVESGGGLVQPGGSLRLSCAASGFSLSSYYMSWVRQAPGKGLEWIGSIYASGSAYYASSAKGRFTISRDNSKNTLYL
QMNSLRAEDTAVYYCARDPINNDIWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKRVEPKSCDKTHTCPPCPAPEAAGAPSV
FLFPPKPKDTLMISRTPEVTCVVVDVSHEDPEVKFNWYVDGVEVHNAKTKPREEQYNSTYRVVSVLTVLHQDWLNGKE
YKCAVSNKALPAPIEKTISKAKGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG
418E4HUMANIZEDHEAVYCHAINVARIABLEHEAVYCHAINAMINOACIDSEQUENCE.
COMPLIMENTARITYDETERMININGREGIONSAREUNDERLINED
SEQ ID NO: 184
EVQLVESGGGLVQPGGSLRLSCAASGFSLSSYYMSWVRQAPGKGLEWIGSIYASGSAYYASSAKGRFTISRDNSKNTLYL
QMNSLRAEDTAVYYCARDPINNDIWGQGTLVTVSS
418H5HUMANIZEDLIGHTCHAINFULL-LENGTHNUCLEOTIDESEQUENCE
SEQ ID NO: 185
GACATCCAGATGACCCAGTCTCCATCTTCCGTGTCTGCATCTGTAGGAGACAGAGTCACCATCACTTGTCAGTCCA
GTCCGAGTGTTTATAAGAACAACCAATTAGCCTGGTATCAGCAGAAACCAGGGAAAGCCCCTAAGCTCCTGATCTA
TCTGGCATCTACTCTGGCATCTGGGGTCCCATCAAGGTTCAGCGGCAGTGGATCTGGGACAGATTTCACTCTCACC
ATCAGCAGCCTGCAGCCTGAAGATTTTGCAACTTACTATTGTGCAGGCGGTTATAGTAGTAGTAGTGATACTGCTT
TCGGCGGAGGGACCAAGGTGGAGATCAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAGAGCAGGACAGCAAGGACAGCACCT
ACAGCCTCAGCAGCACCCTGACGCTGAGCAAAGCAGACTACGAGAAACACAAAGTCTACGCCTGCGAAGTCACCC
ATCAGGGCCTGAGCTCGCCCGTCACAAAGAGCTTCAACAGGGGAGAGTGT
418H5HUMANIZEDLIGHTCHAINVARIABLELIGHTCHAINNUCLEOTIDESEQUENCE
SEQ ID NO: 186
GACATCCAGATGACCCAGTCTCCATCTTCCGTGTCTGCATCTGTAGGAGACAGAGTCACCATCACTTGTCAGTCCA
GTCCGAGTGTTTATAAGAACAACCAATTAGCCTGGTATCAGCAGAAACCAGGGAAAGCCCCTAAGCTCCTGATCTA
TCTGGCATCTACTCTGGCATCTGGGGTCCCATCAAGGTTCAGCGGCAGTGGATCTGGGACAGATTTCACTCTCACC
ATCAGCAGCCTGCAGCCTGAAGATTTTGCAACTTACTATTGTGCAGGCGGTTATAGTAGTAGTAGTGATACTGCTT
TCGGCGGAGGGACCAAGGTGGAGATCAAA
418H5HUMANIZEDLIGHTCHAINFULL-LENGTHAMINOACIDSEQUENCE.HUMANKAPPACONSTANT
DOMAINISUNDERLINED
SEQ ID NO: 187
DIQMTQSPSSVSASVGDRVTITCQSSPSVYKNNQLAWYQQKPGKAPKLLIYLASTLASGVPSRFSGSGSGTDFTLTISSLQ
PEDFATYYCAGGYSSSSDTAFGGGTKVEIKRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC
418H5HUMANIZEDLIGHTCHAINVARIABLELIGHTCHAINAMINOACIDSEQUENCE.COMPIMENTARITY
DETERMININGREGIONSAREUNDERLINED
SEQ ID NO: 188
DIQMTQSPSSVSASVGDRVTITCQSSPSVYKNNQLAWYQQKPGKAPKLLIYLASTLASGVPSRFSGSGSGTDFTLTISSLQ
PEDFATYYCAGGYSSSSDTAFGGGTKVEIK
418H5HUMANIZEDHEAVYCHAINFULL-LENGTHNUCLEOTIDESEQUENCE
SEQ ID NO: 189
GAGGTGCAGCTGGTGGAGTCTGGGGGAGGCTTGGTCCAGCCTGGGGGGTCCCTGAGACTCTCCTGTACAGCCTC
TGGAATCGACCTCAGTGCCTACCACATGAGCTGGGTCCGCCAGGCTCCAGGGAAGGGGCTGGAGTACATCGGGA
TGATTGGTAGTAGTGGTACCATACACTACGCGAACTCCGCGAAAGGCAGATTCACCATCTCCAAAGACAATACCAA
GAACACGGTGGATCTTCAAATGAACAGCCTGAGAGCCGAGGACACGGCTGTGTATTACTGTGCGAGAGACAGTT
ATAATAGTGATTATGCCTTTAACTTATGGGGCCAGGGAACCCTGGTCACCGTCTCGAGCGCTAGCACCAAGGGCCC
ATCGGTCTTCCCCCTGGCACCCTCCTCCAAGAGCACCTCTGGGGGCACAGCGGCCCTGGGCTGCCTGGTCAAGGA
CTACTTCCCCGAACCGGTGACGGTGTCGTGGAACTCAGGCGCCCTGACCAGCGGCGTGCACACCTTCCCGGCTGTC
CTACAGTCCTCAGGACTCTACTCCCTCAGCAGCGTGGTGACCGTGCCCTCCAGCAGCTTGGGCACCCAGACCTACA
TCTGCAACGTGAATCACAAGCCCAGCAACACCAAGGTGGACAAGAGAGTTGAGCCCAAATCTTGTGACAAAACTC
ACACATGCCCACCGTGCCCAGCACCTGAAGCCGCGGGGGCACCGTCAGTCTTCCTCTTCCCCCCAAAACCCAAGGA
CACCCTCATGATCTCCCGGACCCCTGAGGTCACATGCGTGGTGGTGGACGTGAGCCACGAAGACCCTGAGGTCAA
GTTCAACTGGTACGTGGACGGCGTGGAGGTGCATAATGCCAAGACAAAGCCGCGGGAGGAGCAGTACAACAGCA
CGTACCGTGTGGTCAGCGTCCTCACCGTCCTGCACCAGGACTGGCTGAATGGCAAGGAGTACAAGTGCGCGGTCT
CCAACAAAGCCCTCCCAGCCCCCATCGAGAAAACCATCTCCAAAGCCAAAGGGCAGCCCCGAGAACCACAGGTGT
ACACCCTGCCCCCATCCCGGGATGAGCTGACCAAGAACCAGGTCAGCCTGACCTGCCTGGTCAAAGGCTTCTATCC
CAGCGACATCGCCGTGGAGTGGGAGAGCAATGGGCAGCCGGAGAACAACTACAAGACCACGCCTCCCGTGCTGG
ACTCCGACGGCTCCTTCTTCCTCTATAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGGGT
418H5HUMANIZEDHEAVYCHAINVARIABLEHEAVYCHAINNUCLEOTIDESEQUENCE
SEQ ID NO: 190
GAGGTGCAGCTGGTGGAGTCTGGGGGAGGCTTGGTCCAGCCTGGGGGGTCCCTGAGACTCTCCTGTACAGCCTC
TGGAATCGACCTCAGTGCCTACCACATGAGCTGGGTCCGCCAGGCTCCAGGGAAGGGGCTGGAGTACATCGGGA
TGATTGGTAGTAGTGGTACCATACACTACGCGAACTCCGCGAAAGGCAGATTCACCATCTCCAAAGACAATACCAA
GAACACGGTGGATCTTCAAATGAACAGCCTGAGAGCCGAGGACACGGCTGTGTATTACTGTGCGAGAGACAGTT
ATAATAGTGATTATGCCTTTAACTTATGGGGCCAGGGAACCCTGGTCACCGTCTCGAGC
418H5HUMANIZEDHEAVYCHAINFULL-LENGTHAMINOACIDSEQUENCE.HUMANGAMMA-1
CONSTANTDOMAINISUNDERLINED
SEQ ID NO: 191
EVQLVESGGGLVQPGGSLRLSCTASGIDLSAYHMSWVRQAPGKGLEYIGMIGSSGTIHYANSAKGRFTISKDNTKNTVD
LQMNSLRAEDTAVYYCARDSYNSDYAFNLWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKVDKRVEPKSCDKTHTCPPCPAPEAA
GAPSVFLFPPKPKDTLMISRTPEVTCVVVDVSHEDPEVKFNWYVDGVEVHNAKTKPREEQYNSTYRVVSVLTVLHQDW
LNGKEYKCAVSNKALPAPIEKTISKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPG
418H5HUMANIZEDHEAVYCHAINVARIABLEHEAVYCHAINAMINOACIDSEQUENCE.
COMPLIMENTARITYDETERMININGREGIONSAREUNDERLINED
SEQ ID NO: 192
EVQLVESGGGLVQPGGSLRLSCTASGIDLSAYHMSWVRQAPGKGLEYIGMIGSSGTIHYANSAKGRFTISKDNTKNTVD
LQMNSLRAEDTAVYYCARDSYNSDYAFNLWGQGTLVTVSS
419D9HUMANIZEDLIGHTCHAINFULL-LENGTHNUCLEOTIDESEQUENCE
SEQ ID NO: 193
GACATCCAGATGACCCAGTCTCCTTCCACCCTGTCTGCATCTGTAGGAGACAGAGTCACCATCACTTGCCAGTCCA
GTCAGAGTGTTTATAGTAACTGGTTATCCTGGTATCAGCAGAAACCAGGGAAAGCCCCTAAGAGACTGATCTATTC
TGCATCCACTCTGGCATCTGGGGTCCCATCAAGGTTCAGCGGCAGTGGATCTGGGACAGAATTCACTCTCACCATC
AGCAGCCTGCAGCCTGATGATTTTGCAACTTATTACTGCGCAGGCGGTTATAATACTGTTACTGATACTTTTGCTTT
CGGCGGAGGGACCAAGGTGGAGATCAAACGTACGGTGGCTGCACCATCTGTCTTCATCTTCCCGCCATCTGATGA
GCAGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGT
419D9HUMANIZEDLIGHTCHAINVARIABLELIGHTCHAINNUCLEOTIDESEQUENCE
SEQ ID NO: 194
GACATCCAGATGACCCAGTCTCCTTCCACCCTGTCTGCATCTGTAGGAGACAGAGTCACCATCACTTGCCAGTCCA
GTCAGAGTGTTTATAGTAACTGGTTATCCTGGTATCAGCAGAAACCAGGGAAAGCCCCTAAGAGACTGATCTATTC
TGCATCCACTCTGGCATCTGGGGTCCCATCAAGGTTCAGCGGCAGTGGATCTGGGACAGAATTCACTCTCACCATC
AGCAGCCTGCAGCCTGATGATTTTGCAACTTATTACTGCGCAGGCGGTTATAATACTGTTACTGATACTTTTGCTTT
CGGCGGAGGGACCAAGGTGGAGATCAAA
419D9HUMANIZEDLIGHTCHAINFULL-LENGTHAMINOACIDSEQUENCE.HUMANKAPPACONSTANT
DOMAINISUNDERLINED
SEQ ID NO: 195
DIQMTQSPSTLSASVGDRVTITCQSSQSVYSNWLSWYQQKPGKAPKRLIYSASTLASGVPSRFSGSGSGTEFTLTISSLQP
DDFATYYCAGGYNTVTDTFAFGGGTKVEIKRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC
419D9HUMANIZEDLIGHTCHAINVARIABLELIGHTCHAINAMINOACIDSEQUENCE.COMPIMENTARITY
DETERMININGREGIONSAREUNDERLINED
SEQ ID NO: 196
DIQMTQSPSTLSASVGDRVTITCQSSQSVYSNWLSWYQQKPGKAPKRLIYSASTLASGVPSRFSGSGSGTEFTLTISSLQP
DDFATYYCAGGYNTVTDTFAFGGGTKVEIK
419D9HUMANIZEDHEAVYCHAINFULL-LENGTHNUCLEOTIDESEQUENCE
SEQ ID NO: 197
GAGGTGCAGCTGTTGGAGTCTGGGGGAGGCTTGGTACAGCCTGGGGGGTCCCTGAGACTCTCCTGTACAGCCTCT
GGATTCTCCTTCAGTAGCAGGCACTACATGTGTTGGGTCCGCCAGGCTCCAGGGAAGGGGCTGGAGTGGATCGCA
TGCATTTATACTGGTAGTAGTGGTACTCCTCACTACGCGAGCTCCGCGAAAGGCCGGTTCACCATCTCCCAAGACA
ATTCCAAGAACACGGTGACTCTGCAAATGAACAGCCTGAGAGCCGAGGACACGGCCGTATATTACTGTGCGAGAG
AAGGTAACCTGTGGGGCCAGGGAACCCTGGTCACCGTCTCGAGCGCTAGCACCAAGGGCCCATCGGTCTTCCCCC
TGGCACCCTCCTCCAAGAGCACCTCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACCCAGACCTACATCTGCAACGTGAA
TCACAAGCCCAGCAACACCAAGGTGGACAAGAGAGTTGAGCCCAAATCTTGTGACAAAACTCACACATGCCCACC
GTGCCCAGCACCTGAAGCCGCGGGGGCACCGTCAGTCTTCCTCTTCCCCCCAAAACCCAAGGACACCCTCATGATC
TCCCGGACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTGTGG
TCAGCGTCCTCACCGTCCTGCACCAGGACTGGCTGAATGGCAAGGAGTACAAGTGCGCGGTCTCCAACAAAGCCC
TCCCAGCCCCCATCGAGAAAACCATCTCCAAAGCCAAAGGGCAGCCCCGAGAACCACAGGTGTACACCCTGCCCC
CATCCCGGGATGAGCTGACCAAGAACCAGGTCAGCCTGACCTGCCTGGTCAAAGGCTTCTATCCCAGCGACATCG
CCGTGGAGTGGGAGAGCAATGGGCAGCCGGAGAACAACTACAAGACCACGCCTCCCGTGCTGGACTCCGACGGC
TCCTTCTTCCTCTATAGCAAGCTCACCGTGGACAAGAGCAGGTGGCAGCAGGGGAACGTCTTCTCATGCTCCGTGA
TGCATGAGGCTCTGCACAACCACTACACGCAGAAGAGCCTCTCCCTGTCTCCGGGT
419D9HUMANIZEDHEAVYCHAINVARIABLEHEAVYCHAINNUCLEOTIDESEQUENCE
SEQ ID NO: 198
GAGGTGCAGCTGTTGGAGTCTGGGGGAGGCTTGGTACAGCCTGGGGGGTCCCTGAGACTCTCCTGTACAGCCTCT
GGATTCTCCTTCAGTAGCAGGCACTACATGTGTTGGGTCCGCCAGGCTCCAGGGAAGGGGCTGGAGTGGATCGCA
TGCATTTATACTGGTAGTAGTGGTACTCCTCACTACGCGAGCTCCGCGAAAGGCCGGTTCACCATCTCCCAAGACA
ATTCCAAGAACACGGTGACTCTGCAAATGAACAGCCTGAGAGCCGAGGACACGGCCGTATATTACTGTGCGAGAG
AAGGTAACCTGTGGGGCCAGGGAACCCTGGTCACCGTCTCGAGC
419D9HUMANIZEDHEAVYCHAINFULL-LENGTHAMINOACIDSEQUENCE.HUMANGAMMA-1
CONSTANTDOMAINISUNDERLINED
SEQ ID NO: 199
EVQLLESGGGLVQPGGSLRLSCTASGFSFSSRHYMCWVRQAPGKGLEWIACIYTGSSGTPHYASSAKGRFTISQDNSKN
TVTLQMNSLRAEDTAVYYCAREGNLWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKRVEPKSCDKTHTCPPCPAPEAAGAPS
VFLEPPKPKDTLMISRTPEVTCVVVDVSHEDPEVKFNWYVDGVEVHNAKTKPREEQYNSTYRVVSVLTVLHQDWLNG
KEYKCAVSNKALPAPIEKTISKAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
419D9HUMANIZEDHEAVYCHAINVARIABLEHEAVYCHAINAMINOACIDSEQUENCE.
COMPLIMENTARITYDETERMININGREGIONSAREUNDERLINED
SEQ ID NO: 200
EVQLLESGGGLVQPGGSLRLSCTASGFSFSSRHYMCWVRQAPGKGLEWIACIYTGSSGTPHYASSAKGRFTISQDNSKN
TVTLQMNSLRAEDTAVYYCAREGNLWGQGTLVTVSS
420H5HUMANIZEDLIGHTCHAINFULL-LENGTHNUCLEOTIDESEQUENCE
GCCCTTGTGATGACCCAGTCTCCTTCCACCCTGTCTGCATCTGTAGGAGACAGAGTCACCATCAATTGCCAGGCCA
SEQ ID NO: 201
GTGAGGACATTGATACCTATTTAGCCTGGTATCAGCAGAAACCAGGGAAAGCCCCTAAGCTCCTGATCTTTTATGC
ATCCGATCTGGCATCTGGGGTCCCATCAAGGTTCAGCGGCAGTGGATCTGGGACAGAATTCACTCTCACCATCAGC
AGCCTGCAGCCTGATGATTTTGCAACTTATTACTGCCAAGGCGGTTACTATACTAGTAGTGCTGATACGAGGGGTG
CTTTCGGCGGAGGGACCAAGGTGGAGATCAAACGTACGGTGGCTGCACCATCTGTCTTCATCTTCCCGCCATCTGA
TGAGCAGTTGAAATCTGGAACTGCCTCTGTTGTGTGCCTGCTGAATAACTTCTATCCCAGAGAGGCCAAAGTACAG
TGGAAGGTGGATAACGCCCTCCAATCGGGTAACTCCCAGGAGAGTGTCACAGAGCAGGACAGCAAGGACAGCAC
CTACAGCCTCAGCAGCACCCTGACGCTGAGCAAAGCAGACTACGAGAAACACAAAGTCTACGCCTGCGAAGTCAC
CCATCAGGGCCTGAGCTCGCCCGTCACAAAGAGCTTCAACAGGGGAGAGTGT
420H5HUMANIZEDLIGHTCHAINVARIABLELIGHTCHAINNUCLEOTIDESEQUENCE
SEQ ID NO: 202
GCCCTTGTGATGACCCAGTCTCCTTCCACCCTGTCTGCATCTGTAGGAGACAGAGTCACCATCAATTGCCAGGCCA
GTGAGGACATTGATACCTATTTAGCCTGGTATCAGCAGAAACCAGGGAAAGCCCCTAAGCTCCTGATCTTTTATGC
ATCCGATCTGGCATCTGGGGTCCCATCAAGGTTCAGCGGCAGTGGATCTGGGACAGAATTCACTCTCACCATCAGC
AGCCTGCAGCCTGATGATTTTGCAACTTATTACTGCCAAGGCGGTTACTATACTAGTAGTGCTGATACGAGGGGTG
CTTTCGGCGGAGGGACCAAGGTGGAGATCAAA
420H5HUMANIZEDLIGHTCHAINFULL-LENGTHAMINOACIDSEQUENCE.HUMANKAPPACONSTANT
DOMAINISUNDERLINED
SEQ ID NO: 203
ALVMTQSPSTLSASVGDRVTINCQASEDIDTYLAWYQQKPGKAPKLLIFYASDLASGVPSRFSGSGSGTEFTLTISSLQPD
DFATYYCQGGYYTSSADTRGAFGGGTKVEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC
420H5HUMANIZEDLIGHTCHAINVARIABLELIGHTCHAINAMINOACIDSEQUENCE.COMPIMENTARITY
DETERMININGREGIONSAREUNDERLINED
SEQ ID NO: 204
ALVMTQSPSTLSASVGDRVTINCQASEDIDTYLAWYQQKPGKAPKLLIFYASDLASGVPSRFSGSGSGTEFTLTISSLQPD
DFATYYCQGGYYTSSADTRGAFGGGTKVEIK
420H5HUMANIZEDHEAVYCHAINFULL-LENGTHNUCLEOTIDESEQUENCE
SEQ ID NO: 205
CAGTCGCTGGTGGAGTCTGGGGGAGGCTTGGTACAGCCTGGGGGGTCCCTGAGACTCTCCTGTGCAGCCTCTGG
ATTCTCCTTCAGTAGCAACTACTGGATATGCTGGGTCCGCCAGGCTCCAGGGAAGGGGCTGGAGTGGATCGCATG
CATTTATGTTGGTAGTAGTGGTGACACTTACTACGCGAGCTCCGCGAAAGGCCGGTTCACCATCTCCAGAGACAAT
TCCAAGAACACGCTGTATCTGCAAATGAACAGCCTGAGAGCCGAGGACACGGCCGTATATTACTGTGCGAGAGAT
AGTAGTAGTTATTATATGTTTAACTTGTGGGGCCAGGGAACCCTGGTCACCGTCTCGAGCGCTAGCACCAAGGGC
CCATCGGTCTTCCCCCTGGCACCCTCCTCCAAGAGCACCTCTGGGGGCACAGCGGCCCTGGGCTGCCTGGTCAAGG
ACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTA
CATCTGCAACGTGAATCACAAGCCCAGCAACACCAAGGTGGACAAGAGAGTTGAGCCCAAATCTTGTGACAAAAC
TCACACATGCCCACCGTGCCCAGCACCTGAAGCCGCGGGGGCACCGTCAGTCTTCCTCTTCCCCCCAAAACCCAAG
GACACCCTCATGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGCAGTACAACA
GCACGTACCGTGTGGTCAGCGTCCTCACCGTCCTGCACCAGGACTGGCTGAATGGCAAGGAGTACAAGTGCGCGG
TCTCCAACAAAGCCCTCCCAGCCCCCATCGAGAAAACCATCTCCAAAGCCAAAGGGCAGCCCCGAGAACCACAGG
TGTACACCCTGCCCCCATCCCGGGATGAGCTGACCAAGAACCAGGTCAGCCTGACCTGCCTGGTCAAAGGCTTCTA
TCCCAGCGACATCGCCGTGGAGTGGGAGAGCAATGGGCAGCCGGAGAACAACTACAAGACCACGCCTCCCGTGC
TGGACTCCGACGGCTCCTTCTTCCTCTATAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTCCGGGT
420H5HUMANIZEDHEAVYCHAINVARIABLEHEAVYCHAINNUCLEOTIDESEQUENCE
SEQ ID NO: 206
CAGTCGCTGGTGGAGTCTGGGGGAGGCTTGGTACAGCCTGGGGGGTCCCTGAGACTCTCCTGTGCAGCCTCTGG
ATTCTCCTTCAGTAGCAACTACTGGATATGCTGGGTCCGCCAGGCTCCAGGGAAGGGGCTGGAGTGGATCGCATG
CATTTATGTTGGTAGTAGTGGTGACACTTACTACGCGAGCTCCGCGAAAGGCCGGTTCACCATCTCCAGAGACAAT
TCCAAGAACACGCTGTATCTGCAAATGAACAGCCTGAGAGCCGAGGACACGGCCGTATATTACTGTGCGAGAGAT
AGTAGTAGTTATTATATGTTTAACTTGTGGGGCCAGGGAACCCTGGTCACCGTCTCGAGC
420H5HUMANIZEDHEAVYCHAINFULL-LENGTHAMINOACIDSEQUENCE.HUMANGAMMA-1
CONSTANTDOMAINISUNDERLINED
SEQ ID NO: 207
QSLVESGGGLVQPGGSLRLSCAASGFSFSSNYWICWVRQAPGKGLEWIACIYVGSSGDTYYASSAKGRFTISRDNSKNT
LYLQMNSLRAEDTAVYYCARDSSSYYMFNLWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKRVEPKSCDKTHTCPPCPAPEAA
GAPSVFLEPPKPKDTLMISRTPEVTCVVVDVSHEDPEVKFNWYVDGVEVHNAKTKPREEQYNSTYRVVSVLTVLHQDW
LNGKEYKCAVSNKALPAPIEKTISKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPG
420H5HUMANIZEDHEAVYCHAINVARIABLEHEAVYCHAINAMINOACIDSEQUENCE.
COMPLIMENTARITYDETERMININGREGIONSAREUNDERLINED
SEQ ID NO: 208
QSLVESGGGLVQPGGSLRLSCAASGFSFSSNYWICWVRQAPGKGLEWIACIYVGSSGDTYYASSAKGRFTISRDNSKNT
LYLQMNSLRAEDTAVYYCARDSSSYYMFNLWGQGTLVTVSS
70
420H6HUMANIZEDLIGHTCHAINFULL-LENGTHNUCLEOTIDESEQUENCE
SEQ ID NO: 209
GACATCCAGATGACCCAGTCTCCTTCCACCCTGTCTGCATCTGTAGGAGACAGAGTCACCATCACTTGCCAGGCCA
GTCAGAGTGTTTATAGTAACTGGTTATCCTGGTATCAGCAGAAACCAGGGAAAGCCCCTAAGCTCCTGATCTATTC
TGCATCCACTCTGGCATCTGGGGTCCCATCAAGGTTCAGCGGCAGTGGATCTGGGACAGAATTCACTCTCACCATC
AGCAGCCTGCAGCCTGATGATTTTGCAACTTATTACTGCGCAGGCGGTTACAATACTGTAATTGATACTTTTGCTTT
CGGCGGAGGGACCAAGGTGGAGATCAAACGTACGGTGGCTGCACCATCTGTCTTCATCTTCCCGCCATCTGATGA
GCAGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGT
420H6HUMANIZEDLIGHTCHAINVARIABLELIGHTCHAINNUCLEOTIDESEQUENCE
SEQ ID NO: 210
GACATCCAGATGACCCAGTCTCCTTCCACCCTGTCTGCATCTGTAGGAGACAGAGTCACCATCACTTGCCAGGCCA
GTCAGAGTGTTTATAGTAACTGGTTATCCTGGTATCAGCAGAAACCAGGGAAAGCCCCTAAGCTCCTGATCTATTC
TGCATCCACTCTGGCATCTGGGGTCCCATCAAGGTTCAGCGGCAGTGGATCTGGGACAGAATTCACTCTCACCATC
AGCAGCCTGCAGCCTGATGATTTTGCAACTTATTACTGCGCAGGCGGTTACAATACTGTAATTGATACTTTTGCTTT
CGGCGGAGGGACCAAGGTGGAGATCAAA
420H6HUMANIZEDLIGHTCHAINFULL-LENGTHAMINOACIDSEQUENCE.HUMANKAPPACONSTANT
DOMAINISUNDERLINED
SEQ ID NO: 211
DIQMTQSPSTLSASVGDRVTITCQASQSVYSNWLSWYQQKPGKAPKLLIYSASTLASGVPSRFSGSGSGTEFTLTISSLQP
DDFATYYCAGGYNTVIDTFAFGGGTKVEIKRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC
420H6HUMANIZEDLIGHTCHAINVARIABLELIGHTCHAINAMINOACIDSEQUENCE.COMPIMENTARITY
DETERMININGREGIONSAREUNDERLINED
SEQ ID NO: 212
DIQMTQSPSTLSASVGDRVTITCQASQSVYSNWLSWYQQKPGKAPKLLIYSASTLASGVPSRFSGSGSGTEFTLTISSLQP
DDFATYYCAGGYNTVIDTFAFGGGTKVEIK
420H6HUMANIZEDHEAVYCHAINFULL-LENGTHNUCLEOTIDESEQUENCE
SEQ ID NO: 213
GAGGTGCAGCTGTTGGAGTCTGGGGGAGGCTTGGTACAGCCTGGGGGGTCCCTGAGACTCTCCTGTGCAGCCTCT
GGAATCGACTTCAGTAGTAGTTACTACATGTGCTGGGTCCGCCAGGCTCCAGGGAAGGGGCTGGAGTGGATCGC
ATGCATTTATACTGGTGGTAGTGGTACTCCTCACTACGCGAGCTCCGCGAAAGGCCGGTTCACCATCTCCAGAGAC
AATTCCAAGAACACGCTGTATCTGCAAATGAACAGCCTGAGAGCCGAGGACACGGCCGTATATTACTGTGCGAGA
GAAGGTAGCCTGTGGGGCCAGGGAACCCTGGTCACCGTCTCGAGCGCTAGCACCAAGGGCCCATCGGTCTTCCCC
CTGGCACCCTCCTCCAAGAGCACCTCTGGGGGCACAGCGGCCCTGGGCTGCCTGGTCAAGGACTACTTCCCCGAA
CCGGTGACGGTGTCGTGGAACTCAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCACCCAGACCTACATCTGCAACGTGA
ATCACAAGCCCAGCAACACCAAGGTGGACAAGAGAGTTGAGCCCAAATCTTGTGACAAAACTCACACATGCCCAC
CGTGCCCAGCACCTGAAGCCGCGGGGGCACCGTCAGTCTTCCTCTTCCCCCCAAAACCCAAGGACACCCTCATGAT
CTCCCGGACCCCTGAGGTCACATGCGTGGTGGTGGACGTGAGCCACGAAGACCCTGAGGTCAAGTTCAACTGGTA
CGTGGACGGCGTGGAGGTGCATAATGCCAAGACAAAGCCGCGGGAGGAGCAGTACAACAGCACGTACCGTGTG
GTCAGCGTCCTCACCGTCCTGCACCAGGACTGGCTGAATGGCAAGGAGTACAAGTGCGCGGTCTCCAACAAAGCC
CTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATC
GCCGTGGAGTGGGAGAGCAATGGGCAGCCGGAGAACAACTACAAGACCACGCCTCCCGTGCTGGACTCCGACGG
CTCCTTCTTCCTCTATAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGT
420H6HUMANIZEDHEAVYCHAINVARIABLEHEAVYCHAINNUCLEOTIDESEQUENCE
SEQ ID NO: 214
GAGGTGCAGCTGTTGGAGTCTGGGGGAGGCTTGGTACAGCCTGGGGGGTCCCTGAGACTCTCCTGTGCAGCCTCT
GGAATCGACTTCAGTAGTAGTTACTACATGTGCTGGGTCCGCCAGGCTCCAGGGAAGGGGCTGGAGTGGATCGC
ATGCATTTATACTGGTGGTAGTGGTACTCCTCACTACGCGAGCTCCGCGAAAGGCCGGTTCACCATCTCCAGAGAC
AATTCCAAGAACACGCTGTATCTGCAAATGAACAGCCTGAGAGCCGAGGACACGGCCGTATATTACTGTGCGAGA
GAAGGTAGCCTGTGGGGCCAGGGAACCCTGGTCACCGTCTCGAGC
420H6HUMANIZEDHEAVYCHAINFULL-LENGTHAMINOACIDSEQUENCE.HUMANGAMMA-1
CONSTANTDOMAINISUNDERLINED
SEQ ID NO: 215
EVQLLESGGGLVQPGGSLRLSCAASGIDFSSSYYMCWVRQAPGKGLEWIACIYTGGSGTPHYASSAKGRFTISRDNSKN
TLYLQMNSLRAEDTAVYYCAREGSLWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKRVEPKSCDKTHTCPPCPAPEAAGAPSV
FLFPPKPKDTLMISRTPEVTCVVVDVSHEDPEVKFNWYVDGVEVHNAKTKPREEQYNSTYRVVSVLTVLHQDWLNGKE
YKCAVSNKALPAPIEKTISKAKGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG
420H6HUMANIZEDHEAVYCHAINVARIABLEHEAVYCHAINAMINOACIDSEQUENCE.
COMPLIMENTARITYDETERMININGREGIONSAREUNDERLINED
SEQ ID NO: 216
EVQLLESGGGLVQPGGSLRLSCAASGIDFSSSYYMCWVRQAPGKGLEWIACIYTGGSGTPHYASSAKGRFTISRDNSKN
TLYLQMNSLRAEDTAVYYCAREGSLWGQGTLVTVSS
459F1HUMANIZEDLIGHTCHAINFULL-LENGTHNUCLEOTIDESEQUENCE
SEQ ID NO: 217
GACATCCAGATGACCCAGTCTCCTTCCACCCTGTCTGCATCTGTAGGAGACAGAGTCACCATCACTTGCCAGTCCA
GTCAGAGTGTTTATAGTAACTGGTTCTCCTGGTATCAGCAGAAACCAGGGAAAGCCCCTAAGCTCCTGATCTATTC
TGCATCCACTCTGGCATCTGGGGTCCCATCAAGGTTCAGCGGCAGTGGATCTGGGACAGAATTCACTCTCACCATC
AGCAGCCTGCAGCCTGATGATTTTGCAACTTATTACTGCGCAGGCGGTTACAATACTGTAACTGATACTTTTGCTTT
CGGCGGAGGGACCAAGGTGGAGATCAAACGTACGGTGGCTGCACCATCTGTCTTCATCTTCCCGCCATCTGATGA
GCAGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGT
459F1HUMANIZEDLIGHTCHAINVARIABLELIGHTCHAINNUCLEOTIDESEQUENCE
SEQ ID NO: 218
GACATCCAGATGACCCAGTCTCCTTCCACCCTGTCTGCATCTGTAGGAGACAGAGTCACCATCACTTGCCAGTCCA
GTCAGAGTGTTTATAGTAACTGGTTCTCCTGGTATCAGCAGAAACCAGGGAAAGCCCCTAAGCTCCTGATCTATTC
TGCATCCACTCTGGCATCTGGGGTCCCATCAAGGTTCAGCGGCAGTGGATCTGGGACAGAATTCACTCTCACCATC
AGCAGCCTGCAGCCTGATGATTTTGCAACTTATTACTGCGCAGGCGGTTACAATACTGTAACTGATACTTTTGCTTT
CGGCGGAGGGACCAAGGTGGAGATCAAA
459F1HUMANIZEDLIGHTCHAINFULL-LENGTHAMINOACIDSEQUENCE.HUMANKAPPACONSTANT
DOMAINISUNDERLINED
SEQ ID NO: 219
DIQMTQSPSTLSASVGDRVTITCQSSQSVYSNWFSWYQQKPGKAPKLLIYSASTLASGVPSRFSGSGSGTEFTLTISSLQP
DDFATYYCAGGYNTVTDTFAFGGGTKVEIKRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC
459F1HUMANIZEDLIGHTCHAINVARIABLELIGHTCHAINAMINOACIDSEQUENCE.COMPIMENTARITY
DETERMININGREGIONSAREUNDERLINED
SEQ ID NO: 220
DIQMTQSPSTLSASVGDRVTITCQSSQSVYSNWFSWYQQKPGKAPKLLIYSASTLASGVPSRFSGSGSGTEFTLTISSLQP
DDFATYYCAGGYNTVTDTFAFGGGTKVEIK
459F1HUMANIZEDHEAVYCHAINFULL-LENGTHNUCLEOTIDESEQUENCE
SEQ ID NO: 221
GAGGTGCAGCTGTTGGAGTCTGGGGGAGGCTTGGTACAGCCTGGGGGGTCCCTGAGACTCTCCTGTGCAGCCTCT
GGAATCGACTTCAGTAGGAGATACTACATGTGCTGGGTCCGCCAGGCTCCAGGGAAGGGGCTGGAGTGGATCGC
ATGCATGTATACTGGTAGCCGCGATACTCCTCACTACGCGAGCTCCGCGAAAGGCCGGTTCACCATCTCCAGAGAC
AATTCCAAGAACACGCTGTATCTGCAAATGAACAGCCTGAGAGCCGAGGACACGGCCGTATATTACTGTGCGAGA
GAAGGTAACCTGTGGGGCCAGGGAACCCTGGTCACCGTCTCGAGCGCTAGCACCAAGGGCCCATCGGTCTTCCCC
CTGGCACCCTCCTCCAAGAGCACCTCTGGGGGCACAGCGGCCCTGGGCTGCCTGGTCAAGGACTACTTCCCCGAA
CCGGTGACGGTGTCGTGGAACTCAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCACCCAGACCTACATCTGCAACGTGA
ATCACAAGCCCAGCAACACCAAGGTGGACAAGAGAGTTGAGCCCAAATCTTGTGACAAAACTCACACATGCCCAC
CGTGCCCAGCACCTGAAGCCGCGGGGGCACCGTCAGTCTTCCTCTTCCCCCCAAAACCCAAGGACACCCTCATGAT
CTCCCGGACCCCTGAGGTCACATGCGTGGTGGTGGACGTGAGCCACGAAGACCCTGAGGTCAAGTTCAACTGGTA
CGTGGACGGCGTGGAGGTGCATAATGCCAAGACAAAGCCGCGGGAGGAGCAGTACAACAGCACGTACCGTGTG
GTCAGCGTCCTCACCGTCCTGCACCAGGACTGGCTGAATGGCAAGGAGTACAAGTGCGCGGTCTCCAACAAAGCC
CTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATC
GCCGTGGAGTGGGAGAGCAATGGGCAGCCGGAGAACAACTACAAGACCACGCCTCCCGTGCTGGACTCCGACGG
CTCCTTCTTCCTCTATAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGT
459F1HUMANIZEDHEAVYCHAINVARIABLEHEAVYCHAINNUCLEOTIDESEQUENCE
SEQ ID NO: 222
GAGGTGCAGCTGTTGGAGTCTGGGGGAGGCTTGGTACAGCCTGGGGGGTCCCTGAGACTCTCCTGTGCAGCCTCT
GGAATCGACTTCAGTAGGAGATACTACATGTGCTGGGTCCGCCAGGCTCCAGGGAAGGGGCTGGAGTGGATCGC
ATGCATGTATACTGGTAGCCGCGATACTCCTCACTACGCGAGCTCCGCGAAAGGCCGGTTCACCATCTCCAGAGAC
AATTCCAAGAACACGCTGTATCTGCAAATGAACAGCCTGAGAGCCGAGGACACGGCCGTATATTACTGTGCGAGA
GAAGGTAACCTGTGGGGCCAGGGAACCCTGGTCACCGTCTCGAGC
459F1HUMANIZEDHEAVYCHAINFULL-LENGTHAMINOACIDSEQUENCE.HUMANGAMMA-1
CONSTANTDOMAINISUNDERLINED
SEQ ID NO: 223
EVQLLESGGGLVQPGGSLRLSCAASGIDFSRRYYMCWVRQAPGKGLEWIACMYTGSRDTPHYASSAKGRFTISRDNSK
NTLYLQMNSLRAEDTAVYYCAREGNLWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKRVEPKSCDKTHTCPPCPAPEAAGAP
SVFLEPPKPKDTLMISRTPEVTCVVVDVSHEDPEVKFNWYVDGVEVHNAKTKPREEQYNSTYRVVSVLTVLHQDWLNG
KEYKCAVSNKALPAPIEKTISKAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
459F1HUMANIZEDHEAVYCHAINVARIABLEHEAVYCHAINAMINOACIDSEQUENCE.
COMPLIMENTARITYDETERMININGREGIONSAREUNDERLINED
SEQ ID NO: 224
EVQLLESGGGLVQPGGSLRLSCAASGIDFSRRYYMCWVRQAPGKGLEWIACMYTGSRDTPHYASSAKGRFTISRDNSK
NTLYLQMNSLRAEDTAVYYCAREGNLWGQGTLVTVSS
460C3HUMANIZEDLIGHTCHAINFULL-LENGTHNUCLEOTIDESEQUENCE
SEQ ID NO: 225
GACATCCAGATGACCCAGTCTCCTTCCACCCTGTCTGCATCTGTAGGAGACAGAGTCACCATCACTTGCCAGTCCA
GTCAGAGTGTTTATAGTAACTGGTTCTCCTGGTATCAGCAGAAACCAGGGAAAGCCCCTAAGCTCCTGATCTATTC
TGCATCCACTCTGGCATCTGGGGTCCCATCAAGGTTCAGCGGCAGTGGATCTGGGACAGAATTCACTCTCACCATC
AGCAGCCTGCAGCCTGATGATTTTGCAACTTATTACTGCGCAGGCGGTTACAATACTGTTATTGATACTTTTGCTTT
CGGCGGAGGGACCAAGGTGGAGATCAAACGTACGGTGGCTGCACCATCTGTCTTCATCTTCCCGCCATCTGATGA
GCAGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGT
460C3HUMANIZEDLIGHTCHAINVARIABLELIGHTCHAINNUCLEOTIDESEQUENCE
SEQ ID NO: 226
GACATCCAGATGACCCAGTCTCCTTCCACCCTGTCTGCATCTGTAGGAGACAGAGTCACCATCACTTGCCAGTCCA
GTCAGAGTGTTTATAGTAACTGGTTCTCCTGGTATCAGCAGAAACCAGGGAAAGCCCCTAAGCTCCTGATCTATTC
TGCATCCACTCTGGCATCTGGGGTCCCATCAAGGTTCAGCGGCAGTGGATCTGGGACAGAATTCACTCTCACCATC
AGCAGCCTGCAGCCTGATGATTTTGCAACTTATTACTGCGCAGGCGGTTACAATACTGTTATTGATACTTTTGCTTT
CGGCGGAGGGACCAAGGTGGAGATCAAA
460C3HUMANIZEDLIGHTCHAINFULL-LENGTHAMINOACIDSEQUENCE.HUMANKAPPACONSTANT
DOMAINISUNDERLINED
SEQ ID NO: 227
DIQMTQSPSTLSASVGDRVTITCQSSQSVYSNWFSWYQQKPGKAPKLLIYSASTLASGVPSRFSGSGSGTEFTLTISSLQP
DDFATYYCAGGYNTVIDTFAFGGGTKVEIKRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC
460C3HUMANIZEDLIGHTCHAINVARIABLELIGHTCHAINAMINOACIDSEQUENCE.COMPIMENTARITY
DETERMININGREGIONSAREUNDERLINED
SEQ ID NO: 228
DIQMTQSPSTLSASVGDRVTITCQSSQSVYSNWFSWYQQKPGKAPKLLIYSASTLASGVPSRFSGSGSGTEFTLTISSLQP
DDFATYYCAGGYNTVIDTFAFGGGTKVEIK
460C3HUMANIZEDHEAVYCHAINFULL-LENGTHNUCLEOTIDESEQUENCE
SEQ ID NO: 229
GAGGTGCAGCTGTTGGAGTCTGGGGGAGGCTTGGTACAGCCTGGGGGGTCCCTGAGACTCTCCTGTGCAGCCTCT
GGAATCGACTTCAGTAGGAGATACTACATGTGCTGGGTCCGCCAGGCTCCAGGGAAGGGGCTGGAGTGGATCGC
ATGCATATATACTGGTAGCCGCGATACTCCTCACTACGCGAGCTCCGCGAAAGGCCGGTTCACCATCTCCAGAGAC
AATTCCAAGAACACGCTGTATCTGCAAATGAACAGCCTGAGAGCCGAGGACACGGCCGTATATTACTGTGCGAGA
GAAGGTAGCCTGTGGGGCCAGGGAACCCTGGTCACCGTCTCGAGCGCTAGCACCAAGGGCCCATCGGTCTTCCCC
CTGGCACCCTCCTCCAAGAGCACCTCTGGGGGCACAGCGGCCCTGGGCTGCCTGGTCAAGGACTACTTCCCCGAA
CCGGTGACGGTGTCGTGGAACTCAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCACCCAGACCTACATCTGCAACGTGA
ATCACAAGCCCAGCAACACCAAGGTGGACAAGAGAGTTGAGCCCAAATCTTGTGACAAAACTCACACATGCCCAC
CGTGCCCAGCACCTGAAGCCGCGGGGGCACCGTCAGTCTTCCTCTTCCCCCCAAAACCCAAGGACACCCTCATGAT
CTCCCGGACCCCTGAGGTCACATGCGTGGTGGTGGACGTGAGCCACGAAGACCCTGAGGTCAAGTTCAACTGGTA
CGTGGACGGCGTGGAGGTGCATAATGCCAAGACAAAGCCGCGGGAGGAGCAGTACAACAGCACGTACCGTGTG
GTCAGCGTCCTCACCGTCCTGCACCAGGACTGGCTGAATGGCAAGGAGTACAAGTGCGCGGTCTCCAACAAAGCC
CTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATC
GCCGTGGAGTGGGAGAGCAATGGGCAGCCGGAGAACAACTACAAGACCACGCCTCCCGTGCTGGACTCCGACGG
CTCCTTCTTCCTCTATAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGT
460C3HUMANIZEDHEAVYCHAINVARIABLEHEAVYCHAINNUCLEOTIDESEQUENCE
SEQ ID NO: 230
GAGGTGCAGCTGTTGGAGTCTGGGGGAGGCTTGGTACAGCCTGGGGGGTCCCTGAGACTCTCCTGTGCAGCCTCT
GGAATCGACTTCAGTAGGAGATACTACATGTGCTGGGTCCGCCAGGCTCCAGGGAAGGGGCTGGAGTGGATCGC
ATGCATATATACTGGTAGCCGCGATACTCCTCACTACGCGAGCTCCGCGAAAGGCCGGTTCACCATCTCCAGAGAC
AATTCCAAGAACACGCTGTATCTGCAAATGAACAGCCTGAGAGCCGAGGACACGGCCGTATATTACTGTGCGAGA
GAAGGTAGCCTGTGGGGCCAGGGAACCCTGGTCACCGTCTCGAGC
460C3HUMANIZEDHEAVYCHAINFULL-LENGTHAMINOACIDSEQUENCE.HUMANGAMMA-1
CONSTANTDOMAINISUNDERLINED
SEQ ID NO: 231
EVQLLESGGGLVQPGGSLRLSCAASGIDFSRRYYMCWVRQAPGKGLEWIACIYTGSRDTPHYASSAKGRFTISRDNSKN
TLYLQMNSLRAEDTAVYYCAREGSLWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKRVEPKSCDKTHTCPPCPAPEAAGAPSV
FLFPPKPKDTLMISRTPEVTCVVVDVSHEDPEVKFNWYVDGVEVHNAKTKPREEQYNSTYRVVSVLTVLHQDWLNGKE
YKCAVSNKALPAPIEKTISKAKGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG
460C3HUMANIZEDHEAVYCHAINVARIABLEHEAVYCHAINAMINOACIDSEQUENCE.
COMPLIMENTARITYDETERMININGREGIONSAREUNDERLINED
SEQ ID NO: 232
EVQLLESGGGLVQPGGSLRLSCAASGIDFSRRYYMCWVRQAPGKGLEWIACIYTGSRDTPHYASSAKGRFTISRDNSKN
TLYLQMNSLRAEDTAVYYCAREGSLWGQGTLVTVSS
464C11HUMANIZEDLIGHTCHAINFULL-LENGTHNUCLEOTIDESEQUENCE
SEQ ID NO: 233
GACATCCAGATGACCCAGTCTCCTTCCACCCTGTCTGCATCTGTAGGAGACAGAGTCACCATCACTTGCCAGTCCA
GTCAGAGTGTTTATAGTAACAGCTTCTTATCCTGGTATCAGCAGAAACCAGGGAAAGCCCCTAAGCTCCTGATCTA
TTCTGCATCCGATCTGGCATCTGGGGTCCCATCAAGGTTCAGCGGCAGTGGATCTGGGACAGAATTCACTCTCACC
ATCAGCAGCCTGCAGCCTGATGATTTTGCAACTTATTACTGCGCAGGCGTTTATAGTGATAATACTTATGTTTTCGG
CGGAGGGACCAAGGTGGAGATCAAACGTACGGTGGCTGCACCATCTGTCTTCATCTTCCCGCCATCTGATGAGCA
GTTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAG
CCTCAGCAGCACCCTGACGCTGAGCAAAGCAGACTACGAGAAACACAAAGTCTACGCCTGCGAAGTCACCCATCA
GGGCCTGAGCTCGCCCGTCACAAAGAGCTTCAACAGGGGAGAGTGT
464C11HUMANIZEDLIGHTCHAINVARIABLELIGHTCHAINNUCLEOTIDESEQUENCE
SEQ ID NO: 234
GACATCCAGATGACCCAGTCTCCTTCCACCCTGTCTGCATCTGTAGGAGACAGAGTCACCATCACTTGCCAGTCCA
GTCAGAGTGTTTATAGTAACAGCTTCTTATCCTGGTATCAGCAGAAACCAGGGAAAGCCCCTAAGCTCCTGATCTA
TTCTGCATCCGATCTGGCATCTGGGGTCCCATCAAGGTTCAGCGGCAGTGGATCTGGGACAGAATTCACTCTCACC
ATCAGCAGCCTGCAGCCTGATGATTTTGCAACTTATTACTGCGCAGGCGTTTATAGTGATAATACTTATGTTTTCGG
CGGAGGGACCAAGGTGGAGATCAAA
464C11 HUMANIZED LIGHT CHAIN FULL-LENGTH AMINO ACID SEQUENCE.
HUMAN KAPPA CONSTANTDOMAIN ISUNDERLINED
SEQ ID NO: 235
DIQMTQSPSTLSASVGDRVTITCQSSQSVYSNSFLSWYQQKPGKAPKLLIYSASDLASGVPSRFSGSGSGTEFTLTISSLQP
DDFATYYCAGVYSDNTYVFGGGTKVEIKRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
464C11HUMANIZEDLIGHTCHAINVARIABLELIGHTCHAINAMINOACIDSEQUENCE.
COMPIMENTARITYDETERMININGREGIONSAREUNDERLINED
SEQ ID NO: 236
DIQMTQSPSTLSASVGDRVTITCQSSQSVYSNSFLSWYQQKPGKAPKLLIYSASDLASGVPSRFSGSGSGTEFTLT
ISSLQPDDFATYYCAGVYSDNTYVFGGGTKVEIK
464C11HUMANIZEDHEAVYCHAINFULL-LENGTHNUCLEOTIDESEQUENCE
SEQ ID NO: 237
GAGGTGCAGCTGGTGGAGTCTGGGGGAGGCTTGGTACAGCCAGGGCCGTCCCTGAGACTCTCCTGTACAGCTTCT
GGATTCTCCCTCAGTCACTACTGGATGACTTGGGTCCGCCAGGCTCCAGGGAAGGGGCTGGAGTACATCGGATTC
ATTAATGTTGGTGGTGACACATCTTACGCGAGCTCTGTGAAAGGCAGATTCACCATCTCAAGAGATGATTCCAAAA
GCATCGCCTATCTGCAAATGAACAGCCTGAAAACCGAGGACACAGCCGTGTATTACTGTGGCAGAGGTGGTCTGA
CTTTTGGTTGGGACTTGTGGGGCCAGGGAACCCTGGTCACCGTCTCGAGCGCTAGCACCAAGGGCCCATCGGTCT
TCCCCCTGGCACCCTCCTCCAAGAGCACCTCTGGGGGCACAGCGGCCCTGGGCTGCCTGGTCAAGGACTACTTCCC
CGAACCGGTGACGGTGTCGTGGAACTCAGGCGCCCTGACCAGCGGCGTGCACACCTTCCCGGCTGTCCTACAGTC
CTCAGGACTCTACTCCCTCAGCAGCGTGGTGACCGTGCCCTCCAGCAGCTTGGGCACCCAGACCTACATCTGCAAC
GTGAATCACAAGCCCAGCAACACCAAGGTGGACAAGAGAGTTGAGCCCAAATCTTGTGACAAAACTCACACATGC
CCACCGTGCCCAGCACCTGAAGCCGCGGGGGCACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAGGAGCAGTACAACAGCACGTACCG
TGTGGTCAGCGTCCTCACCGTCCTGCACCAGGACTGGCTGAATGGCAAGGAGTACAAGTGCGCGGTCTCCAACAA
AGCCCTCCCAGCCCCCATCGAGAAAACCATCTCCAAAGCCAAAGGGCAGCCCCGAGAACCACAGGTGTACACCCT
GCCCCCATCCCGGGATGAGCTGACCAAGAACCAGGTCAGCCTGACCTGCCTGGTCAAAGGCTTCTATCCCAGCGA
CATCGCCGTGGAGTGGGAGAGCAATGGGCAGCCGGAGAACAACTACAAGACCACGCCTCCCGTGCTGGACTCCG
ACGGCTCCTTCTTCCTCTATAGCAAGCTCACCGTGGACAAGAGCAGGTGGCAGCAGGGGAACGTCTTCTCATGCTC
CGTGATGCATGAGGCTCTGCACAACCACTACACGCAGAAGAGCCTCTCCCTGTCTCCGGGT
464C11HUMANIZEDHEAVYCHAINVARIABLEHEAVYCHAINNUCLEOTIDESEQUENCE
SEQ ID NO: 238
GAGGTGCAGCTGGTGGAGTCTGGGGGAGGCTTGGTACAGCCAGGGCCGTCCCTGAGACTCTCCTGTACAGCTTCT
GGATTCTCCCTCAGTCACTACTGGATGACTTGGGTCCGCCAGGCTCCAGGGAAGGGGCTGGAGTACATCGGATTC
ATTAATGTTGGTGGTGACACATCTTACGCGAGCTCTGTGAAAGGCAGATTCACCATCTCAAGAGATGATTCCAAAA
GCATCGCCTATCTGCAAATGAACAGCCTGAAAACCGAGGACACAGCCGTGTATTACTGTGGCAGAGGTGGTCTGA
CTTTTGGTTGGGACTTGTGGGGCCAGGGAACCCTGGTCACCGTCTCGAGC
464C11HUMANIZEDHEAVYCHAINFULL-LENGTHAMINOACIDSEQUENCE.HUMANGAMMA-1
CONSTANTDOMAINISUNDERLINED
SEQ ID NO: 239
EVQLVESGGGLVQPGPSLRLSCTASGFSLSHYWMTWVRQAPGKGLEYIGFINVGGDTSYASSVKGRFTISRDDSKSIAYL
QMNSLKTEDTAVYYCGRGGLTFGWDLWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKRVEPKSCDKTHTCPPCPAPEAAGAP
SVFLFPPKPKDTLMISRTPEVTCVVVDVSHEDPEVKFNWYVDGVEVHNAKTKPREEQYNSTYRVVSVLTVLHQDWLNG
KEYKCAVSNKALPAPIEKTISKAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
464C11HUMANIZEDHEAVYCHAINVARIABLEHEAVYCHAINAMINOACIDSEQUENCE.
COMPLIMENTARITYDETERMININGREGIONSAREUNDERLINED
SEQ ID NO: 240
EVQLVESGGGLVQPGPSLRLSCTASGFSLSHYWMTWVRQAPGKGLEYIGFINVGGDTSYASSVKGRFTISRDDSKSIAYL
QMNSLKTEDTAVYYCGRGGLTFGWDLWGQGTLVTVSS
466F6HUMANIZEDLIGHTCHAINFULL-LENGTHNUCLEOTIDESEQUENCE
SEQ ID NO: 241
GACGTTGTGATGACCCAGTCTCCATCTTCCGTGTCTGCATCTGTAGGAGACAGAGTCACCATCACCTGTCAGGCCA
GTCAGAACATTAGGACTTACTTATCCTGGTATCAGCAGAAACCAGGGAAAGCCCCTAAGCTCCTGATCTATGCTGC
AGCCAATCTGGCATCTGGGGTCCCATCAAGGTTCAGCGGCAGTGGATCTGGGACAGATTTCACTCTCACCATCAGC
GACCTGGAGCCTGGCGATGCTGCAACTTACTATTGTCAGTCTACCTATCTTGGTACTGATTATGTTGGCGGTGCTTT
CGGCGGAGGGACCAAGGTGGAGATCAAACGTACGGTGGCTGCACCATCTGTCTTCATCTTCCCGCCATCTGATGA
GCAGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGT
466F6HUMANIZEDLIGHTCHAINVARIABLELIGHTCHAINNUCLEOTIDESEQUENCE
SEQ ID NO: 242
GACGTTGTGATGACCCAGTCTCCATCTTCCGTGTCTGCATCTGTAGGAGACAGAGTCACCATCACCTGTCAGGCCA
GTCAGAACATTAGGACTTACTTATCCTGGTATCAGCAGAAACCAGGGAAAGCCCCTAAGCTCCTGATCTATGCTGC
AGCCAATCTGGCATCTGGGGTCCCATCAAGGTTCAGCGGCAGTGGATCTGGGACAGATTTCACTCTCACCATCAGC
GACCTGGAGCCTGGCGATGCTGCAACTTACTATTGTCAGTCTACCTATCTTGGTACTGATTATGTTGGCGGTGCTTT
CGGCGGAGGGACCAAGGTGGAGATCAAA
466F6HUMANIZEDLIGHTCHAINFULL-LENGTHAMINOACIDSEQUENCE.HUMANKAPPACONSTANT
DOMAINISUNDERLINED
SEQ ID NO: 243
DVVMTQSPSSVSASVGDRVTITCQASQNIRTYLSWYQQKPGKAPKLLIYAAANLASGVPSRFSGSGSGTDFTLTISDLEP
GDAATYYCQSTYLGTDYVGGAFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
466F6HUMANIZEDLIGHTCHAINVARIABLELIGHTCHAINAMINOACIDSEQUENCE.COMPIMENTARITY
DETERMININGREGIONSAREUNDERLINED
SEQ ID NO: 244
DVVMTQSPSSVSASVGDRVTITCQASQNIRTYLSWYQQKPGKAPKLLIYAAANLASGVPSRFSGSGSGTDFTLTISDLEP
GDAATYYCQSTYLGTDYVGGAFGGGTKVEIK
466F6HUMANIZEDHEAVYCHAINFULL-LENGTHNUCLEOTIDESEQUENCE
SEQ ID NO: 245
CGGTCGCTGGTGGAGTCTGGGGGAGGCTTGGTCCAGCCTGGGGGGTCCCTGAGACTCTCCTGTACAGCCTCTGGA
TTCACCATCAGTAGCTACCACATGCAGTGGGTCCGCCAGGCTCCAGGGAAGGGGCTGGAGTACATCGGAACCATT
AGTAGTGGTGGTAATGTATACTACGCGAGCTCCGCGAGAGGCAGATTCACCATCTCCAGACCCTCGTCCAAGAAC
ACGGTGGATCTTCAAATGAACAGCCTGAGAGCCGAGGACACGGCTGTGTATTACTGTGCGAGAGACTCTGGTTAT
AGTGATCCTATGTGGGGCCAGGGAACCCTGGTCACCGTCTCGAGCGCTAGCACCAAGGGCCCATCGGTCTTCCCC
CTGGCACCCTCCTCCAAGAGCACCTCTGGGGGCACAGCGGCCCTGGGCTGCCTGGTCAAGGACTACTTCCCCGAA
CCGGTGACGGTGTCGTGGAACTCAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCACCCAGACCTACATCTGCAACGTGA
ATCACAAGCCCAGCAACACCAAGGTGGACAAGAGAGTTGAGCCCAAATCTTGTGACAAAACTCACACATGCCCAC
CGTGCCCAGCACCTGAAGCCGCGGGGGCACCGTCAGTCTTCCTCTTCCCCCCAAAACCCAAGGACACCCTCATGAT
CTCCCGGACCCCTGAGGTCACATGCGTGGTGGTGGACGTGAGCCACGAAGACCCTGAGGTCAAGTTCAACTGGTA
CGTGGACGGCGTGGAGGTGCATAATGCCAAGACAAAGCCGCGGGAGGAGCAGTACAACAGCACGTACCGTGTG
GTCAGCGTCCTCACCGTCCTGCACCAGGACTGGCTGAATGGCAAGGAGTACAAGTGCGCGGTCTCCAACAAAGCC
CTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATC
GCCGTGGAGTGGGAGAGCAATGGGCAGCCGGAGAACAACTACAAGACCACGCCTCCCGTGCTGGACTCCGACGG
CTCCTTCTTCCTCTATAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGT
466F6HUMANIZEDHEAVYCHAINVARIABLEHEAVYCHAINNUCLEOTIDESEQUENCE
SEQ ID NO: 246
CGGTCGCTGGTGGAGTCTGGGGGAGGCTTGGTCCAGCCTGGGGGGTCCCTGAGACTCTCCTGTACAGCCTCTGGA
TTCACCATCAGTAGCTACCACATGCAGTGGGTCCGCCAGGCTCCAGGGAAGGGGCTGGAGTACATCGGAACCATT
AGTAGTGGTGGTAATGTATACTACGCGAGCTCCGCGAGAGGCAGATTCACCATCTCCAGACCCTCGTCCAAGAAC
ACGGTGGATCTTCAAATGAACAGCCTGAGAGCCGAGGACACGGCTGTGTATTACTGTGCGAGAGACTCTGGTTAT
AGTGATCCTATGTGGGGCCAGGGAACCCTGGTCACCGTCTCGAGC
466F6HUMANIZEDHEAVYCHAINFULL-LENGTHAMINOACIDSEQUENCE.HUMANGAMMA-1
CONSTANTDOMAINISUNDERLINED
SEQ ID NO: 247
RSLVESGGGLVQPGGSLRLSCTASGFTISSYHMQWVRQAPGKGLEYIGTISSGGNVYYASSARGRFTISRPSSKNTVDLQ
MNSLRAEDTAVYYCARDSGYSDPMWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKRVEPKSCDKTHTCPPCPAPEAAGAPSV
FLFPPKPKDTLMISRTPEVTCVVVDVSHEDPEVKFNWYVDGVEVHNAKTKPREEQYNSTYRVVSVLTVLHQDWLNGKE
YKCAVSNKALPAPIEKTISKAKGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG
466F6HUMANIZEDHEAVYCHAINVARIABLEHEAVYCHAINAMINOACIDSEQUENCE.
COMPLIMENTARITYDETERMININGREGIONSAREUNDERLINED
SEQ ID NO: 248
RSLVESGGGLVQPGGSLRLSCTASGFTISSYHMQWVRQAPGKGLEYIGTISSGGNVYYASSARGRFTISRPSSKNTVDLQ
MNSLRAEDTAVYYCARDSGYSDPMWGQGTLVTVSS
470B6HUMANIZEDLIGHTCHAINFULL-LENGTHNUCLEOTIDESEQUENCE
SEQ ID NO: 249
GACATCCAGATGACCCAGTCTCCTTCCACCCTGTCTGCATCTGTAGGAGACAGAGTCACCATCACTTGCCAGGCCA
GTCAGAGCATTAGTACTAACTTGAACTGGTATCAGCAGAAACCAGGGAAAGCCCCTAAGCTCCTGATCTATTATGC
ATCCAGTCTGGCATCTGGGGTCCCATCAAGGTTCAGCGGCAGTGGATCTGGGACAGAATTCACTCTCACCATCAGC
AGCCTGCAGCCTGATGATTTTGCAACTTATTACTGCCTAGGTGTTTGGAATTATTGGGGTGATGATGGTACTGATTT
CGGCGGAGGGACCAAGGTGGAGATCAAACGTACGGTGGCTGCACCATCTGTCTTCATCTTCCCGCCATCTGATGA
GCAGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGT
470B6HUMANIZEDLIGHTCHAINVARIABLELIGHTCHAINNUCLEOTIDESEQUENCE
SEQ ID NO: 250
GACATCCAGATGACCCAGTCTCCTTCCACCCTGTCTGCATCTGTAGGAGACAGAGTCACCATCACTTGCCAGGCCA
GTCAGAGCATTAGTACTAACTTGAACTGGTATCAGCAGAAACCAGGGAAAGCCCCTAAGCTCCTGATCTATTATGC
ATCCAGTCTGGCATCTGGGGTCCCATCAAGGTTCAGCGGCAGTGGATCTGGGACAGAATTCACTCTCACCATCAGC
AGCCTGCAGCCTGATGATTTTGCAACTTATTACTGCCTAGGTGTTTGGAATTATTGGGGTGATGATGGTACTGATTT
CGGCGGAGGGACCAAGGTGGAGATCAAA
470B6HUMANIZEDLIGHTCHAINFULL-LENGTHAMINOACIDSEQUENCE.HUMANKAPPACONSTANT
DOMAINISUNDERLINED
SEQ ID NO: 251
DIQMTQSPSTLSASVGDRVTITCQASQSISTNLNWYQQKPGKAPKLLIYYASSLASGVPSRFSGSGSGTEFTLTISSLQPD
DFATYYCLGVWNYWGDDGTDFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
470B6HUMANIZEDLIGHTCHAINVARIABLELIGHTCHAINAMINOACIDSEQUENCE.COMPIMENTARITY
DETERMININGREGIONSAREUNDERLINED
SEQ ID NO: 252
DIQMTQSPSTLSASVGDRVTITCQASQSISTNLNWYQQKPGKAPKLLIYYASSLASGVPSRFSGSGSGTEFTLTISSLQPD
DFATYYCLGVWNYWGDDGTDFGGGTKVEIK
470B6HUMANIZEDHEAVYCHAINFULL-LENGTHNUCLEOTIDESEQUENCE
SEQ ID NO: 253
GAGGTGCAGCTGGTGGAGTCTGGGGGAGGCTTGGTCCAGCCTGGGGGGTCCCTGAGACTCTCCTGTGCAGCCTC
TGGATTCTCCCTCAGTAGCTACTACATGAGCTGGGTCCGCCAGGCTCCAGGGAAGGGGCTGGAGTGGATCGGAA
GTATTTATGGTAGTGGTGCCGCATACTACGCGAGCTCCGCTAAAGGCAGATTCACCATCTCCAGAGACAATTCCAA
GAACACGCTGTATCTTCAAATGAACAGCCTGAGAGCCGAGGACACGGCTGTGTATTACTGTGCGAGAGATCCTAT
TAACAATGCCATGTGGGGCCAGGGAACCCTGGTCACCGTCTCGAGCGCTAGCACCAAGGGCCCATCGGTCTTCCC
CCTGGCACCCTCCTCCAAGAGCACCTCTGGGGGCACAGCGGCCCTGGGCTGCCTGGTCAAGGACTACTTCCCCGA
ACCGGTGACGGTGTCGTGGAACTCAGGCGCCCTGACCAGCGGCGTGCACACCTTCCCGGCTGTCCTACAGTCCTC
AGGACTCTACTCCCTCAGCAGCGTGGTGACCGTGCCCTCCAGCAGCTTGGGCACCCAGACCTACATCTGCAACGTG
AATCACAAGCCCAGCAACACCAAGGTGGACAAGAGAGTTGAGCCCAAATCTTGTGACAAAACTCACACATGCCCA
CCGTGCCCAGCACCTGAAGCCGCGGGGGCACCGTCAGTCTTCCTCTTCCCCCCAAAACCCAAGGACACCCTCATGA
TCTCCCGGACCCCTGAGGTCACATGCGTGGTGGTGGACGTGAGCCACGAAGACCCTGAGGTCAAGTTCAACTGGT
ACGTGGACGGCGTGGAGGTGCATAATGCCAAGACAAAGCCGCGGGAGGAGCAGTACAACAGCACGTACCGTGT
GGTCAGCGTCCTCACCGTCCTGCACCAGGACTGGCTGAATGGCAAGGAGTACAAGTGCGCGGTCTCCAACAAAGC
CCTCCCAGCCCCCATCGAGAAAACCATCTCCAAAGCCAAAGGGCAGCCCCGAGAACCACAGGTGTACACCCTGCC
CCCATCCCGGGATGAGCTGACCAAGAACCAGGTCAGCCTGACCTGCCTGGTCAAAGGCTTCTATCCCAGCGACATC
GCCGTGGAGTGGGAGAGCAATGGGCAGCCGGAGAACAACTACAAGACCACGCCTCCCGTGCTGGACTCCGACGG
CTCCTTCTTCCTCTATAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGT
470B6HUMANIZEDHEAVYCHAINVARIABLEHEAVYCHAINNUCLEOTIDESEQUENCE
SEQ ID NO: 254
GAGGTGCAGCTGGTGGAGTCTGGGGGAGGCTTGGTCCAGCCTGGGGGGTCCCTGAGACTCTCCTGTGCAGCCTC
TGGATTCTCCCTCAGTAGCTACTACATGAGCTGGGTCCGCCAGGCTCCAGGGAAGGGGCTGGAGTGGATCGGAA
GTATTTATGGTAGTGGTGCCGCATACTACGCGAGCTCCGCTAAAGGCAGATTCACCATCTCCAGAGACAATTCCAA
GAACACGCTGTATCTTCAAATGAACAGCCTGAGAGCCGAGGACACGGCTGTGTATTACTGTGCGAGAGATCCTAT
TAACAATGCCATGTGGGGCCAGGGAACCCTGGTCACCGTCTCGAGC
470B6HUMANIZEDHEAVYCHAINFULL-LENGTHAMINOACIDSEQUENCE.HUMANGAMMA-1
CONSTANTDOMAINISUNDERLINED
SEQ ID NO: 255
EVQLVESGGGLVQPGGSLRLSCAASGFSLSSYYMSWVRQAPGKGLEWIGSIYGSGAAYYASSAKGRFTISRDNSKNTLY
LQMNSLRAEDTAVYYCARDPINNAMWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKRVEPKSCDKTHTCPPCPAPEAAGAP
SVFLFPPKPKDTLMISRTPEVTCVVVDVSHEDPEVKFNWYVDGVEVHNAKTKPREEQYNSTYRVVSVLTVLHQDWLNG
KEYKCAVSNKALPAPIEKTISKAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
470B6HUMANIZEDHEAVYCHAINVARIABLEHEAVYCHAINAMINOACIDSEQUENCE.
COMPLIMENTARITYDETERMININGREGIONSAREUNDERLINED
SEQ ID NO: 256
EVQLVESGGGLVQPGGSLRLSCAASGFSLSSYYMSWVRQAPGKGLEWIGSIYGSGAAYYASSAKGRFTISRDNSKNTLY
LQMNSLRAEDTAVYYCARDPINNAMWGQGTLVTVSS
470C6HUMANIZEDLIGHTCHAINFULL-LENGTHNUCLEOTIDESEQUENCE
SEQ ID NO: 257
GCCCTTGTGATGACCCAGTCTCCTTCCACCCTGTCTGCATCTGTAGGAGACAGAGTCACCATCAATTGCCAGGCCA
GTGAGGACATTGATAACTATTTAGCCTGGTATCAGCAGAAACCAGGGAAAGCCCCTAAGCTCCTGATCTTTTATGC
ATCCGATCTGGCATCTGGGGTCCCATCAAGGTTCAGCGGCAGTGGATCTGGGACAGAATTCACTCTCACCATCAGC
AGCCTGCAGCCTGATGATTTTGCAACTTATTACTGCCAAGGCGGTTACTATACTAGTAGTACTGATACGAGGGGTG
CTTTCGGCGGAGGGACCAAGGTGGAGATCAAACGTACGGTGGCTGCACCATCTGTCTTCATCTTCCCGCCATCTGA
TGAGCAGTTGAAATCTGGAACTGCCTCTGTTGTGTGCCTGCTGAATAACTTCTATCCCAGAGAGGCCAAAGTACAG
TGGAAGGTGGATAACGCCCTCCAATCGGGTAACTCCCAGGAGAGTGTCACAGAGCAGGACAGCAAGGACAGCAC
CTACAGCCTCAGCAGCACCCTGACGCTGAGCAAAGCAGACTACGAGAAACACAAAGTCTACGCCTGCGAAGTCAC
CCATCAGGGCCTGAGCTCGCCCGTCACAAAGAGCTTCAACAGGGGAGAGTGT
470C6HUMANIZEDLIGHTCHAINVARIABLELIGHTCHAINNUCLEOTIDESEQUENCE
SEQ ID NO: 258
GCCCTTGTGATGACCCAGTCTCCTTCCACCCTGTCTGCATCTGTAGGAGACAGAGTCACCATCAATTGCCAGGCCA
GTGAGGACATTGATAACTATTTAGCCTGGTATCAGCAGAAACCAGGGAAAGCCCCTAAGCTCCTGATCTITTATGC
ATCCGATCTGGCATCTGGGGTCCCATCAAGGTTCAGCGGCAGTGGATCTGGGACAGAATTCACTCTCACCATCAGC
AGCCTGCAGCCTGATGATTTTGCAACTTATTACTGCCAAGGCGGTTACTATACTAGTAGTACTGATACGAGGGGTG
CTTTCGGCGGAGGGACCAAGGTGGAGATCAAA
470C6HUMANIZEDLIGHTCHAINFULL-LENGTHAMINOACIDSEQUENCE.HUMANKAPPACONSTANT
DOMAINISUNDERLINED
SEQ ID NO: 259
ALVMTQSPSTLSASVGDRVTINCQASEDIDNYLAWYQQKPGKAPKLLIFYASDLASGVPSRFSGSGSGTEFTLTISSLQPD
DFATYYCQGGYYTSSTDTRGAFGGGTKVEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC
470C6HUMANIZEDLIGHTCHAINVARIABLELIGHTCHAINAMINOACIDSEQUENCE.COMPIMENTARITY
DETERMININGREGIONSAREUNDERLINED
SEQ ID NO: 260
ALVMTQSPSTLSASVGDRVTINCQASEDIDNYLAWYQQKPGKAPKLLIFYASDLASGVPSRFSGSGSGTEFTLTISSLQPD
DFATYYCQGGYYTSSTDTRGAFGGGTKVEIK
470C6HUMANIZEDHEAVYCHAINFULL-LENGTHNUCLEOTIDESEQUENCE
SEQ ID NO: 261
CAGTCGCTGGTGGAGTCTGGGGGAGGCTTGGTACAGCCTGGGGGGTCCCTGAGACTCTCCTGTGCAGCCTCTGG
ATTCTCCTTCACTAGCAGCTACTACATGTGCTGGGTCCGCCAGGCTCCAGGGAAGGGGCTGGAGTGGATCGCATG
CATTTATACTGGTAGTAGTGGTAGCACTTACTACGCGAACTCCGCGAAAGGCCGGTTCACCATCTCCAGAGACAAT
TCCAAGAACACGCTGTATCTGCAAATGAACAGCCTGAGAGCCGAGGACACGGCCGTATATTACTGTGCGAGAGAT
AGTAGTAGTTATTATATGTTTAACTTGTGGGGCCAGGGAACCCTGGTCACCGTCTCGAGCGCTAGCACCAAGGGC
CCATCGGTCTTCCCCCTGGCACCCTCCTCCAAGAGCACCTCTGGGGGCACAGCGGCCCTGGGCTGCCTGGTCAAGG
ACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTA
CATCTGCAACGTGAATCACAAGCCCAGCAACACCAAGGTGGACAAGAGAGTTGAGCCCAAATCTTGTGACAAAAC
TCACACATGCCCACCGTGCCCAGCACCTGAAGCCGCGGGGGCACCGTCAGTCTTCCTCTTCCCCCCAAAACCCAAG
GACACCCTCATGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGCAGTACAACA
GCACGTACCGTGTGGTCAGCGTCCTCACCGTCCTGCACCAGGACTGGCTGAATGGCAAGGAGTACAAGTGCGCGG
TCTCCAACAAAGCCCTCCCAGCCCCCATCGAGAAAACCATCTCCAAAGCCAAAGGGCAGCCCCGAGAACCACAGG
TGTACACCCTGCCCCCATCCCGGGATGAGCTGACCAAGAACCAGGTCAGCCTGACCTGCCTGGTCAAAGGCTTCTA
TCCCAGCGACATCGCCGTGGAGTGGGAGAGCAATGGGCAGCCGGAGAACAACTACAAGACCACGCCTCCCGTGC
TGGACTCCGACGGCTCCTTCTTCCTCTATAGCAAGCTCACCGTGGACAAGAGCAGGTGGCAGCAGGGGAACGTCT
TCTCATGCTCCGTGATGCATGAGGCTCTGCACAACCACTACACGCAGAAGAGCCTCTCCCTGTCTCCGGGT
470C6HUMANIZEDHEAVYCHAINVARIABLEHEAVYCHAINNUCLEOTIDESEQUENCE
SEQ ID NO: 262
CAGTCGCTGGTGGAGTCTGGGGGAGGCTTGGTACAGCCTGGGGGGTCCCTGAGACTCTCCTGTGCAGCCTCTGG
ATTCTCCTTCACTAGCAGCTACTACATGTGCTGGGTCCGCCAGGCTCCAGGGAAGGGGCTGGAGTGGATCGCATG
CATTTATACTGGTAGTAGTGGTAGCACTTACTACGCGAACTCCGCGAAAGGCCGGTTCACCATCTCCAGAGACAAT
TCCAAGAACACGCTGTATCTGCAAATGAACAGCCTGAGAGCCGAGGACACGGCCGTATATTACTGTGCGAGAGAT
AGTAGTAGTTATTATATGTTTAACTTGTGGGGCCAGGGAACCCTGGTCACCGTCTCGAGC
470C6HUMANIZEDHEAVYCHAINFULL-LENGTHAMINOACIDSEQUENCE.HUMANGAMMA-1
CONSTANTDOMAINISUNDERLINED
SEQ ID NO: 263
QSLVESGGGLVQPGGSLRLSCAASGFSFTSSYYMCWVRQAPGKGLEWIACIYTGSSGSTYYANSAKGRFTISRDNSKNT
LYLQMNSLRAEDTAVYYCARDSSSYYMFNLWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKRVEPKSCDKTHTCPPCPAPEAA
GAPSVFLFPPKPKDTLMISRTPEVTCVVVDVSHEDPEVKFNWYVDGVEVHNAKTKPREEQYNSTYRVVSVLTVLHQDW
LNGKEYKCAVSNKALPAPIEKTISKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPG
470C6HUMANIZEDHEAVYCHAINVARIABLEHEAVYCHAINAMINOACIDSEQUENCE.
COMPLIMENTARITYDETERMININGREGIONSAREUNDERLINED
SEQ ID NO: 264
QSLVESGGGLVQPGGSLRLSCAASGFSFTSSYYMCWVRQAPGKGLEWIACIYTGSSGSTYYANSAKGRFTISRDNSKNT
LYLQMNSLRAEDTAVYYCARDSSSYYMFNLWGQGTLVTVSS
472E4HUMANIZEDLIGHTCHAINFULL-LENGTHNUCLEOTIDESEQUENCE
SEQ ID NO: 265
GACATCCAGATGACCCAGTCTCCATCCTCCCTGTCTGCATCTGTAGGAGACAGAGTCACCATCACTTGCCAGTCCA
GTCAGAGTGTTTATAATAACAATGCTTTAGCCTGGTATCAGCAGAAACCAGGGAAAGTTCCTAAGCTCCTGATCTA
TTTGGCTTCCACTCTGGCATCTGGGGTCCCATCTCGGTTCAGTGGCAGTGGATCTGGGACAGATTTCACTCTCACCA
TCAGCAGCCTGCAGCCTGAAGATGTTGCAACTTATTACTGTCTAGGTGTTTATAATGATGATGTTGATAATGGTTTC
GGCGGAGGGACCAAGGTGGAGATCAAACGTACGGTGGCTGCACCATCTGTCTTCATCTTCCCGCCATCTGATGAG
CAGTTGAAATCTGGAACTGCCTCTGTTGTGTGCCTGCTGAATAACTTCTATCCCAGAGAGGCCAAAGTACAGTGGA
AGGTGGATAACGCCCTCCAATCGGGTAACTCCCAGGAGAGTGTCACAGAGCAGGACAGCAAGGACAGCACCTAC
AGCCTCAGCAGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAGAGTGT
472E4HUMANIZEDLIGHTCHAINVARIABLELIGHTCHAINNUCLEOTIDESEQUENCE
SEQ ID NO: 266
GACATCCAGATGACCCAGTCTCCATCCTCCCTGTCTGCATCTGTAGGAGACAGAGTCACCATCACTTGCCAGTCCA
GTCAGAGTGTTTATAATAACAATGCTTTAGCCTGGTATCAGCAGAAACCAGGGAAAGTTCCTAAGCTCCTGATCTA
TTTGGCTTCCACTCTGGCATCTGGGGTCCCATCTCGGTTCAGTGGCAGTGGATCTGGGACAGATTTCACTCTCACCA
TCAGCAGCCTGCAGCCTGAAGATGTTGCAACTTATTACTGTCTAGGTGTTTATAATGATGATGTTGATAATGGTTTC
GGCGGAGGGACCAAGGTGGAGATCAAA
472E4HUMANIZEDLIGHTCHAINFULL-LENGTHAMINOACIDSEQUENCE.HUMANKAPPACONSTANT
DOMAINISUNDERLINED
SEQ ID NO: 267
DIQMTQSPSSLSASVGDRVTITCQSSQSVYNNNALAWYQQKPGKVPKLLIYLASTLASGVPSRFSGSGSGTDFTLTISSLQ
PEDVATYYCLGVYNDDVDNGFGGGTKVEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC
472E4HUMANIZEDLIGHTCHAINVARIABLELIGHTCHAINAMINOACIDSEQUENCE.COMPIMENTARITY
DETERMININGREGIONSAREUNDERLINED
SEQ ID NO: 268
DIQMTQSPSSLSASVGDRVTITCQSSQSVYNNNALAWYQQKPGKVPKLLIYLASTLASGVPSRFSGSGSGTDFTLTISSLQ
PEDVATYYCLGVYNDDVDNGFGGGTKVEIK
472E4HUMANIZEDHEAVYCHAINFULL-LENGTHNUCLEOTIDESEQUENCE
SEQ ID NO: 269
GAGGTGCAGCTGGTGGAGTCTGGGGGAGGCTTGGTCCAGCCTGGGGGGTCCCTGAGACTCTCCTGTGCAGCCTC
TGGATTCTCCCTCAATAACAATGCAATAAGCTGGGTCCGCCAGGCTCCAGGGAAGGGGCTGGAGTGGGTTGCAAT
CATACAAAATACTGGTACCACAGACTACGCGAGGTCCGCTAAAGGCAGATTCACCATCTCCAGAGACAATTCCAAG
AACACGCTGTATCTTCAAATGAACAGCCTGAGAGCCGAGGACACGGCTGTGTATTACTGTGCGAGAGGGTATGGT
TTTGAGTCGGAGCTTGTCATCTGGGGCCAGGGAACCCTGGTCACCGTCTCGAGCGCTAGCACCAAGGGCCCATCG
GTCTTCCCCCTGGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGCAGCTTGGGCACCCAGACCTACATCTG
CAACGTGAATCACAAGCCCAGCAACACCAAGGTGGACAAGAGAGTTGAGCCCAAATCTTGTGACAAAACTCACAC
ATGCCCACCGTGCCCAGCACCTGAAGCCGCGGGGGCACCGTCAGTCTTCCTCTTCCCCCCAAAACCCAAGGACACC
CTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGT
ACCGTGTGGTCAGCGTCCTCACCGTCCTGCACCAGGACTGGCTGAATGGCAAGGAGTACAAGTGCGCGGTCTCCA
ACAAAGCCCTCCCAGCCCCCATCGAGAAAACCATCTCCAAAGCCAAAGGGCAGCCCCGAGAACCACAGGTGTACA
CCCTGCCCCCATCCCGGGATGAGCTGACCAAGAACCAGGTCAGCCTGACCTGCCTGGTCAAAGGCTTCTATCCCAG
CGACATCGCCGTGGAGTGGGAGAGCAATGGGCAGCCGGAGAACAACTACAAGACCACGCCTCCCGTGCTGGACT
CCGACGGCTCCTTCTTCCTCTATAGCAAGCTCACCGTGGACAAGAGCAGGTGGCAGCAGGGGAACGTCTTCTCAT
GCTCCGTGATGCATGAGGCTCTGCACAACCACTACACGCAGAAGAGCCTCTCCCTGTCTCCGGGT
472E4HUMANIZEDHEAVYCHAINVARIABLEHEAVYCHAINNUCLEOTIDESEQUENCE
SEQ ID NO: 270
GAGGTGCAGCTGGTGGAGTCTGGGGGAGGCTTGGTCCAGCCTGGGGGGTCCCTGAGACTCTCCTGTGCAGCCTC
TGGATTCTCCCTCAATAACAATGCAATAAGCTGGGTCCGCCAGGCTCCAGGGAAGGGGCTGGAGTGGGTTGCAAT
CATACAAAATACTGGTACCACAGACTACGCGAGGTCCGCTAAAGGCAGATTCACCATCTCCAGAGACAATTCCAAG
AACACGCTGTATCTTCAAATGAACAGCCTGAGAGCCGAGGACACGGCTGTGTATTACTGTGCGAGAGGGTATGGT
TTTGAGTCGGAGCTTGTCATCTGGGGCCAGGGAACCCTGGTCACCGTCTCGAGC
472E4HUMANIZEDHEAVYCHAINFULL-LENGTHAMINOACIDSEQUENCE.HUMANGAMMA-1
CONSTANTDOMAINISUNDERLINED
SEQ ID NO: 271
EVQLVESGGGLVQPGGSLRLSCAASGFSLNNNAISWVRQAPGKGLEWVAIIQNTGTTDYARSAKGRFTISRDNSKNTLY
LQMNSLRAEDTAVYYCARGYGFESELVIWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVDKRVEPKSCDKTHTCPPCPAPEAAG
APSVFLFPPKPKDTLMISRTPEVTCVVVDVSHEDPEVKFNWYVDGVEVHNAKTKPREEQYNSTYRVVSVLTVLHQDWLN
GKEYKCAVSNKALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPG
472E4HUMANIZEDHEAVYCHAINVARIABLEHEAVYCHAINAMINOACIDSEQUENCE.
COMPLIMENTARITYDETERMININGREGIONSAREUNDERLINED
SEQ ID NO: 272
EVQLVESGGGLVQPGGSLRLSCAASGFSLNNNAISWVRQAPGKGLEWVAIIQNTGTTDYARSAKGRFTISRDNSKNTLY
LQMNSLRAEDTAVYYCARGYGFESELVIWGQGTLVTVSS

1-26. (canceled)

27. An isolated monoclonal antibody (mAb) or antigen-binding fragment thereof that binds specifically to human or cynomolgus 4-1BB, comprising a variable light chain comprising-SEQ ID NO: 204, and a variable heavy chain comprising SEQ ID NO: 208.

28. The isolated mAb or antigen-binding fragment thereof according to claim 1, wherein the isolated mAb is an IgG, a humanized antibody, a chimeric antibody, a recombinant antibody, a bispecific antibody, tri-specific antibody, or multi-specific antibody.

29. An isolated mAb or antigen-binding fragment thereof having a binding specificity to human or cynomolgus 4-1BB, comprising a light chain and a heavy chain, wherein the isolated mAb or antigen-binding fragment thereof comprises amino acids 24-34 of SEQ ID NO: 204 as complementarity determining region (CDR) 1, amino acids 50-56 of SEQ ID NO: 204 as CDR2, and amino acids 89-102 of SEQ ID NO: 204 as CDR3 of the light chain, and amino acids 30-34 of SEQ ID NO: 208 as CDR1, amino acids 50-67 of SEQ ID NO: 208 as CDR2, and amino acids 100-109 of SEQ ID NO: 208 as CDR3 of the heavy chain.

30. The isolated mAb or antigen-binding fragment thereof according to claim 1, wherein the antigen-binding fragment is a Fv, a Fab, a F(ab′)2, a scFV or a scFV2 fragment.

31. A pharmaceutical composition, comprising the isolated mAb or antigen-binding fragment thereof claim 27 and a pharmaceutically acceptable carrier.

32. The pharmaceutical composition of claim 31, further comprising a radioisotope, a radionuclide, a toxin, a therapeutic agent, a chemotherapeutic agent or a combination thereof.

33. A pharmaceutical composition, comprising the isolated mAb or antigen-binding fragment thereof claim 29 and a pharmaceutically acceptable carrier.

34. The pharmaceutical composition of claim 33, further comprising a radioisotope, a radionuclide, a toxin, a therapeutic agent, a chemotherapeutic agent or a combination thereof.
